(19) United States
(12) Patent Application Publication
(10) Pub. No.: US 2024/0082270 A1
(43) Pub. Date: Mar. 14, 2024

(54) CANNABIGEROL (CBG) PRODUCTS AND METHODS OF USE

(71) Applicant: Bazelet Health Systems, Inc., Orlando, FL (US)

(72) Inventors: Michael Elzufon, Maple Grove, MN (US); Howard Hoffberg, Reisterstown, MD (US)

(21) Appl. No.: 18/513,636

(22) Filed: Nov. 20, 2023

Related U.S. Application Data
(60) Provisional application No. 63/280,865, filed on Nov. 18, 2021.

(51) Int. Cl. A61K 31/00; A61K 31/05; A61K 31/12; A61K 31/164; A61K 31/165; A61K 31/19; A61K 31/4525; A61K 31/5685; A61K 36/28

(52) U.S. Cl. CPC A61K 31/658; A61K 31/05; A61K 31/12; A61K 31/164; A61K 31/165; A61K 31/19; A61K 31/4525; A61K 31/5685; A61K 36/28

(57) ABSTRACT

A pharmaceutically acceptable formulation comprising a cannabinoid, e.g., CBG, CBGA, CBGV, and CBGVA, or a pharmaceutically acceptable salt or ester thereof, substantially without CBDA synthase products and TCHA synthase products; and at least one additional active composition, e.g., an endocannabinoid, dextromethorphan, melatonin, 5-hydroxy tryptophan, or serotonin. The cannabinoid may be a full spectrum or broad spectrum extract of a plant of genus Cannabis, containing cannabinoids, terpenes, and flavonoids, and substantially without cannabidiol and tetrahydrocannabinol. The composition may be a pharmaceutically acceptable formulation for oral, mucosal (including sublingual), topical, inhaled, vaporized or spray, or smoked administration. The formulation may include absorption or pharmacological (synergistic) exogenous enhancers, e.g., curcumin, resveratrol, quercetin, piperine, butyrate (or its natural derivatives) and other (endo)cannabinoids. The composition may have anti-inflammatory properties beneficial as a prophylaxis or therapy of symptoms due to SARS-Cov2 infection.

### CROSS REFERENCE TO RELATED APPLICATIONS

The present application is a Non-Provisional of U.S. Provisional Patent Application No. 63/280,865, filed Nov. 18, 2021, the entirety of which is expressly incorporated by reference.

### FIELD OF THE INVENTION

The present invention relates to the field of cannabinoids, and more particularly to cannabigerolic acid (CBGA) and cannabigerol (CBG), both cannabinoids, in new formulations and with new uses, including adjunctive care for Covid-19.

### BACKGROUND OF THE INVENTION

Each reference cited herein (including supplemental information) is expressly incorporated herein by reference for all purposes. The bibliography is provided as an appendix to this specification. The incorporated references provide written description of known technology, enable the practice of the invention; provide context for interpretation of the words and phrases, and as a combination with the whole disclose aspects of the invention.

Cannabis: Cannabis contains more than 500 compounds, among them at least 125 cannabinoids; however, most of these “minor” cannabinoids are only produced in trace amounts. Beside cannabinoids, the chemical constituents of Cannabis includes over 120 compounds responsible for its characteristic aroma, which are mainly volatile terpenes and sesquiterpenes which have gained public awareness through the growth and education of medical and recreational Cannabis, as well as volatile thiols or other sulfur-containing compounds that contribute to the plant's skunk-like aroma, with Prenylthiol (3-methyl-2-butene-1-thiol) identified as the primary odorant contributing significantly to the pungent aroma of Cannabis.

Cannabis sativa seeds are chiefly used to make hempseed oil which, in turn, can be used for animal feed, cooking, lamps, lacquers, or paints. The flowers and fruits (and to a lesser extent the leaves, stems, and seeds) contain psychoactive chemical compounds known as cannabinoids that are consumed for recreational, medicinal, and spiritual purposes. When so used, preparations of flowers and fruits (called marijuana) and leaves and preparations derived from resinous extract (e.g., hashish) are consumed by smoking, vaporizing, and oral ingestion. Historically, tinctures, teas, and ointments have also been common preparations. In traditional medicine of India, in particular, Cannabis sativa has been used as hallucinogenic, hypnotic, sedative, analgesic, and anti-inflammatory agent. Organizations and companies operating in Cannabis markets have pushed education and marketing of terpenes in their products as means to differentiate taste and effects of Cannabis. The entourage effect, which describes the synergy of cannabinoids, terpenes, and other plant compounds, has also helped further awareness and demand for terpenes in Cannabis products.

Broadly, there are three main cultivar groups of Cannabis that are cultivated today: cultivars primarily cultivated for their fiber (industrial use), characterized by long stems and little branching; cultivars grown for seed which can be eaten entirely raw or from which hemp oil (generally recognized as safe, GRAS) is extracted; and cultivars grown for medicinal or recreational purposes, characterized by extensive branching to maximize the number of flowers. A distinction is often made between industrial hemp, with concentrations of psychoactive compounds far too low to be useful for that purpose, and marijuana.

Medical Cannabis: Medical marijuana is used to manage diverse medical conditions. Common side effects include dizziness, sedation, confusion, dissociation, and “feeling high”.

In Minnesota, the following are accepted indications for medical Cannabis for adult symptomatic use in which there is a consensus opinion of medical evidence to support its use: Cancer (if the underlying condition or treatment produces one or more of the following: 1. Severe or chronic pain; 2. Nausea or severe vomiting; or 3. Cachexia or severe wasting), Glaucoma, Human Immunodeficiency Virus (HIV Positive)/Acquired Immune Deficiency Syndrome (AIDS), Tourette syndrome, Amyotrophic lateral sclerosis (ALS), Seizures (including those characteristic of epilepsy), Severe and persistent muscle spasms (or spasticity), including those characteristic of multiple sclerosis (MS), Inflammatory bowel disease (IBD), Terminal illness (with a probable life expectancy of under one year, if the illness or its treatment produces one or more of the following: 1. Severe or chronic pain; 2. Nausea or severe vomiting; or 3. Cachexia or severe wasting), Post-traumatic stress disorder (PTSD), Autism spectrum disorder (ASD), obstructive sleep apnea (OSA), Alzheimer's disease (AD), Chronic or Intractable (or severe) pain, Sickle cell disease, Chronic motor or vocal tic disorder. Other states may include other mental health disorders including substance use disorder, depression, insomnia, anxiety, anorexia, arthritis, migraine headaches, decompensated cirrhosis (or hepatitis C virus-HV), or another chronic medical condition which is severe and for which other treatments have been ineffective. Often, there are multiple (or a combination of) symptoms, in which the predominant or accepted conditions is the only one documented for justification (i.e., pain with insomnia). In pediatrics, it may be restricted to intractable seizures, ASD (with self-injurious or aggressive behavior), severe and painful muscle spasms, wasting/cachexia or cancer related severe nausea or vomiting, coordinated by a designated adult caregiver. Other childhood seizure disorders for which CBD products (not THC) may be beneficial includes: neonatal hypoxic ischemic encephalopathy or perinatal brain injury, which may also be accompanied by cerebral spasticity, infantile spasms, Fragile X syndrome tuberous sclerosis, as well as pediatric schizophrenia and neuroblastoma (a common childhood cancer, possibly mediated by vanilloid (TRPV) and peroxisome proliferator-activated (PPAR) receptors).

The Endocannabinoid System (ECS): The EndoCannabinoid System (ECS) has evolved over 500 million years in organisms, and is an amazingly complex premier regulatory center of the body for homeostasis (balance and regulation), activated by any type of stress (physical, biological and mental), affecting mental abilities, emotions, pain, inflammation, immune and metabolic functions primarily with neuromodulation by two CannaBinoid Receptors (CBR, with mounting evidence of more), belonging to the class of G protein-coupled receptors (GPCRs), the most abundant receptor class in the body. GPCRs mediate the cellular response to neurotransmitters and hormones and are mostly responsible for taste, vision, and olfaction. CBR have differences in their amino acid sequence, signaling mechanisms, tissue distribution, and sensitivity to certain agonists and antagonists. ECS includes endocannabinoid transmitters and has complex interactions with other non-ECS pathways, in which modulating receptors are found on the neurons and their supportive cells.

The ECS regulates: movement and cognition, learning and memory, emotion, motivation, mood, sleep, pain, addiction, appetite, cellular and bone metabolism, endocrine/hormonal and temperature regulation, renal, reproduction, intra-ocular pressure, motor control and coordination, neuro-protection, seizures, immunity, inflammation, cell regeneration (healing and wounds), anti-cancer activity, anti-ischemic, smooth muscle tone including cardiovascular and gastrointestinal (GI) regulation (including motility and nausea), as well as many other functions.

An endocannabinoid is a molecule that is, or is equivalent to, a product that naturally occurs in humans, that allosterically or directly modulates or influences CB1R and/or CB2R effects, as an accepted mechanism of action.

There are many interdependent variables that affect neurophysiologic ECS functions of transmitters, including ligands with lipophilic properties (which facilitates crossing blood-brain barrier—BBB more easily to enter central nervous system (CNS), and this permeability can dynamically change with CNS diseases and/or injuries), with passive or active transport, body or cellular degradation/catabolism, complicated by diverse phenotypes of the host (genetics, gender, age and comorbidities). Pathologic (including cancer cells) or dynamic neonatal states may have a different distribution of CBR, as well as variable ECS response to ligands. ECS has a homeostatic function, implying that their activity is to maintain or restore physiologic balance, predominantly with dynamic states of allosteric modulation, with many overlapping or redundant effects. ECS affects normal and cancer cells through modulating mitochondrial morphology and function. It may also direct neurogenic stem cells toward specialized differentiation into neurons. There are many known pharmaceuticals, including experimental functional or physiologic agonists or antagonists that have known activities for the ECS or non-ECS receptors, which assist in identifying mechanism of action, and potential therapeutic benefits.

CBR is derived from rhodopsin-like (lass A) family of GPCRs, and CB1R common underlying mechanism is by inhibiting stimulated adenylate cyclase (AC) activity presynaptically, and thereafter, induce the activation of p42/44 mitogen-activated protein kinase (MAPK), p38 MAPK, c-Jun N-terminal kinase, activator protein (AP)-1, β-arrestins, the neural form of focal adhesion kinase, protein kinase (PK)-B, and potassium (K⁺) and calcium (Ca²⁺)transients. CB1R is the most abundant GPCR in the CNS, expressed constituently (but may also be inducible).

CBR are located in both intra- and extra-cellularly, mitochondrial membranes (modulating cell energetic balance and reactive oxygen species (ROS) production), endoplasmic reticulum (ER, correlating with cellular communication), endosomes (correlated with cell homeostasis), lysosomes (correlated with inflammation and phagocytosis), and nuclear membranes (correlated with apoptosis). However, CBR intracellular locations will likely be inaccessible to membrane-impermeant cannabinoid ligands.

Endogenous endocannabinoids are naturally occurring, lipid-based neurotransmitters. Arachidonic acid (AA, a precursor for eiosanoids including prostaglandins) is a polyunsaturated fatty acid found in membrane phospholipids in several body organs including the brain, and is the precursor for anandamide (AEA) and belongs to the family of N-acylethanolamines (NAE). AEA is synthesized from the precursor membrane lipid N-arachidonyl-phosphatidylethanolamine (NAPE), via a NAPE-specific phospholipase D (PLD), although several other pathways are known to exist. AEA, which binds equally to CB1R and CB2R as a partial agonist, and is described as the “bliss molecule” as it produces feelings of happiness, joy, and even motivation; In animal studies, AEA is found at higher levels in the nucleus accumbens (NAc), a brain structure that regulates motivational behavior, which in turn, stimulates the hypothalamus to secrete oxytocin, a contextual hormone. ECS may act indirectly by increasing oxytocin levels which has been associated with a variety of physiological processes, including emotion (social interaction), appetite, childbirth, analgesia and aging, and has been applied to wide variety of psychological conditions including autism spectrum disorder (ASD), attention deficit hyperactivity disorder (ADHD), anxiety and depression.

Functional CB1R agonists, 2-AG and AEA, are present around gestational week 19 in humans, and likely have a role in critical early behavior processes. CB1R in the developing fetal brain system may also contribute to the proper formation, growth, migration, and wiring of various areas in the developing fetal brain, and following birth, the brain's CB1R expression for location and function with dynamic changes with development until early adulthood. CB1R are located in the neocortex and limbic system, outflow nuclei of the basal ganglia: substantia nigra, pars reticulata and globus pallidus; hippocampus; striatum; cerebellum; and sparsely in the lower brainstem areas (absent in the medulla oblongata, the region responsible for respiratory and cardiovascular functions), contributing to hypothermia, but not to respiratory depression), spinal cord, as well as sites in the periphery (including skeletal muscle, sex organs, pituitary (may decrease vasopressin for diuresis), bladder, proximal convoluted tubules, distal tubules, and intercalated cells of the collecting dud of the kidney (with diuresis), anterior eye and retina, tonsils, thymus, spleen, immune cells, myenteric and submucosal neurons (also, in epithelial cells) throughout the GI tract, liver, pancreatic islet cells, vascular endothelium and peripheral nerves) with larger concentrations in the cell mitochondria which decreases its cell respiration and metabolism, as well as located on the cellular membrane.

CB1R regulates neuron growth by transducing signals by acting through inhibition of AC, MAPK cascades, modulation of ion channels, and intracellular calcium mobilization, as well as provide a basal sympathetic tone, including modulation of glutamate (Glu), gamma-amino butyric acid (GABA), acetylcholine (Ach), noradrenaline (NE), dopamine (DA) and serotonin (5HT); nausea (emesis), which are involved with temperature regulation, appetite, cognition, mood, nociception, motor activity and movement, and with some modulation of immune function.

CB2R are predominantly found in immune-derived cells with varying expression patterns, and is responsible for immunomodulatory and other therapeutic effects. CB2R, by transducing signals, regulates immune leukocyte lines of hematopoietic systems, with the highest density found in B lymphocytes, natural killer cells, macrophages, monocytes, neutrophils, mast cells, other T lymphocytes including helper cells, dendritic cells (in descending order), secondary lymphoid tissues such as spleen, tonsils, Peyer's patches, lymphatic ganglia, and hepatic myofibroblastic cells as well as the neurogenic immune system. CB2R expression is inducible, increased by inflammation, in which they generally inhibit immune cell activation, and reduce pro-inflammatory cytokine production, as well as inducible with cancer cell line proliferation. Additionally, CB2R receptors can be found throughout peripheral organ tissues, such as the lungs, skin, bones, testes, renal glomerulus and GI tract (immune cells, epithelial cells, myenteric and submucosal neurons), as well as in the supportive cells of the CNS (especially brainstem and cerebellum), such as microglia as well as in glial (astrocytes, oligodendrocytes), neural stem/progenitor cells, vascular elements in the brain and nociceptive peripheral nerves in skin. CB2R is involved with regulation of local immune and inflammatory reactions and can inhibits DA firing from the ventral tegmental area and the hippocampus for potential treatment of addiction, and is involved in visceral pain (especially, GI related). Bladder contractility is increased with CBR binding.

The ECS exerts a potent regulation of feeding behavior and energy balance by complex central, and recently highlighted, peripheral mechanisms including CB1R in the GI tract autonomic nerves regulating GI motility and secretion and dorsal root ganglia (DRG), as well as in adipocytes, pancreas, liver and muscle. The ECS interacts with neuropeptides and hormones with an orexigenic (ghrelin, mediated through sympathetic activity, induces hyperglycemia and glucagon secretion) or satiety (cholecystokinin-CCK, which may modulate nocebo hyperalgesia, even if devoid of anxiety; leptin, Glucagon-like peptide 1-GLP-1, Peptide YY-PYY or peptide tyrosine tyrosine, which inhibits ghrelin) action to regulate food intake, and there is a CB1R mediated neurochemical signal through the vagus nerve to central structures (mainly hypothalamic areas, in which the arcuate nucleus has a more permeable BBB for cannabinoid transfer for energy homeostasis, and limbic system especially the DA-rich NAc for behavioral effects of appetite for the sweet food reward system (“munchies”) involved in appetitive drive, which can be bidirectional. Several bioactive forms of CCK have been described which is primarily synthesized in I-cells of small intestine from fat and protein digestion, binding to CCK1 (located in intestines, pancreas, gallbladder for GI contraction, as well as in several brain areas such as the area postrema, nucleus solitarius, hypothalamus, interpeduncular nucleus, substantia nigra, and cranial nerve vagal afferent neurons, and its nodose ganglia; related to post-prandial satiety) and CCK2 (brain: cortex, olfactory bulb, hypothalamus, NAc, amygdala, hippocampus, caudate nucleus, and cerebellum as well as vagal afferents) receptors, the latter involved in the regulation of food intake, pain, stress, anxiety (may be responsible for the “nocebo” response), neuroendocrine control, cardiovascular regulation, control of learning and memory, neuropsychiatric disorders including anxiety-related behaviors, modulation of dependence and withdrawal, and, in general, processes that are controlled by 5HT, DA, and opioid systems. Vagal afferent neurons also express ghrelin type 1 (GHS-1), orexin type 1 (OX-R1), melanin concentrating hormone (MCH) type 1, CB1R, and leptin receptors. Food deprivation increased the expression of both CB1R mRNA and protein (activated by AEA) and CCK1 in the rat vagal nodose ganglia, in which regulating appetite is widely modulated by nutritional status and hormonal/peptidic signals. This effect is prevented by re-feeding or using a CCK1 receptor agonist. There are additive effects with using CB1R antagonist and CCK1 agonist relative to satiety. CCK1 agonist response is attenuated in bred leptin resistant or obese animal models, but adding CB1R antagonist reduced leptin resistance and improved satiety in obese rats. The opposite effect is clinically useful with malnutrition, cachexia and wasting states, or to mitigate appetite suppression following chemotherapy. There are also favorable effects of cannabinoids as direct antimicrobial agents, prebiotics on the microbiome system, as well as direct CB1R (and to a lesser extent, CB2R) binding to the GI tract, which can reduce gastric motility, but may increase intestinal motility, as well as modulation of the vagus nerve output, affecting feeding and energy balance. The sweet receptor (Tlc1) is stimulated by indirectly increasing its expression and suppressing the activity of leptin, the Tlc1 antagonist. It is proposed that the competition of leptin and ECS for Tlc1 is implicated in energy homeostasis. ECS is involved in renal blood flow and blood pressure regulation, renal protection to acute injury (from toxins or ischemia), glomerular protection for proteinuria, and sodium excretion by the tubules, primarily mediated by CB1R.

The intricate network of inter- and intra-ECS interactions with other non-ECS pathways is referred to as the endocannabinoidome, a complex signaling apparatus consisting of more than 100 lipid mediators and 50 proteins. ECS has complex and dynamic interactions, in which cannabinoid substrates also modulate for homeostatic balance, which may allow them to function for CBR modulation acting as partial agonist or reverse agonist, directly or indirectly (including non-ECR pathways) depending on the pathologic setting. There are numerous non-ECS receptors or pathways resulting in additional/synergistic and complementary physiologic homeostasis which can regulate diverse physical, cognitive and psychological functions (some of these receptors, mechanism of action and their function overlap). Most non-ECS receptors can be found on the neurons particularly in the brain's fronto-striatal and fronto-limbic circuitry, and their supportive cells, the microglia (which is also part of the immune system), as well as in many other body tissues. Furthermore, these dynamic CBR and non-ECS binding and distribution may be altered with genetics (and epigenetics), aging, psychophysiological or pathological conditions; and gender differences have also been described.

Endocannabinoids: Endocannabinoids serve as intercellular “lipid messengers”, signaling molecules that are released from one cell and activating the cannabinoid receptors present on other nearby cells. Although in this intercellular signaling role they are similar to the well-known monoamine neurotransmitters such as DA, endocannabinoids differ in numerous ways from them. Endocannabinoids are not stored in vesides or exist as integral constituents of the membrane bilayers that make up cells. Their biosynthesis takes place on demand from lipid precursors in the cytoplasmic membrane through enzyme activation in response to elevations of intracellular calcium. They are produced by receptor-stimulated cleavage of membrane lipid precursors and then released from cells. As hydrophobic molecules, endocannabinoids cannot travel unaided for long distances in the aqueous medium surrounding the cells from which they are released, and therefore, act locally on nearby target cells. Hence, although emanating diffusely from their source cells, they have much more restricted spheres of influence than do hormones, which can affect cells throughout the body.

Endocannabinoids, unlike other neurotransmitters, are retrograde transmitters, acting in the opposite direction from a postsynaptic neuron to presynaptic neuron. They are released from the postsynaptic cell and ad on the presynaptic cell, where the target receptors are densely concentrated on axonal terminals in the zones from which conventional neurotransmitters are released. Activation of CBR temporarily reduces the amount of conventional neurotransmitter released. This ECS permits the postsynaptic cell to control its own incoming synaptic traffic. The ultimate effect on the endocannabinoid-releasing cell depends on the nature of the conventional transmitter being controlled. For instance, when the release of the inhibitory transmitter GABA is reduced, the net effect is an increase in the excitability of the endocannabinoid-releasing cell. On the converse, when release of the excitatory neurotransmitter Glu is reduced, the net effect is a decrease in the excitability of the endocannabinoid-releasing cell.

Endocannabinoids are then removed from the site by cellular uptake processes such as simple diffusion, through membrane associated binding proteins or by a transmembrane carrier protein. Inside the tissue, their metabolism is catalyzed by fatty acid amide hydrolase (FAAH) which is located on cytosolic surfaces of surface endoplasmic reticulum cisternae and mitochondria. In some tissues, however, endocannabinoids also undergo an oxidative catabolism through lipoxygenases, cydooxygenase-2 and cytochrome P450, and palmitoylethanolamide-preferring acid amidase (PAA).

AEA was the first endocannabinoid compound identified as arachidonoyl ethanolamine. AEA binds to the centrally (CB1R) and, to a lesser extent, peripherally (CB2R), where it ads as a partial agonist, which is similar to THC, although its structure is quite different. AEA is about as potent as THC at the CB1R, and is found in nearly all tissues in a wide range of animals and plants, including small amounts in chocolate. Two analogs of AEA, 7,10,13,16-docosatetraenoylethanolamide and homo-γ-linolenoylethanolamine, have similar pharmacology. All of these compounds are members of a family of signaling lipids called N-acylethanolamines (NAE), as bioactive lipid mediators also identified in plant seeds and in molluscs, and can be used as preventive therapy of acute and late-onset neuroinflammation-associated synaptic dysfunction and neurodegeneration. NAEs include non-cannabimimetic endogenous (do not directly bind CBR, but can modulate AEA, and FAAH): Palmitoylethanolamide (PEA or palmidrol is best studied); Oleoylethanolamide (OEA, or N-oleoylethanolamide, cis-9-octadecenamide, oleamide involved with satiety, inhibits gastric emptying and intestinal motility, are released from intestinal enterocytes and lamina propria cells is an endogenous ligand of GPR119, can induce sleep and improve mood; Exogenous administration of OEA inhibits feeding in rodents and can also decrease body weight following long-term administration), which possess anti-inflammatory and anorexigenic effects, respectively; such as) Stearoylethanolamide (SEA, a neuroprotective against lipopolysaccharide (LPS)-induced neuroinflammation in C57BL/6 male mice, by restricting the spreading of peripheral inflammation to the brain, and averting the activation of resident microglia and leukocyte trafficking to the brain parenchyma. Treatment with SEA per se increased the neuronal expression of cannabinoid receptors CB1/2 and brain levels of the most potent endogenous CB1/2 agonist 2-AG in vivo. SEA enhanced the amplitude of synaptic veside release, supported the balanced signal-to-noise ratio in Glu- and GABA-ergic neurotransmission and decreased the excitotoxic risk associated with higher extracellular Glu levels under neuroinflammation. The interference of SEA with the ECS and presynaptic neurotransmitter release may represent an intrinsic neuroprotective mechanism that is triggered by inflammation and glutamate excitotoxicity. SEA is involved with satiety, which can improve inflammation and oxidative stress in obese people); and Linoleylethanolamine (LEA, released from intestines, and increased with feedings, may be useful topically for contact dermatitis, activating GPR119 receptor, along with 2-oleoylglycerol (2-OG, but at a lower potency)). They all can synergistically increase 2-arachidonoylglycerol (2-AG) activity as an “entourage compounds” that can inhibit its metabolization via substrate competition and are PPAR-α ligands. Prolonged intake of a diet high in fat is known to decrease intestinal levels of OEA, PEA, and LEA. Intraperitoneal injection or oral administration of these NAEs into experimental animals have revealed different effects, including inhibition of food intake (OEA, PEA, LEA), inhibition of inflammation (PEA), inhibition of pain (OEA, PEA), inhibition of atherosclerosis (OEA) as well as anticonvulsive (PEA) and neuroprotective effects (PEA, OEA). There are also plant derived NAEs which also have been shown to inhibit FAAH, a catabolic enzyme, particularly, LEA (in chocolate and other plants), contributing to the reward system.

Another endocannabinoid, 2-AG, is a monoacylglycerol that incorporates AA of the glycerol backbone that binds to both the CB1R (more selectively) and CB2R with similar affinity, acting as a full agonist at both sites. 2-AG is present at significantly higher concentrations in the brain than AEA, and there is some controversy over whether 2-AG rather than AEA is chiefly responsible for ECS signaling in vivo. In particular, one in vitro study suggests that 2-AG is capable of stimulating higher G-protein activation than AEA, although the physiological implications of this finding are not yet known. 2AGs precursor, phosphatidylinositol, is converted by phospholipase-1 or phospholipase-γ, to the intermediary lipid 1,2-diacylglycerol (1,2-DAG). The 1,2-DAG is then hydrolyzed by DAG lipases (with 2 isoforms found post-synaptically: DAGLα, inhibited by calcium calmodulin kinase II (CaMKII) and found in the hippocampus pyramidal cells, dentate granule cells, cerebellar purkinje cells, striatum and ventral tegmental area, and can interact with Glu receptors for depolarization induced suppression of excitation (DSE), contributing to synaptic plasticity; DAGLβ's expression pattern is not as well characterized, but appears to higher a higher density in the hippocampus; both are increased in fetal neuro-development, and co-expressed together with CB1R with elongated axons for presynaptic pathfinding; whereas postnatally, DAGLa is more concentrated in dendrites and CB1R in axons across the synaptic deft for postsynaptic neuromodulation) to form 2-AG. If the CB1R function at the neuron is excitatory in nature, then the spatial relationship with DAGLa is much closer (2-AG is rapidly metabolized for a transient and very specific effect and to prevent unintended actions) than if it were inhibitory (longer duration of effect and 2-AG may diffuse to other targets). The DAGL enzymes may be tissue dependent such that in rats with ethanol exposure, the liver content of DAGLb is upregulated and contributes to steatosis with proinflammatory effects on peritoneal macrophages. There may be other CNS development adaptations made by DAGLb (or cells that also contain DAGLa) including ECS synaptic plasticity via DSE. Neuro-Ocular DAGLA-related Syndrome (NODRS) is the first genetic disorder linking ECS in children, described as a mutation of termination variants in the DAGLA gene, which presents with developmental delays, difficulties with balance and walking, abnormal eye movements and head nodding. An alternative pathway for synthesis involves converting 2-arachidonyl lysophospholipid by the enzyme lysophosphotase-C (LYSOPLC) to rapidly convert to 2-AG.

PEA also belongs to the family of NAEs, and is also contained in plants: peanuts, and animals: chicken, egg yolk), is lipophilic and with short duration of effect, binds CB1R for sleep, TRPV1 for REM sleep and vasorelaxation, PPARα for fat metabolism (can vary based on diet and diurnal clock), inhibits FAAH, stimulates mitochondrial respiration, inhibits carrageenan- and prostaglandin-induced hyperalgesia involving the formalin test of persistent pain, visceral hyperalgesia produced by instillation of nerve growth factor (NGF) into the bladder and the sciatic nerve ligature model of neuropathic pain; also, PEA is a CB2R agonist in mast cells, adenosine triphosphate (ATP)-sensitive potassium (K+)-channels, TRP channels, and Nuclear Factor K-light-chain-enhancer of activated B cells (NFkB) family of proteins, and an agonist with G protein-coupled receptor GPR1 expressed predominantly in the pancreas (3-cells) and GI tract (enteroendocrine cells involved in glucagon-like peptide-1 secretion) for anti-inflammatory effects and regeneration. It has been detected in the CNS hypothalamus, white matter, brain stem, cerebellum and brain cortex as well as the pituitary gland and adrenal organs. It may also be consumed as a generally recognized as safe (GRAS) supplement (including animal feeds); and is considered useful for eczema, pain and neurodegeneration. In human meta-analysis trials of exogenous micronized (or ultra-micronized) PEA, it was found to be beneficial as an analgesic, without appreciable side effects.

A third, ether-type endocannabinoid, 2-arachidonyl glyceryl ether (noladin ether) (2-AGE), was isolated from porcine brain. It binds primarily to the CB1R (Kᵢ=21.2 nmol/L) and only weakly to CB2R; and causes sedation, hypothermia, intestinal immobility, and mild antinociception in mice.

N-Arachidonoyl dopamine (NADA) preferentially binds CB1R. Like AEA, NADA is also an agonist for the vanilloid receptor subtype 1 (TRPV1), a member of the vanilloid receptor family. NADA is structurally similar to capsaicin. Vanilloids, especially vanilloid receptor (including all subtypes) act through signal transduction of numerous chemical and physical stimuli and regulate many neural signaling processes and other physiological functions such as temperature sensation, smell, taste, vision, pressure, or pain perception, and dysfunctions can cause channelopathies.

Virodhamine, or O-arachidonoyl-ethanolamine (OAE), is a full agonist at CB2R and as a CB1R antagonist in vivo, producing hypothermia. In rats, virodhamine was found to be present at comparable or slightly lower concentrations than anandamide in the brain, but 2- to 9-fold higher concentrations peripherally.

Lysophosphatidylinositol is the endogenous ligand to novel endocannabinoid receptor GPR55, making it a strong contender for endocannabinoid activity.

Endocannabinoids are catabolized by special enzymes: FAAH-1/2 hydrolysis, for CB2R agonists including AEA and 2-AG, which metabolize intracellularly, such that transporters can allow these AEA and 2-AG to actively diffuse through sodium based channels, which can allow a prolonged (minutes) effect of CB1R binding particularly to GABA-ergic neurons cellular reuptake mechanism that can be blocked by specific inhibitors. Decreased enteric FAAH activity is associated with colonic inertia in slow transit constipation. FAAH is linked with arousability and aversive-memories extinction, and may be correlated with cellular learning and memory. AEA may help memory by helping us forget (extraneous information or “noise”), allowing the brain's ability to weaken unimportant memories and experiences to enable it to function more efficiently. FAAH has shown promise in preclinical studies, ameliorating pain- and anxiety-related behavior. Cannabinoid-induced antinociception is likely mediated in both the CNS and in the periphery. Cannabinoids modulate proalgesic and proinflammatory factors released by immune cells; therefore, peripherally restricted cannabinoids can reduce the production of these factors, mostly via CB2R. Moreover, peripheral CB2R activation has been shown to stimulate the release of endogenous opioids, resulting in reduced nociceptive behavior.

For traumatic brain injury (TBI), 2-AG functions (has a higher concentration in the brain compared to AEA, which ads as CBR partial agonist) not only as an endogenous CBR ligand, but also an immunomodulator by virtue of its being a major precursor for AA, making it a versatile target for the treatment of TBI related pro-inflammatory pathologies. CBR expression is globally reduced in TBI and can disrupt diurnal rhythms (CB1R), and its activation may also help with vasodilation and lowering blood pressure, and can diffuse through BBB (which often is defective and more permeable in TBI or other CNS insults). CB1R is involved in modulation of cerebral edema (as well as neurogenic pulmonary edema or heterotopic ossification) and can mitigate neuron loss in hippocampus CA-3 region. There are complex phenomena, such that with the addition of estradiol, it further reduces proinflammatory astrocytes, and while also increasing cerebral cortex mRNA levels of CB2R; also, adding fatty acid amides in addition to AEA may work in concert to ameliorate pathologies related to TBI. It has been hypothesized that with low dose THC (higher doses may be neurotoxic by increasing Glu release) pre-treatment produced a pre-conditioning effect, where a mildly noxious stimulus becomes protective against a more severe subsequent insult, an effect known to occur in cardiology as well as cerebral ischemia. In addition, TBI-induced behavioral deficits, such as learning and memory, neurological motor impairments, post-traumatic convulsions or seizures, and anxiety also respond to manipulations of the ECS, such that potential modulation can be effective for the underlying disease and symptom control.

Endocannabinoids as they act as chemical messengers when bound to CBR in the brain and body to optimize proper functioning of many diverse domains. Fatty acid-binding protein (FABP) shuttles endocannabinoid compounds for CBR or non-ECS binding, affecting its availability.

Phytocannabinoids

Most phyto-cannabinoids naturally exist from Cannabis L. plants, and other synthetic cannabinoids compounds interact with the ECS. The most notable cannabinoid is the THC (predominantly Delta-9-THC or Δ9-THC isomer), the primary intoxicating compound in Cannabis. CBGA and its varin (V) propyl homologue subtype (produced at lower concentrations in any marijuana or hemp plant, but more commonly found in landrace Cannabis cultivar), cannabigerovarinic acid (CBVA), are “mother cannabinoids,” in which all the precursor cannabinoids acids (A) described below. They are derived from enzymatic synthases reactions between a resorcinol and an isoprenoid group's unique conversion for each cannabinoid, most concentrated in the trichome. The length of the alkyl side chain on the resorcinyl moiety (including A precursors) tends to increase the bioactivity of a particular cannabinoid, of which the following homolog nomenclature is based: orcinoids (with 1 carbon atom), varinoids (with 3 carbon atoms), butols (with 4 carbon atoms), most of the well described cannabinoids (5 carbon atoms) and phorol (7 carbon atoms) groups, which are ordered from least to most potent (mostly based on CB1R binding assays in a cannabinoid tetrad pharmacological test, based on animal behaviors including hypomotility, analgesia, catalepsy and hyporthermia, such that a minimum of 3 carbons is necessary to bind CBR, with the highest activity registered with an 8 carbon side chain, then the affinity decreases, thereafter with higher carbon chains), and potency can correlate with higher lipophilicity.

Most classical cannabinoids are 21-carbon compounds. However, some do not follow this rule, primarily because of variation in the length of the side-chain attached to the aromatic ring. In THC, CBD, and CBN, this side-chain is a pentyl (5-carbon) chain. In the most common homologue, the pentyl chain is replaced with a propyl (3-carbon) chain. Cannabinoids with the propyl side chain are named using the suffix varin and are designated THCV, CBDV, or CBNV, while those with the heptyl side chain are named using the suffix phorol and are designated THCP and CBDP.

The other precursor cannabinoids acidic forms from the “mother compounds”, CBGA/CBGVA are derived by conversion of hemp's enzymatic synthases for each cannabinoid. These include THC, its degradation product cannabinol (CBN, less than 1% in fresh marijuana harvest), as well as CBD, CBG, CBC (not well-studied), all of which form as the result of decarboxylation from the less stable acidic precursor molecules in the plant, THCA, CBNA, CBDA, CBGA and CBCA, respectively; they may also be unidirectionally converted by heating. THC/THCA binds strongly to CB1R, and to a lesser extent, CB2R, whereas the binding affinities or allosteric modulation of the other minor cannabinoids (including CBD/CBDA, CBG/CBGA, and CBC/CBCA) functionally act as CB2R agonists, and to a much lesser extent, CB1R modulators (may be antagonists). Each cannabinoid's distinct pharmacologic and therapeutic effects are related to the diversity of these unique molecules by modulating, with variable and dynamic affinities, with both the ECS and the other heterogeneous non-ECS receptors, which can be just as crucial for to its desirable effects.

Cannabis literature often incorrectly cites CBG as being the “mother of all cannabinoids,” instead of CBGA, the acidic precursor of CBG. They also often combine CBGA and CBG together as “total CBG”, even though they have distinct pharmacologic properties and metabolism. CBGA and CBG similarly both bind to receptors of the brain, nerves, skin and immune system, as well as other cells, and have both been evaluated in diverse scientific (basic, in vitro, in vivo or clinical) studies. CBD is another major constituent of some Cannabis plants, followed by CBC and CBG. CBD and THC are produced independently in the Cannabis plant from the precursor CBGA. It was reported in 2020 that cannabinoids (not THC) can be found in other plants such as rhododendron, licorice and liverwort, Trema micrantha blume and orientalis and earlier in Echinacea.

There is a hypothesized “entourage” effect of the combination of all cannabinoids taken as a “full” (with THC) or “broad” spectrum (without THC) of ingredients has a greater effect that each individual cannabinoid alone for a therapeutic response. In addition, the route of administration as well as the dose intervals, may contribute to the clinical effect, such that vaporized CBG/CBGA potentially may have minimal psychotropic properties (predictably, topical use would have a negligible effect) in some preparations, due to a more rapid onset and shorter duration of effect. The most notable benefit of Cannabis in any form is its safety, with no reports of lethal overdose with any of the cannabinoids.

A “full-spectrum” hemp extract is the initial slushy preparation, after harvest and drying, containing parts of the plant biomass that possesses intrinsic cannabinoids and terpenes (mostly from the flowers, leaves or stalks), while routinely separating other components (including seeds, lignin or chlorophyll), usually mixed with a carrier oil, to form a concentrate that preserves the full cannabinoid and terpene proportions of the raw hemp plant in order to derive additional botanical products for consumption. Due diligence is made during this process to preserve the integrity of the extracted phytochemicals to prevent degradation or contamination. A raw extract derived only from flowers (without seeds) has the highest intrinsic concentration of the active cannabinoids. A “broad” spectrum extract preferentially removes a specific component (i.e., THC, even from legal hemp) from this concentrate. An isolate preferentially purifies one phytochemical component of this extract, without any other appreciable substances, but devoid of the entourage effect.

Psychoactive THC vs. non-psychoactive other cannabinoids: THC, Δ9-THC and Δ8-THC, through intracellular CBR activation, induce AEA and 2-AG synthesis produced naturally in the body and brain. These cannabinoids produce the effects associated with Cannabis by binding to the CB1R in the brain. THC, CBN, its acidic precursors (THCA), isomers and derivatives/analogs as well as human active metabolites, are all considered “psychoactive.” THC can be subjected to misuse, overuse, abuse, toxicity, chemical dependency, addiction or diversion, and have been associated with accidental injuries, physical or medical impairments, mental health changes or substance use disorder, overdose toxicity, and other medical problems (especially if smoked) with impaired mentation and motor function. Most other cannabinoids are “non-psychoactive,” including CBD, CBG, and CBC (presumably ordered from least to greatest potency, including their acidic and varinolic derivatives) by binding to both CBR and non-ECS brain receptors, in which they modulate (or provide “balance” to) mentation or cognition, behaviors, energy or sleep, with negligible risk of chemical dependency or “addiction.” It appears that CBG, CBD and probably CBC, with chronic use, and even at higher doses, does not affect physiological measures, and does not significantly alter psychomotor or psychological functions. Both CBG/CBGA have overlapping CBR and multiple non-ECS effects; acts as a 5HT1A antagonist, whereas CBD is an indirect 5HT1A agonist. They both have been evaluated for anti-inflammatory, antimicrobial, neuroprotective (also anticonvulsant), anti-cancer and antioxidant properties, which can clinically assist in improving well-being as well as ameliorating symptoms with the following conditions: cardiovascular, metabolic, GI, mental health, sleep, fatigue, autoimmune, inflammatory, neurologic, dermatologic, oncologic, ocular, pain and others. There have been both human and veterinary therapeutic applications for these products, with varying routes of administration. THC has similar binding properties as CBD, except no 5HT1/2, GPC3/6/8, TRPs, GABA, GlyR, or acetyl choline effects, but possesses TRP4 agonist, adrenoreceptor Ala agonist as a vasoconstrictor (whereas CBG is a potent α2-adrenoceptor agonist, as a unique effect; with vasodilation), and is a significant CBR partial agonist (with greater effect on CB1R).

THC is 20 times more anti-inflammatory than aspirin, twice as anti-inflammatory as hydrocortisone with anti-emetic properties. CBD also has powerful analgesic and anti-inflammatory effects mediated by both cyclooxygenase and lipoxygenase inhibition. Its anti-inflammatory effect is several hundred times more potent than aspirin. Sativa strains are described by patients as uplifting, energetic, creative, euphoria, spacey, cerebrally-focused effects, and better for day use, while indica strains are typically described as calming, relaxing, sedative, full body effects such as “body buzz”, and better for night use. In a survey of 1271 medical Cannabis patients (90.5% White, 62.5% male), those with headaches preferred Sativa hybrids with high THC/THCA, low CBD/CBDA, strain with predominant terpenes β-caryophyllene and β-myrcene potent analgesic, anti-inflammatory, of THC, with anti-inflammatory and analgesic properties of β-caryophyllene and β-myrcene as an entourage effect. Indica strains were preferred in the insomnia/sleep disorders group, Saiva strains in the mental health condition/PTSD group, and Hybrid strains in the GI/Crohn's Disease group.

Classical cannabinoids are structurally or biochemically related to THC. Nonclassical cannabinoids (cannabimimetics) include aminoalkylindoles, 1,5-diarylpyrazoles, quinolines, and arylsulfonamides as well as eicosanoids related to endocannabinoids. Cannabinoid production starts when an enzyme causes geranyl pyrophosphate and olivetolic acid to combine and form CBGA. Next, CBGA is independently converted to: CBG, THCA, CBDA or CBCA by four separate synthase, FAD-dependent dehydrogenase enzymes. There is no evidence for enzymatic conversion of CBDA or CBD to THCA or THC, but may convert with acidic conditions and higher temperatures. The naturally occurring, but rare, propyl varin homologues (THCVA, CBDVA and CBCVA), are much less studied, but have an analogous pathway that is based on CBGVA from divarinolic acid instead of olivetolic acid. In addition, each of the compounds above may be in different forms depending on the position of the double bond in the alicyclic carbon ring. There is potential for confusion because there are different numbering systems used to describe the position of this double bond. Under the dibenzopyran numbering system widely used today, the major form of THC is called Δ9-THC, while the minor form is called A-THC. Under the alternate terpene numbering system, these same compounds are called Δ1-THC and Δ6-THC, respectively.

THC/THCA can be converted to CBN/CBNA (about 90% less psychoactive), which can be more sedating, but less euphoric, by heat, ultraviolet radiation, oxidization, or as a degradation product, and what's left of THC, may degrade into Δ8-THC (about 50% less potent), and to a much lesser extent, cannabitriol (CBT), presumably all these degradation products are minimally psychoactive. CBN, in turn, can photo-chemically convert to mildly psychoactive cannabinodiol (CBND) and cannabinidiol (CBDL). The presence of THCA (invariably it also contains some Δ9-THC content) and possibly its degradation product, CBNA, both are generally less psychoactive, as well as its V derivatives (and their A forms), which may be more energizing, by binding less strongly to CB1R receptors (also CB2R) as compared to THC, but they can also bind variably to the non-ECS receptors. Analogous to CBN and CBNA, psychoactive cannabinvarin (CBV) or CBVA is an oxidized product of THCV/THCVA respectively, bind to CB1R (primarily), and is less potent. CB2R agonists can mediate diametrically opposite effects on cAMP levels on mast cell membranes. Exogenous cannabinoids extraction needs to free from alcohol, which may precipitate and allergic response in those with mast cell activation syndrome (MCAS).

CBG and its acid precursor, CBGA can be classified as one of the major cannabinoids, as a phenolic (acid based) terpene oil, which is then combined with several plant derived additives to enhance effect, absorption and flavor, to form the primary ingredient of the novel proprietary food product, PECSA™ (the Bazelet Health System company's proprietary Phyto-EndoCannabinoid System Activating compound).

THC (most commonly, the Δ9-isomer, found in up to 30% in chemovars), its degradation product, CBN (found in <1% in fresh marijuana harvest), as well as CBD (found in up to 20% for chemovars), cannabichromene (CBC, cannabinoid found in up to 4% in chemovars), CBG (found in up to 1% in medical marijuana, but found in up to 20% in chemovars), all of which are decarboxylated from the less stable acidic (A) precursor molecule in the plant, THCA, CBNA, CBDA, CBCA and CBGA, respectively; they may also be converted by heating. Less well studied minor phyto-cannabinoids (not related to THC) as follows: from CBC degradation: cannabicydol (CBL) and cannabicitran (CBTC); CBDVA derivatives: cannabielsoin (CBE) and cannabielsoinic acid (CBEA); CBD derivatives: cannabimovone (CBM, found in higher concentrations in Italian cultivars, with higher affinity for TRPV), cannabifuran (CBF), cannabioxepane (CBO), cannabidiol dimethyl ether (CBDD).

The distinct “Δ9-THC” isomer (the most common form found in any Cannabis plant), and is illegal federally (Drug Enforcement Agency-DEA, Schedule I, but medical marijuana is currently legal in 37 states and the District of Columbia-DC, of which 19 states plus D.C. have additionally legislatively cleared Cannabis for recreational use as well) does always not differentiate the term “THC” (its isomers are less specifically regulated) in much of the nomenclature or scientific literature. Δ9-THC includes racemic mixtures (“trans” is found in buds and is more potent whereas “cis” form is more common in the hemp fiber). THC may have other isomers or variants (including tetrahydrocannabiphorol-THC-P, which is 33 times more potent than Δ9-THC, and found to exist naturally in the Italian FM2 Cannabis chemovar; Δ8-THC from degradation, which is less potent but stabler; or tetrahydrocannabutol—THCB), its natural degradation products, and derivatives, V and A forms, analogs (including FDA approved drugs based on controlled substance act-CSA, as well as illegal analogs as DEA Sch I), but are not chemically, Δ9-THC. THCA is less potent CB1R agonist (less psychoactive), with less effects on locomotion or temperature regulation than THC, with more serotonin binding, making it a distinct compound with antioxidant and anti-inflammatory effects. THC also has active metabolites (usually from liver), 11-OH-THC which is even more potent than Δ9-THC; It appears in higher concentrations in blood when Cannabis is ingested than when it is inhaled; however, serum levels may not directly correlate with brain levels for euphoria, and acute vs. chronic exposure may alter this effect. THC can be excreted with some active metabolites. Cannabidibutol (CBDB) is a butyl homologue of CBD, which might actually be derived from microbial w-oxidation and decarboxylation of their corresponding five-term homologs. Cannabidiphorol (CBDP) is an alkyl CBD homologue, possibly more potent than CBD. THC-mediated activation of CB1R signaling pathways converge on p38 Mitogen-activated protein kinase (MAPK), TNFα, and NFκB outputs creating a pro-inflammatory and atherogenic environment in endothelial cells. Copaiba, black pepper or any essential oil that contain β-caryophyllene, which can by itself, activate the ECS(CB2R agonist) without any psychotrophic effects.

Microbiome: In addition to the “body-mind-spirit” connection, the endocannabinoidome system is intimately involved with the “gut feeling” or microbiota-gut-brain axis (MGBA) by mediating immune, humoral, neural or metabolic pathways that affect CNS functions and behaviors and can affect brain development and synaptic formation involved with social activities (in zebrafish). Dysregulation of the ECS has been connected to GI disorders such as inflammatory bowel disease (IBD), irritable bowel syndrome (IBS), as well as obesity, and intestinal dysbiosis, which in turn, has been associated with neurodegenerative disease and cancer. Gut dysbiosis is associated with poorer prognosis with sepsis, stress response (including PTSD) and cardiovascular disease. Symbiotic microbiomes (at least 10 times more numerous than human cells) affected include the skin, respiratory, oral conjunctival and urogenital mucosal membranes, in which there are bidirectional effects with the ECS system and with phytochemicals, including hemp derived.

CED: Clinical Endocannabinoid Deficiency (CED) Syndrome really represents dysfunction in the homeostatic endocannabinoidome system, in terms of available endocannabinoids could result in a wide range of common diseases and conditions, including fibromyalgia, cognitive dysfunction, fatigue, migraine headaches, neuro-degeneration (including Alzheimer's disease and Parkinson's disease), and mental health (depression, anxiety, PTSD, sleep), with symptoms manifested as “reduced food intake, heightened anxiety, increased arousal and wakefulness, deficits in extinction of aversive memories, and super sensitivity to stress,” and a diverse variety of GI conditions including IBD, IBS, as well as skin, respiratory conditions, endometriosis, vitality and even cancer. This may really represent Clinical Endocannabidiome Dysfunction, with several simultaneous disruptions of ECS/non-ECS pathways, resulting in a relative deficiency or even a relative upregulation of cannabinoids (imbalance) in certain regions of the brain or periphery (including GI) affecting the delicate balance this holistic complex system, with built in redundancies or multidimensional mechanisms of effect. Appropriate intervention is to normalize into the therapeutic range (may be individualized, contextual and dynamic) of transmitters, modulation and binding, which can often be subtle, present with insidious and nonspecific symptoms with delayed recognition from both the affected individual and observers. Activation of the ECS (by increased levels of AEA, or NAEs) are significantly increased in exercise, which can be perceived as the “runner's high,” (euphoria and reduced anxiety) and this experience was not blocked by the opioid antagonist, naltrexone.

FAAH-1/2 hydrolysis, for CB2R agonists including AEA (forming AA and ethanolamine), which is degraded post-synaptically (and to a lesser extent by cyclooxygenase-COX-2 to prostaglandin-ethanolamides). FAAH is a dimeric integral membrane protein, found on the cytoplasmic face of smooth endoplasmic reticulum (ER), mitochondria and, less frequently, the cell membrane; its most active in the brain (especially hippocampus and cortex) and liver. There is a very minor effect for FAAH metabolism of 2-AG, potentially if it is up-regulated. There is a more complex system for 2-AG (usually forming arachidonic acid and glycerol or to other minor metabolites by COX-2, lipooxygenases cytochrome P450, bioactive phosphorylation by monoacylglycerol (MAG) kinases or MAG acyl transferases, and can act as CBR antagonist), serine hydrolase α-β-hydrolase domain 6 (ABHD6, embedded in the cell membrane postsynaptically in hippocampus, glia, and dendritic spines of prefrontal cortex, also in liver, kidney and ovary) and serine hydrolase α-β-hydrolase domain (ABHD12, more abundant, embedded in the Golgi membrane, with its active site facing the lumen postsynaptically; and utilized as a second line catabolism with excessive levels of 2-AE or MAGL is not present, found in hippocampus and cerebellum; a mutation of its gene is linked to a neurodegenerative disease in humans characterized by polyneuropathy, hearing loss, ataxia, retinitis pigmentosa and cataracts) for its CB2R function; and chiefly by monoacylglycerollipase (MAGL, with at least 2 isoforms) for its CB1R function, found intracellularly in presynaptic neurons and degrades only after it is released pre-synaptically. Both AEA and 2-AG metabolize intracellularly, such that transporters can allow these endocannabinoids to actively diffuse (possibly with protein carriers) through sodium based channels, which can allow a prolonged (minutes) effect of CB1R binding (its activation inhibits transmitter release by inhibiting Ca2+ channels) particularly to GABA-ergic neurons cellular reuptake mechanism that can be blocked by specific inhibitors. Complete pharmacological or genetic inactivation of MAGL increases endocannabinoid tone and causes CB1R desensitization, tolerance to CB1R agonists and down-regulation of CB1R. It is likely that inhibition of 2-AG degradation via MAGL attenuates AA-based lipid production, which also results in reduction of several eicosanoids, including PGE₂, PGD₂, PGF_{2α} and thromboxane B₂. MAGL inhibitors prevented the rise in brain eicosanoids and inflammatory cytokines seen following LPS injection, without affecting basal cytokine levels. Moreover, the reduction in activated cytokines was not reversed by CB1R antagonists, but was mimicked by COX-1 blockade suggesting they were due to COX-1 metabolites and not CB1R activation. MAGL inhibitors may be an effective therapeutic option for neuroinflammatory conditions. COX-2 is a prostaglandin-endoperoxide synthase that is essential in the synthesis of prostaglandins from free AA but it also metabolizes 2-AG to prostaglandin E₂-glycerol ester (PGE₂-G). COX-2 expression is induced by various inflammatory and other injurious stimuli including CNS insults such as ischemia, trauma and seizures, and is a major producer of prostaglandins during an inflammatory response.

PGE₂-G is a multifunctional signaling molecule whose effects include immune system modulation, hyperalgesia and enhanced neuronal activity. COX-2 inhibition was also shown to prolong depolarization suppression of inhibition (DSI; which is GABA related) in the hippocampus, which is dependent on both MAGL and COX-2, and can limit the retrograde signaling actions of 2-AG. COX-2 oxygenation of 2-AG has been implicated in glutamate-induced excitotoxicity through PGE₂-G activation of caspase 3, ERK, p38 mitogen-activated protein kinase, IP₃ and NF-κB signaling. Taken together, the data suggest that antagonism of the PGE₂-G receptor or novel inhibitors of COX-2-mediated PGE₂-G formation could be used to treat neurodegenerative and inflammatory diseases. This selective inhibition of 2-AG oxygenation appears to be therapeutically beneficial, as it was efficacious in a mouse model of anxiety and would avoid the effects of impaired prostaglandin formation. FAAH is linked with arousability and aversive-memories extinction, and may be correlated with cellular learning and memory. AEA may help memory by helping us forget (“noise”), thus, allowing the brain's ability to weaken unimportant memories and experiences enables it to function more efficiently.

Anxiolytic and mood: CB2R agonists have anxiolytic properties including pharmacologically-induced anxiety, a simulated public speaking task as well as in patients with social anxiety disorder (SAD). Clinical studies show full spectrum CBD (30 mg/day, containing small amount of THC) sublingually, administered for 4 weeks improved anxiety along with secondary outcomes of mood, sleep, quality of life, and cognition (specifically executive function) following treatment in as little as 4 weeks, without serious adverse events (minor side effects included sleepiness/fatigue, increased energy, and dry mouth).

Analgesic and anxiolytic effects of cannabinoids have shown to be dose-dependent in studies of chronic pain and anxiety. A possible mechanism of action through which CBD may be beneficial for fibromyalgia (FM) could be through enhancement of endogenous AEA levels through inhibition of intracellular fatty-acid binding proteins that are responsible for the transport of AEA to its catabolizing enzyme FAAH. Other possible mechanisms include modulation of serotonergic (5HT) transmission (particularly positive allosteric modulation of 5-HTA), desensitization of TRPV1 and antagonism of GPR55, with some studies reporting more than 60 targets. Inhaled Cannabis has been shown to be the most beneficial in terms of pain, quality of life and sleep. Some dietary fatty acids, which can also be found in plants, can modulate the ECS by influencing the availability of phospholipid biosynthetic precursors of endocannabinoids.

ECS regulates cognitive abilities, mood, stress, and sleep with neuro-modulation in four domains: 1. Mood (euphoria, unfounded laughter; paranoid or anxious reactions at high doses), 2. Perception (disturbance of the perception of time and space), 3. Somatic symptoms (fatigue, problems with motor coordination, dizziness), 4. Cognitive impairment (confusion, impaired concentration, impaired short-term, and working memory). A decreased ECS may entail a depressive-like phenotype (EDS); however, cannibinoids can also adversely affect psychopharmacologic or mental health therapy. Current guidelines recommend caution in using THC in patients with anxiety or mood disorders. There is limited evidence from clinical trials that cannabinoids are effective therapy for sleep disorders associated with concomitant conditions. There is evidence for a possible role of Cannabis as a substitute for substance use disorders (SUD), also in the context of the risks of opioid use (e.g., opioid-related modality). In animal models, pharmacological or genetic disruption of ECS may mimic the classic stress response, by CB1R activation of the hypothalamic-pituitary-adrenal axis (HPA), increased anxiety, excessive vigilance, agitation, inhibition of feeding behavior, decreased response to rewarding stimuli, and impaired cognitive flexibility. Regulation of the stress-response mechanism in shod-term stress causes ECS inhibition, while long-term stress stimulates ECS, which alleviates the negative effects of the stressful situation.

Chronic stress seems to reduce the CB1R's ability to buffer stress and may induce psychopathology, including anxiety and depression, and may promote psychiatric disorders. CB1R inhibits the release of excitatory amino acids (Glu) as well as GABA which consequently regulate the release of other transmitters: ACh, DA, histamine, 5HT, NE, prostanoids, and opioid peptides. CBR are present at very high levels on inhibitory (GABAergic) interneurons and at a lesser extent on excitatory (Glu) terminals, as well as on neurons expressing dopaminergic D1 receptors, playing a specific role in the repertoire of different emotional behaviors, including social and cognitive activity, which are affected in psychiatric disorders. THC may induce euphoria, cognitive impairment, and intensify negative emotional states, including anxiety. Other non-THC based cannabinoids may function as a negative allosteric modulator (NAM) of the CBR, reducing its activation, and binds to non-ECS receptors. CBD may inhibit the transpod of AEA, OEA and PEA to FAAH by blocking fatty acid-binding proteins (FABPs).

Cannabinoids may have therapeutic potential in both depression and bipolar disorder (the latter more difficult to treat due to its dual depressive and manic/hypomanic components, which can cycle rapidly). There are reports that Cannabis can be a mood stabilizer in bipolar disorder and an adjuvant to lithium therapy (it allows for dose reduction). CBD at therapeutic doses have anxiolytic effects in animal models of generalized anxiety disorder, social phobia, panic disorder, obsessive-compulsive (OCD) syndrome, and post-traumatic stress disorder (PTSD) and in humans, including those with SAD by binding to the limbic and paralimbic areas of the brain. Higher CBD dosing may also attenuate the observed benefits in animal studies, so that optimization of dosing is being studied. THC has a pro-psychotic effect in those that can be genetically susceptible (peripheral blood mononuclear cells of schizophrenic subjects, selective alterations of DNA methylation at the promoter of the gene coding for CB1R were found in an animal model of schizophrenia, induced by prenatal methylazoxymethanol acetate-MAM, with as associated alteration in D2/3 receptors, and can be used as a prefrontal codex biomarker for schizophrenia), while CBD (and possibly combined with a very low “balanced” THC dose) reduces the occurrence of such disorders, can attenuate THC-induced psychosis and can be a useful adjunct for Cannabis use disorder (CUD). Patients with opioid use disorder (OUD) on medication assisted treatment who used marijuana before the treatment needed less methadone experienced less withdrawal symptoms (based on Clinical Opiate Withdrawal Scale-COWS). A similar effect was seen in a Canadian study on alcohol and illicit psychoactive substances. In a randomized clinical trial, CBD was found to reduce cue-induced craving and anxiety as a treatment option for OUD.

Substance use disorders (SUD): Stress (which involves the physical arousal of the hypothalamic-pituitary-axis-HPA and the autonomic sympathetic nervous system) is a risk factor for initiation and maintenance of Cannabis (THC) use as a mechanism of coping, as a bidirectional phenomenon. This occurs particularly, if there are psychiatric co-morbidities (e.g., PTSD, depression or anxiety), sex and life adversities (including partner violence, job loss, lack of housing and other social disparities), especially at an earlier age. Acute THC may activate the acute stress response, whereas chronic stress may reduce the quantity of CB1R, an effect that may be mediated by activation of glucocorticoid receptors, with a blunted cortisol stress response; and habitual Cannabis use may be associated with higher cortisol release. There are sex differences in clinical effects, metabolism, menstrual hormonal cycle (estradiol levels may reinforcing use, particularly in those with premenstrual dysphoric disorder-PMDD), side effects and progression to habitual use. Prenatal Cannabis exposure (PCE) is harmful (with poor maternal awareness on this subject) with predisposing socio-economic disparities, as it is linked to low birth weight, preterm birth, and admission to neonatal intensive care; and in utero exposure has been linked to anxiety-like behavior in adolescents and adults as well as to dose-dependent reductions in dopamine receptors.

Endorphins, produced by the pituitary gland acts as p and 6 opioid allosteric modulators, as it interacts with CB1R to enhance the rewarding dysphoric effects contributing to Cannabis dependence and a bidirectional effect with opioids, contributing to opioid withdrawal symptoms.

Clinical reports regarding the effects of CB2R agonists (particularly CBD) have been favorable on the regulation of the reinforcing, motivational and withdrawal-related effects of habituating drugs, through the regulation of dopaminergic (mediated by Glu neurotransmission), opioidergic, 5HT, and ECS as well as hippocampal neurogenesis. In mice, CBD down-regulates the gene expression of CB1R and GPR55 whereas it up-regulates CB2R in the nucleus accumbens with THC withdrawal. CBD may functionally regulate the activity of the mesolimbic DA system and counteract the effects of dysregulated dopaminergic transmission induced by drugs such as amphetamine, cocaine, alcohol, or Cannabis, and possibly nicotine (only a temporary benefit) as well as improving sleep, cognition, neurogenesis and presents anxiolytic, antidepressant, antipsychotic, and neuroprotective effects, and there may be a gender difference in responses. There are no specific treatments for Cannabis, cocaine, or amphetamine-type use disorders. CBD restored Cannabis-induced anatomical disturbances in the subicular and CA1 subfields of the hippocampus related to chronic THC and disrupted the salience network. It may reduce the negative psychotropic effects of THC and inhibited THC-induced paranoia, psychosis and episodic memory deficits. For recreational Cannabis users and for those patients taking medicinal Cannabis, a more balanced CBD to THC concentration can improve therapeutic endpoints while minimizing side effects. In mice, CBD alone or in combination with THC reduced ethanol-induced locomotor sensitization; the combination THC and CBD significantly inhibited the expression of sensitization to ethanol and the combination of CBD plus naltrexone (opioid antagonist) was the only treatment able to reduce motivation and ethanol intake (may be modulated by 5HT1A and p opioid receptors in NAc). CBD has neuroprotective actions in rodent models of ethanol binge intoxication and might also present protective actions against alcohol-induced liver disease, attenuating hepatic steatosis and metabolic dysregulation by anti-inflammatory and antioxidant mechanisms. THC and especially CBD plus THC combination significantly attenuated morphine (opioid) withdrawal signs (possibly via 5HT1A). CBD alone, blocked place conditioning behavior and reinstatement induced by a priming dose of morphine, cocaine (via CB2R, 5HT1A and TRPV1), (met)amphetamine (without affecting the learning process of place conditioning) or stress exposure. In humans, CBD also attenuated drug cue-induced physiological measures of heart rate and salivary cortisol levels in heroin abstinent patients. In rats, CBD was associated with the normalization of methamphetamine-induced increase of gene expression of cytokines (interleukin-1β, interleukin-6, interleukin-10, and tumor necrosis factor α(TNF-α)) in the prefrontal cortex (PFC) and hippocampus (but not in sleep deprivation) resulting in a neuroprotective effect. In mice, CBD blunted the motor behavioral response induced by a challenge dose of cocaine plus caffeine and blocked the increase of dopamine transporter (DAT) and TH gene expressions in the VTA of mice exposed to the cocaine withdrawal, and reduced seizures. However, other animal studies have not shown CBD to be helpful for cocaine withdrawal.

In mice, CBD abolishes memory impairment and microglial reactivity induced by nicotine withdrawal, and in humans, helped with attentional bias and anxiety. There are currently human clinical trials for cannabinoids for mitigation of withdrawal or cravings with psychoactive substances. Direct cannabinoid receptor ligands are compounds that show high binding affinities (in the lower nanomolar—nM range) for cannabinoid receptors and exert discrete functional effects (i.e., agonism, neutral antagonism or inverse agonism). By contrast, indirect ligands target either key proteins within the ECS that regulate tissue levels of endocannabinoids or allosteric sites on the CB1R, which modulate the ECS.

PTSD: The Veterans Administration (VA) estimates that 11 to 20 percent of recent veterans of the wars in Iraq and Afghanistan, and as many as one-third of all veterans, suffer from PTSD. There may be other superimposed comorbidities including TBI (traumatic brain injury, including blasts or explosions), with pre-existing mental health, substance abuse and adverse childhood experiences (ACE). PTSD features in humans have been attributed to neuronal dysfunction within the medial prefrontal cortex, anterior cingulate cortex and hippocampus and with diminished connectivity between the ventromedial prefrontal cortex and amygdala. Furthermore, low ECS tone contributes to the amygdala hyperactivation as well as the anxiety and hyperarousal symptoms characteristic of PTSD. Symptoms includes sleep disturbances (with intrusive dreams or nightmares), memory and cognitive impairments, altered pain sensitivity, as well as depression, anxiety, emotional numbing and suicidality, along with dissociation, which is more prominent in females. It has been hypothesized that ECS is tied to the development of PTSD, possibly through a corticotropin-releasing hormone-mediated reduction of AEA in several brain regions. PTSD was associated with increased expression of CB1R, and reduced peripheral levels of the AEA as well as a compensatory increase of CB1R availability, which was linked to excessive threat processing and with features of anxious arousal (associated with adrenal hyperactivity, with disruption of fear memory consolidation, decreasing salience of ordinarily significant stimuli, or facilitating the extinction of fear memories) suggestive of a stress endophenotype underlying PTSD. Cannabinoids may be useful in psychological conditions that involve elevated inflammatory processes mediated by CB2R within the brain, including a subset of depressed individuals. Therapies can best be accomplished by determining the optimal ratio of the CBR (e.g., THC in relation to CBD) that are most effective at promoting therapeutic effects while minimizing adverse effects. Low doses of THC analogs have led to improvements in self-reported subjective sleep quality, decreased frequency of nightmares, and improvements in self-reported overall well-being among those with PTSD. In a 3 week trial of US veterans, all treatment groups (randomized, with varying proportions of THC and CBD, including placebo), showed good tolerability and significant improvements in PTSD symptoms during three weeks of treatment using smoked medical Cannabis. The adverse events were generally mild to moderate, and did not significantly differ by treatment condition. In an extension of this study, over the course of a year, Cannabis users reported a greater decrease in the severity of their PTSD symptoms. They also were more than 2.5 times as likely to no longer meet the diagnostic criteria for PTSD as those who did not use Cannabis.

ADHD: CBD reversed all THC-induced changes (except for sucrose preference, which it enhanced) when co-administered, which supports evidence of CBD as a potential antipsychotic, antidepressant and anxiolytic. CBD did not affect amphetamine-induced hyper locomotion in a rat model (which was previously observed in another rat model of positive symptoms in bipolar by assessing gamma activity), which may be modulated by brain derived neurotropic factor (BDNF). It has also been concluded that early-life disruptions of the ECS, including adolescent cannabinoid exposure, can lead to long-term baseline oscillatory changes implicated in the etiology of mental illnesses.

A 2020 study of 112 adult patients with ADHD who used medical Cannabis found that those who took a higher dose of medical Cannabis components, like CBD, took fewer other ADHD medications (psychostimulants including amphetamine); other studies have shown less side effects from conventional drugs when medical Cannabis is added (advised only in those over 21 years). Cannabis can play a complimentary role in the therapeutic regimen of ADHD as demonstrated in case reports of young male adults, using CBD:THC (20:1) from a medical source in Canada, improving their symptoms and quality of life were substantial, such as the ability to keep emotions in check (3 patients) or to obtain and excel at a new job with more responsibility (2 patients). Any formal intervention with medical Cannabis needs to be very closely monitored with informed consent. However, nonpsychoactive cannabinoids can be tried more liberally primarily for other target of symptom control.

Developmental: ECS responds early to neuronal damage, working to prevent Glu excitotoxicity and regulate the inflammatory response. While there are no current human studies, results from mice and pig models demonstrate that CBD can reduce the density of necrotic neurons and modulate cytokine release. Low dose CBD administration in mice was also shown to be associated with increased levels of biomarkers of neuronal differentiation and hippocampal neurogenesis in several models in mice. ECS in the various locations in the CNS are significantly higher during juvenile and pubescence, and drastically decreases in adulthood. CBR are also notably abundant in the cortico limbic brain regions in adolescent rats. Further studies have shown that cannabinoids exposure during developmental stages impairs learning and memory development with anxiety, and the timing and duration of exposure might play a crucial role in its long-term trajectory. Cannabis treatment for seizures has along history, dating back at least 4000 years. Children with epilepsy who receiving pure CBD (specifically, Lennox-Gastaut syndrome, characterized by multiple drug-resistant seizure with a unique electroencephalographic pattern, and Dravet syndrome, presenting with pleomorphic seizure activity, usually before the one year of age for which a branded pharmaceutical product was FDA approved in 2018) with 50% reduction in seizures rate and greater caregiver global impression of change. These infants suffer from cognitive decline, motor, and behavioral abnormalities. This anticonvulsant effect may be due to binding of GPR55 antagonism by CBD.

The ECS is also expressed in human placenta and modulates endothelial nitric oxide (NO) production, which contributes to multiple functions in the body including vasodilation via smooth muscle relaxation and increased platelet aggregation, which may contribute to hypercoagulation and thrombosis. Cannabinoids, by binding to CB1R, increases NO synthase, thereby increasing NO levels, which improves regulation of body temperature, cardiovascular parameters, neuroprotection, mitochondrial energy, anti-aging, antimicrobial, anti-inflammatory, and sexual or erectile function.

Analgesia: Recent meta-analyses of clinical trials that have examined the use of medical Cannabis in chronic pain present a moderate amount of evidence that Cannabis/cannabinoids exhibit analgesic activity, especially in chronic or neuropathic pain, (and perhaps cancer, particularly with neuropathic components including chemotherapy induced adverse effects), hypothesized through an anti-inflammatory and immunologic action of CB1R alone, or CBR combined agonists, with considerable involvement of non-ECS systems (primarily COX-2, TRPV1, GPR55, α2 adrenergic and PPARs), as well as improved secondary outcomes including mood, anxiety, sleep and energy. The unique α-2 adrenergic effect of CBG may lower blood pressure and cause sedation; its serotonin effect may affect appetite, cause GI symptoms, sedation, and perhaps sexual dysfunction. THC has additive analgesic efficacy with kappa opioid receptor agonists, which may be useful for chronic pruritus. THC's effect may be mitigated by kappa antagonism, but this blocking does not alter the psychoactive effects of THC. Other studies support that amygdala activity contributes to inter-individual response to cannabinoid analgesia centrally, and may modulate differential effects on the sensory (e.g., intensity; quality) versus affective (e.g., unpleasantness; suffering) components of pain. Data from studies investigating viscerally induced pain due to colorectal distension indicate that peripheral CB1R mediate the analgesic effects of cannabinoids on visceral pain from the GI tract. CB2R agonists can evoke analgesia by triggering the release of beta-endorphin in response to the stimulation of CB2R expressed in human keratinocytes and may exert its effects via inhibition of anandamide deactivation or otherwise enhancing anandamide signaling; in addition, CB2R agonists suppresses neuronal activity in the dorsal horn via reduction in C-fiber activity and wind-up involving wide dynamic range (WDR) neurons. These effects can be due to a combination of actions: inhibition of the release of neurotransmitters and neuropeptides from presynaptic nerve endings, modulation of postsynaptic neuron excitability, activation of descending inhibitory pain pathways, and reduction of neural inflammation. Many studies have outcomes based on pain relief, but not necessarily evaluating function. However, the data for long-term outcomes or harms are lacking, with a controversial opioid sparing effect. In a survey taken in 2018, the majority of 2183 Florida participants (79%) that used medical Cannabis (55% took it daily) through state approved dispensaries) for a variety of conditions including anxiety disorders, chronic pain, depression, insomnia and PTSD reported either cessation or reduction in pain medication (opioid) use following initiation of medical Cannabis and 11.47% described improved functioning (although there may be a contributing factor of involuntary opioid reduction or limited access, based on regulatory policies). most of the participants (90.6%) found medical Cannabis to be very or extremely helpful in treating their medical condition and most (88.7%) said it was very or extremely important to their quality of life. 68.7% of these subjects experienced at least one side-effect, the most common of which were dry mouth, increased appetite and drowsiness Other recent retrospective studies have shown that of 550 subjects enrolled in Cannabis clinics, 48% reported a significant decrease in pain, and most said they had better quality of life (87%) and better physical function (80%) while using medical Cannabis; however, 24.8% enrolled in the study reported losing access to prescription medication or medical care as a result of their Cannabis use or after testing positive for THC. The most commonly reported adverse effect for THC based drugs was dizziness (15.5%), followed by drowsiness, faintness, fatigue, headache, problems with memory and concentration, the ability to think and make decisions, sensory changes, including lack of balance and slower reaction times (increased motor vehicle accidents), nausea, dry mouth, tachycardia, hypertension, conjunctival injection, muscle relaxation, with a tolerance effect for adverse symptoms. They can also cause significant psychiatric symptoms or dysphoria, but are devoid of lethal overdoses.

In some studies involving ketorolac, a nonsteroidal analgesic, the placebo analgesic response is not blocked by naloxone (as it would be if it were an opioid) but is blocked by rimonabant, a CB1R antagonist, which suggests the involvement of endocannabinoids in placebo analgesia. This effect may also involve the CBR ligand precursors of the lipidic pathway (involving AA, AE, PG and thromboxane) as important in the modulation of the placebo response in pain. Dopamine also plays a role in placebo analgesia responsiveness.

The trigeminovascular system (GVS) evokes the onset of migraine (also associated with seizures, GI, sleep and mental health disorders), and is intimately involved in its pathophysiology. During a migraine attack, prolonged activation of the TGVS peripherally results in comprising meningeal trigeminal nerves (has CB1R), arteries, veins (has CB2R), along with dural mast cells (MC, also found in epithelium, mucosa, vascular and nerves, which has CB2R, GPR55 receptors, and can release serotonin), which in turn causes CNS central sensitization, leading to the persistent nociceptive signaling as mediated by CGRP. In chronic migraines, CB2R is up-regulated, and may combine with CB1R to form heteromerization, contributing to glial related neuroinflammation. Cannabis has been used clinically in those with migraine and nausea to inhibit cortical spreading depression (CSD), underlying the migraine aura. It is also postulated that CED (from genetic or acquired etiologies) results in reduced CBR, which can be ameliorated with exogenous cannabinoids, including CB2R agonists with anxiolytic, mood stabilizing, sedating, anticonvulsive, analgesic, antiemetic, and anti-inflammatory effects. Sleep: CB1R activating compounds appear to produce modest sleep improvement, by decreasing sleep latency but could impair sleep quality long-term. Endocannabinoids show circadian fluctuations in healthy humans showed the highest AEA plasma levels occurred upon waking and the lowest just before sleep onset, and this was altered by sleep disruption, whereas 2-AG levels did not show prominent circadian fluctuation, as modulated by CB1R. The interactions between diurnal fluctuations in AEA and CB1R levels in this brain stem region may contribute to transitions in or maintenance of sleep-wake states. In the rodent hypothalamus, AEA levels are highest during the light phase of sleep, and 2-AG may promote wakefulness. This may reflect their complex role in sleep stability (and not altering “sleep-homeostasis”), in which awake-behavior related learning, e.g., associative learning related to action control, and sleep-behavior related learning, e.g., hippocampal-dependent memory consolidation still occur. A decrease in total sleep time, sleep efficiency, non-rapid eye movement (REM) and REM sleep were observed during abstinence in heavy Cannabis users. When CB2R agonists were used alone, there were no significant effects on sleepiness, but when combined with relatively small amounts of CB1R agonists, they improved sleep. A recent study found that neurons in the suprachiasmatic nucleus, a hypothalamic region critical for controlling the circadian rhythm, release CBR binding may also activate intracellular astrocyte signaling (most likely CB2R) which subsequently influences circadian dock timing. The tolerance effect of chronic CB1R agonists binding to the hypothalamus for sleep may also be associated with impaired fear memory, spatial learning and memory in rodents as well as a cross tolerance effect with the loss of synaptic depression induced by a μ-opioid receptor agonist. CB1R agonists may be useful for OSA, and reducing nightmares associated with PTSD, possibly due to additional glutaminergic and/or serotonergic modulation.

Sexual function: Female sexual arousal was correlated with decreased AEA and 2-AG concentrations. Modulation of the estrogen receptors is currently being considered for the prevention and treatment of a wide variety of pathological conditions, including osteoporosis, metabolic and cardiovascular diseases, inflammation, and neurodegenerative disorders, with an additional focus on cancer.

ECS in the preoptic/hypothalamic neurons (involved with corticotropin-releasing hormone (CRH), thyrotropin-releasing hormone (RH), oxytocin (OT), and vasopressin (VP), proopiomelanocortin (POMC), and gonadotropin-releasing hormone (GnRH)). Kisspeptin (KP) neurons are located in the preoptic area (POA), the arcuate nucleus (ARC), and the medial amygdaloid nucleus (ME) form afferents projecting to POA, supraoptic (SON), and paraventricular (PVH) nuclei of the hypothalamus which stimulating GnRH release, as well as alternative pathways involving serotonin, dopamine, GABA, glutamate and nitric oxide, as well as releasing other neuropeptides (neurokinin B, dynorphin, and galanin). It is widely expressed in limbic behavioral brain regions (1) by deactivating the left inferior frontal gyrus involving with processing language, internal monologue, working memory, inhibitory control, and empathy, (2) deactivating the postcentral and right supramarginal gyri, and temporoparietal junction, reducing a woman's focus on herself or having negative body image, (3) activation of hippocampal activity in those women who were more bothered by their low desire and (4) activation of the posterior cingulate cortex can serve to increase feelings of romantic love and cognitive reward processing including autobiographical memory, thereby reducing sexual aversion.

Kisspeptin release is modulated by CB1R, which in turn, is influenced by estrogen, but is also involved in male reproductive behavior (rodent lumbar lordosis and erections). Estrogen exposure reduces the level of CB1R in the rostral periventricular area of the third ventride and arcuate nucleus. This indicates a strong gonadal cycle-dependent role of ECS in the neuronal circuits involving KP. Oxytocin, DA, NE, 5HT and melanocortin have been studied as potential therapies for female hypoactive sexual desire disorder. Also, exogenous KP results in small increases in circulating luteinizing hormone (LH) and follicle stimulating hormone (FSH) levels, and allows women to experience a more positive body image, improving desire sexual activity and gaining satisfaction from sexual experiences through effective modulation of sexual attraction brain processing, as confirmed with functional magnetic resonance imaging (fMRI) neuroimaging, psychometric, and hormonal analyses.

Cannabinoids through CB1R, appear to modulate the release of gonadotropin-releasing hormone (GnRH) through their effect on hypothalamic (located at the CA1 region) GnRH-releasing neurons, with increased cortisol (with circadian rhythm, associated with stress and inflammation), while it decreases growth hormone (GH), thyroid hormone (both associated with energy and metabolism), prolactin, luteinizing hormone (LH) and follicle-stimulating hormone (FSH) concentrations (all associated with reproductive function), resulting in reduced testosterone production by the Leydig cells of the testis (and from the ovaries, adrenals and peripheral sensitized tissue in women) and lower circulating estrogen and progesterone levels, which can lead to anovulation and loss of libido. Estrogens decrease FAAH activity in the mouse uterus, and this leads to higher cannabinoid concentrations. Reduced progesterone affects reduces fertility by preventing the preparation of uterine lining prior to ovulation, and prevents GABA secretion, causing increased stress. During pregnancy, progesterone is secreted by the placenta for lactation, and this effect can be reduced with CBR ligands. Giving an exogenous precursor to progesterone (also found in female oral contraceptives), pregnenolone, to rodents can mitigate the adverse effects of CB1R binding and in humans, even reduce euphoria from other mind-altering drugs, and in another study, was associated with a slightly lower rate of depression. There is little direct evidence of interactions between cannabinoids and the androgen system. However, CB2R agonists through activation of PKA, PKC and MAPK, can lead to diverse cellular effects, including smooth muscle relaxation, neuromuscular signal transmission and neuronal plasticity.

In females, THC prolongs the estrous cycle and decreases the pro-estrous surge of LH inhibiting ovulation the effect produced by THC on animal sexual behavior occurs through engagement of CB1R in the hypothalamic region, and can reduce sexual motor posturing in rats, along with estrogen, progesterone and DA (DIB receptors). In humans, AEA levels fluctuate during the ovarian cyde in both the hypothalamus and the pituitary gland.

Animal data suggest that in female rats, CB1R initiates signal transduction with membrane DA and intracellular progesterone receptors to initiate female sexual responses. The dopaminergic reward pathway is also stimulated by CB1R, contributing to “addictive” behaviors including overeating, smoking, and substance use disorders.

Appetite and Metabolic syndrome: CB1R agonists may exhibit increased hedonic responses to sugar (did not alter taste perception), by increasing dopaminergic incentive-motivational properties of food, odor sensitivity and appetite, accounting for the “munchies,” which is not observed with non-psychoactive cannabinoids. There is a bimodal response of CB1R: for GABAergic pathways it promotes satiety, and for glutaminergic pathways it induces hunger.

In metabolic syndromes, some studies have shown CB1R mRNA to be less abundant in primary cultured adipocytes derived from white adipose tissue of obese children and visceral adipose tissue of obese adults. The high expression of CB1R levels are increased in the striatum and hypothalamus of streptozotocin-treated (diabetes mellitus induced) rats and is associated with an increase in plasma insulin concentration with a decrease in CB1 mRNA levels in the pancreas, white adipose tissue and dorsal root ganglia. CBR in atherosclerotic plaques indicates an important role of the ECS in atherosclerosis. A higher expression of CB1R is also associated with cardiovascular risk factors, such as obesity and dyslipidemia and CB1R agonists have been shown to increase the amount of reactive oxygen species, and thus to induce the apoptosis of endothelial cells in coronary arteries. CB2R agonists reduce the accumulation of lipids in human foam cells. Cannabinoids also lower the expression of gene duster of differentiation (CD)-36 receptor, also known as platelet glycoprotein 4), which promotes the release of pro-inflammatory cytokines and increases its own expression.

Cognitive: Beta-amyloid fragments induce a dose-dependent memory deficit, especially with Alzheimer's Disease (AD), and this effect may be associated with CB1R. Animal studies have shown that ultra-low doses of THC slow down the formation of plaque and tangles and reduce the inflammation caused by their presence, thus supporting the treatment of dementia, although a clinical study using low dose THC did not significantly reduce dementia-related neuropsychiatric symptoms. In post-mortem brain tissue of AD patients and in experimental models of AD, a decrease in neuronal cannabinoid CB1R, an increase in glial cannabinoid CB2R (inducible), and over-expression of free acid amide hydrolase in astrocytes hint its potential role in inflammatory processes and in neuroprotection.

CBN (CB1R agonist) protects mitochondria related oxytosis (or ferroptosis), which is triggered by the gradual loss of glutathione, causing neural cell damage and death vialipid oxidation oxytosis, which is thought to occur in the aging brain and AD. CBD is able to improve iron-induced memory deficits, and regulate markers of synaptic viability and mitochondrial dynamics (preventing injury; also in another study, CBD upregulates the anti-apoptotic protein Bd-in the mitochondria membrane in multiple sclerosis) in the hippocampus of iron-overloaded rats. CBD reversed iron-induced effects, recovering apoptotic proteins Caspase 9, APAF1 (completely reversible), Caspase 3 and cleaved PARP (a reliable marker for apoptosis), all proteins involved in apoptotic pathways, but did not reverse iron binding onto cytochrome C proteins.

There is less CB1R and/or signaling in ageing rats in a brain-region-specific manner: the reduction was most prominent in the cerebellum and cerebral cortex, whereas it was less pronounced but still significant in the limbic and hypothalamic structures as well as in the hippocampus. In CB1R deficient mice there was premature ageing of the skin and brain (impaired learning ability) accompanied by neuroinflammatory changes in the hippocampus, but not in the cortex or striatum, suggestive that glial activation by CB1R effect on GABA-ergic neurons in the forebrain, and independent of CB2R. CB1R agonists attenuated spatial memory impairment, reduced the number of activated microglia and triggered neurogenesis in aged rats. In humans, there is a sex-dependent increase in CB1R binding during ageing in the basal ganglia, lateral temporal cortex and in limbic areas.

Activating the CB2R with CBD has increased brain cell activity and helped reduce brain cell damage commonly associated with vascular dementia. CBD can be an effective anti-inflammatory agent, reduce motor symptoms (tremor, rigidity, bradykinesia) and maintain circadian (sleep) rhythms in Lewy Body dementia, although it does not significantly inhibit acetylcholine, which this disease preys on. There are limited studies on frontotemporal dementia (FTD, associated with hereditary amyotrophic lateral sclerosis-ALS, with depression, bizarre eating behaviors or psychosis) but may improve the symptoms: sleep disturbances, reduce anxiety, attack inflammation, and reduce motor symptoms such as tremor, rigidity, and bradykinesia.

In various CNS diseases, CB2R agonists are involved in neuroprotection: neonatal ischemia (CBD alone), Huntington's disease (HD)(CBD combined with THC) or Parkinson's disease (PD)(CBD probably combined with THCV) believed to be modulated by: FAAH or MAGL enzyme inhibition, TRPV1 and TRPV2, glutamate homeostasis, affecting K+ and L-type Ca2+ channels, 5-HTA (CBD activates the 5-HT1A receptor at concentrations above 10 μM, but at the much lower concentration of 100 nM it synergistically enhances other 5-HT1A receptor agonists receptors), PPAR-γ activation inhibits NFκB/Nr1-2 signaling activity (which in turn, reduces the expression of pro-inflammatory enzymes (i.e., iNOS, COX-2), pro-inflammatory cytokines and metalloproteases).

CB1R levels were reduced in the caudate nucleus, anterior dorsal putamen and external segment of the globus pallidus, relative to controls or other brain regions of PD (a neurodegenerative disorder of dopamine deficiency in the basal ganglia) as well as HD (chorea). CB1R levels may correlate with disease progression, suggesting that CB1R normally performs a neuroprotective role in the striatum and loss of this receptor correlates with HD pathogenesis. In contrast, CB1R mRNA expression does not appear to change in AD, and cannabinoid effect may be related to neuroinflammatory etiologies, mediated through CB2R.

CB1R levels can be modulated by pro-inflammatory peptides, estrogen, insulin, atypical antipsychotics, methamphetamine, ethanol, retinoic acid and, more importantly, all cannabinoids. Acute treatment of rats with methamphetamine is associated with increases in steady-state CB1 mRNA levels in the prefrontal cortex, caudate and putamen, basolateral amygdala, CA1 hippocampal region and perirhinal cortex, relative to other brain regions and is associated with increased dopamine neurotransmission. Ethanol exposure appears to alter CB1R expression during early development and adulthood and may lead to chronic alterations in neurotransmission and gene expression that are normally facilitated by CB1R. Mice fed alcohol develop fatty liver increased hepatic CB1R, which is up-regulated resulting in increased lipogenesis and decreases fatty acid oxidation, which may also be co-modulated with estrogen receptor and RARγ. Cannabinoids modulate steady-state CB1 mRNA abundance. Chronic treatment with CBR agonists has been shown to decrease CB1 mRNA levels in the CNS of rodents. Repeated exposure (potency, duration of effect and frequency of exposure) to CBR agonists differentially decreases CB1 mRNA levels in the caudate and putamen of adult male rats, whereas it increased CB1 mRNA levels in the rat cerebellum and hippocampus, and this may also be estrogen receptor and RARγ-dependent. There is CBR interaction, in which CB2R activation of signal transducer (at threshold concentrations) stimulates signal transducer activator of transcription (STAT-5/6), which in turn, leads to inducing CB1R promoter activity. Cannabinoid treatment therefore, as in various pathological conditions, is associated with malleable context-specific regulation of CB1R expression, in which repeated exposure to CB1R agonists is associated with receptor desensitization and tachyphylaxic (tolerance) effects.

In mouse models of AD, CB1R agonists can reverse the cognitive impairments and the associated biochemical derangements, associated with increases in the levels of malondialdehyde (MDA), superoxide dismutase (SOD), glutathione (GSH) and neurogenesis markers: Nestin and glial fibrillary acidic protein (GFAP).

Using experimental allergic encephalomyelitis (EAE), an animal model of multiple sclerosis (MS, an autoimmune central demyelization CNS disease), CB1R agonists are immunologically and neuroprotective by inhibiting Glu-induced excitotoxicity involved with K+ and particularly Ca2+ channels. CB1R tonically regulates NMDA Glu receptor activity in vitro and support the in vivo observation that NMDA-induced and ischemic excitotoxicity.

The Glu-N-methyl-d-aspartate (NMDA) receptor inhibitor depresses Glu post-synaptic transmission via CB1R activation, affecting Glu release, inhibiting receptors and transporters function, reducing enzyme activity, and disrupting Glu synaptic plasticity after prolonged exposure modulating allodynia and hyperalgesia. This receptor may modulate breathing, locomotion, learning, memory formation, and neuroplasticity. Glu is involved in the detoxification of ammonia and serves as a precursor for the synthesis of proteins; by limiting its release, it reduces the neuron's exposure to a potentially toxic byproduct in the extracellular compartment, which can be associated with mental or cognitive fatigue in the prefrontal cortex. Glu plays an important role in a myriad of key functions like learning and memory as well as breathing, however, when overstimulated, the neurons that can become damaged and may contribute to a neurodegenerative conditions. GABA (primarily (A) β (2/3) receptor subunits, involved in inhibitory activity for anxiety, sedation, motor spasticity, memory, dopamine regulation, also GABA(B).

For some CNS mediated movement disorders, orally administered cannabinoids (nabiximols and nabilone, with CBD/THC content:1:1) can improve patient-reported subjective symptoms of muscle spasticity in persons with multiple sclerosis. CBD has also reduced FosB expression following cryogenic spinal cord injury and lowered iNos expression in a mouse model of tauopathy.

Bone health: Parathyroid hormone receptor (PTHR) activation for CBR: in CB2R-deficient mice show a markedly accelerated age-related bone loss. Activation of CB2R attenuates ovariectomy-induced bone loss in mice by restraining bone resorption and enhancing bone formation. Activation of CB1R in sympathetic nerve terminals in bone inhibits norepinephrine release, thus balancing the tonic sympathetic restraint of bone formation. Low levels of CB1R were also reported in osteoclasts. Cannabinoids may be used for treating accelerated osteoclastic bone resorption, including osteoporosis (primary and secondary) and boney metastasis (the latter, by inhibiting the release of tumor-derived factors, such as parathyroid hormone-related protein (PTHrP), interleukin 6 (11-6), tumor necrosis factor (NF), and transforming growth factor β(TGF-β), thus de-activating osteoclastic differentiation and bone-resorbing activity, which in turn, inhibits the release of bone-derived growth factors, cytokines, bone extracellular matrix components and prevents osteoblastic lesions, which presumably will also have an analgesic effect).

AEA and 2-AG are responsible for most of the pharmacological effects associated with CBRs in mammalian cells, and can be produced in bone marrow. The differentiation of human osteoclasts (OCs) was related to the increased AEA level and decreased 2-AG level. CB1R, CB2R and TRPV1 have been identified in human OCs and GPR55, and found to be expressed in both human osteoblasts (OBs) and OCs. Mouse OBs and OCs express CBR, CB2R, GPR55 and TRPV1. Studies of the innervation of the mouse bone have shown that CB1 and TRPV1 are expressed in sympathetic nerve fibers. CB1R activation inhibits bone production, whereas CB2R activation in OCs suppresses osteolysis activity, thereby preserving the bone tissue. In addition, OCs also express AEA and 2-AG, but with CB2R instead of CB1. Although norepinephrine is directly responsible for the activities of OCs and OBs, ECS is the main regulatory system of the bone by balancing the homeostatic relationship between hyperactive OCs and inactive OBs in osteoporosis, leading to increased bone resorption without compensatory bone formation. CBR agonists promote differentiation of mouse mesenchymal stem cells (MSCs) into OBs, and CB1R inactivation increased bone mass and prevented bone loss due to ovariectomy in a mouse model. CB2R was modulated in the genetic and phrenological processes, thus affecting bone cell activity in remodeling in both healthy individuals and patients. CB2R agonists increased bone mass by enhancing the number and activity of OBs, inhibiting the proliferation and regulation of OCs, and stimulating fibroblastic colony formation by myeloid cells. GPR55 receptor agonists promote bone loss by inhibiting OC formation and stimulates B function. TRPV1 agonist-induced overexpression of CB2R might be critical to reduce calciumentry into OCs, which could lead to over-activation of cells and increase bone resorption and bone loss. TRPV1 agonists together with CB2R agonists were reported useful for the treatment of osteoporosis. CBD enhanced the biomechanical properties of rat mid-femoral fracture healing. Micro-computed tomography (μCT) showed that the fracture callus size was transiently reduced by either CBD or THC 4 weeks after fracture but reached control level after 6 and 8 weeks. The callus material density was unaffected by CBD and/or THC. In contrast, CBD stimulated mRNA expression of Plod1 in primary OB cultures to encode an enzyme that catalyzes lysine hydroxylation, which in turn was involved in collagen cross-linking and stabilization. These data show that CBD can improve fracture healing and plays a critical mechanical role of collagen cross-linked enzymes. The bones of rats treated with CBD alone not only healed faster but the previous fracture was less likely to break in the future because of a strengthened fracture callus. Therefore, CBD provides research directions for the treatment and prognosis of fractures. CBD was known to inhibit FAAH, knowing that it could prevent the enzyme from breaking down bone forming compounds. An effective function of bone anabolic-antiresorptive is shared by many skeletal FAAs, which in turn, assist in the process of bone metabolism by interacting with CBR. Inhibition of the fatty acid amides (FAAs) degrading enzyme, FAAH, in which CBD is known to inhibit, may prove to be an effective therapeutic strategy for the treatment of bone fractures. At risk population: thin stature, chronic corticosteroid exposure, those taking PPIs, chemotherapy regimens, malnutrition, Vit D deficiency.

Low-grade, chronic inflammation has been found to correlate with increased bone loss and fracture risk, but increased BMD does not necessarily equate to decreased fracture risk. Aspirin (irreversible effect on platelets) and NSAIDs (reversible effect on platelets) works as a cyclooxygenase (COX) inhibitor via suppression of prostaglandin through both the COX-1 and COX-2 pathways, resulting in anti-inflammatory effects, such that aspirin use can decrease bone loss and controversially reduce fracture risk, by reducing chronic inflammation, but may increase the risk of bleeding after trauma or falls including elderly, postmenopausal females (primary osteoporosis usually developing within 10 to 15 years after menopause, and affects >10% of women after age 60 resulting in decreased quality of life and morbidity), recent boney injuries or fractures and those with osteopenia who take biphosphanates (greater bone loss found in the femoral neck, increasing the risk of hip fracture). Some NSAIDs selectively inhibit only the COX-2 pathway (minimal effect on platelets, and less GI upset or ulcers, but still carries a risk of increased cardiac disease), which, in turn, may decrease osteoclastic bone resorption and promote increased bone mineral density (BMD). However, in another study, NSAIDs use significantly increased risk for osteoporotic fractures in women aged 75 years and older during the β-year study period. The risk for nonunion in healing fractures was higher among patients receiving treatment with NSAIDs for >4 weeks, but its use for <2 weeks did not demonstrate increased risk for nonunion, and most surgeons recommend discontinuing its use 2 weeks prior to and following spinal fusion surgeries, (along with smoking cessation) such that alternative analgesics are prescribed postoperatively. Prolonged aspirin or NSAID use may also negate the effect of bisphosphonates, delay fracture healing, and increase risk for falling among older adults, but may used for acute myocardial infarction, secondary prevention of strokes and cardiovascular disease, and may prevent several cancers (usually after taking for 5 years). NSAIDs also can increase risks of upper and lower GI bleeds, GI upset and ulcers, hypertension, sodium retention, acute and chronic kidney disease, atrial fibrillation, liver disease, rare psychosis, affect gut microbiome, and possibly urologic cancers, especially with chronic use. CB2R agonists also inhibit COX pathways, have anti-inflammatory effects, and do not carry the risks of bleeding or significant GI upset.

GI: CB1R are found in the colonic epithelium, in the plasma cells of the lamina propria, in the smooth muscle (reduces peristalsis) and in the submucosal myenteric plexus, whereas CB2R have been identified in goblet cells of the epithelium, in Paneth cells, in macrophages, and in plasma cells of IBD patients. In a meta-analysis, cannabinoids were administered therapeutically in 37/104 experiments, prophylactically in 19/104 experiments, and prophylactically and therapeutically in 48/104 experiments. The results favored the cannabinoids in all of the last group including decreased severity of disease with improved remission, with a significant improvement in the perception of health, quality of life, social functioning, pain intensity and depression. About 15% of IBD patients (some involve pediatric) use medical Cannabis to relieve symptoms such as nausea, abdominal pain, diarrhea, and increased appetite. CBD by mediating CBR (also, with a minor effect on CB2R have in the enteric nervous system, ENS, inhibiting peristalsis of the upper GI tract (or intestinal in the context of colitis), and there is a known genetic association of the CB2R-63 variant with CD in children), reduced both luminal and crypt epithelial damage, inhibits the release of ROS and production of NO by neutrophils, with an increase in IL-12 and IL-17A (anti-inflammatory cytokine related to CB2R) and a decrease in IL-10 (pro-inflammatory cytokine); it also has been shown to inhibit JAK/STAT activators, including IFN-γ and inflammatory and immune response TNF, by may suppress T-cell-mediated antitumor immunity through the inhibition of the JAK1-STATs signaling, whereas CBD has potential contribute to drug interactions by affecting CYP drug metabolism. FAAH or MAGL blockers may be another strategy to improve IBD. THC reduces the levels of cytokines involved in the recruitment of neutrophils with reduction of myeloperoxidase (MPO) activity. CBG has been shown to reduce generation of inflammatory cytokines, reduce production of reactive oxygen species, reduce the number of macrophages and reduce the number of mast cells in experimental models of IBD. Non-ECS mechanisms including GPR55, PPAR-γ, and TRVP also contribute to cannabinoid effects on IBD.

Auditory: CBR1 are present in brain (including cochlear nuclei) and auditory structures (cochlea) for neural processing, whereas, CB2R (found in hair cells, which also includes TRPV1 receptors, as well as auditory ganglion and auditory nerve cells which may promote neurogenesis) for a protective and inflammatory mechanisms in the cochlea apparatus, and peripheral nerves as well neuroinflammation of the auditory cortex pathways (including brainstem). Mice bred without CB1R have high frequency hearing loss, and CB1R agonists mitigate this, possibly by improving attentional modulation and reduced anxiety. In another mouse model, with breeding for the absence of an enzyme catabolizing 2-AG (they did not manifest with hearing loss) displays significant brain region-dependent changes and macrophage induced increase lipopolysaccharide (LPS)-induced cytokine production.

Tinnitus has multiple diverse etiologies (including Covid-19) with an absence of a causative hypothesis, but bears similarities with neuropathic pain and epilepsy, both of which can be modulated by cannabinoids. The association between tinnitus and marijuana (and even greater occurrence with synthetic illicit cannabinoids) use in humans has been studied with contrasting results, in which experts have concluded that it was not possible to differentiate between causal association (Cannabis use increases tinnitus prevalence in one study's participants, in which 20%, or 20× risk, developed tinnitus within 1 day or worsen pre-existing symptoms), reverse causal association (tinnitus sufferers use more Cannabis than non-sufferers), and association due to external common cause (i.e., comorbid depression or social isolation, anxiety, which increases both tinnitus risk and Cannabis use, in which chronic tinnitus is associated with changes in attentional, memory, and limbic circuits). CB1R are present in brain (including cochlear nuclei) and auditory structures (cochlea) for neural processing, whereas, CB2R (found in hair cells, which also includes TRPV1 receptors, as well as auditory ganglion and auditory nerve cells which may promote neurogenesis) for a protective and inflammatory mechanisms in the cochlea apparatus, and peripheral nerves as well neuroinflammation of the auditory cortex pathways (including brainstem). Mice bred without CB1R have high frequency hearing loss, and CB1R agonists mitigate this, possibly by improving attentional modulation and reduced anxiety. In another mouse model, with breeding for the absence of an enzyme catabolizing 2-AG (they did not manifest with hearing loss) displays significant brain region-dependent changes and macrophage induced increase lipopolysaccharide (LPS)-induced cytokine production Pruritis: According to a 2020 review in the Journal of the American Academy of Dermatology, “increased endocannabinoids in neural tissue specifically decreases histaminergic itch and direct effects of cannabinoids on neuronal receptors increase the nociceptive threshold.” A treatment approach to chronic itching as a symptom, primarily related to allergies (may include contact, or systemic: immunologic, infectious, toxic, environmental or food), neurogenic or psychiatric etiologies, suggests that cannabinoids (often, topicals) may be good at relieving itch due to its ability to turn on and off skin nerves as well as its ability to positively impact keratinocytes (the primary skin cell in the upper layer of skin) and mast cells with CB2R binding that are responsible for immune responses in conjunction with integrative CB1R/TRMV1, and TRPV1 binding, which can be administered topically. In addition, the antimicrobial effects may ameliorate the underlying cause of infectious disease related pruritus (including shingles, HIV), as well as the itch symptoms associated with pregnancy, systemic chronic diseases including fibromyalgia, chronic fatigue syndrome, chronic autoimmune, diabetes, other endocrine, renal (uremic itch is common for those undergoing hemodialysis) or liver disease, several neoplastic syndromes including mastocytosis or mast cell activation syndrome, blood dyscrasias, leukemia, multiple other cancers (skin, breast, lung, stomach, colon, prostate, pancreatic), or as a side effect of medications including chemotherapy, radiation, UV light or sunburn.

Bladder: As related to wake control, ACh shows decreased turnover and increased levels in basal forebrain region. Detrusor contractions inhibition in mouse bladder that were induced by acetylcholine, with ranked potency CBG=THCV>CBD>CBDV, but not CBC. CBG has been shown to reduce acetylcholine-induced contractions in the human bladder.

Antiinflammatory effects due to CB2R: CB2R activation reduces inflammatory-joint disease markers are found in the human synovial tissue of patients with both rheumatoid arthritis and osteoarthritis, including pro-inflammatory cytokines (interferon [IFN]-c, interleukin (IL-12, IL-15, IL-17, IL-18), chemokines, chemical mediators, such as nitric oxide synthetase (NOS)-2, cyclooxygenase-2 (COX-2), matrix metalloproteinases (MMPs) and various other arachidonic acid metabolic by-products. CB1R presynaptic activation can reduce inflammatory biochemical mediators (bradykinin (BK), serotonin (5-HT), prostaglandins (PG) etc.) and the up-regulation of pain mediator nerve growth factor (NGF). The substance P (SP) and calcitonin gene-related peptide (CGRP) vasoactive neuropeptides, released from sensory nerve, have also role in inflammation. CBR demonstrate anti-nociceptive activity, whether used singly or in combination, with CB2R activity believed to affect microglial cells (a type of glial cells that protect and defend neurons from pathogens that have permeated the BBB) and thereby reduce neuro-inflammatory mechanisms. The CB2R is thought to be particularly important in central neuronal pain circuits, as agonist activity induces dopamine release in mid-brain areas, contributing to descending pain control and the placebo effect. THC may inhibit CGRP release, causing mesenteric vasodilation in rats, as postulated through CB1R agonist and probable TRPV1 antagonist effects.

Activated T lymphocytes and neutrophils found in the sinusoidal endothelium, also contribute to inflammatory microenvironment leading to hepatic stellate cell (HSC) activation and fibrogenesis. Hepatic Kupffer also activates HSC by expressing high CB2R, which mediates an anti-inflammatory effect by suppression of TNF-α and IFN-γ and stimulation anti-inflammatory cytokines such as IL-10 and also inhibit macrophage migration at sites of inflammation leading to anti-fibrogenic effects and decreases the neutrophil adhesion. Autoimmune liver disease (ALD) includes a spectrum of autoimmune hepatitis (AIH, with genetic, and possible etiologies including environmental toxins and prior viral hepatitis A, B, or C, cytomegalovirus (CMV), paramyxovirus, and most recently retrovirus infections, which can be simulated by conanavalin A), primary biliary cirrhosis (PBC), primary sclerosing cholangitis (PSC) which may be combined in “overlap” syndromes, have low levels of circulating T-cells (especially CD⁴⁺) and impaired suppressor cell function. They can present with symptoms of fatigue and myalgia in AIH or fatigue and pruritus in PBC and PSC and may progress to cirrhosis requiring liver transplantation. CD⁴⁺ T-cells increase with THC in disease models, presumably through CB2R activation, which may be helpful initially. ECS also can upregulate CBR on hepatocytes, such that in human chronic HCV (and simulated AIH), in which a sustained immune response against the virus may be crucial for regulating the infection, and therefore, smoking marijuana (at higher dosing, chronically), may suppress this through its CB1R effects, contributing to chronic infection, steatosis and liver fibrosis.

CB2R agonists (via ECS and multiple non-ECS pathways) can ameliorate the progression of osteoarthritis (OA), by reducing proinflammatory cytokines, primarily IL-1 and TNFα, as well as IL-6, IL-8, IL-15 and IL-18, CCL2/3/4/5/19/21 and CXCL12 produced by macrophages involved synovitis and A progression in chondrocytes. In rheumatoid arthritis (RA), progression is correlated with TNFα, IL-6, IL-1, IL-17, IL-23, IL-21, IL-12, GM-CSF, CXCL, and type 1 interferons (IFNs), with elevated levels of circulating levels of IFN-γ, TNFα, IL-17 and IL-12. These biomarkers can be reduced with anti-inflammatory therapies including CB2R agonists. Cannabinoids may also modulate central sensitization, which can commonly occur from peripheral arthropathies or other painful conditions (including fibromyalgia or soft tissue musculoskeletal syndromes), which may also involve CB1R (analgesic effect). They also prevent osteoporosis by strengthening and increasing bone growth, with decreases receptor activator of nuclear factor KB(NFκB) ligand/osteoprotegerin ratio (RANKL/OPG, which may also modulate neuroinflammation), an important indicator of bone homeostasis and remodeling favoring osteoclastic activity. CB2R may reduce cancer-induced osteolytic destruction and improve fracture healing with a concomitant reduction in pain.

It has been shown that with CB2R activation, neutrophil and macrophage infiltrations were reduced, and expressions of monocyte chemotactic protein (MCP)-1, stromal cell-derived factor (SDF)-1, Interleukin (IL)-6, IL-1(3, tumor necrosis factor (NF)-α, transforming growth factor (TGF)-31 and vascular endothelial growth factor (VEGF)-A were decreased. Keratinocyte proliferation and migration were enhanced. Wound re-epithelialization was accelerated. Fibroblast accumulation and fibroblast-to-myofibroblast transformation were attenuated, and expression of pro-collagen I was decreased.

Neuroinflammation: Cannabinoids (including endocannabinoids) can reduce inflammation, and since they cross the BBB, may be effective neuroprotective agents. Neuroinflammatory processes involve abnormal protein dynamics, oxidative stress with reactive oxygen species, mitochondrial dysfunction, DNA damage, dysfunction of neurotrophins, and all are considered common pathophysiological mechanisms. Neuroinflammation is a defense mechanism that initially protects the brain by removing or inhibiting diverse pathogens. This inflammatory response can have beneficial effects by promoting tissue repair and removing cellular debris (autophagy, a normal physiologic phenomenon for recycling intracellular contents using enzymes contained in lysosomal organelles and providing continuing nutrition to the cell). Sustained inflammatory responses can overwhelm the system, and become detrimental, as they inhibit regeneration. Inflammatory stimulation can persist due to endogenous (e.g., genetic mutation and protein aggregation) or environmental (e.g., infection, trauma, and drugs) factors. The persistent inflammatory responses involve microglia functioning as (1) detecting changes in their CNS environment using their sensomes, (2) migrating to injured sites, remodeling synapses, and maintaining myelin homeostasis (3) protecting against injurious stimuli, as well as the involvement of astrocytes and can lead to neurodegenerative diseases. microglias actually possess a spectrum of polar phenotypes: neurotoxic, M1 (classical activation) and M2 (alternative activation) neuroprotective. Astrocytes can be either pro-inflammatory or immunoregulatory mediators based on the type and stage of neurodegenerative diseases and the regional location in the CNS. M1 Microglia induced neuronal damage involves pathogen-associated molecular patterns (PAMPs) and damage-associated molecular patterns (DAMPs) which are modulated by their cellular receptors such as toll-like receptors (TLRs), nuclear oligomerization domain-like receptors, and viral receptors resulting in the detrimental production of proinflammatory cytokines (tumor necrosis factor (TNF)-α, interleukin (IL)-1β, IL-16 IL-6, nitric oxide (N0), proteases, and chemokines, including the C-C motif chemokine ligand 2 (CCL2) and IL-18); whereas, the activation of neuroprotective M2 microglia (by IL-4, IL-10, IL-13, and transforming growth factor-β(TGF-β) results in beneficial cytokines (FIZZ1, Chitinase-β-Like-3 (Chi313), Arginase 1, Ym1, CD206, insulin-like growth factor 1 (IGF-1), and Frizzled class receptor 1 (Fzd1)). As a proposed sequence of events, there is an abnormal phenotypic messenger RNA based protein involved, with toxic properties, which accumulates, based on normal degenerated mitochondrial catabolism from the axon terminal is released intracellularly, through a leaky lysosome as the result of a faulty repair mechanism, which is then released, extracellularly, into the synapse. The enzyme involved in this regulatory pathway produces phosphatidylinositol β-kinase, a lipid which in turn, activates protective proteins for repair and stabilization of the lysosome membrane; this effect is modulated by CB1R binding, which can be applied to neurodegenerative (i.e., Tau in AD), brain neoplasm (astrocytoma) or addiction models. The extracellular release of a toxic protein induces a glial cell reaction (neuroinflammation) with a cascade of proinflammatory cytokines released (and/or inhibition of anti-inflammatory cytokines), and causes dysfunctional phagocytosis, preventing re-absorption, thus causing neuronal toxicity at the intercellular site. There is also (often, in combination) a dysfunctional component involved with mitochondrial breakdown during autophagy in which the exosomes (subcellular packet of RNA, which can penetrate BBB) are then re-absorbed by the cell membrane, with retrograde transport into the neuronal nucleus, causing cell injury and subsequent death. Both pathologic processes, over time, with differing contributing factors of dysfunctional autophagy, leads to the clinical manifestations, with a positive feedback loop (vicious cycle) in which contiguous neurons become pathologically involved, until there is not enough reserve of unaffected neurons to carry on their function. Glial cells, including astrocytes, oligodendrocytes, and microglia, can regulate neuronal activities, with diverse functions, including neuroimmunologic effects.

Antimicrobial: Several studies have correlated CBR deficiency with increased inflammation and tissue damage following influenza virus infection, and their activation can impair immune responses induced by the virus. Such data and effects of ECS on viruses such as: immunodeficiency viruses (human-HIV and simian), hepatitis C virus (HCV, but not B), respiratory syncytial virus (RSV), borna disease virus or vaccinia virus, orthopoxvirus, Kaposi's sarcoma-associated herpesvirus (KSHV), herpes simplex virus (HSV), Theiler's murine encephalomyelitis virus (TMEV) and others. These studies can indicate that cannabinoids may serve as antimicrobial and anti-inflammatory therapeutic agents by modulating immune cell migration and cytokine/chemokine secretion.

The use of cannabinoids as antimicrobial adjuncts, rather than as standalone therapeutics, has been considered. For example, the combination of polymyxin B and CBG is efficient at killing E. coli and CBD seems to enhance the efficacy of polymyxin B against multiple strains of A. baumanii, K. pneumoniae and P. aeruginosa. CBD alone or combined with bacitracin topically has shown efficacy against S. aureus and L. monocytogenes. Silver (also antimicrobial) nanoparticles loaded collagen hydrogels enhanced with C. sativa oil extract exhibited prolonged (7 day) and efficacious anti-microbial activity against both gram-positive (S. aureus ATCC 29213) and Gram-negative (P. aeruginosa ATCC 27853) bacteria, with the Cannabis oil substantially decreasing epithelial cell cytotoxicity. CBD also has enhanced the bactericidal action of selected antibiotics against gram-negative bacteria. This same concept could be applied to anti-viral therapy.

Oral administration of probiotic, Lactobacillus acidophilus was shown to combat inflammation and nociception through increasing the expression of the CB2R in intestinal epithelial cells, suggesting that they might work together to halt inflammation and nociception. In support of this, it has recently been shown that THC reduces inflammation and adiposity in mice by increasing the accumulation of mucin-degrading bacteria, Akkermansia municiphila. Akkermansia municiphila supplementation was shown to reduce systemic inflammation in mice, further supporting the notion that microbiota contributes to the anti-inflammatory and analgesic effects of oral cannabinoids.

CB2R agonists can slow planktonic growth inhibit Vibrio harveyi with reduced biofilm formation and bacterial motility. The mechanism of action of CBG has been elucidated by uniquely disrupting the integrity of the cytoplasmic membrane of methicillin resistant Staphylococcus aureus (MRSA, gram positive bacteria) persisters (after exposure to conventional antibiotics such as gentamicin, ciprofloxacin and vancomycin) of gram-positive bacteria and demonstrate in vivo efficacy in a murine systemic infection model caused by MRSA. Unlike most other cannabinoids which are also effective against Gram-negative organisms whose outer membrane is permeabilized, CBG acts on the inner cell membrane, and represses MRSA biofilm formation. Comparing all cannabinoids, CBG exhibited the most potent anti-biofilm activity and most intensively inhibited the growth of gram-negative pathogens such as A. baumannii, E. coli, Klebsiella pneumoniae, and Pseudomonas aeruginosa. CB2R agonists exhibit antifungal activity to Candida albicanse, Cryptococcus, neoformans A, tinea pedis pathogens, Trichophyton interdigitale and Trichophyton rubrum.

CB2R agonists (i.e., CBD) can act as an anti-inflammatory and antimicrobial effects on both gram-positive bacteria (Bacillus subtilis, Bacillus pumilus, S. aureus, and Micrococcus flavus) and gram-negative bacteria (Proteus vulgaris and Bordetella bronchiseptica). The synthetic cannabinoid HU-210 has an inhibitory effect on quorum sensing (OS) and QS-dependent properties, such as bioluminescence, biofilm formation, and swimming motility of Vibrio harveyi, without affecting its growth), together with its ability to interfere with the Auto inducer-2 (AI-2) quorum sensing signal cascade (ads to communicate and to regulate various functions including biofilm formation, stress response and virulence factor expression) helps stimulate the bone, restore, and enhance oral health, and also has shown some promise against oral bacteria (causing cavities), in conjunction with oral hygiene including at-home brushing, flossing, mouthwash, dentifrices and dental scaling. Cannabinoid-infused (CBD or CBG, both <1% by weight) mouthwash has been found as effective as chlorhexidine (0.2%, CHX, a prescription antimicrobial used in mouthwash, which has been found to be more potent than hydrogen peroxide (H₂O₂) 1.5%) at an inhibiting bacterial activity, and another study found that cannabinoids (CBC, CBN are both more effective, followed by CBD, CBG, and CBGA) were effective in reducing bacteria counts in dental plaques (both soft and calcified hard complex biofilm, by adherence of primary colonizing bacterial species to the enamel salivary pellide followed by secondary colonizers through interspecies interactions and communications, commonly associated with gingivitis, periodontal disease, dental cavities, bad breath (halitosis), bleeding gums, tooth decay, and tooth loss, as well as systemic inflammatory conditions), and may be a safer alternative than antibiotics to reduce the development of drug resistance. Several other natural herbal extracts, such as pomegranate, algae, triphala, tulsipatra, neem, aloe vera, and cinnamon, and 4 essential oils, eucalyptol, menthol, methyl salicylate, and thymol, have been reported to be effective against dental plaque bacteria, and thus may be combined with a cannabinoid.

Other: Both CBG and CBD, as modulated through CB2R binding, increase the free radical scavenger, with a short duration of action, nitric oxide (NO). NO can be produced constitutively by endothelial cells or can be inducible (from infection or acute inflammation) resulting in smooth and skeletal muscle relaxation, vasodilation (and is considered essential for penile tumescence, but also has effects for cardiovascular circulation including angina, Reynaud's disease, pulmonary hypertension and placental perfusion), cellular metabolism including chondrocytes (preventing resorption and inhibits proteoglycan degradation), inhibits platelet function and mitigates atherosclerosis, gastro protection and GI motility, has inhibitory GABA neurotransmitter function (found in the accessory olfactory bulb and granule cells of the cerebellum, hippocampus, and during fetal development, which can increase 5HT and DA, also oxytocin, LHRH, osmoregulator peptide, CRH and ACTH, and is involved with learning, memory, mood, sleep cyde, appetite, respiration, thermal regulation and opioid analgesia); it also rapidly reduces free oxygen radicals with antioxidant and anti-inflammatory effects, resulting in a cascade of immune reactions with antimicrobial action.

COVID-19 effects: This Product has intended mechanism of effect that can enhance the care and treatment of Covid-19 particularly with effects on the respiratory tract, which is a serious disease associated with morbidity that has substantial impact on day-to-day functioning. Several proposed actions may include acting as a direct antimicrobial, improving microbiome, improving defensive immunologic/anti-inflammatory response, and preventing sequelae (“long Covid-19”) by neuroprotection. They may also symptomatically help with sleep, anxiety, depression, pain, fatigue, cognition, GI, sexual, respiratory, cardiac, liver, renal, mucosal, endocrine, appetite, metabolic, withanti-ageing and protective effects.

Angiotensin 2 converting enzyme (ACE2) is a trans-membrane type I glycoprotein, zinc dependent, metalloenzyme and carboxypeptidase, which provides homeostatic regulation, found in heart (endothelium of coronary arteries, fibroblasts, cardiomyocytes, epicardial adipocytes), vessels (vascular endothelial and smooth cells), skeletal muscles, gut (enterocytes of the small intestine and colon), lung (macrophages, tracheal and bronchial epithelial cells, type 2 pneumocytes), kidney (luminal surface of tubular epithelial cells), testis (spermatogonia, Leydig cells, and Sertoli cells), adipocytes (and their resident macrophages), and brain (neurons and glial cells). This organ- and cell-specific expression of ACE2 supports its role in the regulation of cardiovascular and renal function, particularly the renin-angiotensin-aldosterone-system (RAAS) as a potent sympathetic vasoconstrictor, hypercoagulable, aldosterone releaser (from the adrenals related to potassium levels), oxidant, and modulates multiple signaling pathways. Angiotensinogen produced in the liver is enzymatically cleaved by renin, (produced by the kidney), into angiotensin I (inactive), which is then converted into bioactive angiotensin II, via ACE2, and subsequently, converted into angiotensin 1-7. Inhibition of ACE2 has been an important mechanism to facilitate the action of antihypertensive drugs but is also significantly involved in the immune system. Its inhibition may lead to protect from pathological muscle remodeling and muscle insulin resistance. ACE2 protects against aging-associated muscle wasting (sarcopenia). Bioactive peptides, found in plants including hempseed have known ACE2 inhibitory effects, which can be used as a metric for potency. ECS activation reduces angiotensin II levels, through renin-angiotension system, lowering blood pressure, and affects SARS-CoV-2 entry.

In a preclinical study, ACE2 receptor blockade (ARB) promoted muscle regeneration and reduced fibrosis in damaged skeletal muscle that can potentially improve lower extremity perfusion and repair ischemia-related skeletal muscle damage. In mice, compared with placebo, telmisartan (a potent ARB), increased type I muscle fibers, oxygen consumption, and running endurance, with gastrocnemius muscle biopsy measures (satellite cell number; type I muscle fiber composition; peroxisome proliferator activated receptor [PPAR]δ; PPAR γ coactivator 1α; adenosine monophosphate-activated protein kinase quantity and activity; and the relative gene expression of PPAR δ, PPARγ coactivator 1a, adenosine monophosphate-activated protein kinase, FOX03A, and SIRT1) showing regeneration. CB1R antagonists may enhance angiotensin II-mediated responses (mainly vasoconstrictor effects) or reduce them (mainly CNS-mediated effects), such that cannabinoids modulate ACE2. This is a bidirectional effect, due to heteromerization of CB1R and ACE2 receptors, with therapeutic application for Covid-19 interventions. ACE2 receptors have been abundantly found on the surface of host gateway tissues (heart, lung, liver, kidney, but also oral, nasal mucosal epithelium) to the Covid-19 virus (SARS-CoV-2). ACE2 (specifically, the N-terminal portion of the viral protein unit S1 binds to a pocket of the ACE2 receptor) and trans-membrane protease serine 2 (MPRSS2), the latter primes the viral spike proteins, are correlated with viral virulence. ARBs have also been associated with reduced seizure risk.

ACE2 expression declines with age (in males>females, with androgen sensitivity), diabetes, obesity, atherosclerosis, hypertension, metabolic syndrome resulting in a pro-inflammatory state that may explain the increased severity and comorbid complications observed in older adults who exhibit reduced basal serum ACE2 levels. As a result of ACE2 down-regulation, SARS-CoV-2 infection in older adults induces aggressive secretion of pro-inflammatory cytokines (presumably the virus binds more efficiently to the host's cell membrane receptors, but with less dilution of effect, since there are less total receptors to bind initially, but then the virus upregulates this receptor expression to perpetuate infectivity). This is associated with decreased activation of Sirtuin I (SIRT1, a nuclear nicotinamide adenine dinucleotide-NAD+-dependent protein deacetylase) genetic expression of ACE2 receptors (its activation is also modulated by Vitamin C, Vitamin B3, Vitamin D, resveratrol, curcumin, caloric restriction, exercise, potassium, nicotine, metformin, antihypertensive ACE2 inhibitor drugs; and probably, melatonin, catechins and quercetin (the latter three, found in hemp)). Once causing host infection, SARS-CoV-2 then enters endothelial cells and other immune cells, causing an up-regulation of ACE2 expression, potentially leading a viscous cyde with a cytokine storm. CBD induces neuronal hippocampal autophagy and improves neuronal health associated with SIRT1 mediated longevity and is likely to affect other cells including those with ACE2 receptors.

The gut microbiota has important roles in viral entry of ACE2 expression, immune homeostasis, and crosstalk between the gut and lungs in which Covid-19 infection was associated with altered intestinal microbiota and correlated with inflammatory and immune responses, implicating a ‘gut-lung axis’. Animal models of influenza virus infection have uncovered mechanisms by which the microbiome influences antiviral immunity, and in turn, the viral infection was shown to disrupt the intestinal barrier of mice by damaging the gut microbiota. COVID-19 patients lose commensal taxa of the gut microbiome during hospitalization, and persistent microbiome alterations are found in patients with long-term complications from COVID-19. Secondary bacterial infections occur in 12-14% of COVID-19 patients. In those receiving broad spectrum antibiotics have an almost 2-fold increased risk of toxic shock. Gut microbiome dysbiosis (especially with Enterococcus) in antibiotic-treated COVID-19 patients is associated with microbial translocation and bacteremia. Cannabinoids potentially allow an antimicrobial effect, along with optimizing gut microbiome resulting as an advantage in the management of Covid-19. Cannabinoids potentially allow an antimicrobial effect, along with optimizing gut microbiome, which is advantageous in the management of Covid-19 especially for sepsis and physiologic stress.

Oral mucosa shows increased expression of ACE2 receptors and TMPRSS2 in the human tongue epithelia and saliva during severe acute respiratory syndrome of Covid-19. In females treated for tongue cancer and Covid-19, who were alcoholics, the expression of TMPRSS2 was higher in the stratified squamous epithelia of human tongue tissue, salivary glands, taste buds, and perineurium than in murine tissue, whereas it is the reverse for ACE2 receptor assays, which may suggest that specific ratios of these receptors may affect viral virulence. Further analysis revealed there was no significant difference in ACE-2 receptor expression in human tongue tissue according to age, sex, alcohol intake, or smoking, whereas TMPRSS2 expression in the tongue was significantly up-regulated in this subset of patients, which may explain the etiology of dysgeusia (loss of taste). Smoking habit was associated with a potential increase in TMPRSS2 expression. Arterial hypertension is associated with an increased incidence of epilepsy, with the RAAS appearing to act as a central mediating signaling pathway. Results from animal studies suggest that ACE2 blockers (which also can induce ACE2 receptors through CB1R; however, this does not result in any clinically greater risk for COV-SAR2 invasion) could inhibit epileptic seizures. In animal studies, losartan displays an anti-epileptogenic effed by inhibiting the albumin-induced transforming growth factor β(TGFB) signaling cascade, which has been implicated in enhancing neuronal excitability and excitatory synaptogenesis after blood-brain barrier damage and inflammation. CB1R agonists (including non-psychoactive cannabinoids and hemp seed oil) can reduce blood pressure because CB1R are expressed in smooth muscle and endothelial cells, through its modulation of vasoactive angiotensin II. CBD and/or unique high-CBD/low-THC Cannabis extracts downregulate ACE2 and TMPRSS2 proteins expression in COVID-19 gateway tissues, through miR-200c-3p- and let-7a-5p-targeting via AKT (pivotal role in the production of cytokines, and when combined with PI3K, has a role in cell proliferation, cell survival, the cell cyde, apoptosis, glycogen metabolism, and inflammation) pathway downregulated AE2 and TMPRSS2 in lung fibroblast WI-38 cells through AKT-mediated inhibition.

TGFB actively regulates immunosuppressive lymphocyte CB2R expression in an autocrine and paracrine manner, in which THC can induce its cytokine release. This cascade can lead to brain hypo-perfusion and accelerate cognitive decline in the elderly, which can worsen pre-existing cognitive dysfunction, mediated through prohibitins, a class of mitochondrial proteins. The neuroinvasive potential of SARS-CoV-2 may result in senescence of several different CNS cell types, including glial cells (oligodendrocytes, astrocytes, ependymal, along with their progenitor cells that provide physical and metabolic support to neurons, including insulation and communication, and nutrient and waste transport in the CNS; whereas, satellite and Schwann cells have a similar function in peripheral nervous system), and neural stem cells (particularly, hippocampal progenitor cells modulated by IL-12 and IL-13) that can differentiate into neurons that integrate into the granule layer. SIRT1 regulates many endocrine functions, protects organisms from oxidative stress-related cellular events, promotes DNA stability, and decreases various age-related disorders, such as neurodegenerative disease, metabolic abnormalities, and cancer. Cannabinoids may target ACE2 receptors because they are smaller molecules (improve oral absorption), lipophilic and can enter the BBB. ECS activation reduces the levels of transforming growth factor (TGFB1), and modulates fibrogenesis, useful in wound healing.

A higher concentration of IL-6 is found in hospitalized Covid-19 patients with delirium, which ads by inducing the infected hippocampal cells to produce IL-12 and IL-13, a target for antiviral drugs. COVID-19 may affect the CNS in at least six (often, combined) mechanisms: (1) the immune response to SARS-CoV-2 in the respiratory system (or any other point of entry) may cause neuroinflammation (increasing cytokines, chemokines and immune cell trafficking in the brain and inducing reactive states of resident microglia and other immune cells in the brain and brain borders)(2) SARS-CoV-2 directly infect the CNS (rare)(3) SARS-CoV-2 may evoke an autoimmune response against the CNS (4) reactivation of latent herpes viruses like EBV (possibly shingles) or super infection (pneumonias) may trigger neuropathology (5) cerebrovascular and thrombotic disease may contribute to ischemic CNS changes, disrupt BBB function, and further contributing to neuroinflammation and/or ischemia of neural cells. (6) poly-systemic manifestations including pulmonary and multiorgan dysfunction occurring in severe COVID causing sepsis, hypoxemia, hypotension and metabolic disturbances that can negatively affect neural cells. These mechanisms and onset or duration may occur variably in some individuals. For example, neuroinflammation triggered by the immune response to the respiratory system infection and consequent dysregulation of neural homeostasis and plasticity is likely a more common mechanistic principle that occurs even after mild disease in the acute phase, while direct brain infection is likely an uncommon mechanism associated with severe COVID-19.

Cannabinoids (especially nonpsychoactive) have a unique role in at least symptomatic care for all of the above scenarios, and holistic wellness. CB2R agonists can ad by down-regulating both ACE2 and TMPRSS2 proteins, by reducing cytokine interferon-gamma (IFN-γ) and paracrine mediator, TNF-α, and this effed can be achieved topically, possibly used as a mouthwash.

Cannabinoids can inhibit SARS-CoV-2 entry and replication in several ways. They can bind to and inhibit SARS-CoV-2Mpro by blocking translation; they also act as CB2R agonists, reducing pro-inflammatory cytokine levels in lung cells The SARS-CoV-2 genome encodes several proteins (already identified 25 proteins) that the virus needs to infect humans and replicate itself Among these, SARS-CoV-2Mpro, the glycoprotein (S), notorious spike (S) protein, which recognizes human ACE2 in the initial stage of infection, chymotrypsin-like main protease, papain-like protease, the RNA polymerase, which synthesizes viral RNA, two proteases, which leave viral and human proteins, and the RNA-cleaving endo-ribonuclease are known to play an important role in the progress of SARS-CoV-2. Exogenous CBD administration has been shown to suppress inflammatory transcription factors such as AP-1, NF-κB, and nuclear factor of activated T-cells (NFAT). As a result, cytokines such as IL-6, IL-1b, IL-1a, granulocyte macrophage colony-stimulating factor (GM-CSF), and TNFα are suppressed in various cells and tissues, particularly M2 phenotype macrophages (rich in luster of differentiation-CD16 with poor migration ability, as compared to the, pro-inflammatory, migratory M1 “classic” macrophages, which are CD14-rich). Differentiation of T-helper (Th)17 cells, also shown to be suppressed by CBD, is promoted by IL-6. In murine models of chronic asthma, as a result of CBD administration, cytokine levels of IL-4, IL-5, IL-6, IL-13, and TNFα have been shown to decrease, thereby reducing fibrosis and airway inflammation. Four bioactive peptides (GVLY, IEE, LGV, and RVR) extracted from hemp seed oil had ACE-inhibitory activity, a potential agent to inhibit entry of SARS-CoV2 into the cells. For adult respiratory distress syndrome (ARDS), found in severe Covid cases, CBD may increase apelin levels and reduce cytokines.

Human participants, mice, and organoid cultures, the researchers found that long COVID was tied with a decline in serotonin, in turn, disrupts connectivity between the peripheral nervous system and the brain, causing neurologic symptoms due to reduced vagus nerve function, among other roles. A viral reservoir lingering in the gut appeared to trigger inflammation that decreased intestinal absorption of tryptophan, serotonin's precursor molecule, resulting in memory loss and hippocampal dysfunction. Non-psychoactive cannabinoids modulate serontonin levels.

A retrospective cohort study in 2022 suggests that for the active Cannabis users (4% of sample) who were hospitalized with COVID-19, they were associated with lower levels of inflammatory markers on admission; furthermore, they had better clinical outcomes compared with non-users including received less COVID-19 adjunctive therapies and had a decreased need for intensive care units (ICU) or mechanical ventilation care. However, another study found that COVID-19 patients with substance use orders (grouping them all together but not isolating CUD) have worse outcomes compared to general COVID-19 patients, including increased hospitalization and death. A 2022 laboratory study from researchers at Oregon State University, notably, found that certain cannabinoids can potentially prevent COVID-19 from entering human cells. But as doctors at UCLA have noted, that study focused on CBGA and CBDA under lab conditions, but did not assess marijuana smoking by patients themselves. It has been hypothesized that marijuana's has the potential to inhibit viral entry into cells and prevent the release of proinflammatory cytokines, published in a 2023 study, with significantly lower rates of intubation (6.8% vs 12%), acute respiratory distress syndrome (ARDS)(2.1% vs 6%), acute respiratory failure (25% vs 52.9%) and severe sepsis with multiorgan failure (5.8% vs 12%). They also had lower in-hospital cardiac arrest (1.2% vs 2.7%) and mortality (2.9% vs 13.5%). A prior 2022 study came to a different conclusion, finding that Cannabis use was associated with a lower chance of getting COVID but also with more serious infections.

GABA can reduce disease severity, viral load in the lungs, and death rates in SARS-CoV-2-infected mice, with beneficial effects still observed with viral variants; it can also inhibit autoimmune diseases such as type 1 diabetes, multiple sclerosis, and rheumatoid arthritis in mouse models. CB2R agonists can increase GABA levels. GABA receptor activation of immune cells inhibits the inflammatory actions for antimicrobial and autoimmune effects. GABAA receptors are found on both presynaptic and postsynaptic neuronal cell membranes and are primarily involved with smaller interneurons (contained in 20-60% of brain nuclei, which communicates with the larger purkinje and pyramidal neurons) that also contain CBR. These GABAergic neurons also have other specific receptors that bind benzodiazepines, alcohol and barbiturates, with synergistic effects that are non-competitive. The simultaneous binding of at least two GABA agonists to the extracellular site are required to allow the pore to open, causing chloride ions to enter the cell with hyperpolarization of the cell membrane and inhibition of action potentials. The coordination and magnitude of the synaptic communication determines the change of voltage in the post-synaptic cell and the strength of the signal. A reduction in the number of presynaptic cells or incoordination of firing results in weakening of the signal. Synaptic plasticity occurs as the result of strengthening of these synapses over time for long term potentiation, and requires coordination of firing of the pre and post synaptic cells within a window of 20 msec. There is a balance in the regulation of excitation and inhibition that allows the brain to physically adapt for learning and memory. Changes in neuronal activity and excitation by glutamate release may initiate off-setting activation of inhibitory inputs through GABA interneurons. In both activation and inhibition of the synaptic signal, retrograde ECS modulation by either depolarization-induced suppression of inhibition (DSI, associated with GABA), or excitation (DSE, associated with glutamate), likely mediates synaptic depression. Since ECS and GABA neurons are collocated in many areas of the brain (particularly in the prefrontal cortex and corpus striatum, hippocampus (60% of neurons co-express CB1R; and sustained CB1R agonists downregulate GABA(A), and is accompanied by neuronal hyperexcitability believed to be explained by both presynaptic GABA release mechanisms and postsynaptic GABA(A) receptor function), hypothalamus, and cerebellum, but less so in the globus pallidus in rodents), this close proximity may explain how CB1R binding influences the GABA system. In addition, a subpopulation of CB1R in dose proximity to the Ca2+ channel, namely basket cells (multipolar GABAergic interneurons, and less so for the dendritic-layer microglial cells found in the meninges and paraventricular regions, which are activated when it detects antigen, thus initiating neuroinflammation as the first line of defense) participate in the ECS modulation of GABA release.

Exposure of rats to CB1R agonists in adolescence will result in disinhibition of the prefrontal cortex when they become adults, which could be reversed with a GABA agonist. It has been concluded in normal neural development; ECS may downregulate GABA effects in the prefrontal cortex and may explain the neuropsychiatric and substance abuse effects of cannabinoids in youths vs. adults. In humans, reduced levels of GABA and glutamate in the anterior cingulate cortex (ACC, which surrounds the corpus callosum and communicates with the prefrontal, parietal and limbic brain structures) of adolescents were observed in habitual users of marijuana. This may have an application for GABA therapy in those with substance use disorders including Cannabis. Although gabapentin (a calcium channel modulator) is a structural analog of GABA, it does not alter GABA activity or receptors although it may increase GABA synthesis and non-synaptic GABA release and has been used clinically to reduce recreational THC consumption. Blocking the GABA(A) receptor accentuated the psychological effects of THC including psychoses and anxiety. A GABA reuptake inhibitor tiagabine taken alone, or with THC, enhanced the discriminative-stimulus, self-report and performance of subjects who were trained to detect the difference between THC and placebo, which was determined to be modulated by both GABA receptors; also GABA(B), part of the GPCR superfamily, resulting in inhibition of AC and cAMP formation, inactivating voltage-gated Ca2+ channels and K+ channels (which also binds badofen for motor spasticity and movement disorders, seizure effects), found in hippocampus, thalamus, basal ganglia, hypothalamus, brainstem, spinal cord, sympathetic ganglia, peripheral nerves, gut, gallbladder, urinary bladder, heart, blood vessels, platelets, lung, liver, kidney, synovial joints, uterus, oviduct, ovary, adrenal gland, thyroid, and pancreas; has circuits connecting to NAc which interacts with dopamine pathway and inhibits the reward system; GABA(B) receptors are involved at least in part with the effect of elevated GABA on cannabinoid-related behaviors, in which badofen, a GABA(B) agonist enhanced the effects of THC. The main precursor of GABA (an amino acid, found initially in bacteria, plants; and animal's CNS and retina) is glutamic acid, an excitatory neurotransmitter itself. GABA is synthesized by the irreversible single-step α-decarboxylation of glutamic acid by the enzyme glutamic acid decarboxylase (GAD, with 2 subtypes) in a coupled process with the conversion of cytosol glutamate to storage of intravesicular GABA via vesicular transports systems (VGAT) for the sequestration. When GABAergic cells receive a depolarizing stimulus, vesicular fusion and exocytosis occurs and GABA is released into the synaptic deft. GABA signaling is primarily terminated by its reuptake into both neuronal and glial cells through membrane transporter systems. Through this uptake system the presynaptic cytosol and vesides can reuse GABA.

Astrocytes also express membrane transporters systems for GABA and play a significant role in GABA metabolism. When reuptake occurs in these non-neuronal cells or non-GABAergic cells, the availability of GABA as a neurotransmitter is reduced. GABA is catabolized by the enzyme GABA Transaminase (GABA-T which is widely expressed in both central and peripheral systems unlike GAD), and be converted to glutamate, which in turn, may be recycled to form GABA by GAD; this may help limit exogenous GABA from influencing CNS activities. GABA also gets catabolized to succinate semialdehyde, which then enters the Krebs cyde for further metabolism. ECS acts in a retrograde fashion to neuromodulate the forward direction of chemical communication. Because of their highly lipophilic properties, endocannabinoids are not stored in vesides but are synthesized from membrane lipids only when required. Once released, the endocannabinoid diffuses to its' receptor target on the presynaptic neuron and helps regulate overall neurotransmission. In the brain, the presynaptic receptor is predominantly CB1R with limited CB2R found in microglia and other tissue. Eventually the endocannabinoid is released by the CBR and taken up by either the pre- or postsynaptic neuron for final degradation. The endocannabinoids are synthesized in the post-synaptic membrane only after the cell is activated and then rapidly degraded after binding to the presynaptic CBR; the effect of stimulation is localized and limited in duration, similar to GABA and other neurotransmitters. In addition, although the binding of AEA and 2-AG (more abundant) is primarily to the CB1R, they also activate non-ECS receptors, including GABAergic neurons. There can be multiple stimuli to elicit AEA release including lipopolysaccharide (LPS) which can include endotoxins in the outer membrane of gram-negative bacteria that plays a critical role in the protection of the microbe. Exposure to macrophages activates LPS to defend the bacteria and numerous lipid mediators including AEA are released. 2-AG serves an important role not just in the formation of an endocannabinoid but also in the production of proinflammatory molecules. DSI was first reported in the Purkinje cells of the cerebellum and later in hippocampal pyramidal cells, through activation of the CB1R and is considered the classic example how endocannabinoids regulate neuronal behavior through retrograde signaling and suppression of GABA release in presynaptic neurons.

A high-fat diet results in an increased proportion of lipopolysaccharide (LPS)-expressing bacteria. LPS have been previously shown to promote the synthesis of ECS ligands via the activation of the LPS receptor, which increases gut permeability through the down-regulation of the tight junction proteins, thereby increasing the plasma LPS levels. Heightened plasma LPS hyper-activates ECS in adipose tissue, promotes inflammation and adipogenesis (inducible through CB2R). Prolonged promotion of pro-inflammatory states and fat mass development leads to the dysregulation of adipose tissue metabolism, a hallmark of metabolic syndrome. Hence, the down-regulation of ECS in the gut has been suggested as a viable therapeutic approach against metabolic syndrome; this can possibly be accomplished by altering the gut microbiota. Short-chain fatty acids (SCFAs, e.g., C1: formic, C2: acetic, C3: propionic, C4: butyric, isobutyric, C5: valeric, isovaleric, and methybutric acids) are a lass of the well-known beneficial metabolites produced by gut microbiota by fermenting fiber rich plant-derived foods and absorbed via enterohepatic circulation of the portal vein in the intestines. SCFAs are signaling molecules involved in the modulation of various host metabolic processes that stimulate free fatty acid receptors in the liver and adipose tissues, which is known to promote fatty acid oxidation and inhibition of fat accumulations. SCFA particularly butyrate, can enhance host metabolic processes by reducing blood glucose levels and promoting energy metabolism, downregulates the levels of the ECS ligands, AEA and 2-AG, by regulating the expression of enzymes involved in the biosynthesis and degradation (accomplished by increasing the MAG1 levels) of the ligands and is a potent histone deacetylase inhibitor that globally promotes and represses gene expression to effectively modulate ECR. The natural bacterium, Clostridium acetobutylicum and Treponema denticola and modified E. coli butyrate-producers were able to reduce the up-regulated ECS expression in the gut as the result of high fat diet.

A retrospective cohort study suggests that for the active Cannabis users (4% of sample) who were hospitalized with COVID-19, they were associated with lower levels of inflammatory markers on admission; furthermore, they had better clinical outcomes compared with non-users including received less COVID-19 adjunctive therapies and had a decreased need for intensive care units (ICU) or mechanical ventilation care. However, another study found that COVID-19 patients with substance use orders (grouping them all together but not isolating CUD) have worse outcomes compared to general COVID-19 patients, including increased hospitalization and death.

CBDA and CBGA have been found to be allosteric as well as their orthosteric ligands with micromolar affinity for the viral spike protein. In follow-up virus neutralization assays, they both prevented infection of human epithelial cells by a pseudovirus expressing the SARS-CoV-2 spike protein and prevented entry of live SARS-CoV-2 into cells. CBGA and CBDA were equally effective against the SARS-CoV-2 alpha variant B.1.1.7 and the beta variant B.1.351. However, critics are skeptical of this conclusion due to reproducibility of results with higher doses and duration required for effect.

There are also innovative products being developed with cannabinoid infused textiles for antimicrobial resistance, or wound care salves or biofilms.

Predisposed individuals may include those who have an inadequate vaccination status, immunosuppression, mental health (including social isolation) and substance use disorders, along with well-known pre-existing medical conditions (which are usually associated with proinflammatory states). In addition, there are that can be simultaneous secondary mental health effects (which may be organic), that may further contribute to anxiety, depression (adjustment or endogenous), PTSD, uro-sexual, fatigue, pain (with predictions that a cohort of chronic pain disabled sufferers will arise, who could be potentially become opioid reliant), sleep disorders, brain “fog,” or subtle cognitive dysfunction, appetite and seizure disorders, and loss of sensory function, especially anosmia, but also taste, tinnitus or visual disturbances (which may be a source of Covid-19 entry with conjunctivitis or photophobia, these symptoms may occur in 11% of Covid-19, with rare blindness due to retinal vein occlusion or retinopathy).

Sensory deprivation may further contribute to cognitive disturbances and dementia (and there is a correlation of reduced retinal layer thickness with predisposition to neurodegenerative diseases). Mask wearing or communication occurring in virtual settings for those who are hearing impaired, may further confound their communication deficits and perpetuate their isolation, with reduced access to a hearing assessment and adaptive equipment. This is also modulated by accelerated progression of pre-existing medical conditions, iatrogenic factors (including adverse effects of medications or drug interactions), nutrition, gut microbiome and other socioeconomic factors (support system, home environment, access for psychiatric or neurologic/rehabilitative comprehensive care, and health care disparities), particularly with “long” covid (estimated at 20%, some with substantial disabilities) or those with residual permanent organ system impairments. There is an expanding high demand for assessment and care of these residual conditions, with socioeconomic implications for both treatment (reactive) and/or prevention (proactive).

CB2R agonists can act by down-regulating both ACE2 and TMPRSS2 proteins, by reducing cytokine interferon-gamma (IFN-γ) and paracrine mediator, TNF-a, and this effect can be achieved topically, possibly used as a mouthwash.

A full spectrum extract from C. sativa Arbel strain containing CBD, CBG and THCV, and multiple terpenes substantially reduced (dose dependently) interleukin (IL)-6 and -8 levels in an alveolar epithelial (Δ549) cell line, as well as reducing ACE2 expression on the cell membrane. These effects were more robust with a full spectrum extract suggesting an entourage effect, whereas a CBD only extract resulted in a narrow therapeutic range, with lower and higher dosing having less of an effect.

It is also hypothesized that COVID-19 patients are potentially vulnerable to a significant disease-drug interaction (including CBD CYP metabolism), and therefore, suitable dosing guidelines with therapeutic drug monitoring should be implemented to assure optimal clinical outcomes.

Melaleuca cajuputi essential oil (TA) extracted from fresh cajeput leaves through steam distilling. The inhibitory capability of active compounds in the TA over the ACE2 protein in human body—the host receptor for SARS-CoV-2 and the main protease (PDB6LU7) of the SARS-CoV-2 using docking simulation has been studied. The results indicate that the ACE2 and PDB6LU7 proteins were strongly inhibited by 10 out of 24 compounds accounting for 70.9% in the TA. The anti-coronavirus activity contained in TA terpene content, using docking simulation is expressed in this order, which has a comparable distribution as hemp: Terpineol (TA2)≅Guaiol (TA5)≅Linalool (TA19)≅Cineol (TA1)≅β-Selinenol (TA3)≅α-Eudesmol (TA4)≅γ-Eudesmol (TA7). Interestingly, the synergistic interactions of these 10 substances of the TA exhibit excellent inhibition into the ACE2 and the main protease (PDB6LU7) of the SARS-CoV-2 virus.

Binding of the SARS-CoV-2 virus to angiotensin-converting enzyme 2 (ACE2) leads to the internalization of ACE2 and activation of angiotensin II resulting in the activation of NFκB. Subsequently, cytokines IL-6, TNFα, IL-1β, and IL-10 will be produced which might lead to local lung dysfunction with severe T-lymphocyte apoptosis, with lymphocytopenia, as well as a rise in blood pressure, which contributes to lung injury and deterioration of pulmonary function, as occurs in acute respiratory distress syndrome (ARDS). Researchers from Italy in 2017 examined the potential of a hemp seed protein isolate that was prepared from defatted hemp seed by alkaline solubilization/acid precipitation as inhibitors for angiotensin-converting enzyme 2 (ACE-2). All four peptides extracted from hemp seed oil had ACE-inhibitory activity, a potential agent to inhibit entry of SARS-CoV2 into the cells.

It was reported that exogenously added 2-AG leads to the attenuation of lymphocyte proliferation through the decrease of lymphocytes T helper cells (Th)1- and Th17-associated cytokines IL-6, IL-2, and tumor necrosis factor (TNF)α. Moreover, activated lymphocytic B and T cells that produce high levels of 2-AG inhibit in a feedback loop T cell activation and proliferation, making exogenously applied 2-AG a putative candidate for therapeutic usage in Th1- or TH17-dependent diseases. Upon antigen activation by pathogens, macrophages, and dendritic cells produce and release 2-AG, which results in the upregulation of 2-AG levels in the serum and lymph nodes of mice during vaccination CB2 dependently. In a murine immunization model, transient administration of CB2 antagonist AM630 or inverse antagonist JTE907 increased the intensity of antigen-specific immune responses by upregulation of immunomodulatory genes in secondary lymphoid tissue. AEA inhibited macrophage-mediated killing of the TNFα-sensitive mouse alveolar macrophage cell line L929. Correa et al. presented evidence that AEA inhibited expression of pro-inflammatory cytokines like IL-12 and IL-23 in vitro models of immune disorders and increased the anti-inflammatory cytokine IL-10 in activated mouse microglia. In a model of acute intestinal inflammation, it was shown that the transporter p-glycoprotein helped the influx of endocannabinoids into the intestinal lumen, which inhibited the migration of neutrophils by counteracting the pro-inflammatory neutrophil chemoattractant eicosanoid hepoxilin A3. Similarly, the migration-related transcriptional profile of neutrophils was enhanced in CB2^{-/-} mice.

CB1R and CB2R were expressed by bone marrow derived hematopoietic stem cells and CD34⁺ cells. AEA and 2-AG were detected in the microenvironment of peripheral blood and bone marrow, which were secreted by bone marrow mesenchymal stem cells. Migration of hematopoietic stem cells was stimulated by AEA and 2-AG and blocked by CB receptor antagonists rendering endocannabinoids putative candidates for the enhancement of the migration of hematopoietic stem cells extracts of the phyto-cannabinoids CBD and THC could attenuate the proliferation of activated lymphocytes and the secretion of pro-inflammatory IL-17, thereby increasing secretion of the anti-inflammatory IL-10. Additionally, the endocannabinoid AEA and the phyto-cannabinoid THC could also induce immunosuppression in B cells as was examined in both primary and secondary in vitro plaque-forming cell assays of antibody formation. Many reports have shown that exogenously applied CBD suppresses transcription factors involved in inflammation like Nuclear Factor of activated T-cells (NFAT), Activator protein (AP 4, and NF-κB, which results in abroad repression of cytokines like interleukin (IL)-6, IL-1β, IL-1α, granulocyte-macrophage colony-stimulating factor (GM-CSF), and TNFα in diverse cells and tissues. These cytokines have a central role in the development of cytokine release storm (CRS) in COVID-19. IL-6 promotes the differentiation of Th17 cells, which was shown to be suppressed by CBD. Moreover, CBD was shown to inhibit type II interferon (IFNγ).

These anti-inflammatory actions of Cannabis might be beneficial for the prevention of cytokine storm (CRS) before the host inflammatory response turns pathological during the transition from mild to critical disease in COVID-19 patients.

Cannabinoid acids from hemp (Cannabis sativa) found to be allosteric as well as orthosteric ligands with micromolar affinity for the spike protein. In follow-up virus neutralization assays, CBGA/CBDA prevented infection of human epithelial cells by a pseudovirus expressing the SARS-CoV-2 spike protein and prevented entry of live SARS-CoV-2 into cells. were found to be allosteric as well as orthosteric ligands with micromolar affinity for the spike protein. CBGA and CBDA were equally effective against the SARS-CoV-2 alpha variant B.1.1.7 and the beta variant B.1.351. Orally bioavailable and with along history of safe human use, these cannabinoids, isolated or in hemp extracts, have the potential to prevent as well as treat infection by SARS-CoV-2. Cannabinoids Block Cellular Entry of SARS-CoV-2 and the Emerging Variants

Thus, full spectrum Panakeia extract (without THC or CBD), administered using alternative routes of Cannabis administration like vaporizing or edibles, is a novel anti-inflammatory therapy, predicted to prevent infection with SARS-CoV-2, or mitigate symptoms of Covid-19, both in the respiratory and digestive tracts via inhibition of hyperinflammation.

Researchers have reported that extracts high in CBD has been found to alter gene expression and inflammation and harbor anti-cancer and anti-inflammatory properties. The Canadian researchers, from a range of institutions including the University of Lethbridge and University of Calgary, developed over 800 new C. sativa cultivars and hypothesized that high-CBD rich C. sativa extracts may be used to down-regulate ACE2 expressions in target COVID-19 tissues. The ACE2 receptor that SARS-CoV-2 and other coronaviruses use to access host cells is expressed in a range of tissues, including the lung, nasal mucosa, kidney and GI tract. One recent study reported high levels of ACE2 expression in oral epithelial tissues and suggested that the oral cavity could be an important target for prevention strategies. Some C. sativa extracts down-regulate serine protease Transmembrane Serine Protease 2 (MPRSS2), another critical protein required for SARS-CoV-2 (the virus that causes Covid-19 infection) entry into host cells.

CBD and its metabolite, 7-OH-CBD by CYP2D6, but not congeneric cannabinoids, potently 5 block SARS-CoV-2 replication in lung epithelial cells. CBD acts after cellular infection, inhibiting viral gene expression and reversing many effects of SARS-CoV-2 on host gene transcription. CBD induces interferon expression and up-regulates its antiviral signaling pathway. A cohort of human patients previously taking CBD had significantly lower SARS-CoV-2 infection incidence.

An extract fraction from C. sativa Arbel strain (designated as Full spectrum CBD=FCBD) substantially reduced (dose dependently) interleukin (IL)-6 and -8 levels in an alveolar epithelial (Δ549) cell line. FCBD contains CBD, CBG THCV, and multiple terpenes. Treatments with FCBD and a FCBD formulation using phytocannabinoid standards (FCBD: std (with isolated CBD)) reduced IL-6, IL-8, C-C Motif Chemokine Ligands (CCLs) 2 and 7, and ACE2 expression in the Δ549 cell line. Treatment with FCBD induced macrophage (differentiated KG1 cell line) polarization and phagocytosis in vitro, and increased CD36 (a marker of human adipocyte progenitors stem cells, which is found in immune cells). and type II receptor for the Fc region of Immunoglobulin (Ig)G (FcγRII) expression. FCBD treatment also substantially increased IL-6 and IL-8 expression in macrophages. FCBD:std, while maintaining anti-inflammatory activity in alveolar epithelial cells, led to reduced phagocytosis and pro-inflammatory IL secretion in macrophages in comparison to FCBD. The phytocannabinoid formulation may show superior activity versus the Cannabis-derived fraction (isolated CBD) for reduction of lung inflammation. This study suggested that the “entourage effect” was more effective with a validated dose response curve, whereas pure CBD extract only allowed a narrow therapeutic range, with lower and higher dosing having less of an effect.

This oil is similar in composition as the full spectrum CBG extract, and also suggests an entourage effect is present.

It is also hypothesized that COVID-19 patients are potentially vulnerable to a significant disease-drug interaction, and therefore, suitable dosing guidelines with therapeutic drug monitoring should be implemented to assure optimal clinical outcomes.

Patients may also be on therapeutics for pre-existing comorbidities; especially epidemiological studies have indicated that individuals with hypertension, hyperglycemia, and obesity are more vulnerable to COVID-19 than the average population. This complicates the drug-disease interaction profile of the patients as both the investigational drugs (e.g., remdesivir (as a substrate for CYP2C8, CYP2D6, and CYP3A4 as well as an inhibitor of CYP3A4), dexamethasone, affecting CYP3A4, and 26 metabolism) and the agents for comorbidities can be affected by compromised CYP-mediated hepatic metabolism which affects the hepatic clearance of xenobiotics. Overall, it is imperative that healthcare professionals pay attention to the COVID-19 and CYP-driven drug metabolism interactions with the goal to adjust the dose or discontinue the affected drugs as appropriate.

CYP activity (usually in the liver but can also be present in other organs including kidneys, lung and brain) are modulated in an isoform-specific manner by SARS-CoV-2 infection. Cannabinoid metabolism by CYP is greatest with CBD(A), potentially with drug interactions, including specific Covid-19 therapies, whereas CBG(A) has a lesser effect.

Antineoplastic Activity: Cannabinoids seem to be capable of affecting cancer cells' viability, growth, migration, and invasion through different mechanisms including limiting tumor cells' proliferation and angiogenesis, blocking tumor-related signaling pathways, inducing autophagy, and manipulating immune responses in cancer (undifferentiated or primitive) cells, many containing CBR on the cell membrane surface. One of the major anticancer mechanisms employed by cannabinoids is inhibiting cancer cells' proliferation by promoting various cell-death pathways (apoptosis) and limiting several tumor-growth-related processes. The ECS regulates cell fate and division during oncogenesis and can be manipulated pharmacologically, and that the level of CB1R expression and activity negatively correlates with cell division. For example, in breast cancer, CB2R agonism inhibits cell cyde progression, whereas in fibrosarcoma cells, CB1R antagonism up-regulates the cell cycles, and down-regulates cyclins E and D. Up- and down-regulation of CB1R levels influences cell growth, just as cannabinoid treatment affects cell growth.

In the brain, 17β-estradiol (a more potent estrogen) regulates CB1R expression in a region-dependent manner (but also retinoic acid receptor (RARγ) and peroxisome proliferator-activated receptor (PPARγ) to co-localize), providing a possible explanation for gender-related differences in sensitivity for the central effects of cannabinoids, increases NO in endothelium, and increases the expression of CB2R in osteoclasts hi vitro, as well as the expression of CB1 receptors in human colon cancer. Tamoxifen, an estrogen receptor (ER) blocker, has been demonstrated to act as an inverse CB1R and CB2R agonist in breast cancer cells. Cannabinoid receptor agonists signal through the inhibition of the AC and protein PKA pathways. Endocannabinoids decrease the expression of epidermal growth factor (EGF) receptors and significantly inhibit the EGF-induced proliferation, migration and invasion of non-small cell lung cancer cell lines.

CB2R are linked via to AC protein kinase and MAPK, such that cannabinoid agonists through MAPK inhibit cyclic adenosine monophosphate (cAMP), binding deoxyribonucleic acid (DNA) sequences with apoptosis and preventing proliferation, which can be useful in B-cell lymphomas. Endocannabinoids inhibit the mitogenic action of prolactin, which is among its others functions also an important inducer of carcinogenesis in breast cancer and both estrogens and endocannabinoids regulate the expression of prolactin receptors. Cannabinoids through CB2R cause a reduction in the proteolytic matrix metalloproteinases MMP1, MMP2, and MMP9, and inhibition of angiopoietin 2, as well as vascular endothelial growth factor (VEGF) expression, through CB1R in target tissues with ER (breast, bone, heart and skin), as well as thyroid, inhibits angiogenesis. Cannabinoids cause the down-regulation of phosphatidylinositol-4,5-bisphosphate β-kinase (PI3K-Akt) and ER-K1/2 kinase signaling, with increased cyclin D1 protein expression, which in turn inhibits proliferation and induces apoptosis. Cannabinoid agonists inhibit AP-1-mediated transcriptional activities, the latter being induced in several types of tumors, and activates ERK, which can treat colon cancer and affects osteoblast lifespan. There are some inconsistent studies that CB1R may stimulate breast cancer by suppressing the body's anti-tumor immunity, whereas CB2R may inhibit tumor growth and breast cancer metastasis. In endometrial cancer, plasma AEA concentrations were significantly higher inpatients, and AEA can decrease CBR, but this distribution may not occur in healthy females. In prostate cancer cells, MAPK activation that has been characterized as both androgen-dependent and -independent. CB1R agonists may reduce testosterone secretion and if exposed chronically, cause testes degeneration, and may have anti-androgenic effects. CB1 messenger ribonucleic acid (mRNA) levels are increased in non-Hodgkin lymphoma. Up-regulation of CB1R in alveolar rhabdosarcoma increases the metastatic ability of the cells, but not mitosis, and CBR agonists may induce autophagy in osteosarcoma.

Anti-tumor activity of CB2R agonists seems to hinge on its regulation of reactive oxygen species (ROS), endoplasmic reticulum (ER) stress, and immune modulation with anti-proliferative and pro-apoptotic effects on a wide variety of cancer types, and they interact with other non-ECS receptors for this effect.

CB2R agonists can be a potent inhibitor of exosomes and microvesides (EMV) release, involved in intercellular communication through transfer of proteins and genetic material which may explain chemotherapy resistance, from cancer cell lines: prostate cancer (PC3), hepatocellular carcinoma (HEPG2), breast adenocarcinoma (MDA-MB-231) and gliobastoma multiforme. Cannabinoids significantly reduced exosome release in all three cancer cell lines, and also significantly, albeit more variably, inhibited microveside release, and this mechanism involves changes in mitochondrial function, including modulation of STAT3 and prohibitin expression, such that its simultaneous use can sensitize cancer cells to chemotherapy.

Another study that investigated the synergistic effect of a mixture of different cannabinoids, including THC, CBG, CBN and CBD on MDA-MB-231 and MCF-7 human breast cancer cell lines revealed that the combination of these chemicals can exert cell-cyde arrest in G2 phase following apoptosis without having any adverse cytotoxic effects on normal cells. Also, nuclear fragmentation besides cytoplasmic vacuolation, lysosome increased size and lipid accumulation was observed following cannabinoid administration. Plus, CBD and Δ9-THC were shown to decrease viability, induce caspase-3 activation, apoptosis and suppress invasion in transitional cell bladder cancer carcinoma. Interestingly, these cannabinoids showed a synergistic effect when they were combined with other cannabinoid agents such as CBC or CBV. However, when they were combined with chemotherapeutic agents, they demonstrated a range of responses, from synergistic to antagonistic effects in bladder cancer cells dependent on their concentration. In addition, evidence revealed that CBD or CBD-rich Cannabis extract was capable of inhibiting the growth of MDA-MB-231 breast carcinoma xenograft tumors in athymic mice or rat v-K-ras-transformed thyroid epithelial cells models and suppressing cancer cells' migration and metastasis to lungs without affecting normal cells. The observed effect was assumed to be carried out via direct or indirect activation of cannabinoid CB2R, vanilloid transient receptor and potential vanilloid type-1 receptors. Meanwhile, CBD was able to induce cell death by direct/indirect activation of CB2/TRPV receptors in breast cancer cells by increasing intracellular Ca and ROS levels in these cells. Treatment of endometrial cancer cell lines (Ishikawa and Hec50co cells) with CBD, AEA, THC induced reduction of cell viability. Moreover, AEA and CBD induced activation of caspase3/7, apoptosis in cancer cells by inducing PARP cleavage, increasing intracellular ceramide level, Ca 2+ influx, NF-κB pathway and COX-2, and decreasing cell-cyde promoting factors such as cyclin D, cyclin E, CDK2, CDK4, RB, and E2F1, which is accompanied by an increase of annexin V on cancer cells' surface. Cannabinoids also suppress cancer cells' migration and invasion by decreasing MMP9.

Cannabinoids: Cannabinoids can be administered by smoking, vaporizing, oral ingestion, transdermal patch, intravenous injection, sublingual absorption, or rectal suppository. Once in the body, most cannabinoids are metabolized in the liver, especially by cytochrome P450 mixed-function oxidases, mainly CYP 2C9. Thus, supplementing with CYP 2C9 inhibitors may lead to extended intoxication. Some metabolites are also stored in fat which can be detected in the body several weeks after administration, in addition to being metabolized in the liver. Δ9-THC is metabolized to 11-hydroxy-Δ9-THC, which is then metabolized to 9-carboxy-THC. These metabolites are the chemicals recognized by common antibody-based “drug toxicology tests”; in the case of THC or others, these loads do not represent intoxication (compare to ethanol breath tests that measure instantaneous blood alcohol levels), but an integration of past consumption over an approximately month-long window. This is because they are fat-soluble, lipophilic molecules that accumulate in fatty tissues.

Cannabinoids can be separated from the plant by extraction with organic solvents. Hydrocarbons and alcohols are often used as solvents. However, some solvents are flammable, and many are toxic. Butane may be used, which evaporates extremely quickly. Supercritical solvent extraction with carbon dioxide is an alternative technique. Once extracted, isolated components can be separated using wiped film vacuum distillation or other distillation techniques. Also, techniques such as SPE or SPME are found useful in the extraction of these compounds.

The cannabinoid drugs (CB1R agonists), including experimental analogs, have been classified as follows:
1. Classical cannabinoids (THC, other constituents of Cannabis; and their structurally related synthetic analogues e.g. HU-210, AM-906, AM-411, 0-1184); HU-210 is CSA DEA Sch II, as an analog of THC
2. Nonclassical cannabinoids (cydohexylphenols or β-arylcydohexanols such as CP-47,497-C8, CP-55,940, CP-55,244)
3. Hybrid cannabinoids (combinations of structural features of classical and non-classical cannabinoids, e.g. AM-4030)
4. Aminoalkylindoles (AAIs), which can be further divided into naphtoylindoles (e.g. JWH-018, JWH-073, JWH-398, JWH-015, JWH-122, JWH-210, JWH-081, JWH-200, WIN-55,212); phenylacetylindoles (e.g., JWH-250, JWH-251); naphthylmethylindoles and benzoylindoles (e.g., pravadoline, AM-694, RSC-4); JWH-018, JWH-073, CP-47,497, CP-47,497-C8 and JWH-200 are illegal through CSA DEA Sch I
5. Eicosanoids (endocannabinoids such as AEA, and their synthetic analogs (e.g. methanandamide)
6. Others, diarylpyrazoles (selective CB1R antagonist Rimonabani®), naphtoylpyrroles (JWH-307), naphthylmethylindenes or derivatives of naphthalene-1-yl-(4-pentyloxynaphthalen-1-yl)methanone (CRA-13). In most cases with the classical, non-classical and hybrid cannabinoids, one specific stereoisomer is much more potent than the other(s), whereas most of the AA1s, eiosanoids and ‘others’ do not possess an asymmetric center.

There are multiple synthetic analogs of CBD. Sesquicannabigerol is another minor constituent of natural CBG, and its affinity at CB2R is 5-fold higher than CBG. Some cannabinoid analogs have the terpene, limonene as a chain.

There are natural analogs of CBG (bibenzyl-CBG) from H. umbraculigerum and some liverworts. Experimental semisynthetic cannabigerol CBG)-like drugs in development, utilizing CBG as the starting material: 1″,1″-Dimethylheptyl-monomethoxy cannabigerol (HUM-223) by substituting dimethylheptyl pentyl (DMH) at position 5 of CBG resulting in a monomethoxy CBG-DMH; and Monomethoxycannabigeroyl-β-morpholinoproprionate (HUM-234) is synthesized by substituting morpholinopropionic acid from monomethoxy-CBG (a derivative of HUM-223). HUM-233 has increased its bioavailability, and has reduces TNF-α levels. HUM-234 increases TNF-α at higher doses. Cannabigerol quinone (VCE-003.2), JWH-0133 and recombinant yeast production (represents synthetic analogs of CBGA) possess anti-inflammatory and analgesic properties in animal models. In addition, unlike CBG, HUM-234 also prevents obesity in mice fed a high-fat diet (HFD) suggesting that HUM-234 and related compounds may have potential to treat metabolic disorders including liver disease. Synthetic dimethylheptyl homolog of cannabigerol (CBG-DMH) displays hypotensive potential.

There are several examples of bacteria, fungi and yeasts genetically modified organisms (GMO) to produce drugs or precursors thereof. Microorganisms capable of producing natural oils, such as algae, are more difficult to manipulate genetically and their use may require complex precursors such as CBG. These microorganisms are called “recombinants”, as their genome has been manipulated to include external DNA and provide them with the enzymes necessary for the transformation of simple substrates into complex products, so the extraction and purification process are simpler. Cannabinoid production in recombinant organisms allows stereo-selectivity of biosynthesis which is capable of producing only the active isomer without the need for further purification. Yeast, feeding on sugar, can successfully produce acidic precursors easily. They first engineered yeast's native mevalonate pathway to provide a high flux of geranyl pyrophosphate (GPP) and introduced a hexanoyl-CoA biosynthetic pathway combining genes from five different bacteria using synthetic hexanoic acid (not found in Cannabis). They then introduced Cannabis genes encoding the enzymes involved in olivetolic acid (OA) biosynthesis, a previously undiscovered prenyl transferase enzyme and cannabinoid synthases. Once they had yeast-producing CBGA, they added another enzyme to convert CBGA to THCA and a different enzyme to create a pathway to CBDA. The synthases converted CBGA to the cannabinoid acids, THCA and CBDA, which, upon exposure to heat, decarboxylate to THC and CBD, respectively. They also added enzymes that made the yeast produce two other natural cannabinoids, CBDV (cannabidivarin) and THCV (tetrahydrocannabivarin). Some companies are estimating the cost of cannabinoid production through these techniques at around $1000 a kilogram, between 4 and 5 times cheaper than outdoor cultivation, and about 30 times cheaper than conventional chemical synthesis.

CBG derivatives include CBGM, a monomethyl ester; Sesquicannabigerol, more potent than CBG CBGA derivatives: CBGAM, a monomethyl ester; Each cannabinoid has unique pharmacokinetics (absorption, distribution, metabolism and excretion) and its pharmacodynamics effects are related to the diversity of these unique molecules by modulating, with variable affinities, with the ECS, and more importantly, to other heterogeneous non-ECS receptors (found in the brain and many other body locations, with significant and dynamic variability and interactions with ECS), for which ongoing studies are being done to fully characterize them.

Bioavailability: Bioavailability usually refers to the absorption of drug into the serum, but sometimes, cannot be extrapolated to predict the actual tissue penetration including CNS, and does not always correlate with its therapeutic effects.

Cannabinoids are lipophilic which allows them to readily cross the BBB (less so with acidic forms), but they also may assist in its repair, by binding to CB2R, particularly with neuro-inflammatory conditions. There is a lag in brain concentration of cannabinoids (peaks at 6 hours after oral CBD in rats) with serum levels (peaks at 1 hour after oral CBD in rats). Most data is based on CBD effects. Their lipophilicity results in along half-life (about 15-48 hours, and up to 5 days for high chronic dosing), with detection in adipose tissue or hair lasting for several weeks (it can take up to five half-lives for a substance to completely be eliminated). Ingestion of cannabinoids with lipids or high calorie meal improves bioavailability. High fat meals also potentially inhibit the activity of drug efflux transporters present on the apical membrane of enterocytes, and stimulate the release of biliary secretion, which further inhibits efflux transporter activity.

While eating a short term ketogenic diet which converts more “white” fat cells into “brown,” it can increase oral absorption of lipid soluble cannabinoids and is sometimes tried for weight loss/obesity, metabolic syndrome, seizure control, or neurodegenerative conditions and systemic inflammation. Since cannabinoids and their metabolites are stored primarily in fat cells, shifting the body into ketosis and burning those fats would more rapidly mobilize cannabinoid metabolites. Also, cannabinoid tinctures are dissolved in healthy oils including short (SCT, ie, butyrate) or medium chain triglycerides (MCT, i.e., coconut or palm) which are also beneficial (anti-tumor, cardio/neuro-protective and metabolism). In studies, rat intestines exposed to both SCT and MCT increased the responsiveness of the ECS with increased CBR.

Based on dog data, CBGA is orally absorbed significantly better than CBG, with or without food. There were no adverse effects as monitored by blood testing, after 2 weeks of high twice daily dosing. Orally ingested CBGA/CBG will deliver long-lasting but relatively mild effects, whereas if inhaled, there are much stronger effects that persist for a significantly shorter duration. CBG oil taken sublingually can have an onset within 15 minutes, lasting 2-3 hours, while CBG gummies (oral) will have an onset in about 45 minutes, with a longer duration (up to 7 hours).

Oral ingestion of CBG has a bioavailability range of 13-19% (lower for smokers), sublingual administration has a range of 13-56%, higher with sub-buccal application and possibly lower with nasal spray, suppositories, rectal: 13.5% to 40%, (submucosal; there are CBR in colonic epithelium and sex organs including vagina) and for inhalation of cannabinoids, smoking has a range of 2-56%; and vaporization, a range of 50-80%, and for topical applications, a range of 5-10% with a longer duration of effect. A chemically modified version of THC, THC-hemisuccinate (THC-HS), is bioavailable in the rectum. Succinate, an intermediate compound in the breakdown of sugar, is slightly water soluble, which allows it to be absorbed through the rectal mucosa, and is 70-80% bioavailable, almost 2.5 times higher than an orally ingested THC. CBG metabolism increases and has a better absorption from the body when paired with CBGV.

CB1R agonists can also affect GI motility by retarding gastric emptying and inhibits colonic tone and phasic pressure activity, which would not predictably occur with CB2R agonists.

When inhaled, THC and its metabolites enter the bloodstream rapidly via the lungs; they achieve peak levels within 6 to 10 minutes and reach the brain and various organs. The bioavailability of inhaled THC is 10% to 35%. After THC is absorbed, it travels to the liver where most of it is eliminated or metabolized to 11-OH-THC (psychoactive) or 11-COOH-THC. The remaining THC and its metabolites enter the circulation. The bioavailability of ingested THC is only 4% to 12%. THC is highly lipid-soluble and is therefore rapidly taken up by fat tissue. THC, through cytochrome (CYP) P450 enzymes, is a CYPA2 inducer that may lead to reduced drug concentration via increased metabolism and consequently decreased drug effect. The plasma half-life of THC is 1 to 3 days in occasional users and 5 to 13 days in chronic users. More than 65% of Cannabis is excreted in the feces and approximately 20% is excreted in urine.

Cannabidiol (CBD): CBD appears particularly complex, with over 65 identified molecular targets, and different mechanisms proposed to explain its actions. On CB1R/CB2R, CBD has a very low affinity and little agonist activity; on the other hand, it seems to antagonize CB1R/CB2R synthetic agonist action; ads as negative allosteric modulator of CB1R; antagonist/inverse agonist of CB2R (considered allosteric modulator); inhibiting FAAH activity, thus increasing endogenous AEA levels, with a neuroprotective effect after cerebral hypoxia-ischemia in immature pigs involves CB2R activation; antagonist/inverse agonist of GPCR3, GPCR6, GPCR18, and GPCR55; allosteric; agonist of 5HT1A receptor (positive allosteric modulator, which then modulates dopamine release in the nucleus accumbens, with anti-depressant and anxiolytic effects), a partial agonist of 5HT2A (same pathway as psychedelic drugs affecting perception, cognition and mood), and an allosteric inhibitor of 5HT3A activates PPARγ agonist; activates adenosine A1 and A2A receptors (reuptake inhibitor); inhibits a nicotinic acetylcholine receptors; allosterically modulates p and 6 opioid receptors; TRPA1 agonist, TRPV1/2/3 agonist, TRPM8 antagonist; GABAa allosteric modulator activation; GlyRa1/3 allosteric modulator activation; vagal mediated cholinergic anti-inflammatory response signaling through the JAK2/STAT3 pathway, decreasing levels of proinflammatory cytokines, such as TNF-α, IL-1β, and IL-6 and increasing levels of anti-inflammatory cytokines such as IL-10. CBDA has GPR55 inhibition, weak TRPA1 agonist, TRP3/4 antagonist, TRPMB antagonist, AEA uptake inhibitor and DABL inhibitor. CBDV is similar to CBD for its binding of GPR55, TRPs, AEA, and is also DAGL inhibitor (without significant binding of CBR or other receptors described above). The relative binding affinities (potency) have not been fully characterized. Other cannabinoids may have similar binding effects but have not been adequately studied. CBD up-regulates ERK1/2 and CREB phosphorylation, as well as BDNF expression involved in hippocampal neurogenesis; it significantly reduced AMPA GluR1/2 protein levels in striatal pathway of the mesolimbic system, which indirectly inhibits GABA transmission. CBDA is an PPAR agonist, but CBD and THC are only PPARγ agonists.

Chronic neuropathic pain is associated with inflammation, mainly at the glial level. Preclinical studies demonstrate the beneficial effect of CBD treatment on autoimmune neuroinflammation by suppressing the expression of proinflammatory chemoattractants and regulating the activity of inflammatory macrophages. Recent evidence shows that medical Cannabis or cannabinoids result in little to very little improvement in pain relief, physical functioning, and quality of sleep among chronic pain patients. However, this evidence was collected in chronic noncancer and cancer-related pain without establishing a specific route of administration or specifically identifying neuropathic pain. Transdermal administration of cannabinoids may be a more effective alternative to the oral or inhaled route for the management of this challenging neuropathic pain condition. In a study in murine models, it was shown that the application of CBD in transdermal gel achieves a significant plasma concentration at steady state, suggesting the efficacy of this pathway administration.

In prospective trials and systematic reviews administered orally and inhaled, cannabinoids can cause some relief of neuropathic pain in patients with neuropathic pain. The clinical use of Cannabis derivatives is already approved based on clinical evidence in entities such as spasticity in multiple sclerosis, seizures associated with two rare and severe forms of epilepsy, Lennox-Gastaut syndrome (LGS) and Dravet syndrome (DS), and cachexia-anorexia syndrome in cancer and HIV.

CBD inhibits two different kinds of sodium channels found in the membranes of nociceptors, the specialized neurons that sense and communicate pain. This inhibition prevents sodium from rushing inside nociceptors, which keeps the neurons in an inactive state and stops them from firing and transmitting a “pain” message via an electrical signal. CBD activates the potassium channel, allowing potassium ions to flow inside nociceptors. This influx of potassium reduces the firing activity of the neurons, thus blocking pain signaling. In fact, flupirtine, a pain medicine with restricted use due to liver toxicity, works by the same mechanism.

CBD can improve social interaction, anxiety, appetite, concentration, and reducing psychomotor agitation, all useful in mitigating symptoms in children with autism spectrum disorder (ASD) β-arrestin regulates the number of CBR on the membrane surface, which lead to desensitization or a diminished response (tolerance effect). (β-arrestin binding induces CBR internalization and switches receptor coupling, especially for CB1R, also activating MAP kinase pathways linked to nuclear effects, affecting cAMP levels homeostatically. Rapid desensitization results in tachyphylaxis, whereas a more gradual desensitization results in tolerance, physical dependence (and thus withdrawal signs) and, potentially, cannabinoid resistance. Dimerization (coalescence of two) CBR has been demonstrated to allosterically modulate opioid receptor activity. Any target that affects the location, affinity and concentration of CBR, their synthesis and degradation will affect the system.

CBD has associated with a reduction in mammalian male testis size, the number of germ and Sertoli cells in spermatogenesis, fertilization rates, and plasma concentrations of hypothalamic, pituitary and gonadal hormones, and chronic doses of CBD may result in impaired sexual behavior in mice.

Women who sleepless than seven hours per night are 15% less likely to become pregnant. Cortisol is one of the primary culprits involving sleep and fertility by suppressing melatonin levels and estrogen boosting follide maturation and therefore ovarian function. CBD used as a sleep aid may improve fertility (although it is not recommended in pregnancy). CBD is largely understood to have a positive effect on a person's mood due to its impact on several neurotransmitters in the brain, specifically anandamide and adenosine. By inhibiting the adenosine Δ2A receptor, CBD helps to reduce anxiety and depression, thereby improving one's mood.

CBDA and CBD have overlapping, but distinct properties, in which CBD, in general, is more potent as an antioxidant, anti-inflammatory through CB2R, TRP activity, COX-2, lipid metabolism with anticancer/apoptosis effects, inhibits GPR55 expression, but CBDA has greater bioavailability from GI absorption, PPARγ activity (also 3/6, including cancer and appetite effects), significantly better anti-emetic effect on motion sickness or toxins based on 5HT1A binding (modulates nerve cell signaling, governing motor skills, sleeping, eating, digestion, and emotions), which also allows antinoception, anxiolytic and sleep, and may have be advantageous for less phase I liver metabolism (reduced potential drug interactions) and as a comparable antimicrobial and anticonvulsant (although CBGA was more potent)/neuroprotective agent (preventing the accumulation of neurotoxins and prions). CBDA has been found abundantly in hemp pollen (with a predominance of acid cannabinoids, as compared to THCA), and is also present in hemp seed. Varin derivatives may also convey improved anticonvulsant activity.

The bioavailability of CBD via inhalation is 11% to 45% (mean 31%), whereas that of oral CBD is 6%. CBD has high lipophilicity and therefore is rapidly distributed in the brain, adipose tissue, and other organs. CBD is hydroxylated to 7-OH-CBD (active) and 7-COOH-CBD (inactive) primarily by CYP3Δ4 and CYP2C9 in the liver that are excreted either intact or as glucuronide conjugates, and is excreted mainly in feces and less in urine. The plasma half-life of CBD is 18 to 32 hours.

Cannabigerol (CBG): There are significantly less studies regarding CBG/CBGA, compared to THC/CBD; however, some of the mechanisms of action can be predicted, based on its class effects in basic science, in silico, animal and clinical research. CBGA has demonstrated dose-dependent high potency against the most significant immunologic calcium transport process, store-operated calcium (Ca2+) entry (SOCE) by blocking Calcium Release-Activated Calcium (CRAC) currents decreasing Nuclear Factor of Activated T-cells (NFAT) activation and IL-2 production in human T lymphocytes modulated through CB2R, as one explanation for its benefit on chronic inflammation; whereas, THC full spectrum extract had a 70% effect (CBGA has greatest effect, followed by THCA, CBDA, CBDV; and pure CBD actually blocked this effect) suggestive of a moderate entourage effect (<10%) amongst the different cannabinoids, when a similar protocol was studied with different proportions.

CBG is extremely active as a neuroprotectant, improving motor deficits and preserving neurons that control involuntary movements against mice simulated Huntington's disease, by inhibiting neuroinflammation and acting as an antioxidant. The mice who received CBG significantly recovered their rotarod performance and the series of genes linked to the disease got partially normalized by the treatment. A CBG analog, VCE-003, acted as immunosuppressive agent for potential therapeutic agent for the treatment of human diseases with both inflammatory and autoimmune components including animal simulated multiple sclerosis, and was neuroprotective against inflammation-driven neuronal damage in an in vivo model of Parkinson's' disease in 2 different studies.

CBG also may act to increase appetite, with higher CB2R binding affinity in the brain (whereas CBD may reduce appetite, and is more sedating), but both may improve metabolic syndrome with insulin resistance. Occasional physiologic tolerance has been observed for CBD (which may actually be due its serotonin binding) and potentially CBG, but can be easily “reset,” with 3-7 days of abstinence, CBD withdrawal can occasionally occur and consists of mild GI symptoms such as nausea, vomiting, diarrhea and stomach cramps; and this may also occur with chronic CBG (and probably CBC) use. Psychological dependency (including insomnia, anxiety or mental distress) from withdrawing from any substance may occur, even if it is considered a non-habituating product; the reported symptoms may be a manifestation of the original complaints, or as a rebound phenomenon that are no longer being controlled, following abrupt cessation. No physical withdrawal symptoms were observed in short term use of pharmaceutical CBD, with doses at up to 1500 mg/day for 6 weeks. Based on review of the literature, and anecdotal experience, it appears that CBG, CBD and probably CBC, with chronic use, and even at higher doses, does not affect physiological measures, and does not significantly alter psychomotor or psychological functions. Physical withdrawal of CBG/CBD is easily tolerated and may occur if used chronically (but may also be related to non-ECS binding, or it could be related to residual THC content, which is present with most legal hemp, except Panakeia, as described below. Physical withdrawal (and often, with physiologic tolerance) can occur with any FDA approved DEA Sch substances (including THC and CBD), other non-controlled habituating substances (i.e., nicotine or caffeine) or non-habituating substances (i.e., antidepressants (especially those with serotonergic action), anticonvulsants, steroids, anti-histamines, anti-spasticity/muscle relaxant agents or anti-diarrheal agents), or with “rebound phenomena” (i.e., certain decongestants, especially in nasal spray, headache medications, laxatives, or gastric acid reflux inhibitors, proton pump or histamine-2 blockers). Certain herbal/or nutritional supplements (i.e., melatonin, valerian, lemon balm, or St. Johns wort) may also produce withdrawal symptoms.

CBG inhibits pro-inflammatory cytokine (Interleukin-11,-6,-8, tumor necrosis factor-TNF a and myeloperoxidase-MPO activity) which modulate metabolism, inflammation and anti-neoplastic effects. When administered topically, CBG increased lipogenesis in sebocytes, hyaluronan synthase (HAS2), fibroblast growth factor (FGF2), toll-like receptors (TLR2) for dermatologic anti-aging effect on wrinkles, skin regeneration, wound healing acne (antimicrobial effects), respectively, which was more efficacious compared to topical CBD. CBG is amongst the most potent cannabinoid with antimicrobial properties studied and have a distinct mechanism of action for gram positive bacteria. CB2R agonists, show impressive bactericidal activities against some important, therapeutically problematic Gram-negative pathogens, such as E. coli and P. aeruginosa, methicillin-resistant S. aureus strains (MRSA), other Gram-positive bacteria, acid-fast bacteria (tuberculosis), Salmonella enterica, Yersinia enterocoliticae, and L. monocytogenes. There also is suppression of specific neurodegenerative disease-associated gut bacteria by CB2R agonists, including Porphyromonas gingivalis or Helicobacter pylori (which can improve peptic ulcer disease outcome) by favorably altering the gut microbiome. The sources of cannabinoids' antimicrobial activities in addition to leaves and flower, may include plant roots, such that extracted p-coumaroyl tyramine demonstrates potent activity against E. coli; hexane extracts of hemp seeds have shown anti-Propioni bacterium acnes efficacy aligned with a suppressed innate response to infection in human keratinocytic cells. Cannabinoids may be incorporated into dental polish, which can assist in plaque removal and reduced gingival Filifactor alocis.

In specially bred mice, phyto-cannabinoids (CBD, THC, CBN), may be responsible for the reduction of cytokines production, that in turn counteract the rise of iNOS levels and the downstream cascade of events triggered by the inflammatory process, reducing thus oxidative stress. THC may inhibit the proliferation of mouse lymphocytes and decreased the production of interleukin-2 (IL-2), interferon-α (IFNα), reduce the secretion of pro-inflammatory cytokines like IL-1a, IL-1b and tumor necrosis factor-alpha (TNFα) in microglial cells. The antiproliferative and anti-inflammatory effects of CBD treatment in patients with acne scars can be associated to specific sebostatic actions of CBD: a) normalize the pathologically elevated lipogenesis induced by “pro-acne” agents, b) suppress cell proliferation and c) prevent the actions of toll-like receptor (TLR) activation or “pro-acne” agents to elevate proinflammatory cytokine levels. In another study, CBD and CBN were shown to exert a suppressive effect on human keratinocyte proliferation. CBD application may attenuate ovalbumin induced allergic airway inflammation in experimental mouse model of atopic dermatitis with reduced number of lung-infiltrating immune cells and of the serum Immunoglobulin (IgE and IgG) levels. In a retrospective study of 20 patients with skin disorders: psoriasis (n:5 patients), atopic dermatitis (n:5) and resulting outcome scars including acne vulgaris (n: 10) who administered topical CBD-enriched ointment (containing other botanicals) to lesioned skin areas twice daily for three months treatment. The results showed objective improvement (without adverse events, particularly in contrast with retinoic acid skin products) of skin evaluations (hydration, and transepidermal water loss by inhibiting the enzyme 5-α-reductase, subsequently inhibiting the excessive skin sebum secretion, as well as elasticity, which may be due to topical absorption of essential fatty acids, including α-linolenic acid, γ-linolenic acid, linoleic and oleic acids, and phytosterols (β-sitosterol)), which was also supported by photographic data and investigators' clinical assessment, with collaboration by clinical questionnaires confirming improved quality of life.

They can bind to receptors of the brain, nerves, skin and immune system and other cells. CBGA and CBG have both been evaluated in diverse scientific (in vitro, in vivo or clinical) studies and can potentially assist in improving well-being as well as ameliorating symptoms.

Both CBG and CBGA (similar to CBD(A) and CBC(A)) primarily modulate CB2R, along with other essential body receptors (non-ECS binding), and each has its own unique metabolic pathways. CBGA documented effects include: GPR55 inhibition, TRPA1 weak agonist, TRPV3 antagonist, TRPV4 antagonist, TRPM8 antagonist, AEA uptake inhibitor, DAG1 inhibitor, and others. CBG documented effects include: α2-adrenoceptor agonist (appears to be a unique effect of CBG), CB1R weak partial agonist, CB2R weak partial agonist, GPR55 antagonist, 5HT1A antagonist (CBD is an indirect agonist), TRPA agonist, TRPV1 agonist, TRPV2 agonist, TRPV4 antagonist, TRPMB antagonist, GABA reuptake inhibitor, Voltage-gated sodium channels (NaV) blocker, AEA uptake inhibitor, MAGL inhibitor, upregulated dopamine D4 receptor, increases NO, inhibitor of enoyl acyl carrier protein reductase, high antimicrobial potency, and others.

Clonidine, dexmedetomidine, lofexidine and tizanidine are pharmacologic agents currently available, which act as α2-adrenoceptor preganglionic agonists, in which they have been used clinically useful for analgesia (including complex regional pain syndrome-CRPS), anesthesia (with reduced likelihood of emergence delirium postoperatively or following pediatric conscious sedation), opioid dependency (useful for both withdrawal or abstinence) or other chemical dependency (SUD, withdrawal from tobacco, alcohol, benzodiazepine, and possibly for stress induced cravings for cocaine (or psychostimulants), ADHD (particularly in children), hypertension (or sympathetic hyperactivity, with vasodilation), anxiety or panic disorders, spasticity/muscle relaxant, but can result in sedation, hypotension, dry mouth or other adverse effects. Clinical researchers are currently have identified 6 potential molecules (2 of which have been used in experimentally studies) that can have a similar mechanism of action, and based on animal studies, relieved pain behaviors in neuropathic, inflammatory, and acute thermal nociception assays, without sedation, which would predictably have an opioid sparing effect, without dependency risk. CBG has a similar pharmacologic effect and its chemical structure has some common features with compound “4622.” CBGA and CBG are PPARα and PPARγ agonists.

CBG and CBGA have both been evaluated for anti-inflammatory, antimicrobial, anti-cancer, neuro-protective (also anticonvulsant) and antioxidant properties; they can bind to receptors of the brain, nerves, skin and immune system and other cells, which can potentially assist in improving well-being as well as ameliorating symptoms including cardiovascular, metabolic, GI, mental health, sleep, fatigue, autoimmune, inflammatory, neurologic, dermatologic, oncologic, eye and pain as well as anti-aging effects. Specifically, conditions that may be candidates for Cannabis supplementation (including cannabinoids, terpenes and hemp-derived nutritional products) for a goal amelioration of symptom control may include: pain (somatic-musculoskeletal, visceral, neuropathic and nociceptive, nocioplastic, or mixed, with peripheral or central sensitization with opioid sparing effects), psychiatric, mood, anxiety, PTSD, sleep, addiction, disorders, neurodegenerative disease/dementias (all types, including AD)/MS/cerebrovascular disorders/ALS/HD/PD), ischemic cardiovascular diseases, brain injury/concussion/cerebral palsy, myalgic encephalomyelitis/chronic fatigue syndrome (CFS), inflammatory or autoimmune disease (including rheumatoid, psoriasis arthritis, lupus-SLE), cachexia, nausea and vomiting, glaucoma, movement disorders/spasticity), osteoarthritis, gout, metabolic bone disease and osteoporosis or osteopenia, asthma, allergy, Inflammatory bowel disease (IBD, Crohn's disease or ulcerative colitis), irritable bowel syndrome (IBS)/dysbiosis/GI motility disorders, hypertension, diabetes, obesity, immunosuppression, toxic exposures, seizures, gastro protection, hyperlipidemia, neurogenic bladder dysfunction, cancer (many types), nephritis and renal ischemia, pelvic pain (including endometriosis) or menstrual disorders, sexual dysfunction, periodontal disease and gingivitis, skin dermatitis/acne/eczema/wounds, as well as respiratory illness (bacterial, fungal, parasitic infections, superinfections, viral including Covid-19 and its long term sequelae). This list is likely to be further expanded and evolving, based on ongoing basic science, animal and clinical research. There have been both human and veterinary therapeutic applications for these products, with varying routes of administration.

CBGA has been found in animal studies to be more effective for seizures triggered by a febrile event than CBD, and the acidic form, CBGA, has greater neuronal permeability. CBGA enhances receptor activation at low GABA concentrations, whereas prolonged exposure at higher concentrations increased the increased the desensitizing properties of the GABA-A receptor in mice. Neither CBG nor CBGV affected thresholds for hyperthermia-induced seizures, whereas the administration of THCV resulted in seizures at lower temperature thresholds in mice. However, when CBGA is combined with dobazam, an anticonvulsant with GABA, the effects were additive. It was also predicted that CBGA may be less useful for focal seizures.

CBG has an antioxidant effect and reduced nitrotyrosine, SOD1 and inducible nitric oxide synthase (iNOS) protein levels and restored nuclear factor erythroid 2-related factor 2 (NRF2) levels in motor neurons and macrophages resulted in a reduction of IL-1β, TNF-α, interferon (IFN)-γ and PPARγ protein levels as well as inhibited apoptosis, as shown by the reduction of caspase 3 activation and apoptosis regulator gene (BAX, also known as bd-2-like protein expression), while Bd-2 levels increased.

In rats, CBD and CBG exerted antioxidant effects in astrocytes (which are less sensitive to oxidative stress than neurons and they can ad by transferring healthy mitochondria to a damaged neuron) with decreased apoptosis and restored the cortex level of 5-HT depleted by neurotoxic stimuli (positively correlated) but acted differently for neuroinflammatory function. CBD restored the basal levels of β-hydroxykinurenine and kynurenic acid. CBG was less effective than CBD in restoring the levels of proteins involved in neurotransmitter exocytosis (however, the CBD assay was 1000× more concentrated than CBG). The anti-oxidative response of cannabinoids to hydrogen peroxide toxicity was seen at the lowest concentrations of CBG only, and at all concentrations of CBD. CBG has a higher binding affinity to neurokinin 3 receptors (NK3R involved in the pathophysiology of some neurological disorders, such as epilepsy and schizophrenia), related to CBG having more potency in blunting neurotransmitter depletion. CBD may be more selective with 5HT modulation than CBG to explain some of its anti-oxidant effects. A mouse model with CBG administered concomitantly to β-nitropropionate reported neuroprotective effects in the CBG treatment group with improved motor deficits, preserved striatal neurons, attenuated up-regulation of proinflammatory markers and microgliosis, as well as improvement of systemic antioxidant defenses. This animal model can be applied clinically for neurodegenerative conditions.

Cannabichrome (CBC): CBGA is converted to CBCA, as the result of a recessive gene-produced enzyme, CBCA-synthase in the trichome, which is then decarboxylated to form CBC (the third most abundant cannabinoid). CBC modulates CB1R, and binds to CB2R; it has been shown to significantly interact with TRPA1, TRPV1-4 as an agonist, and TRPMB as a weak antagonist; with high potency for GABA receptors, which may be useful to anxiety, sleep and spasticity; it may inhibit NO production, with anti-inflammatory and antimicrobial properties; CBC also inhibits NAAA, MAGL and enhances AEA cellular uptake, and suppresses excessive lipid production in the sebaceous (oil-producing) glands. CBC may be up to 10 times more potent than CBD for symptomatic management of stress and anxiety. CBC does not chemically crystallize into a powdered isolate (unlike CBG, CBD and CBN). Instead, distillate is the most concentrated form of CBC extract, usually coupled with CBD, 3:1. CBC may have the potential to be adulterated: it has been proposed that first, converting CBC with citral and 1,3-cyclohexadione into an isomeric perhydro-CBC analog, then, via ethylenediaminediacetate (EDDA), one may synthesize a perhydro-THC analog (with CB1R binding), as well as the hypothesis that CBC may thermally isomerize to THC during the process of smoking or vaporization.

Varin (V) Cannabinoids: When the side chain is a pentyl (5-carbon) chain the compound produced will be CBG. However, if the pentyl chain is replaced with a propyl (β-carbon) chain the CBG-type varin compound formed is CBGV (cannabigeroldivarin). The propyl variant will be formed if a 10-carbon precursor is reacted at the first stage of the biosynthetic pathway rather than a 12-carbon compound. CBGVA, in turn, converts to tetrahydrocannabivarin (THCV, found mainly in indica or special strains of sativa and not generally found in Panakeia due to lack of decarboxylation enzyme, synthase, a patented strain devoid of THCA/THCVA), Cannabidivarin (CBDV and not generally found in Panakeia due to lack of decarboxylation enzyme, cannabidiol synthase). THCV and CBDV have neutral antagonist activity at CB1R, which is a primarily binding site receptor for THC, which can be used as an anorexic agent in rats. Cannabichromevarin (CBCV) are considered homologous, varin cannabinoids (with smaller carbon side chains) and may be contained in Panakeia; Similarly, cannabigerovarin (CBGV) from CBGVA, has been researched for both its anti-inflammatory and anti-cancer effects and is minor component of most strains of Cannabis sativa, is also contained in the Panakeia biomass extract; it contributes to the entourage effect. Although these later two varins are chemically distinct from their associated cannabinoids, they probably have similar biologic effects as their conventional cannabinoid counterparts.

An analogous process, as described above, occurs for the propyl based, V derivatives, found at much lower concentrations, in hemp plants, including Panakeia (<2%), except for specialized C. indica cultivars, where the THCV concentration approaches 4-7% (and a chemovar reportedly averaging 15% THCV, along with THC content of 19% and CBD<1%), with CBDV up to 4% (representing 40-55% of cannabinoid content of a specific cultivar's flower). The THCV cultivars (and possibly other cannabinoid V cultivars) may have a slight stimulating effect with appetite suppression (for THCV, it may be related to CB1R antagonist effect at lower dosing, which may transition to CB1R agonist at higher titers); unlike the food cravings often accompanied by THC (if taken together, this may counteract the “munchies”). There are legal hemp CBDV cultivars that contain up to 6%, along with higher quantities of CBD, with possibly greater binding to vanilloid receptors. CBGV and/or CBGA may be more energizing than CBG or CBD, and when taken together, may improve their combined bioavailability.

Non-Cannabis Phytocannabinoids: Exogenous phyto-cannabinoids are chemically defined as meroterpenoids, the largest group of bioactive plant substances derived from isoprenoid precursors, with a phenolic moiety as the result of phenylpropanoid metabolism, with a resorcinyl core typically decorated with a para-positioned predominantly alkyl, but may also have isoprenyl, or aralkyl side chains instead. They are found in primarily in the C satival hemp or marijuana based plants (and to a lesser extent, Tremaorientais, a related species of the Cannabaceae family found in Thailand, with detectable cannabinoids that can be extracted, exhibiting antimicrobial effects); however, Cannabis uniquely produces Δ9-THC and its natural isomers.

The term “phyto-cannabinoids” includes, but is not limited to, cannabinoids from Cannabis and N-alkylamides from Echinacea. The term “terpenes” includes, but is not limited to, pinene, limonene, α-terpinene, terpinen-4-ol, carvacrol, carvone, 1,8-cineole, p-cymene, fenchone, β-myrcene, cannaflavin A, cannaflavin B, nerolidol, phytol and squalene. The term “terpenoids” includes, but is not limited to, cannabinoids, limonene oxide, pulegone-1,2 epoxide, salviorin A, hyperforin, and pyrethrins. As used herein, the term “lipids” includes, but is not limited to, of olive oil, sesame oil, coconut oil, vegetable oil, milk, butter, liposomes, glycerine, polyethylene glycol, ethyl acetate, d-limonene, butylene glycol, propylene glycol, ethylhexyl palmitate. N-alkylamides includes, but is not limited to, dodeca-2E,4E,8Z,1aZ-tetraenoic acid isobutylamide and dodeca-2E,4E-dienoic acid isobutylamide.

Among plants producing cannabinoid-like compounds, Helichrysum umbraculigerum, a South-African species of perennial is also a major producer of CBG. Other plants containing cannabinoids-like compounds are the Chinese rhododendron and liverwort Radula marginata in New Zealand.

Cannabinoids from Cannabis are not the only lipid based exogenous compounds interacting with the ECS. In the last few years, other plants have been found to produce cannabinoid-like compounds and several non-traditional cannabinoid plant natural products have been reported to act as cannabinoid receptor ligands. Consequently as “phyto-cannabinoid” is described any plant-derived natural product capable of either directly interacting with cannabinoid receptors or sharing chemical similarity with cannabinoids or both.

Among phyto-cannabinoids different from traditional Cannabis cannabinoids, that have been reported to interact with ECS, unsaturated fatty acid N-alkylamides (N-alkylamides) from the medicinal plant Echinacea, a species of herbaceous perennial plant in the family Asteraceae, have been demonstrated to bind to the CB2R more strongly than the endogenous cannabinoids. The interaction of N-alkylamides with the ECS has been proven to modulate induced immune response.

Certain N-alkylamides (alkamides) from Echinacea spp. containing N-isobutylamides have been shown to interact functionally with the human CB2R with low nM to μM Kᵢ values, leading to an increase in intracellular calcium, which possess anti-inflammatory effects similar to AEA (e.g., inhibition of TNF-α) and also target PPAR-gamma. The polyacetylenic polyyne, falcarinol, which is found in different plants of the Apiaceae family (e.g., in carrots, parsley or ginseng) shows significant binding interactions with CBR, but appears to selectively undergo an alkylation reaction with the CB1R, leading to relatively potent inverse agonistic and pro-inflammatory effects in human skin. These N-isobutylamides selectively at at the CB2R over the CBR, leading to an increase in intracellular calcium which could be blocked by the selective CB2R inverse agonist SR44528 ((1S-endo)-5-(4-Chloro-β-methylphenyl)-1-((4-methylphenyl)methyl)-N-(1,3,3-trimethylbicyclo (2.2.1)hept-2-yl)-1H-pyrazole-β-carboxamide), but they do not modulate the Gαi signaling pathway. Intriguingly, CB2R-binding N-alkylamides show similar anti-inflammatory effects as anandamide (e.g., inhibition of TNF-α) at low nM concentrations. Certain Echinacea N-alkylamides inhibit anandamide reuptake in vitro. Like anandamide, N-alkylamides also target PPARγ. Different Echinacea N-isobutylamides are orally bioavailable resulting in nM plasma levels in humans.

Other constituents from Echinacea purpurea act as weak CB1R antagonists, include: Echinacea purpurea, Echinacea angustifolia, Acmella oleracea, Helichrysum umbraculigerum, and Radula marginata. The best-known cannabinoids that are not derived from Cannabis are the lipophilic alkamides (alkylamides) from Echinacea species, most notably the cis/trans isomers dodeca-2E,4E,8Z,1aE/Z-tetraenoic-acid-isobutylamide. At least 25 different alkylamides have been identified, and some of them have shown affinities to the CB2R. In some Echinacea species, cannabinoids are found throughout the plant structure, but are most concentrated in the roots and flowers. Yangonin found in the Kava plant has significant affinity to the CB1R. Tea (Camellia sinensis) catechins have an affinity for human cannabinoid receptors. A widespread dietary terpene, beta-caryophyllene, a component from the essential oil of Cannabis and other medicinal plants, has also been identified as a selective agonist of peripheral CB2R, in vivo. Black truffles contain AEA. Perrottetinene, a moderately psychoactive cannabinoid, has been isolated from different Radula varieties. Most of the phytocannabinoids are nearly insoluble in water but are soluble in lipids, alcohols, and other non-polar organic solvents.

Cannabis is also closely related to hops, which also contains cannabinoids, terpenes and terpenoids, and possesses a similar natural flavor, but intrinsically lack synthase enzymes to produce any THC.

Numerous plants (non-Cannabis species and/or terpenes/terpenoids that are highly expressed, but not usually contained in Cannabis plants) have bioactive terpenoids with a cannabinoid backbone, and possess cannabinomimetic binding; they also do not contain THC and may act as insect deterrents or for antimicrobial action: rhododendron (epidotes with small leaves and glandular scales), licorice, dove, black pepper, broccoli, ginseng, carrots, Helidrysum umbraculigerum, liverwort, kava, bastard indigo, echinacea, Apiaceae family, Mirabilis jalapa, Lithophragm glabrum, Cordia verbenacea, Eucalyptus globus, Syzygium aromaticum, Senna didymobotrya, salvinorin A, celastrol, falcarinol, pristimerin, citral, Cymbopogon citratus and in some Citrus genus plants (Citrus limon), and fungi: Saccharomyces cerevriae, Asteraceae, Nectriaceae and Mycorrhizal fungi, or others; some of these terpenes/terpenoids may also be synthesized (by laboratory or recombinant technologies). Phyto-cannabinoids (and some terpenes/terpenoids) can modulate endo-cannabinoid effects, and also bind to non-ECS receptors, but they have different affinities, proportional binding, modulating effects and potencies. Often as a diverse class, they possess antioxidant, anti-inflammatory, anti-neoplastic and antimicrobial properties, by binding both to the CNS and peripheral tissues.

The polyacetylenic polyyne falcarinol, which is found in different plants of the Apiaceae family (e.g., in carrots) shows significant binding interactions with both cannabinoid receptors, but appears to selectively undergo an alkylation reaction with the CB1R (Kᵢ value<1 μM), leading to relatively potent inverse agonistic and pro-inflammatory effects in human skin.

The bicyclic sesquiterpene, β-caryophyllene (trans-isomer), which is a plant volatile, and very frequently found in plants, has been shown to selectively target the CB2R at nM concentrations (Kᵢ=155 nM) and to at as a full agonist. Remarkably, β-caryophyllene is also a major compound in Cannabis sativa L essential oil. Thus, Cannabis produces two entirely different chemical scaffolds able to differentially target CBR. While studies on the pharmacokinetics of β-caryophyllene are still ongoing, it is already clear that this cyclobutane-ring containing terpene is readily bioavailable, and, unlike many polyphenolic natural products, is not metabolized immediately but shows a Tₘₐₓ>1 h after one single oral administration. Orally administered β-caryophyllene (<5 mg-kg⁻¹) produces strong anti-inflammatory and analgesic effects in wild-type mice but not in CB2 receptor knockout mice, which is a clear indication that it may be a functional CB2R ligand. Ongoing studies show that β-caryophyllene is effective at reducing neuropathic pain in a CB2R-dependent manner. Therefore, the FDA approved food additive β-caryophyllene has the potential to become an attractive candidate for clinical trials targeting the CB2R. Interestingly, the diterpene salvinorin A from Salvia divinorum has been reported to be a selective high-affinity K-opioid receptor (KOP) agonist, but recent data also suggest that it may interact with a putative CBR/KOP heterodimer which may be formed during inflammatory conditions (Fichna et al., 2009). Binding experiments have shown that salvinorin A has very low affinity for homomeric cannabinoid receptors and does not inhibit endocannabinoid degradation.

More recently, two naturally occurring quinonoid triterpenoids, pristimerin and euphol, were found to inhibit acylglycerol lipase, MAG1, with high potency (IC₅₀=93 nM and 315 nM respectively) through a reversible mechanism.

Catechin-derivatives (e.g., epigallocatechin β-gallate and (−)-epigallocatechin) were shown to bind to human CBR rather non-selectively at high μM concentrations (Korte et al., 2010). Catechins are very widespread plant secondary metabolites which may provide nutritional health benefits, and can assist with weight loss.

Plants produce fatty acid amides, some of which are able to inhibit the degradation of anandamide but do not generally bind with significant affinity to CBR. At present, the only phytocannabinoid that has been discovered to also exist in plants other than Cannabis is β-caryophyllene, which is among the most abundant plant essential oil components, and could be considered as a true CB2R-selective Cannabis constituent.

Terpenes: Terpenes are aromatic compounds found in many plant species, and their distribution gives each Cannabis cultivar a unique aroma and flavor; they are contained in Cannabis extracts, have complex plant biosynthesis. The terpene/flavonoid content of any hemp plant can vary based on the growing milieu including geographic (altitude, temperature, humidity, light exposure, watering), and plant variety. The lots tend to be quite homogenous (e.g., if one lot is cultivated outdoors in certain conditions or in greenhouse).

They have a common precursor as cannabinoids, geranyl diphosphate (GPP), with higher concentrations of 10 primary and 20 secondary terpenes (with >200 terpenes and >200 terpenoids identified, depending on the variety of hemp, and the typical terpene/terpenoid content in hemp range from 0.125% to 0.278% weight in moist leaves or 1.283% to 2.141% in the inflorescence on a dry basis)) found primarily in the trichomes of female plant, and their distribution and proportions can vary in cultivars and can be maximized with higher photoperiods, Photosynthetic Photon Flux Density (PPF), temperature, humidity, altitude, soil pH, salinity, stress, reduced mineral content with relative nitrogen lack just prior to harvest, pest and pathogens pressure, watering regimen, pruning, plant density, plant genetics/hybridization (most significant), as well as harvest and extraction methods, among a myriad of other factors. Terpenes are more highly volatile (may be oxidized into terpenoids between 119° C. to 198° C., or evaporate with drying, and each has a unique aroma Therefore, standardization with respect to these compounds can be difficult, with relatively large inter-batch differences, which may affect flavor and perceived effects.

An incomplete list of terpenes includes: β-myrcene (abundantly found), β-phellandrene, d-limonene, linalool, piperidine, p-cymene, α- and β-pinene, terpinolene, nerolidol, guaiol, geraniol, eudesmol, citral, β-elemene, cis-ocimene, α-terpineol, eucalyptol, phytol, curcumene, pulegone, ocimene, valencene, burneol, camphene, carene, squalene, cineole, β-Farnesene, fenchone, butylated hydroxytoluene (BHT) 1,6-Dioxacyclododecane-7,12-dione (may be harmful if swallowed, eye, skin and respiratory irritation and specific target organ toxicity) and bisabolol; sesquiterpenes: β-caryophyllene, and its closely related, α-humulene, g-cadinene, eudesma3,7(11)-diene, and d-guaiene), even if taken alone, some have been associated with a wide and diverse range of effects including anti-cancer, anti-inflammatory, antioxidant, wound healing, immunological, antimicrobial effects, sugar or lipid metabolism, cardiovascular or digestive disorders, appetite, anti-aging, neuroprotective, as well psychological benefit for analgesia, anxiety, depression, or substance misuse. Some terpenes (terpinene, guaiol, phellandrene, carene, and sabinene) have an activation effect which may increase anxiety. Some have GRAS designation (pinene, caryophyllene, bisabolol, limonene, linalool, and myrcene, and others), and used in aromatherapy scents, although some can have toxic effects with excessive exposure including asthma, or cancer risk.

Fresh Cannabis terpenoid content is composed mainly of 92% monoterpenes, with 7% sesquiterpenes. After drying and curing, there is an expected loss of terpenes overall and a “significantly greater loss of monoterpenes than sesquiterpenes.” Most of this loss happens after the first week of drying, especially when using steam-distilled oil, in which the content drops from 0.29% to 0.20% of the original plant material. There is a drastic change that occurs in the major terpenes found in the cultivars prior to harvest, at harvest, and after harvest (drying) and after curing. Monoterpenes such as β-myrcene and α-pinene, show reductions in their percentage out of the overall essential oil (ED) terpene composition while sesquiterpenes such as β-caryophyllene and α-humulene show an increase in their percentage in overall ED terpene composition. α-pinene, for example, had a larger reduction in the 3 weeks prior to harvest and linalool seems to be the least affected by the processing steps. Cannabis also includes salviorin A, hyperforin, and pyrethrins. Terpenes may work in combinations with other components of Cannabis, to provide an entourage effect, and they are found in other botanicals as a potential source. They may act by reducing toxicities of chemotherapeutic drugs and it is possible that they act through other CBR-dependent signaling pathways that do not involve potassium channels, or ad through other non-ECS pathways). Myrcene has been studied in breast, cervical, lung, colon carcinoma, and leukemia; caryophyllene for lung cancer, ovarian, biliary, skin cancer, gliobastoma, and pre-cancerous skin conditions; humulene for hepatocellular, and ovarian carcinoma; limonene for bladder, prostatic, colon, lung, breast, hepatocellular, cancer, and in animals, T cell lymphomas and melanoma; pinene for hepatocellular and lung cancer; linalool for hepatocellular and skin carcinoma, lymphoma, glioma, sarcoma; bisabolol for glioma, non-small cell lung, pancreatic, endometrial (and possibly ovarian, breast and prostate when using chamomile) carcinoma, lymphoid leukemia; elemene for non-small cell lung cancer, hepatocarcinoma, metastatic brain cancer, and leukemia; eudesmol for biliary, lung and colon cancer; eucalyptol for prostatic, colon and ovarian carcinoma; borneol for glioblastoma, squamous esophageal, hepatocellular carcinoma and melanoma; terpineol for glioblatoma and colon carcinoma; terpinene for hepatocellular carcinoma and leukemia; valencene for ovarian carcinoma; geraniol for colon, prostatic and hepatocellular carcinoma; nerolidol for breast, small intestine, colon carcinoma, leukemia; guaiol for non-small cell lung carcinoma; camphene for melanoma; phellandrene for hepatocellular carcinoma; carene for cervical cancer; cadinene for ovarian carcinoma; thujone for lung cancer and gliobastoma; cymene for blood, renal, lung, prostate, liver, breast cancers; gurjunene for lymphoma; farnesene for breast cancer. In addition, flavonoids also have anti-neoplastic activity: kaempferal for leukemia, breast, ovarian, colon, liver, stomach, bladder, oral, prostate, lung and bone cancers, neuroblastoma, lymphoblastic leukemia; apigenin for colon, hepatocellular, esophageal carcinoma, glioma; cannflavins for pancreatic carcinoma; silymarin for Burkitt's lymphoma, melanoma, prostatic, hepatocellular carcinoma; luteolin for breast, prostate, oral, lung, kidney, cervical, placental, ovarian, skin, liver, esophageal, bladder, colon cancer, melanoma and glioblastoma; orientin for colorectal (also polyps), esophageal squamous and transitional cell bladder carcinoma; vitexin for melanoma, colon, hepatocellular, prostate cancer and osteosarcoma; quercetin for breast, prostate, leukemia, ovarian, gastric, colon, lung adenocarcinoma, liver cancer, non-small cell lung cancer, osteosarcoma, melanoma, glioma, myeloid leukemia.

Limonene has a strong lemony scent also found in citrus fruits, thought to have anti-cancer, immune potentiator, antimutagenic, anti-inflammatory, fast acting mood-enhancing effects, reduced social anxiety, boost confidence, but with body relaxation, possibly dissolves gallstones, improves digestion, GERD and peristalsis, possible anti-breast cancer.

d-Limonene, found in citrus products has a high bioavailability, may increase serotonin in the prefrontal cortex, and dopamine (DA) in hippocampus as mediated via 5-HT1A, contributing to mood/anxiety, GI protection, antioxidant, anti-cancer (breast) and dermophytes. p3-Myrcene is very abundant in nature; also found in hops, mangos, sweet basil, lemongrass, parsley bay leaves and wild thyme; This terpene has an earthy, musky, almost fruity tone. may enhance the effects of THC. It possesses analgesic, anti-inflammatory, antibiotic (by enhancing transdermal absorption and is an effective antimicrobial agent), antimutagenic and sedating/relaxing, antinociceptive activity (i.e., induces insensitivity to pain) in rodents, acting both centrally and peripherally and may involve the mediation of endogenous opioids. β-myrcene to inhibit certain forms of the cytochrome P-450 (2B subfamily) enzymes. In humans, dermatitis, conjunctivitis, and somnolence have all been reported following exposure to β-myrcene. In a single case report, chronic exposure to β-myrcene fumes caused severe and lingering asthma-like symptoms in a hops inspector for a brewery. B-Myrcene, when administered orally to pregnant Wistar rats, induced a significant reduction in maternal weight gain, with additional effects for visceral malformations and delayed ossification. β-myrcene is commonly found in hops preparations (Humulus lupulus), and activates TRPV1 and has anti-inflammatory activity, via prostaglandin E-2 (PGE-2), blocks hepatic carcinogenesis by aflatoxin (a toxin found in decayed peanuts), analgesic in mice (but this action can be blocked by naloxone, an opioid antagonist, perhaps via the α-2 adrenoreceptor and is muscle relaxant and sedating, which may act as a sleep aid. β-myrcene is currently being studied as a possible human carcinogen and genotoxic effects especially after metabolic activation using human hepatic HepG2/C3A cells. The FDA took regulatory action in 2018 to no longer allow the use of the food additive, myrcene, a synthetic flavoring agent, based on results from National Toxicology Program carcinogenicity studies in rats (kidney cancer).

Linalool is a relaxing scent, also found in lavender (Lavandula angustifolia), conveys anxiolytic activity, wound and skin rejuvenation, antinoceptive, anticonvulsant, believed to be related to GABA and glutaminergic effects and also has anesthetic effects. It's supposed sedative effects, helpful for aiding sleep, psychosis, epilepsy, immune potentiator, antibacterial, antifungal, anti-anxiety, antidepressant, analgesic, flatulence relieving, as well as to have beneficial immunomodulatory effects on wound healing, anticancer with higher exposures, may cause eye and skin irritant with 7% of people found to be allergic undergoing patch testing in Europe were to the oxidized form of linalool, GRAS. Linalool, has analgesic effects, and is a known NMDA receptor antagonist.

Pinene has a pine-like scent, found in pine nuts, orange peels, rosemary, dill, and basil pine odor and acts as a bronchodilator, anti-inflammatory, antibiotic, AChE inhibitor, antianxiety, analgesic, anesthetic-like effects, antineoplastic: inhibits melanoma, non-small cell lung carcinoma, ovarian cancer, neuroblastoma, lymphoma and hepatocellular carcinoma, antioxidant benefits, anticoagulant properties, GI benefits including pancreatitis, dental pain some guidance on inhalation hazards but not in concentrations you could expect to find in Cannabis, GRAS. α-Pinene, the most widely encountered terpenoid in nature confers an insect-repellent role, anti-inflammatory via PGE-1, bronchodilator in humans at low exposure levels and an acetylcholinesterase inhibitor (improves memory). α-Caryophyllene (or Humulene, an isomer of β-caryophyllene) has a spicy taste, found in hops, sage, ginseng, and loves; also in caraway cinnamon, oregano lavender rosemary basil and black pepper, acting on the body's CB2R pathways. Anti-inflammatory, Cytoprotective (gastric mucosa), appetite suppressant, Antimalarial, potentiates opioids, anti-aging protective role in a number of nervous system-related disorders including pain, anxiety, spasm, convulsions, depression, alcoholism, and Alzheimer's disease; potential anticancer, may help gut and bowel disorders (such as IBS, colitis) to inhibit TRPM8, an archetypical cold-activated ion channel of mammals, GRAS Caryophyllene oxide, in which the alkene group of caryophyllene has become an epoxide, is the component responsible for Cannabis identification by drug-sniffing dogs and is also an approved food flavoring. humulene's anti-inflammatory, anti-cancer, and anti-appetite forces amplify when combined with its cousin, beta-caryophyllene (β-caryophyllene), the “dietary cannabinoid” of terpenes.

β-caryophyllene is generally the most common sesquiterpenoid encountered in Cannabis inhibiting insect herbivory, antimalarial effects, anti-inflammatory via PGE-1 with analgesic effects, and may reduce contact dermatitis. Its oxide is antifungal, insecticidal, anti-feedant and anti-platelet aggregation. Orally administered β-caryophyllene has a high bioavailability, with maximal onset after 1 hour; It is a CB2R agonist, which inhibits the pathways triggered by activation of the toll-like receptor complex CD14/TLR4/MD2, which typically leads to the expression of pro-inflammatory cytokines e.g., IL-1 beta, IL-6, IL-8, and TNF alpha) and promotes a Th1 immune response that plays a critical role in neuro-inflammation, sensitization, and pain. β-caryophyllene activates PPARγ and also inhibits TRPM8, may be gastroprotective, and its oxide has been used for Cannabis identification by drug-sniffing dogs. β-Caryophyllene is found in Cannabis sativa [3.8-37.5% of Cannabis flower essential oil], as well as Black caraway (Carum nigrum) [7.8%], loves (Syzygium aromaticum) [1.7-19.5% of dove bud essential oil], Hops (Humulus lupulus) [5.1-14.5%], Basil (Ocimum spp), Oregano (Origanum vulgare) [4.9-15.7%], Black pepper (Piper nigrum) [7.29%] Lavender (Lavandula angustifolia) [4.62-7.55% of lavender oil], Rosemary (Rosmarinus officinalis)[4] [0.1-8.3%], True cinnamon (Cinnamomum zeylanicum) [6.9-11.1%], Malabathrum (Cinnamomum tamala) [25.3%][34], Ylang-ylang (Cananga odorata) [3.1-10.7%], and Copaiba oil (Copaifera) essential oils, and when combined, may have synergistic effects and improved bioavailability.

Friedelin is a terpenoid, with anti-inflammatory, antimicrobial, gastroprotective, adaptogen properties.

Terpinene is exotic, smells like turpentine, found in spices marjoram and cardamom, may increase anxiety, has antibacterial and antimicrobial properties for topical application and as a treatment of acne or atopic dermatitis.

Guaiol has a sweet, fruity flavor similar to plums pine-like aroma, found in cypress pines ginger, ginseng, and valerian, is actually sesquiterpenoid alcohol instead of an oil, may increase significant anxiety-causing effects (also seen with other minor terpines in Cannabis—phellandrene, carene, and sabinene). There is also current controversy whether it also extolls anti-analgesic effects (avoid if you're taking CBD or Cannabis for pain relief). Guaiol is also a diuretic. It may lower high blood pressure, has anti-tussive effects, anticancer benefits for non-small cell lung cancer with anti-inflammatory effect, antioxidant, antiparasitic/microbial bacterial fungal effects, and insect repellent, it reduces inflammation in the lungs to treat coughing as well as in your limbs and other organs, making it above average at treating inflammatory conditions as diverse as arthritis, constipation, gout, sore throat, and even syphilis. It can strengthen the effects of chemotherapy and potentially reduce tumors and possibly stimulate menstruation and was even used as an abortifacient. Melting Point: 91.00 to 93.00° C. at 760.00 mm Hg Boiling Point: 309.00 to 310.00° C. at 760.00 mm Hg Vapor Pressure: 0.000054 mmHg at 25.00° C. (est) Flash Point: 236.00° F. TCC (113.33° C.) log P (o/w): 4.782 (estimated) Shelf Life: 12 month(s) or longer if stored properly. Storage: refrigerate in tightly sealed containers. Soluble in: alcohol, water, 3.61 mg/I at 25° C. (est.), Insoluble in: water.

Nerolidol has sweet and flowery scent, tastes similar to citrus found in jasmine, ginger, lavender, and the tea-tree antioxidant effects “in counterbalancing the effect of radical free oxygen (ROS) by protecting the cells against oxidative damage to lipids, proteins, and DNA, significant anti-anxiety cross the skin barrier up to 1956% more efficiently, making it more effect if used in analgesic topical creams, anti-microbial and antifungal effects possible anti-cancer properties. Nerolidol is a sesquiterpene alcohol with sedative properties t diminished experimentally induced formation of colon adenomas, enhances skin penetration of topical 5-fluorouracil, a chemotherapy agent, antifungal, antiprotozoal, antimalarial, and antileshmanial.

Bisabolol has flowery scent with a mild and sweet taste found in chamomile, anti-inflammatory and anti-microbial effects which make it a useful addition to creams eczema and psoriasis anti-irritant and skin healing benefits but may cause contact allergies.

Carene has fresh piney, with bitter after taste and fir needles, musky earth, and damp woodlands combination scent found in basil, anti-inflammatory, antifungal, and antibacterial treat acute skin inflammation may improve bone health used in cosmetics. natural antihistamine effect significant anxiety-causing effects.

Eucalyptol (makes up less than 0.06% of the complete terpene profile): minty smell that is also slightly spicy and cooling taste, found in sweet basil, bay leaves, wormwood, rosemary, and common sage, pain-relieving as an agonist of TRPMB channels and anti-inflammatory properties increase in gastric mucus production which can be protective of the stomach and intestines respiratory effects significant anxiety-causing effects insecticidal properties antimicrobial anti-fungal toxic if ingested in larger quantities. It has the properties of: AChE inhibitor, Increases cerebral blood flow, Stimulant, Antibiotic, Antiviral, Anti-inflammatory, Antinociceptive. Eucalyptol is a TRPM8 agonist.

Camphene (found in small quantities) has a musky, earth, damp, woodland smell and can be found in ginger camphor oil and citronella oil; it has antitumor against melanoma and antioxidant properties, inhibits lung inflammatory diseases by reducing oxidative stress, may lower triglycerides and cholesterol with topical use may help inflammatory skin conditions such as psoriasis and eczema.

Borneol taste and smell are spicy and minty; helps ovarian cancer, help regain consciousness after traumatic brain injury (TBI), used as an adjunct to cross BBB, ads as digestive aid, help with heart function and aid in circulation, at higher concentrations, is an eye and skin irritant.

Terpineol has a sweet and slightly fruity flavor floral scent similar to lilacs for a strong sedative effect, has cytotoxic activity in the small cell lung carcinoma, anti-inflammatory and pain-relieving effects, antifungal as a natural food preservative for fresh fruits.

Valencene has a fruity smell, found in Valencia oranges, used in cosmetics and perfumes, antimicrobial inset repellant for ticks and mosquitoes.

Geraniol has a fruity taste, found in rose oil and citronella oil, antioxidant potential, exceptionally high bioavailability 92% after 30 min ingestion, is sedating, may help protect the brain from neurological disorders such as dementia or AD, may at as a “multi-target agent” against cancer, can be used as a scenting agent in bath products or sweet-smelling insect repellent for mosquitos; Bees use geraniol to mark the location of nectar-producing flowers.

Ocimene has a sweet, herbaceous, and woody aroma, found in a wide variety of fruits and herbs, including mint, parsley, pepper, basil, mangoes, orchids, has anti-inflammatory effects with strong antifungal and antiviral properties. γ-Eudesmol is sweet and waxy tasting compound, can be found in a number of food items such as rosemary, ginkgo nuts, mango, and common thyme; it has antiproliferative, antioxidant and antibacterial properties. α-Eudesmol found in orange mint and wild celery, both in the methanol family, has antimicrobial activities.

Guaienol from woodsmoke, celery seeds, tobacco leaves, orange leaves, and lemon peels flavor of many substances such as whisky and roasted coffee and can be synthesized to vanillin); can be used expectorant, antiseptic, and local anesthetic. γ-Curcumene is also found pepper (spice), lovage, wild carrot, and rosemary, it ads as an antifeedant and antifungal and is one of the major chemical constituents of turmeric, with known anti-inflammatory, analgesic and antioxidant properties.

Pulegone may also be obtained from the essential oils of a variety of plants such as Nepeta cataria (catnip), Mentha piperita, pennyroyal and spearmint; it may be beneficial for IBS and other digestive conditions, as well as pain relief. It contributes to peppermint oil, which is generally safe, but it can be toxic when taken in very large doses. and may at as a carcinogen that causes hepatic carcinomas, pulmonary metaplasia, and other neoplasms with oral administration in rodents.

trans-caryophyllene has a sharp smell that rises from cracked pepper, found in oil of Mentha longifolia and Commiphora gileadensis; it has antibacterial, anti-inflammatory, antimicrobial, antileishmanial compound and antioxidant properties, with Anxiety Relief, Cholesterol Reduction, Osteoporosis Prevention, Seizure Management has anti-spasmodic activity on rat tracheal smooth muscle. β-Elemene (1-methyl-1-vinyl-2,4-diisopropenyl-cyclohexane) is a derivative terpenoid found in Cannabis sativa, which may arise due to oxidation or due to thermal- or UV-induced rearrangements during processing or storage. However, β-elemene is present not only in Cannabis sativa but also from Curcuma rhizome, and it is commonly used in traditional Chinese medicine due to its anticancer properties with no reported severe side effects. In this way, this compound has been extensively studied as an anticancer agent in vitro and in vivo and has been demonstrated to be a promising drug for the treatment of a wide variety of tumors-. β-elemene shows the ability to modulate essential biological functions, such as inflammation, oxidative stress, immunology response, cell division, as well as endothelial regulation.

Citral, (2E)-3,7-dimethylocta-2,6-dienal, is the main compound of essential oils that have been used mainly in popular medicine in eastern countries. It is the major compound extracted from Cymbopogon citratus, popularly known as lemongrass, but it can also be extracted from different plants including lemon myrtle and Lindera citriodora. This essential oil has been used as ingredient in foods because of its lemon-like fragrance. However, citral has gained attention in the last years due to its antimicrobial properties against Cronobacter sakazakii, a foodborne pathogen clinically associated with neonatal infections such as meningitis, septicemia, and/or necrotizing enteritis. Its reported antimicrobial activity also extends to Staphylococcus aureus, Candida albicans, Enterobacter cloacae, Listeria monocytogenes, Aeromonas spp., and Streptococcus pyogenes.

Celastrol,2R,4aS,6aR,6aS,14aS,14bR-10-hydroxy-2,4a, 6a, 6a, 9,14α-hexamethyl-11-oxo-1,3,4,5,6,13,14,14b-octahydropicene-2-carboxylicacid, is a pentacydictriterpenoid isolated from Tripterygium wilfordii root extracts and used in traditional Chinese medicine for treatment of chronic diseases, including neurodegenerative disorders (e.g., amyotrophiclateral sclerosis, AD, and PD), type 2 diabetes, obesity, atherosclerosis, cancer, inflammatory and autoimmune diseases (e.g., SLE, MS or IBD), psoriasis, and rheumatoid arthritis (RA).

An example of terpene content in a full-spectrum Cannabis extract is usually detectable above 0.05% volume/weight (v/w) threshold includes, β-myrcene (0.47% v/w), β-caryophyllene (0.05% v/w), d-limonene (0.14% v/w) and α-pinene (0.049% v/w). Most animal studies are based on terpenes or terpenoids are derived from other plant or synthetic sources, resulting in clinical effects that may be extrapolated to humans (few studies that have been conclusive, but have significant clinical dietary applications, since most of these compounds or their derivatives are GRAS).

In mouse studies, some terpenes (α-humulene, geraniol, linalool, and β-pinene) bind to CB1R, and lowered pain sensitivity, while attenuating cannabinoid-related side effects including lowered body temperature, reduced movement and catalepsy (a freezing behavior related to THC) and β-caryophyllene exhibits CB2R agonism. Many other terpenes bind to non-ECS receptors for synergistic physiologic effects.

Mice exposed to terpenoid odors inhaled from ambient air suggests there may be a direct pharmacological effect on the brain, resulting in activity changes (i.e., linalool with 73% reduction in motility, terpineol 45% reduction, pinene 13.77% increase, limonene, and 35.25% increase).

Vape oil contains small amounts of terpenes (1-7%). The most common terpenes and natural extracts found were Caryophyllene (12 samples), Alpha-Bisabolol (11 samples), Linalool (10 samples), Alpha-Humulene (9 samples), Caryophyllene oxide (8 samples), D-Limonene (8 samples), Phytol (8 samples), Fenchol (6 samples), Nerolidol (6 samples), Selina-3,7(11)-diene (6 samples), Squalene (6 samples), Vitamin E (6 samples), Beta-Myrcene (5 samples), and Gamma-Selinene (5 samples). Among these commonly found terpenes, Caryophyllene, D-Limonene, Alpha-Humulene were found at higher percentage compared to other terpenes.

Terpenoids are modified terpenes and are pharmacologically versatile: they are lipophilic, interact with cell membranes, neuronal and muscle ion channels, neurotransmitter receptors, G-protein coupled (odorant) receptors, second messenger systems and enzymes, with antioxidant and anti-inflammatory effects. Additionally, most terpenoids are non-sensitizing to skin when they are contained in fresh extracts or essential oils, but may cause allergic reactions (rash, urticarial) at very low rates when oxidized (shelf life and storage dependent).

Other Cannabis Constituents: Cannabis constituents other that cannabinoids and terpenes include bioflavonoids (approximately 3% dry weight), flavones and flavonoid glycosides (including quercetin, terpinolene and kaempferol); antioxidants, found primarily in the buds and leaves (also contain fiber, vitamins and minerals); phyto-sterols (including steroids, and apigenin, a phyto-estrogen); alkaloids (a diverse class, including indolic spermatidine: annhydrocannabisativine, for neurodegenerative, anticonvulsive, psychiatric, antimicrobial and anti-neoplastic effects by functioning as adenosine receptor agonists, anti-oxidant, anti-amyloid, acetylcholinestrase and butyrylcholinesterase inhibitor, inhibitor of α-synuclein aggregation, DO agonist, and NMDAR antagonist); piperidines (neoechinulin for anti-inflammatory, anti-mutagenic and antimicrobial effects) polyphenols (cannflavins and stilbenoids), with multiple antioxidant effects, and p-coumaric acid and ethyl p-coumarate as the main phenolic compounds contained in the hemp root, with antimicrobial activity; phytomelatonin (13.43-30.40 ng/g), aerial parts (leaves, fruits, flowers, 1.16-4.85 ng/g. and roots and possibly interact with the auxin receptor. Auxin is involved in modulating the development of roots and leaves, in the interactions seems to protect against senescence and protection from oxidative stress; pigments (anthocyanins and carotenoids), as well as essential nutrients including nitrogenous compounds, amino acids (particularly methionine and lysine). Melatonin can also act as a preservative and induce cold tolerance by activating C-repeat binding factors (CBFs) as transcriptional factors for cold acclimation in post-harvest crop; feruloyltyramine, a phenolic amide compound found primarily in seeds, an anti-inflammatory, antioxidative, and antiproliferative properties, which NO) production, and potently inhibits COX-1/2 effect on platelets; biogenic amines increased with lacto-fermentation: spermidine, having anti-inflammatory and anti-antioxidant properties, enhance respiration and metabolic function may reduce the risk of diseases like cancer, metabolic disease, heart disease and neurodegeneration and advanced non-alcoholic fatty liver disease; proteins (plant metabolic enzymes, edestin, albumin, and vicilin-like protein); glycoproteins (water soluble, linkages connected via serine-lgalactoside); sugars (common monosaccharides: fructose, glucose, mannose; selected disaccharides: sucrose, maltose; and several polysaccharides: cellulose, pectin; as well as several sugar alcohols: mannitol, sorbitol, and glycerol); hydrocarbons, lactones, ketones, simple alcohol, aldehydes, esters, fatty acids (α-linolenic acid, oleic acid, and linoleic acid), lipopolysaccharides, cannabisin F, alignanamide in hemp seeds, with neuroprotective effects, minerals (calcium, molybdenum, iron, potassium, zinc, manganese, copper, sodium and magnesium in the order of decreasing frequency), vitamins (K, C and folate, tocopherols), etc., Plant alkaloids, including neoechinulin (has radical scavenging, anti-inflammatory, antiviral, anti-neurotrophic factor-like, anticancer, pro-apoptotic, and anti-apoptotic properties), may attenuate the development of neurodegenerative diseases through their vast mode of action including inhibiting the activity of acetyl-cholinesterase (AChE) enzyme, by increasing the level of GABA, and by acting as antagonist of NMDA, as well as having anti-inflammatory, anti-neoplastic, and antimicrobial effects.

Bioflavonoids can be subdivided into six major subclasses: flavones, flavanols, flavanones, flavanols, isoflavones and anthocyanidins (about 3% dry weight Cannabis). Flavones and flavonoid glycosides are the largest class of polyphenols, including quercetin, terpinolene and kaempferol, up to 2.5% dry weight) antioxidants, found primarily in the buds and leaves (also contain fiber, vitamins and minerals).

Cannflavins a flavone unique to Cannabis, inhibits PGE-2 thirty times more potently than aspirin, may at as an anti-inflammatory agent, as well as anti-neoplastic and offers neuroprotection. It may also exhibit antimicrobial activity, especially by binding viral proteins.

Apigenin, a yellow pigment flavonoid phytoestrogen, also found in Cannabis, inhibits TNF-α, a mechanism germane to multiple sclerosis and rheumatoid arthritis.

Kaempferol may have anti-neoplastic properties and is an antioxidant, suppressing amylase and glucosidase, two enzymes that break down carbohydrates and useful in sugar metabolism.

β-sitosterol, a phytosterol found in Cannabis, may have anti-neoplastic or anti-inflammatory activity, and reduces cholesterol, possibly effective in prostatism. It reduced topical inflammation 65% and chronic edema 41% in skin models.

Luteolin is a flavonoid with anti-inflammatory and neuroprotective properties, exerts its effects through neurotrophic and antioxidant mechanisms. Luteolin increases the expression of the BDNF, which supports neuronal survival, growth, and plasticity; BDNF is essential for learning and memory. Luteolin also promotes the antioxidant response by reducing intracellular ROS in neurons. The reduction in circulating ROS diminishes the overall inflammatory milieu and reduces the proportion of M1 pro-inflammatory microglial cells. The decreased ROS also improves mitochondrial function, ameliorating the brain environment and facilitating the recovery of neuronal connections. Luteolin may have anxiolytic and neuro-protective properties.

Glycosides may have antioxidant, anti-diabetes, anti-inflammation, anti-microbial, and anti-neoplastic activities.

Quercetin has antioxidant and anti-inflammatory, anti-neoplastic, may improve glycemic control, help prevent heart disease, and allergic symptoms.

Tannin are astringent polyphenolic biomolecules that bind to proteins and precipitate proteins on a macro basis. As organic compounds, they are identified as bitter-tasting and a dark color pigment, having antioxidant, anti-inflammatory, anti-fibrotic, anti-microbial, anti-diabetic, may be utilized in tanning and re-tanning, useful for industrial hemp applications.

Boiling points ° C. (which can affect extraction specifications): Cannabinoids: CBGA 180; CBG105; THC-9 157; CBN 185; CBD 160-180; THC-8 175-178); CBDA 120-130; CBN 185; CBC 220; THCV<220.

Terpenes: β-myrcene 168; 3-caryophyllene 160; alpha-humulene 106; trans alpha-humulene 167; d-Limonene 176; Linalool 198; pulegone 224; 1,8-cineole 176; α-pinene 155; α-terpineol 219; terpineol-4-ol 89; p-cymene 177; eucolyptal 176; Butylated hydroxytoluene 265.

Flavonoids: apigenin 556; quercetin 642; cannaflavin A>145(dec.); β-sitosterol 502.

Melatonin: Melatonin (formed from precursor serotonin) has antioxidant, anti-inflammatory, immunostimulant, and neuroprotective functions in the body, which can impair viral infections, play a role in circadian rhythm maintenance, and be effective against diabetes mellitus and cardiovascular diseases. It is also involved in the activation of glutathione-synthesizing enzymes. It reduces the pro-inflammatory response of macrophages (preventing pro-inflammatory M1 macrophages and suppressing NF-κB activation which can improve long Covid-19), activates nuclear erythroid 2-related factor 2. Melatonin is also known to balance inflammatory responses by decreasing the levels of pro-inflammatory cytokines such as interleukins (IL) 1β, 6, and 8, and tumor necrosis factor (TNF)-α, and increasing anti-inflammatory cytokine IL-10. Melatonin can control neuroinflammation by interacting with Δ340 and Aβ42 and increasing protein degradation, which may be useful in AD. It also exhibits therapeutic activity against the various symptoms of ME/CFS, such as oxidative stress, pro-inflammatory state, mitochondrial and bioenergetic dysregulation, and disruption of the gut/mucosal barrier. Melatonin has been shown to be effective in patients with autism, attention-deficit/hyperactivity disorder, and neurocognitive disorders.

Melatonin, its receptors (MT1 and MT2 affect protein kinase activity through inhibition of adenylyl (cAMP) and guanylyl (cGMP) cyclase, respectively, and its precursor is tryptophan, in which this amino acid cannot be synthesized in humans) and the ECS are closely related, in they modulate sleep in the sleep-arousal circadian rhythm (including jet lag and shift work delayed sleep phase syndrome. They both exert beneficial effects on the neuroinflammatory processes, in which a variety of immune cells participate, especially glial cells, proinflammatory cytokines, receptors, particularly affecting subcellular organelles including mitochondria, which are mainly responsible for maintaining redox balance at the cellular level that precede the onset of neurodegenerative pathologies such as Parkinson's and Alzheimer's diseases and even following (long) Covid-19. Some of these neuroprotective effects are fundamentally related to its anti-inflammatory and antioxidative actions at the mitochondrial organelle level of ECS cells and is mainly secreted from the pineal gland (when no light is present), although there are many other secondary sources including; retina, gut, skin, platelets and bone marrow; it can also be produced in plants (in fruits: tomatoes, cherries, olives, apple, banana, pomegranate, mulberry, kiwi, pineapple, strawberry, cranberry, cucumber, grapes and red wine, walnuts, with less so in vegetables: radish, onion, lentil, pepper, beetroot, kidney bean, ginger garlic cabbage, carrot, sunflower, and oils, barley, mustard oil, lupine, maize, rice, coffee, pyrethrum maruna, St. John's wort and even hemp), particularly from its seeds.

Melatonin prevents Nrf2 degradation and augments its nuclear accumulation by inhibiting proteasomal, enhanced GSH levels and GPX and GST activities to lower oxidative damage but also reduced TMT-induced pyroptosis and I/R-mediated apoptosis, raised Nrf2 levels in peripheral blood mononuclear cells of patients undergoing coronary artery bypass grafting (CABG), beneficial effects on patients' glycemic and blood pressure control, serum high-sensitivity C-reactive protein (hs-CRP) levels, total cholesterol/HD cholesterol ratio, total cholesterol, and mental health parameters.

Melatonin induces unsaturated/saturated fatty acid (unSFA/SFA) ratio, improving cellular fluidity. Melatonin also regulates arginine metabolisms via increased polyamines, proline and γ-aminobutyric acid (GABA) contents. Furthermore, melatonin possibly encodes calcium-dependent protein kinases (CDPK) and mitogen-activated protein kinases (MAPK) cascades.

Many types of inflammatory disease states may induce melatonin suppression by the pineal gland, which promotes aerobic glycolysis in the mitochondrial resulting in a cytokine storm, and exogenous melatonin (sometimes in synergy with antioxidant vitamins and minerals) counteracts this, along with stabilizing the mitochrondrial membrane, with resistance to ischemia and can assist with neuro-regeneration following traumatic brain injury. It may also improve hereditary mitochondrial diseases, reduces blood pressure, bone metabolism, temperature regulation, mood, degenerative joint disease, acute hepatitis and has anti-neoplastic (breast, prostate cancer, ovarian, gastric, pancreatic and colorectal cancer) activities, as well as mitigate toxicity from chemotherapy or irradiation. Norepinephrine-induced (probably proinflammation-induced) melatonin biosynthesis is inhibited by THC. There have been various confirming the effect of melatonin (contained in hemp seed) on prolonging the shelf-life of produce. Melatonin (E.G., 100 μm) attenuates chilling injury to fresh fruits and vegetables stored at low temperatures (below 10° C.) by activating C-repeat binding factors (CBFs) as transcriptional factors. Melatonin is produced by plants, algae, and animals, with potential of exogenous melatonin to improve cultivation yields by 7-30%, depending on the stressor, with a larger effect obtained in hydroponic systems, to enhance food and feed varies among species, genera, and families, and strongly depends on the concentration of melatonin and treatment duration.

Dextromethorphan: DXM is a cough suppressant in over-the-counter cold and cough medicines. It affects NMDA, and sigma-1 receptors in the brain, all of which have been implicated in the pathophysiology of depression. In 2022, the FDA approved a formulation of it combined with bupropion named Auvelity to serve as a rapid acting antidepressant in patients with major depressive disorder. It is in the morphinan class of medications with sedative, dissociative, and stimulant properties (at lower doses). DXM does not have a significant affinity for the mu-opioid receptor activity typical of morphinan compounds and exerts its therapeutic effects through several other receptors. When exceeding approved dosages, DXM ads as a dissociative hallucinogen. It has multiple mechanisms of action, including actions as a nonselective serotonin reuptake inhibitor and a sigma-1 receptor agonist. DXM and its major metabolite, dextrorphan, also block the NMDA receptor at high doses, which produces effects similar to other dissociative anesthetics such as ketamine, nitrous oxide, and phencyclidine. See en.wikipedia.org/wiki/Dextromethorphan DXM is a prodrug of dextrorphan, which is the actual mediator of most of its dissociative effects through acting as a more potent NMDA receptor antagonist than DXM itself. See, en.wikipedia.org/wiki/Dextromethorphan.

Combination liquid cough syrups; the common OTC formulation contains 15 mg/5 mL of DM; recommended adult dosing is 2 tsp (10 ml) every 4 hours. Sustained-release cough syrup suspensions; another OTC product contains 30 mg/5 mL Liquid filled capsules containing 15 or 30 mg of DXM. Oral strips containing 7.5 or 15 mg of DXM. Lozenges containing 5, 7.5.10 mg of DXM. The recommended dosing for DM is 0.5 mg/kg up to 30 mg, administered three or four times a day. Some animal studies have suggested that to reach the potential neuroprotective effects requires the ingestion of doses higher than typically used for antitussive effects (60 to 120 mg/d).

Curcumin/tumeric: Curcumalonga L. (Turmeric) is used to treat inflammation, arthritis, diabetic wounds, anorexia, microbial infections, muscular disorders, cancer, hepatic disorders, diabetes, biliary disorders, sinusitis and cough. Curcumin exhibits several pharmacological properties such as neuroprotedive, hepatoprotedive, cardiovascular protective, antidiabetic, anticoagulant, anti-inflammatory, antioxidant, antifungal, antiviral, antibacterial, antineoplastic, antifertility and immunostimulant activities in animals preventing neurodegenerative disorders. In Wistar rats, curcuma oil has been documented to minimize inflammation of the endothelial cells in post-myocardial ischemia/reperfusion. Curcumin prevents the accumulation of Ab or facilitates its disaggregation at low concentration levels (IC₅₀=0.81-1 μM). Curcumin has also been shown to protect against Ab neurotoxicity by downregulating Ab synthesis via inhibition of the presenilin 1 (PS1) expression and glycogen synthase kinase-3beta (GSK3b) expression (Caesar et al., 2012; Zhang et al., 2011). In SH-SY5Y neuroblastoma cells treated with 6-hydroxydopamine, curcumin has been indicated to arbitrate neuroprotective activity by attenuating quinoprotein development, expression of p-p38 mitogen-activated protein kinases (MAPKs), and activation of caspase-3. Curcumin substantially increased cognitive function in a streptozotocin (STZ) model of sporadic AD by restoring downregulated IGF-1 levels. Furthermore, another research found that curcumin enhanced the neurotoxicity of 6-hydroxydopamine (6-OHDA) via its anti-inflammatory action and by restoring the expression of SOD-1. In mice, oral administrated aqueous extract of C. longa led to the inhibition of brain monoamine oxidase-A. Curcumin, as the major bioactive compound from C. longa displays the neuroprotective properties against ethanol-induced brain injury. The curcumin enhanced/increased locomotive function in a homocysteine rat model with PD. Concerning epilepsy, in the experimental seizure models, curcumin has shown antiepileptic effects by its antioxidant activity. Furthermore, curcumin is effective in preventing brain injury (cerebral stroke), in addition to antiepileptic effects candesartan's neuroprotective activity on brain ischemia via the repression of blood flow and oxidative stress. Curcumin pretreatment decreased infarction and brain lesions and increased neurological function in rats following Traumatic Brain Injury (TBI). In rats experiencing TBI, curcumin derivatives administration enhanced locomotive and cognitive functioning. Curcuma is primarily recognized as an anxiolytic and mood-stabilizing nootropic useful in treating anxiety and depression. Curcuminoids, such as curcumin (approximately 5% of turmeric) 6-12 g/d may be used in conjunction with black pepper and a fatty liquid medium. The main problem with Curcuma, however, is its low curcumin bioavailability and difficulty in crossing the BBB, meaning that very little of the curcumin in the spice makes it into the bloodstream, and even less is absorbed by the brain.

The steam distilled essential oil of curcuma does not contain curcumin, but rather, Turmerone, ar-Turmerone, Zingiberene, Alpha-Phellandrene, Beta-Sesquiphellandrene, and ar-Curcumene. ar-Turmerone, has been shown to have powerful cardiovascular effects (antiplatelet aggregation and hypoglycemic activity), as well as antimutagenic properties and anti-carcinogenic properties. Some derivations also contain cineole, a compound found in ginger, helichrysum, rosemary, teatree, and peppermint oils.

Tumeric, which also has a low oral bioavailability, similarly, has also shown enhanced absorption with piperine. The bioavailability of cannabinoids can be increased by using supplemental terpenes (Limonene, Alpha-pinene, Menthol, Myrcene and Beta-caryophyllene). However, when consumed with some supplemental herbs (Chamomile flowers; Spicy peppers-capsaicin; turmeric and black pepper-piperine), the compound's bioavailability increases even more.

Piperine (pepper): White pepper contains piperine (consists of approximately 4.0-4.5%, with at least 98% purity). Piperine as an active chemical constituent, which seems to have many effects in the body with diverse biological activities, such as anti-inflammatory, analgesic, anticancer, antiviral, anti-larvicidal, pesticide, anti-Alzheimer's, antidepressant, improve breathing, hypotension, vascular cell modulation, antipyretic, anti-ulcer/anti-emetic, anti-spasmotic, antidiabetic, improves lipid metabolism and may improve weight loss, anti-vitiligo agents, anti-allergic agent (by reducing mast cell activation), antidepressant and most importantly, as the bioavailability enhancer, including inhibition of the gastric emptying of solid and/or liquid diets and the GI transit; has been studied to improve seizure control with anticonvulsants (CBD and CBGA, which has known anti-seizure activity). The ripe berries for white pepper are processed using a procedure known as “retting.” Color is only a superficial difference between black and white pepper. The flavor profile of white pepper differs from black pepper with a distinctive earthy, barnyard-like flavor and aroma that is not typically found in black pepper.

Piperine is an alkaloid naturally found in black pepper (fruit or berries of Piper nigrum L with a 2.5%-3.0% yield) with a myriad of pharmacological attributes. Piperine's most far-reaching indication is drug absorption enhancement, with supportive data regarding its ability to inhibit first pass effect mechanisms and secondarily as a metabolic inducer. There was no significant difference in piperine's effect, when given chronically or in a single dose regimen. Both groups resulted in approximate 2.5-fold increase in oral bioavailability of CBD compared to control group without piperine. There is limited data on CBG/CBGA bioavailability. However, based on its chemical structure and lipophilicity, it can be anticipated that similar bioavailability properties can be extrapolated to full spectrum or isolated CBG products.

Piperine's most far reaching indication is drug absorption enhancement, with supportive data regarding its ability to inhibit first pass effect mechanisms and secondarily as a metabolic inducer. There was no significant difference in piperine's effect, when given chronically or in a single dose regimen. Both groups resulted in approximate 2.5-fold increase in oral bioavailability of CBD compared to control group without piperine. There is limited data on CBG/CBGA bioavailability. However, based on its chemical structure and lipophilicity, it can be anticipated that similar bioavailability properties can be extrapolated to full spectrum or isolated CBG products. Piperine may also convey additional antioxidant and anti-inflammatory as well as other effects.

Piper nigrum (2.5-15 mg per dose), contains a minimum of 95% piperine and has GRAS status. It has been shown to significantly enhance the bioavailability of several supplement nutrients, such as p3-carotene, L(+)-Selenomethionine, Se-methyl-L-selenocysteine, vitamin B6, vitamin C, coenzyme Q10 (CoQ10), curcumin (tumeric), resveratrol, ginseng and elemental iron through their increased absorption.

Quercetin: Quercetin (1000 mg/d) is a flavonoid found in red wine, onions, green tea, apples, and berries known for its antioxidant and anticancer properties at the progression stage. Quercetin is a molecule of interest to reduce the interaction between the SARS-Cov2 virus and the ACE-2 receptor. In addition to inhibition of ACE2 binding and 3CLpro activity, quercetin has reduced the frequency and duration of hospitalization, invasive oxygen therapy rate among COVID-19 patients and reduced extra-articular manifestations (EMS), pain and tumor necrosis factor-alpha (TNF-α) levels in plasma among rheumatoid arthritis (RA) patients and the degree of upper respiratory tract infections. It has been demonstrated that if piperine (20 mg/kg) is administered in combination with quercetin (20, 40 and 80 mg/kg), it enhances the neuroprotective effect of quercetin as well as attenuates the cognitive deficits and oxidative stress as in case of AD. Quercetin and epicatechin (EC) decrease atherosclerosis by augmenting nitric oxide (NO) activity and decreasing serological endothelin-1 levels. Quercetin, which can be obtained from red onion or grape powder, may reduce obesity and adipocyte macrophage-mediated insulin resistance by adipose tissue. Quercetin restores intestinal microbiome balance after antibiotic use.

Pectin: Soy protein isolate/pectin binary complex particles stabilize an emulsion (olive oil served as dispersed phase) containing quercetin. Modified citrus pectin (MCP), a soluble dietary fiber found in citrus fruit, is a direct Gal-3 inhibitor that hinds to the carbohydrate recognition domain of Gal-3. In an animal model of acute kidney injury, MCP was shown to decrease Gal-3 expression and renal fibrosis. MCP has been tested in human clinical trials of solid tumors and lead intoxication. Modified citrus pectin ameliorates myocardial fibrosis and inflammation via suppressing galectin-3 and TLR4/MyD88/NF-κB signaling pathway. MCP ameliorated cardiac dysfunction, decreased myocardial injury, reduced collagen deposition, and downregulated the expression of Gal-3, TLR4 and MyD88, thereby inhibiting NF-κB-p65 activation. MCP also decreased the expression of IL-1β, IL-18 and TNF-α.

The pro-inflammatory, “alarm protein” galectin-3 (Gal-3) is a natural protein that forms the backbone, scaffolding structure for biofilms that ramps up in in response to illness, infection, injury, stress (physical-mental-emotional), aging and other factors, as a driver of unhealthy inflammation, tumor growth and metastasis, fibrosis, and immune suppression. Modified Citrus Pectin (MCP) works to break down the Gal-3 biofilm formations, increasing the effectiveness of other drugs and therapies, from chemotherapy and antibiotic treatments, to nutrients, botanicals, and more with applications for those with chronic infections (including Lyme disease), heart disease and kidney failure, chemo-resistant cancer, and immune suppression in cancer. Trametes versicolor mushroom showed the highest antibiofilm and antibacterial activity. Purified from kelp seaweed, sodium alginates can also help break the biofilms formed by certain bacteria.

Resveratrol: Resveratrol a polyphenol is found in nuts, red grapes, blueberries, cranberries and dark chocolate typically found in heat shock protein of wine. Pterostilbene (PT)—an analog of resveratrol is found in blueberries. Resveratrol has been shown to affect the expression of HSP27, HSP70 and HSP90 in the spleen and thymus and is known to be neuroprotective (helping to preserve neuron structure and function). Resveratrol is a stilbene found in grapes, wine and blueberries that possesses potent antioxidant activity to protect the myocardium from oxidative damage, raised cardiac GSH contents and suppressed oxidant responses in DOX-processed rats, involves the AMPK signaling pathway, the Nrf2 signaling pathway, the Sirt1 pathway, noncoding RNA (miR-149) and the KAT5 gene, increasing SLC7A11/GSH and GPX4, raised erythrocyte PPAR-γ and Sirt1 expression and serum TAC and attenuated the total/HD cholesterol ratio in CHD patients with type 2 diabetes Resveratrol activates metabolism by intestinal microbes, inhibits colonic CB2 messenger ribonucleic acid (mRNA) expression, and restores intestinal barrier function, thereby increasing insulin sensitivity. Resveratrol activates metabolism by intestinal microbes, inhibits colonic (B2 messenger ribonucleic acid (mRNA) expression, and restores intestinal barrier function, thereby increasing insulin sensitivity Resveratrol, epigallocatechin gallate, sulforaphane and piperine are among the molecules studied for their contribution to the synergism seen in the anti-cancer behavior of their combination with curcumin, sometimes used combinations with conventional chemo-agents such as doxorubicin, docetaxel, gemcitabine, celebrex, paclitaxel, camptothecin, 5-fluorouracil and cisplatin, as well as radiation therapy.

Salvia: The diterpene salvinorin A (0.18% in dry biomass) from Salvia divinorum may interact with a putative CB1R/K-opioid receptor (KOR) heterodimer which may be formed during inflammatory (bowel) conditions (but not in normal gut) and may attenuate endocannabinoid degradation, which has potentially application as an analgesic and for substance abuse (cocaine). It can have psychoactive effects (illegal in 13 states) related to its potent D2 dopamine receptor agonist, without serotonin effects. It also contains divinatorins and salvinicins, which can inhibit intestinal motility via KOR and CB1R. It has been also been used as a diuretic and improves cerebral ischemia in animal models.

Catechin: Catechin-derivatives are widespread plant secondary metabolites (e.g., epigallocatechin β-gallate and (−)-epigallocatechin, abundant in tea, but also found in hemp) were shown to bind to human CBR non-selectively. Similarly, hydrophilic phenylpropanoids (e.g., epigallocatechin 3-O-gallate, curcumin, resveratrol, including hemp) can be atypical cannabinoid receptor ligands. Certain flavonoids inhibit FAAH, which is the enzyme responsible for the breakdown of the AEA. Both the isoflavonoid, genistein (found in hemp), and the flavonoids: kaempferol (found in hemp), 7-hydroxyflavone and 3,7-dihydroxyflavone have been shown to inhibit AEA hydrolysis in rat brain homogenates, albeit at relatively high concentrations and the flavonoids, trans-resveratrol, curcumin, catechins and kaempferol-type have relatively poor bioavailability. The coumarin derivative rutamarin from the medicinal plant Ruta graveolens L and 3,3′-diindolylmethane (DIM, a weak CB2R partial agonist) have an anticarcinogenic, metabolite, indole-β-carbinol (also found in Brassica vegetables).

Ancient Cannabis antidotes to counteract THC toxicity have been incorporated into the Ayurvedic tradition of India (and other traditional herbal cultures) include: Lemon (Citrus limon), containing limonene; Pine nuts (Pinus spp.), containing pinene; Calamus plant roots (Acorus calamus) containing eugenol, camphor, as well as beta-asarone, an acetylcholinesterase inhibitor or potential hallucinogen; and Black pepper (Piper nigrum), contains pinene and limonene, as well as enhancing bioavailability. All of these traditional plans contain terpenes common to hemp (except asarone).

Epigallocatechin-β-gallate (EGCG, has low bioavailability, excessive consumption may be hepatotoxic, and metabolizes to gallic acid (GA, which itself has neuroprotective effects on oxidative stress-induced cognitive impairment and the expression of the Nrf2/Keap1 gene in ASD model; with gallic acid-loaded nanophytosomes (GNP) administration, there were improvements in learning and memory deficits by reducing oxidative stress, enhancing antioxidant enzyme activity, and modulating the Keap1/Nrf2 gene expression) and epigallocatechin (EGC, which in turn is degraded via colonic bacteria Adlercreutzia equolifaciens and Flavonifractor plautii MT42 to EGC-M5 in several steps, which then is absorbed and undergoes glucuronidation in the intestinal mucosa and/or liver, distributed throughout the body, crossing BBB, to promote neuritogenesis and potentially exerting a relevant activity against the degenerative processes of neurodegenerative diseases, and finally excreted in urine; EGCG increases the abundance of Bifidobacterium spp., determined via in vitro studies or in Drosophila models of Parkinson's disease, and enriches the population of short-chain fatty acid (SCFA)-producing bacteria such as Akkermansia spp., resulting in an improvement in the production of acetate propionate, and butyrate in a sodium dextran sulfate (SDS)-induced colitis mouse model. By degrading mucin, it releases nutrients such as monosaccharides, amino acids, and SCFAs, which are used by other bacteria in the microbiota, stimulating their metabolic functions. Akkermansia spp. is involved in the regulation of glucose metabolism and adipose tissue homeostasis. EGCG also increases in vitro bacteria of the Bacteroides genus (Bacteroides uniformis, Bacteroides stercoris, Bacteroides thetaiotaomicron, and Bacteroides cellulosilyticus) and Lachnodostridium spp. in male C57BL/6N mice Akkermansia spp. and in ovariectomized (OVX) mice fed a high-fat diet (HFD) Prevotella spp. Administering EGCG to increase Bifidobacterium spp. has been found to enhance the intestinal microbiota, as supported by previous animal studies that showed an elevated abundance of Bifidobacterium spp. in response to a diet enriched with EGCG, and EGCG can reduce B. ovatus, the main intestinal commensal responsible for a systemic antibody response in inflammatory bowel disease. B. fragilis has been shown to restore the balance of T-cell populations in mice affected by ASD, while B. uniformis enhances the production of anti-inflammatory cytokines and improve metabolic and immune dysfunction. Moreover, this genus can improve social behaviors and physiological abnormalities in individuals with ASD, indicating that an increase in Bacteroides abundance caused by EGCG could enhance the quality of life in patients. Lachnodostridium genus, it has been seen to possess anti-inflammatory properties and contribute to the maintenance of intestinal balance by producing butyric acid, and this metabolite aids in the elimination of intestinal gases, and its absence is associated with irritable bowel syndrome, and an increase in Lachnodostridium spp. mediated by EGCG can be seen as having a positive impact on the microbiota of ASD patients. EGCG particularly reduces Clostridia, as a Gram-positive bacilli that, when in excess, can lead to infection (C. difficile enterocolitis) in the large intestine. SCFAs generated from the fermentation of dietary fiber, is an essential metabolite in the human colon as the preferred energy source for colonic epithelial cells, maintains intestinal barrier functions and has immunomodulatory and anti-inflammatory properties, although excessive propionate is associated with oxidative stress (there is also an imbalance in glutathione levels and a decreased storage capacity contribute to increased susceptibility to oxidative stress in ASD children, along with increased free radical production, influenced by amyloid precursor proteins (APP)), with GI issues and neuroinflammation in ASD. Children with ASD have lower levels of fecal butyrate, which regulates gut health, intestinal homeostasis, modulates the expression of neurotransmitter genes, and positively modulates the impaired mitochondrial function in ASD, characterized by reduced activity of complex IV in the electron transport chain, with improvements in oxidative phosphorylation and beta-oxidation.

Maternal treatment with butyrate in the BTBR mouse model of ASD rescues social and partially repetitive behavior deficits in the offspring. Other altered fecal metabolite profiles in ASD children (not present in neurotypical children) show increased in p-cresol (an uremic toxin produced by certain strains of Clostridium spp., which has negative biological effects and appears to adversely affect the homeostasis of colonic epithelial cells in children with ASD; induces DNA damage in vitro and negatively affects the integrity of colonic epithelial cells; an EGCG-enriched diet reduces plasma and urinary concentrations of p-cresol in mice by suppressing the abundance of Firmicutes at the phylum level and Clostridia), caprate, and aspartate and a reduction in GABA, nicotinate, glutamine, and thymine. However, excessively high levels of this SCFA metabolites with an abundance of Clostridia to produces butyrate or butyric acid, along with other SCFAs, can pathologically increase intestinal permeability, which may allow the passage of toxic substances into the bloodstream, potentially leading to inflammation, such that the C. perfringens gene that produces its toxin (CPB2) have been linked to GI complications in ASD and are correlated with disease severity as well as with greater antibiotic resistance in those with ASD, whereas an increase in C. difficile has a notably negative impact on ASD, and effective treatments against this bacterium using oral vancomycin have shown improvements in behavior and communication.

Thus, EGCG's inhibitory effect on this bacterial group, as observed in rats and resulting in decreased levels of Clostridium spp., for homeostasis (bidirectional communication between the gut and the brain) is beneficial. Succinate and butyrate are particularly important and can be altered in individuals with ASD due to microbial activity, with an imbalanced ratio of succinate production/consumption can cause intestinal disturbances and impact the gut-brain axis, with disrupted calcium homeostasis and dysregulated metabolic functions in ASD. EGCG treatment also leads to a decrease in enterobacteria, which are commonly present in the body but can cause infections when their growth is uncontrolled, particularly with Salmonella typhi(typhoid fever), Shigella dysenteriae (dysentery and causes infantile gastroenteritis), and Yersinia pestis (plague). EGCG increases the abundance of beneficial Lactobacillus acidophilus, Bifidobacterium longum, Akkermansia muciniphila, and Prevotella ruminicola, to restore the balance of the intestinal microbiota when included to the diet, reducing oxidative stress in the intestine and the brain. Increased levels of IL-4o, IL-10, have been seen in children with ASD, with notable changes in IFN-α, IL-7, IFN-γ-inducible protein-10, and IL-8 are relevant to innate immunity, and are associated with social behavioral impairments and cognitive development in children with ASD. This effect, in turn, affects BDNF levels, which plays important roles in the formation, branching, and connectivity of synaptic connections during development, as well as in synaptic plasticity, as it is involved in learning and memory. Polyphenols, particularly EGCG, play a role as antioxidants and promote neurogenesis and plasticity in a Down syndrome mouse model; another polyphenol, cyanidin-β-glucoside reduces intestinal inflammation in human intestinal cells, by affecting inflammatory cascades like nuclear factor kappa B(NF-κB), activator protein-1 (AP-1), and Janus kinase-signal transducer and activator of transcription (JAK/STAT); and another polyphenol, resveratrol in vitro modulates inflammatory markers, including nitric oxide (NO), tumor necrosis factor-alpha (TNF-α), ionized calcium-binding adapter molecule 1 (Iba1), prostaglandin E2 (PGE2), inducible nitric oxide synthase (iNOS), and cydooxygenase-(COX-2). In a study on rat pups with autistic traits, an administered polyphenol-probiotic complex with high efficacy in regulating APP levels and crossing the BBB, reversed autistic behaviors and modulated biochemical changes in IL-6, TNF-α, BDNF, 5-HT, AchE, and the granular layer. EGCG demonstrated rapid BBB permeation, while cyanidin-β-glucoside (C3G) showed slower permeation, and quercetin did not cross the BBB. Oral EGCG reduces its levels via the inhibition of intracellular Ca2+ levels and the activation of ERK1/2 and NF-kappaB pathways. This is because it has been seen in the colon that the treatment of TNF-α-stimulated HT29 cells (human colon carcinoma cell lines) with EGCG inhibits IL-8 production by regulating genes in inflammatory pathways, suggesting its potential for preventing or mitigating colonic disorders in ASD. EGCG negatively regulates the inflammatory response in inflamed intestinal epithelial cells, largely via a post-transcriptional regulatory mechanism to diminish reactive oxygen species (ROS), specifically hydrogen peroxide (H₂O₂) and superoxide (O2-), which are both reported in the intestine of children with ASD. Liposomal delivery aims to improve the poor stability of polyphenols against temperature, light, pH, oxygen, and low permeability across intestinal membranes. Both stability and oral bioavailability of EGCG are enhanced via liposomal encapsulation, as it provides protection against degradation when passing through the GI tract, especially when formulated as dual-drug-loaded PEGylated PLGA nanoparticles (EGCG/AA NPs) enhancing its neuroprotective activity as seen in both in vivo and in vitro models of Parkinson's disease. Among individuals with non-AFLD (NAFLD), epigallocatechin gallate (EGCG) has restored intestinal microbiome balance, prevented hepatic TG assimilation, and improved situin gene activity. Among colitis patients, EGCG lowers cyclooxygenase-2 (COX-2) levels, increases cell proliferation, facilitates epidermal growth fader (EGF) mediated epithelial repair, reduces colonic damage, reduces malonaldehyde levels, and increases antioxidant enzymatic activity. In AD, epigallocate chin (EGC) and epicatechin gallate (ECG) decrease amyloid-β accumulation, microglial inflammation, reactive oxygen species (ROS) production, and neurotoxicity. Cocoa extracts decrease amyloid-β oligomerization. n addition, EGCG protects microglia by nuclear factor kappa B (NF-κB) pathway inhibition and nuclear factor-like 2/heme oxygenase 1 (NRF-2/H-1) pathway activation.

Eugenol (clove oil): Eugenol, a methoxyphenol component of dove oil, (Cloves yield from 16% to 19% of oil, the phenol content of which varies from 84% to 95%). It can suppress cydooxygenase-2 expression, while eugenol dimers prevent NFκB activation and inflammatory cytokine expression in lipopolysaccharide-stimulated (LPS) macrophages. Eugenol treatment reduced LPS-induced lung inflammation, improving lung function. Our results suggest that eugenol exhibits in vivo anti-inflammatory action in IPS-induced lung injury. Clove essential oil, its major constituent eugenol, improve diabetes-induced erectile dysfunction in rats, and cove oil caused corpus cavernosus relaxation via K⁺ channels independently NO signaling pathway, from phosphodiesterase inhibition, which may be useful in diabetic men with erectile dysfunction. The inhibition of α-amylase and α-glucosidase activities, inhibition of pro-oxidant induced lipid peroxidation in rat pancreas and antioxidant activities could be possible mechanisms for the use of eugenol in the management and prevention of oxidative stress induced type-2 diabetes. It has been reported as having antioxidant, antitumor, antiviral, antileishmanial, anthelmintic, and hypotensive properties in humans/animals. It inhibits a number of enzymes, including monoamine oxidase, 5-lipoxygenase, and inducible nitric oxide synthase, with reported reduction of NF-κB activity. A number of enzymes are also apparently activated by eugenol, these including p38 MAP kinases and UDP-glucuronyl transferases. Additionally, micromolar concentrations of eugenol have been reported to induce apoptosis in HL-60 leukemia cells (40 μmol l⁻¹) and also in mast cells, whereas at higher concentration (700 μmol l⁻¹) it leads to the translocation of phospho-Ser15-p53 into mitochondria and its interaction with Bd-2 and Bd-xL. Eugenol (4)(at 0.5-2.5 μmol⁻¹ concentration) also inhibits melanoma growth by inhibition of E2F1 transcription.

Phosphodiesterase inhibitors (PDEIs) have pharmacological properties including cardiotonic, vasodilator, smooth muscle relaxant, antidepressant, antithrombotic, bronchodilator, anti-inflammatory and enhancer of cognitive function and can be used as therapeutic agents for various diseases such as dementia, depression, schizophrenia, congestive heart failure, asthma, chronic obstructive pulmonary disease, diabetes, rheumatoid arthritis, multiple sclerosis, Crohn's disease, erectile dysfunction in men, and persistent pulmonary hypertension of the newborn. Some pharmacologically active substances that come from plants Eugenol also inhibits phosphodiesterae (PDE) 1/4. They mainly belong to alkaloids, flavonoids, and saponins.

PDE1 isozymes are present in the CNS, heart, kidney, lung, and smooth muscle. PDE1 inhibitors are possible therapeutic targets in dementia and memory loss. PDE2 is expressed in adrenal gland, heart, lung, liver, and platelets. Disease targets for PDE2 inhibitors are sepsis and acute respiratory distress syndrome, which may include COVID-19. PDE4, a cAMP POE, is the predominant isoenzyme in the majority of inflammatory cells. It is expressed in the airways smooth muscle, brain, cardiovascular tissues, and kidney so its disease targets are allergic rhinitis, psoriasis, multiple sclerosis, depression, Alzheimer's disease, schizophrenia, memory loss, cancer, and dermatitis.

Cordyceps: An aqueous extract of Cordyceps sinensis, an edible mushroom growing in Himalayan regions may be considered for promoting tolerance to high altitudes. It has been demonstrated to have a protective effect of hypoxia-induced oxidative stress in lung epithelium cells by attenuating hypoxia induced reactive oxygen species (ROS) generation, oxidation of lipids (by inhibited the hypoxia-induced lipid peroxidation), and proteins (by inhibited the formation of protein carbonyls levels) and maintained antioxidant status (increased reduced glutathione-GSH levels) via induction of antioxidant gene HO1 (heme oxygenase-1), MT (metallothionein), and Nrf2 (nuclear factor erythroid-derived 2-like 2) by maintaining higher cellular Nrf2, hypoxia inducible factor-1 (HIF1) and lowering NFκB levels. NFκB activates genes particularly involved in the inflammatory response, as well as in modulating the cellular response to oxidative injury, and plays an important role in the innate and adaptive immunity and cellular survival through the induction of genetic networks. At the same time decrease in NFκB levels resulted in lower expression of proinflammatory cytokines like tumor necrosis factor-α(TNFα) and increased expression of anti-inflammatory cytokine transforming growth factor beta (TGFβ). The high amount of phenols, flavonoids, nucleosides (adenosine), nucleobases (adenine and uracil), and polysaccharides found in Cordyceps sinensis absorb and neutralize free radicals, quench singlet and triplet oxygen, or decompose peroxides.

Cordyceps sinensis fungus appears to also lower blood glucose and plasma insulin, and improves glucose metabolism by enhancing insulin sensitivity. Cordyceps sinensis has previously been investigated in animal and in vitro studies for antiaging effects, activity on sexual function, and immune modulation, among other potential uses. In a prior clinical study of elderly patients with chronic fatigue, results indicated that most of the subjects treated with Cordyceps sinensis pure mycelium reported a significant clinical improvement in the areas of fatigue, cold intolerance, dizziness, frequent nocturia, tinnitus, hyposexuality, and amnesia.

Cordycepin, or 3′-deoxyadenosine, is a derivative of the nucleoside adenosine, differing from the latter by the replacement of the hydroxy group in the 3′ position with a hydrogen. It was initially extracted from the fungus Cordyceps militaris, en.wikipedia.org/wiki/Cordycepin but can now be produced synthetically. It is also found in other Cordyceps species as well as Ophibcordyceps sinensis. It has displayed cytotoxicity against some leukemic cell lines produce rapid, robust imipramine-like antidepressant effects in animal models of depression are dependent on enhancement of AMPA receptor signaling.

Cordycepin showed strong binding affinity with SARS-CoV-2 spike protein (−145.3) and main proteases (−180.5) that further corroborate therapeutic potential against COVID-19 as a polyadenylation inhibitor to bind to SARS-CoV-2 RBD domain of spike protein to inhibit replication. Cordycepin can modulate multiple pathways associated with apoptosis, cancer, hepatitis B, tuberculosis, influenza A, herpes simplex infection, HIV, murine leukemia virus, EBV and many more. It has been used in plant medicine for cancer, asthma, TB, diabetics, cough and cold, erectile dysfunction, female BHP, hepatitis.

Liverwort: Liverwort is a green moss-like nonvascular plant producing spores (but grows symbiotically with fungi) containing monomeric allyl-/propenylphenols including eugenol, with tannin, sugar, and mucilage. Some people use liverwort for treating varicose veins, lowering cholesterol, stimulating venous blood circulation (varicose veins or hemorrhoids), and “purifying” blood. Women use liverwort for relieving symptoms of menopause.

Other uses include strengthening nerves, stimulating metabolism, promoting relaxation, and as a general tonic. Liverwort is used for treating gallstones and liver conditions including jaundice, liver enlargement, hepatitis, and liver cirrhosis. It is also used for treating stomach and digestive tract discomfort, stimulating appetite, relieving sensation of fullness, regulating bowel function, and stimulating the pancreas. Liverwort may be prepared by brewing 1.5-3 grams of the dried plant in 150 mL boiling water for 5-10 minutes and then straining as a tea tid. Chlorophyll from liverwort that has been degraded into pheophorbide a (pheoA), which has been shown to prevent SARS-CoV-2 viral entry into cells. Its antiviral effects were also effective against other positive-strand RNA viruses such as West Nile, HCV, and other coronaviruses.

Echinacea: Echinacea has three species: Echinacea angustifolia; Echinacea pallida; Echinacea purpurea. It is a purple coneflower a Native American medicinal plant named for the prickly scales in its large conical seed head which contains polysaccharides, glycoproteins, volatile oils, caffeic acid, alkamides, phenolic acids, rosmarinic acid, polyacetylenes and flavonoids. Several laboratory and animal studies suggest that echinacea contains active substances that boost immune function, relieve pain, reduce inflammation, and have hormonal, antiviral, and antioxidant effects. For this reason, professional herbalists may recommend echinacea to treat urinary tract infections, vaginal yeast (candida) infections, ear infections (also known as otitis media), athlete's foot, sinusitis, hay fever (also called allergic rhinitis), as well as slow-healing wounds. Preliminary studies in the lab suggest echinacea may help inhibit colon tumors when combined with dichloric acid. One study even suggests that echinacea extract exerted an antiviral action on the development of recurrent cold sores triggered by the herpes simplex virus (HSV1) when taken prior to infection. Some studies have shown that for the common cold (virus), it can make you feel better faster. A controversial review of 14 clinical trials found that echinacea reduced the odds of developing a cold by 58% and the duration of a cold by 1 to 4 days. It has shown to provide reduced inflammation, improved immunity and lower blood sugar levels. Recommended dosing: Dry powdered extract: 300-500 mg of Echinacea purpurea, three times daily. Liquid extract tinctures: 2.5 ml, three times daily, or up to 10 ml daily.

In 2020, Signer et al. published in vitro data revealing a broad virucidal activity for Echinacea purpurea (hydroethanolic extract (65% v/v) of freshly harvested Echinacea purpurea (L.) Moench (95% aerial parts and 5% root) in pharmaceutical quality according to good manufacturing practices (GMP)) against a broad number of coronaviruses ranging from the typical common cold CoV-229E to highly pathogenic SARS-CoV-2 viruses. Echinacea significantly reduced expression of inflammatory cytokines tumor necrosis factor TNF-α and interleukin IL-1-1 by up to 24% compared to baseline, and increased levels of the anti-inflammatory cytokine IL-10 and reduces reactive oxygen species (ROS) including hydrogen peroxide (11202), superoxide (O2-), and hydroxyl (OH) radicals. Additionally, there was an increase of up to 50% in the production of the immune-response modulating and antiviral interferon IFN-γ. Several of these bioactive N-alkylamides are structurally similar to endocannabinoids (e.g., anandamide), which influence the cytokine milieu in an anti-inflammatory manner at low nanomolar concentrations. It can reduce inflammatory processes through synergistic immunopharmacological targeting of CB receptors, mild inhibition of the fatty acid amide hydrolase (FAAH), or endocannabinoid transport. Echinacea purpurea has been shown to broadly inhibit coronaviruses and SARS-CoV-2 in vitro. It reduced the incidence of enveloped virus infections from 47 to 29 (p=0.0038) whereas 11 and 13 coronavirus detections (229E, OC43, NL63) were counted (p>0.05). or the incidence was 5.5% for Echinacea extract (2400 mg/d) vs. 14.6% for placebo (VitC50 mg/d) subjects was seen (p=0.010, X 2 test) Respiratory symptoms during coronavirus infections were significantly lower. In a prior study, prevention with Echinacea appeared to reduce the symptomatic development only in children rather than in adults.

Endocannabinoids: PEA's primary mechanism of action is its direct activation on PPAR-α, which initiates a cascade of events that causes the suppression of pain and inflammatory signals. PEA also ads on various other anti-inflammatory receptors (CB1R>CB2R, GPR55, TRPV1 etc.) and inhibits mast cell degranulation, thereby exerting anti-inflammatory, analgesic, and immuno-modulatory effects. PEA has a dual mechanism of action: to reduce inflammation/pain and to activate muscle protein synthesis, which can be depleted with age and stress.

PEA has extensively documented anti-inflammatory, analgesic, antimicrobial, immunomodulatory and neuroprotective effects therapeutic benefits in many applications, including immunity, brain health, allergy, pain modulation, joint health, sleep and recovery, with application to allergic reactions, gut microbiome, antimicrobial (including influenza, common cold, malaria, tick borne diseases), chronic pain, joint pain, psychopathologies and neurodegeneration. They also contribute to enhanced muscle recovery and improved cognition, mood and sleep. Once absorbed, PEA is rapidly metabolized and excreted with a relatively short half-life; levels of PEA in human plasma return to baseline within two hours of ingestion. PEA's poor oral bioavailability (a solid at room temperature), is a major obstacle in early research, but has now been overcome by advanced delivery systems, including emulsification and micronization (which can improve absorption by 1.75×, with a faster onset and longer duration of effect), and is now licensed as food supplements. Endogenous PEA is generally insufficient to counter chronic allostatic load as seen in chronic inflammatory disorders.

PEA degradation is catalyzed by cysteine hydrolase N-acylethanolamine acid amidase (NAAA). The activation of PPAR-α by exogenous PEA attenuates pain and inflammation, and stimulates mitochondrial respiration. PEA shows efficacy in a variety of pain models in animals including carrageenan- and prostaglandin-induced hyperalgesia the formalin test of persistent pain, visceral hyperalgesia produced by instillation of nerve growth factor into the bladder and the sciatic nerve ligature model of neuropathic pain, whereas the acute thermal pain response is not affected. It may mitigate the transformation from acute to chronic pain states. PEA suppresses inflammation in mast cells (MC) activation (including those in the meninges), by downregulating NGF, COX-2, TNF-α and iNOS and inhibits microglia and astrocyte activation. There are several studies on adults and children with migraines confirming a potential use of PEA (600-1200 mg/d for 3 months) as a migraine prophylactic and a possible treatment for tension-type headaches (TTH). In a murine model of endometriosis plus ureteral calculosis, administration of 10 mg/kg/d PEA significantly reduced viscero-visceral hyperalgesia, likely through the down-modulation of MC activity in endometrial cysts, thereby reducing central sensitization. Clinical trials have already demonstrated the beneficial effect of PEA plus polydatin or transpolydatin, in the treatment of secondary dysmenorrhea associated with endometriosis. PEA is endogenously produced on-demand in all tissues, as a protective response to injury, inflammation and pain. Chronic inflammatory conditions create lower levels of PEA. The exogenous administration of PEA may in such cases serve to replenish levels of endogenous PEA, restoring its protective, anti-inflammatory and analgesic effects. PEA was more effective than ibuprofen in relieving the pain of temporomandibular joint (MJ) osteoarthritis. A second placebo-controlled trial demonstrated the effectiveness of PEA on patients with knee osteoarthritis (by 40-50% with 300-600 mg/d) after 8 weeks. In a study regarding muscle recovery after strenuous exercise, found that the group consuming 176.5 mg of a high-bioavailability form of PEA in liquid form had significantly lower myoglobin and blood lactate levels than the placebo group. These shifts signify reduced muscle protein breakdown (increase in protein kinase B(Akt) phosphorylation, a kinase known to induce protein synthesis), and increased aerobic energy metabolism, respectively, findings associated with enhanced and more rapid recovery and the ability to maintain higher exercise intensities for longer.

Other proposed mechanism(s) of action of PEA involve effects upon mast cells, CB2R-like cannabinoid receptors, adenosine triphosphate (ATP)-sensitive potassium (K+)-channels, TRP channels, and NFκB family of proteins. Transient Receptor Potential (RP) Cation Channels, including melastatin-TRPMB antagonist receptor, is a “cold or menthol” receptor. It is also an agonist with G protein-coupled receptor 119 (GPR119), an orphan receptor expressed predominantly in the pancreas (β-cells) and GI tract (enteroendocrine cells involved in glucagon-like peptide-1 secretion) and will, at least in theory, affect endocannabinoid signaling by acting as a competing substrate for the endocannabinoid homologue anandamide (N-AEA); these actions are shared by the endogenous N-OEA, and N-SEA (NAEs). The pharmacokinetic properties of PEA suggests that the compound has a high volume of distribution. Perhaps the most intriguing finding was the concentration of label in the hypothalamus after oral dosing of PEA tritiated in the acyl side chain in rodents. PEA may potentially be useful in a wide range of therapeutic areas, including eczema, pain and neurodegeneration and at the same time to be essentially devoid of unwanted effects in humans veterinary use (skin conditions, Redonyl™, [Innovet]) and as a nutraceutical in humans (Normast™, Pelvilen™ [Epitech]), PeaPure™ [JP Russel Science Ltd]) in some European countries (e.g., Italy, Spain; it is sold as a food supplement in other countries, such as the Netherlands). It also is a constituent of a cream (Physiogel AI™, Stiefel) marketed for dry skin.

PEA exerts its effects by modulating histamine release and microglial state. PEA reduces mast cell degranulation (through the autacoid local injury antagonism mechanism. PEA also confers neuroprotection in the CNS by shifting microglia from the M1 pro-inflammatory phenotype to M2 anti-inflammatory phenotype. M2 microglia attenuates neuroinflammation and improve remyelination, promoting the recovery of olfactory pathways and memory. These functions are clinically and anatomically interrelated, as evident in several neurodegenerative disorders affecting smell and memory. PEA also blocks peripheral mast cell activation and signaling pathways from the periphery to the brain; mast-cell-microglia crosstalk plays a pivotal role in neuroinflammation. PEA also modulates interleukin chemistry. PEA was shown to inhibit release of histamine, prostaglandin D₂ and TNF-α induced by allergens in isolated canine skin MC. PEA directly activates the nuclear receptor peroxisome proliferator-activated receptor α(PPAR-α), which regulates genic expression. Exposure to PEA stimulates E. coli uptake in macrophages and microglia likely via CB2R chemotaxis and PPAR-α induced phagocytosis. Its action on PPAR-α is responsible for an increased expression of the CB2R and the activation of transient receptor potential TRPV1. Moreover, PEA ads through an entourage effect, increasing the availability of AEA and 2-AG, which, in turn, directly interacts with CBR, CB2R and TRPV1. Both mechanisms can contribute to olfactory and memory effects. PEA's ‘entourage effect’ enhances the physiological effects of endocannabinoids such as AEA by preventing their enzymatic-mediated hydrolysis by fatty acid amide hydrolase (FAAH), which results in TRPV1 and CB2 stimulation. This provides another route to the activation of macrophages, neutrophils and other immune cells, thus contributing to PEA's anti-infective properties. AEA also has anti-inflammatory and pro-apoptotic activities through which it can inhibit TNF-α and NF-κB. PEA is considered to constitute a “parallel” ECS without the adverse effects of synthetic endocannabinoids. “Leaky gut” caused by chronic intestinal inflammation is likely associated with neuropsychiatric and neurodegenerative disorders via intestinal cytokine formation. Additionally, bacterial toxins such as LPS entering the systemic circulation through a compromised gut epithelium can traverse the BBB and act as ligands for receptors in the brain. These vectors initiate neuroinflammatory cascades, a hallmark of neurodegenerative disease and psychiatric disorders. PEA's anti-inflammatory action via PPAR-α in the gut epithelium has the potential to prevent neuroinflammatory responses by maintaining integrity of the gut barrier. In a murine model of colitis, PEA attenuated inflammation and intestinal permeability and stimulated colonic cell proliferation in a PPAR-α- and CB2-dependent manner. Other murine models of IBD found that higher levels of PEA levels were associated with less severe colonic inflammation and reduced proinflammatory cytokine production and immune infiltration. PEA binds to GPR119 receptors in the gut and influences the secretion of satietogenic hormone GLP-1, which alleviates cognitive deficits in patients with a mood disorder. In an induced inflammation state, such as vitamin D deficiency in mice, intraperitoneal administration of PEA increases the level of commensal bacteria such as Akkermansia muciniphila (protective effects against obesity and diabetes), ubaderium (microbiome regulatory properties) and Enterobacteriaceae. Moreover, exogenous administration of PEA relieves chronic and acute GI inflammation via its action on PPAR-α in the colon. Neuroprotective actions (seen with murine models of AD, HD, ASD, stroke, TBI) attenuated gliosis and neuroinflammation by inhibiting apoptosis and autophagy by modulating the (bd-2-associated X protein) bax/bd-2 and Akt/mTOR/p70S6K pathways; limiting necrotic processes; targeting NMDA receptors, thereby protecting cells against glutamate toxicity; modulating synaptic homeostasis; promoting neurogenesis; and down-regulating the development of cerebral edema and local inflammatory cascades. PEA's inhibition of pro-inflammatory cytokines likely contributes to its ability to prevent cortical spreading depression in pre-clinical models and its therapeutic effects in migraine. PEA levels are increased by acute psychosocial stress, presumably as a protective response to aversive and/or hazardous situations and a modifier of the induced Cell Danger Response (CDR); but are unaffected or reduced in depression. PEA levels were found to be higher in individuals suffering from PTSD compared to trauma-exposed individuals without PTSD and correlated with a greater symptom severity. This suggests that endogenous NAE levels are insufficient to restore homeostasis in chronic aversive states, which may explain why PTSD patients self-medicate with Cannabis and why, in animal models of AD and TBI, PEA administration improves memory function and reduces anxiety, aggressiveness and depression. PEA was shown to increase hippocampal neurogenesis and neuroplasticity in an established murine model of autism (ASD), and prevented the decrease in brain-derived neurotrophic factor (BDNF) and glial cell line-derived neurotrophic factor (GDNF) in a murine model of cerebral ischemia. When used as an add-on therapy to levodopa in PD patients, PEA was found to improve motor and non-motor symptoms, including mood deficits, fatigue, sleep and mental tasks, after continuous administration for one year. patients with major depressive disorder (MDD) receiving PEA 600 mg (or 1200 mg in severe depression) in addition to citalopram for 6 weeks twice daily demonstrated significantly greater improvements in depressive scores and symptoms compared to citalopram plus placebo group. There was also an anxiolytic effect (perioperatively and with knee osteoarthritis) at the higher doses, with improved sleep. High levels of AEA are linked to wakefulness in healthy individuals and defining levels in the elderly are associated with circadian rhythm imbalance and cognitive impairment. Via the entourage effect, PEA may, therefore, support the sleep-wake balance in healthy adults. Three case studies on children with ASD reported PEA administration (600-700 mg per day for several months to a year) improved behavior, sociability and cognition. Topical application of a cream with a unique lamellar matrix containing PEA significantly reduced intensities of erythema, pruritus, excoriation, scaling, lichenification and dryness; and in experimental model of contact allergic dermatitis, PEA increased ear skin PEA levels, and increased TRPV1, PPAR-α and NAPE-PLD in ear keratinocytes, with CB2R agonist effects, and restoration of 2-AG levels. PEA's role as an immunomodulator rather than an immunosuppressant.

Parosmia can arise from both central and peripheral damage to the olfactory pathways, and persistent smell alteration may reflect perturbations in acetylcholine-mediated signal transmission with neuroinflammation. Ultra-micronized PEA 700 mg/luteolin 70 mg combination per day sublingually for 4 weeks was effective for the quantitative or qualitative measures of olfactory dysfunction or relief from mental clouding in patients affected by long COVID. In preclinical studies of neuroinflammatory disorders, co-ultra-micronized PEA and luteolin (also found in Cannabis) improve neuroprotection compared with either molecule alone (entourage effect). In stroke patients, this same combination in conjunction with standard rehabilitation therapy for 60 days significantly improved neurological status, cognitive abilities and degree of spasticity, pain and independence.

PEA Levagen®, (300 mg/d), is a next generation anti-inflammatory analgesics. It is an endogenous fatty acid amide produced by the body in response to stressors, pain and inflammation as a biological response and repair mechanism.

PEA shows efficacy in a variety of pain models including carrageenan- and prostaglandin-induced hyperalgesia the formalin test of persistent pain, visceral hyperalgesia produced by instillation of nerve growth factor into the bladder, and the sciatic nerve ligature model of neuropathic pain 14, whereas the acute thermal pain response is not affected. The proposed mechanism(s) of action of PEA involve effects upon mast cells, CB2R-like, ATP-sensitive K+-channels, TRP channels, and NFkB, although the most robust evidence is for an action of PEA upon the nuclear receptor peroxisome proliferator-activated receptor α (PPARα). These are by no means the only actions of PEA: it can also, for example, interact as an agonist with GPR119, an orphan receptor involved in glucagon-like peptide-1 secretion and will, at least in theory, affect endocannabinoid signalling by acting as a competing substrate for the endocannabinoid homologue anandamide (N-arachidonoylethanolamine). Some of these actions are shared by the endogenous NAEs N-oleoylethanolamine and N-stearoylethanolamine. The efficacy of PEA in the six blinded RCTs including molar extraction, vestibulodynia (using transelectrical nerve stimulation as control), and sciatic pain; two of the studies with NSAID comparator groups (pelvic and TMJ pain); in one, the patients fared better with celecoxib than with PEA+transpolydatin, whilst in the other, the patients fared equally well with PEA and ibuprofen over the first eight days, after which the effect of ibuprofen plateaued out, whilst those patients treated with PEA continued to improve. PEA is also relatively short-lived in human plasma, returning to baseline after 2 hours of ingestion in rats. PEA has a high volume of distribution, with approximately 0.95% of the administered PEA found in the brain: hypothalamus, white matter, brain stem, cerebellum and brain cortex, as well as in the pituitary gland and adrenal organs. The current clinical data argue against ‘very common’ or ‘common’ serious adverse drug reactions (ADRs) being found with PEA (although most studies are for 60 day duration). The process of micronization results in smaller particles and hence a larger total surface area. This allows the GI milieu more access to free surfaces on the drug particle and hence a faster dissolution can be achieved. This may lead to a better adsorption of the drug molecules, with a study on rodents showing that orally administered micronized and ultramicronized PEA are more efficacious than unmicronized PEA in the carrageenan model of inflammatory pain.

Both PEA and CBD were anti-inflammatory in acute and chronic IBD and appendicitis explants. IFNγ and TNFα treatment increased phosphoprotein and cytokine levels in cultured colonic cell lines, Caco-2 and colonic explants from elective bowel cancer, inflammatory bowel disease (IBD) or acute appendicitis resections. Phosphoprotein levels were significantly reduced by PEA or CBD in Caco-2 cultures and colonic explants. CBD and PEA prevented increases in cytokine production in explant colon, but not in Caco-2 cells. CBD effects were blocked by the CB2R antagonist AM630 and TRPV1 antagonist SB366791, whereas PEA effects were blocked by the PPARα antagonist GW6471.

PEA and CBD are anti-inflammatory in the human colon. A two-week treatment with CBD increased circulating levels of AEA, PEA, and OEA in the serum of schizophrenia patients; PEA is safe and effective in reducing the incidence and symptoms associated with upper respiratory tract infections (URTIs). N-PEA, and endocannabinoids, has been compounded into creams and shown to reduce pruritus within days in patients with atopic dermatitis (mild to moderate), lichen simplex chronicus, prurigo nodularis, and uremic pruritus. A small case series of postherpetic neuralgia demonstrated reduction in mean pain scores by 87.8% in 5 of 8 patients treated with a cream containing N-PEA, a CBR agonist.

Black seed oil: Black seed oil, derived from the Nigella Sativa plant, contains 15 amino acids, including essential amino acids (histidine, isoleucine, leucine, lysine, methionine, phenylalanine, threonine, and tryptophan), as well as arginine (improve circulation, immunity and metabolism); Thymoquinone (TQ), Thymol (both antifungal effects) and, Thymohydroquinone (THQ, potent natural acetylcholinesterase (AChE) inhibitors, which can improve cognition). It has a wide array of benefits for both internal and external use. The primary uses of black seed oil include reducing inflammation, fighting bacteria and damaging free radicals, supporting optimal immune system, lessen allergic reactions, respiratory conditions (including asthma, cough, bronchitis, emphysema, flu or other viral upper respiratory infections and congestion), gut (including gas, colic, diarrhea, dysentery, constipation, and hemorrhoids) and liver function and healing, regulating blood sugar (with partial regeneration of pancreatic beta-cells), lowering lipid levels, and lowering blood pressure, and speeding the healing process for wounds and skin conditions, and possibly anti-cancer effects.

The product can also be modified with additional additives with other natural plant products that enhance other mechanism of effect, including fungi, algae (Chlorella vulgaris and Chlorella pyrenoidosa) with essential vitamins minerals, lipids, bioactive peptides, and fiber.

Lion's mane: Fungi or mushrooms may be employed for their fermentation effects and probiotics, anti-inflammatory, antioxidant, and anticancer activity. Lion's mane mushroom 250 mg/d may enhance the brain through neurological growth. In animal models, its active compounds have demonstrated the ability to delay neuronal death in neurodegenerative diseases such as ischemic stroke, Parkinson's disease, AD, and depression, and it has been shown to promote nerve regeneration and functional recovery in neuropathic pain or presbycusis. In addition, lion's mane has been shown to prevent the loss of spatial short-term and visual recognition memory induced by AR25-35 in mice. Similarly, it has been shown in vitro that lion's mane fruiting body extract induces neurite outgrowth of neuron cells NG1a08-15 and PCI2 cells, promotes nerve growth factor (NGF) mRNA expression, and modulates the secretion of NGF from 1321N1 human astrocyte cells. Other fungi for antimicrobial effects include: Agaricus subrufescens Peck, Agaricus blazei Murill, Cordyceps sinensis (Berk.) Sacc., Ganoderma lucidum (Curtis.) P. Karst., Grifola frondosa (Dicks.) Gray, Hericium erinaceus (Bull.) Pers., Inonotus obliguus (Arch. Ex Pers.) Pilát., Lentinula edodes (Berk.) Pegler, Pleurotus ostreatus (Jacq.) P. Kumm., Poria cocos F. A. Wolf, and Trametes versicolor(L.) Lloyd, turkey tail (Trametes versicolor) and agarikon (Fomitopsis officinalis) both mushroom species containing polysaccharides (also: terpenoids, lectins, glycoproteins, lentinan, galactomannan), investigated for antimicrobial effects including Covid.

Algae (chlorella/spudina): Algae (chlorella) may be used for its nutritional benefits including fiber, iodine, Bitamin B₁₂, Iron, small amounts of magnesium, zinc, copper, potassium, calcium, folic acid and other B vitamins, Vitamin C, Omega 3, DHA cardioprotedive oil (250 mg/d), and Proteins with 9 essential amino acids.

Algae (spirulina) 15 ml dried contains phycocyanin, an antioxidant, Protein: 4 grams Vitamin B, (thiamine): 1% of the RDA Vitamin B₂ (riboflavin): 15% of the RDA, and Vitamin B₃ (niacin): 4% of the RDA.

Alginic acid made from certain brown algae; Agar extracted from a number of related species of red algae; Ammonium alginate from certain brown algae; Calcium alginate from certain brown algae; Potassium alginate, the potassium salt of alginic acid, derived from certain brown algae; Sodium alginate, the sodium salt of alginic acid, derived from certain brown algae.

Red algae, to be used dried as a flavor enhancer, are seaweeds of the species: Gloiopeltis furcate.

Alginate from seaweed 1000 mg Algin is used to lower cholesterol levels and to reduce the amount of heavy chemicals including strontium, barium, tin, cadmium, manganese, zinc, and mercury that are taken up by the body. Algin is also used for the prevention and treatment of high blood pressure. In foods, algin is used in candy, gelatins, puddings, condiments, relishes, processed vegetables, fish products, and imitation dairy products. In manufacturing, algin is used as a binding agent in tablets, as a binding and soothing agent in throat lozenges, and as a film in peel-off facial masks. Alginate is a natural polysaccharide containing β-(1,4)-d-mannuronic acid (M) and α-(1,4)-1-guluronic acid (G) monomers. Nakazono et al. (2016) studied the antiobesity effects of enzymatically digested alginate oligomers (EAOs) and acid-hydrolyzed alginate oligomers (AAOs) in mice. Results demonstrated that the antiobesity activity of EAOs was superior to that of AAOs. Alginate may be produced from brown algae (Phaeophyceae), including Laminaria hyperborea, Laminaria digitata, Laminaria japonica, Ascophyllum nodosum, and Macrocystis pyrifera by treatment with aqueous alkali solutions, typically with NaOH. The extract is filtered, and either sodium or calcium chloride is added to the filtrate in order to precipitate alginate. This alginate salt can be transformed into alginic acid by treatment with dilute HC. After further purification and conversion, water-soluble sodium alginate power is produced. On a dry weight basis, the alginate contents are 22-30% for Ascophyllum nodosum and 25-44% for Laminaria digitata. More than 200 different alginates are currently being manufactured Amphiphilic derivatives of sodium alginate have been prepared by conjugation of long alkyl chains (i.e., dodecyl, otadecyl) to the alginate backbone via ester bond formation. Aqueous solutions of these alginate derivatives exhibited the typical rheological properties of physically cross-linked, gel-like networks in the semidilute regime, which could be useful for cartilage repair and regeneration. Alginate is inherently non-degradable in mammals, as they lack the enzyme (i.e., alginase) which can cleave the polymer chains, but ionically cross-linked alginate gels can be dissolved by release of the divalent ions cross-linking the gel into the surrounding media due to exchange reactions with monovalent cations such as sodium ions. The controlled and localized delivery of antineoplastic agents has also been achieved using partially oxidized alginate gels Alginate has also been widely exploited in many drug delivery applications in combination with chitosan, as the combination forms ionic complexes.

Alginate gels have also been utilized to form a matrix in which depots releasing small drugs can be incorporated; Amoxicillin-loaded chitosan/poly (γ-glutamic acid) nanoparticles have been incorporated into alginate/Ca2+ hydrogels for effective treatment of H. pylori infection. The alginate gel outer layer protected the amoxicillin-loaded nanoparticles in the gastric environment, and facilitated amoxicillin interactions specifically with intercellular spaces, which is the infection site of H. pylori Alginate is an excellent candidate for delivery of protein drugs, since proteins can be incorporated into alginate-based formulations under relatively mild conditions that minimize their denaturation, and the gels can protect them from degradation until their release. e, insulin-loaded alginate microspheres were prepared by blending alginate with anionic polymers (e.g., cellulose acetate phthalate, polyphosphate, dextran sulfate), followed by chitosan-coating in order to protect insulin at gastric pH and obtain its sustained release at intestinal pH. Alginate microspheres have also been coated with Bombyx mori silk fibroin using layer-by-layer deposition techniques, which provided mechanically stable shells as well as a diffusion barrier to the encapsulated proteins modern dressings (e.g., alginate dressings) provide a moist wound environment and facilitate wound healing. Alginate dressings are typically produced by ionic cross-linking of an alginate solution with calcium ions to form a gel, followed by processing to form freeze-dried porous sheets (i.e., foam), and fibrous non-woven dressings. Alginate gels releasing stromal cell-derived factor-1 were also effective in accelerating wound closure rates and reducing scar formation in pigs with acute surgical wounds. Incorporation of silver into alginate dressings increased antimicrobial activity and improved the binding affinity for elastase, matrix metalloproteases-2 (MMP-2), and proinflammatory cytokines (e.g., TNF-α, IL-8). The addition of silver into alginate dressings also enhanced the antioxidant capacity. Alginate fibers cross-linked with zinc ions have also been proposed for wound dressings, as zinc ions may generate immunomodulatory and antimicrobial effects, as well as enhanced keratinocyte migration and increased levels of endogenous growth factors. Blends of alginate, chitin/chitosan, and fucoidan gels have been reported to provide a moist healing environment in rats, with an ease of application and removal The presence of RGD peptides in alginate gels allows one to control the phenotype of interacting myoblasts, chondrocytes, osteoblasts, ovarian follide, as well as bone marrow stromal cells-. For example, the adhesion and proliferation of myoblasts cultured on alginate gels were dramatically enhanced by chemical conjugation of RGD peptides to the alginate backbone, compared with non-modified alginate gels (FIG. 13). Alginate gels have been widely explored over the past several decades as a vehicle to deliver proteins or cell populations that can direct the regeneration or engineering of various tissues and organs in the body. Alginate gels have proved to be useful for transplanting chondrogenic cells to restore damaged cartilage in animal models. Early studies utilized a suspension of chondrocytes in an alginate solution mixed with calcium sulfate, and injected into molds of facial implants in order to produce pre-shaped cartilage skeletal muscle regeneration include cell transplantation, growth factor delivery, or a combination of both approaches, and alginate gels have found potential in these strategies. A combined delivery of VEGF and insulin-like growth factor-1 (IGF-1) from alginate gels was used to modulate both angiogenesis and myogenesis Propylene Glycol Alginate in Beer PGA has emulsifying, thickening, swelling, acid resistance, and stabilizing properties due to the lipophilicity and hydrophilicity properties in the molecular structure. PGA is ideally suited for stabilizing acid protein beverages such as beer. Indeed, the beer foam stabilizer is a typical application of high esterification degree PGA and its general dosage is 40-100 mg/kg.

Alginate is a biomaterial that has found numerous applications in biomedical science and engineering due to its favorable properties, including biocompatibility and ease of gelation. Alginate hydrogels have been particularly attractive in wound healing, drug delivery, and tissue engineering applications Gaviscon can eliminate the “acid pocket” in GERD patients. Considering that EGJ length was unchanged throughout, this effect was likely attributable to the alginate “raft” displacing gastric contents away from the EGJ. These findings suggest the alginate-antacid and alginate-antacid formulation (Gaviscon Double Action Liquid) can eliminate the postprandial “acid pocket” in symptomatic GERD patients formulation to be a well-targeted postprandial GERD therapy. This Product can also be modified with additional additives including Lactobacillus to facilitate gut health for oral ingestion for immune, cardiac anticancer metabolic mood neurodegeneration antiaging, sarcopenia benefits.

Chitosan: Chitosan is a derivative of chitin, the second most abundant natural polymer in the world, and has a repeating structure of (1,4) linked β-D-glucosamine, with an apparent pK of 6.5. Traditionally, commercial products are composed of 80% β-D-glucosamine and 20% N-acetyl-β-D-glucosamine. Chitosan is a cationic polymer and has been widely used in the areas of food, cosmetics, biomedical and pharmaceutical applications, due to its biocompatibility and other favorable properties Magnetic alginate-chitosan beads loaded with albendazole (ABZ) were also prepared for passive targeting to the g GI tract using physical capture mechanisms (e.g., magnetic field, pH). The beads showed unique pH-dependent swelling behaviors and a continuous release of ABZ. Chitosan-treated alginate microparticles containing all-trans retinoic acid (ATRA) have also been shown to enhance dermal localization and achieved the sustained release of ATRA into the skin. Metronidazole was also entrapped into chitosan-treated alginate beads by an ionotropic gelation method, and the beads were effective in eradication of Helicobacter pylori when orally administrated into mice. Targeted binding support for heavy metals, pesticides, and biotoxins in the gut, helps break up biofilms, provides prebiotic nourishment.

Probiotics: Oral administration of probiotic, Lactobacillus acidophilus (1 billion CFUs/d) was shown to combat inflammation and nociception through increasing the expression of the CB2R in intestinal epithelial cells, suggesting that they might work together to halt inflammation and nociception. In support of this, it has recently been shown that THC reduces inflammation and adiposity in mice by increasing the accumulation of mucin-degrading bacteria, Akkermansia municiphila, which was shown to reduce systemic inflammation in mice, further supporting the notion that microbiota contributes to the anti-inflammatory and analgesic effects of oral cannabinoids.

Other desirable probiotics: B. longum, B. bifidum and L. fermentum, Bifidobacterium Infantis, B. bifidum W23 B. lactis W51 E. faecium W54 L. acidophilus W37 L. acidophilus W55 L. paracasei W20 L. plantarum W62 L. rhamnosus W71 L. salivarius W57 L. fermentum L. casei, Bacillus subtilis HU58™ Bacillus clausii, Lachnospiraceae S. boulardi, Enterobacteriaceae can be considered mucotrop, Streptococcus salivarius subsp, thermophilus, Latobacillus casei, L. plantarum, L. acidophilus, L. delbrueckii subsp. bulgaricus, Bifidobacteria longum, B. infantis, and B. breve Sacharomyces boulardii CNCM1-745 (yeast) supports regeneration of the intestinal microbiota after diarrheic dysbiosis. Yeast producing saccharomyces may be modified to produce cerevistae isoamyl acetate isoamyl acetate, the source of the banana-like flavor, which may be a flavor enhancer for hops fermentation (may be alcohol free).

These probiotics increase intestinal levels of Butyrate, a short chain fatty acid SCFA, which is formed due to bacterial digestion of a diet rich in fiber. This, in turn, can protect against autoimmune, mast cell activation syndrome (MCAS), inflammatory states (IBD), neuroinflammation, leaky gut syndrome, with antiaging and improved energy benefits. Dysregulation of the ECS has been connected to digestive disorders such as inflammatory bowel disease (IBD, microbes should not attract the attention of the immune system, but if the immune system fails to ignore them and targets them instead, this can cause inflammation and the development IBD, as modulated by immunosuppressive regulatory T cells), irritable bowel syndrome, as well as obesity, and intestinal dysbiosis, which in turn, has been associated with neurodegenerative disease and cancer. For breast cancer, has been hypothesized in animal models, that mast cell behavior changes in the infiltrated tumor tissue with dysbiosis such that there are changes in the tissue architecture of the diseased tumor region, so neoplastic cells can more easily metastasize.

The GI tract harbors the largest population of mast cells in the body. Mast cells react to both external and internal stimuli thanks to the variety of receptors they express and carry out effector and regulatory tasks by means of the mediators of different natures they produce. Mast cells are fundamental elements of the intestinal barrier as they regulate epithelial function and integrity. Mast calls modulate both innate and adaptive mucosal immunity, and maintain neuroimmune interactions, which are key to the functioning of the gut. Disruption of the intestinal barrier is associated with an increased passage of luminal antigens into the mucosa, which facilitates mucosal mast cell activation, and ultimately, a myriad of disease states.

GI phospholipids play an important role in mucosal barrier function. It's estimated that 90 percent of the gut mucosa is comprised of phosphatidylcholine. The health of the intestinal microbiome is linked to a number of diseases including Crohn's, Colitis, neurodegenerative diseases, cardiovascular disease, and other types of autoimmune diseases.

Lactobacillus reuteri is found in different body sites, including the GI tract, urinary tract, skin, and breast milk. L. reuteri strains can reduce the production of pro-inflammatory cytokines while promoting regulatory T cell development and function. Third, bearing the ability to strengthen the intestinal barrier, the colonization of L. reuteri may decrease the microbial translocation from the gut lumen to the tissues. Microbial translocation across the intestinal epithelium has been hypothesized as an initiator of inflammation. Other GI bacteria include Faecalibacterium, Akkermansia, Dialister, Bacillus coagulans, Bacteroides, Bifidobacterium infantis. Elevated IgG responses in patients with inflammatory bowel disease (IBD) including taxa within the clades Collinsella, Bifidobacterium, Lachnospiraceae, and Ruminococcaceae.

Starches quickly turned into glucose in your small intestine, but resistant (nondigestible) starches with prebiotic effects, which are fermented by your gut microbes into SCFAs (acetate, butyrate, and propionate), which can lower blood sugar levels, improve insulin sensitivity and reduce serum cholesterol, and may aid in weight loss. Resistant starches can also nourish bacteria (like Ruminococcus bromii) that, in turn, produce fuel for butyrate-producing bacteria (like Eubacterium recale and Faecalibacterium prausnitzii). Classification levels: Type 1: legumes, seeds, grains; Type 2: iaw potatoes (not reoommended to consume), green bananas, plantain, corn; Type 3: cooked and cooled potatoes, and rice, bread, cornflakes; Type 4 chemically-modified starches in processed bread, crackers, cakes. Potato starch becomes resistant when cooled (type 3), with a higher dietary fiber load in the colon. A clinical study of type 4 resistant starch lowered all types of cholesterol (good and bad) as well as improved body at percentage and caused a small reduction in waist size.

DHEA: Dehydroepiandrosterone (DHEA), also known as androstenolone, is an endogenous steroid hormone precursor, DHEA functions as a metabolic intermediate in the biosynthesis of the androgen and estrogen sex steroids both in the gonads and in various other tissues. However, DHEA also has a variety of potential biological effects in its own right, binding to an array of nuclear and cell surface receptors, and acting as a neurosteroid and modulator of neurotrophic factor receptors. DHEA and other adrenal androgens such as androstenedione, although relatively weak androgens, are responsible for the androgenic effects of adrenarche, such as early pubic and axillary hair growth, adult-type body odor, increased oiliness of hair and skin, and mild acne.

DHEA is potentiated locally via conversion into testosterone and dihydrotestosterone (DHT) in the skin and hair follides. DHEA is a weak estrogen. In addition, it is transformed into potent estrogens such as estradiol in certain tissues such as the vagina, and thereby produces estrogenic effects in such tissues. As a neurosteroid and neurotrophin, DHEA has important effects in the central nervous system. DHEA is an endogenous precursor to testosterone and DHT. DHEA has also been found to bind to (and activate) the ERα and ERβ estrogen receptors. DHEA is designated a dietary supplement by FDA, and has psychotropic actions, affecting serotonin, dopamine and modulation of NMDA receptors.

Lipoic acid: Lipoic acid (LA), also known as α-lipoic acid (ALA) and thiodic acid; is an organosulfur compound derived from caprylic acid (octanoic acid). ALA is made in animals normally, and is essential for aerobic metabolism. It is also manufactured and is available as a dietary supplement where it is marketed as an antioxidant Dihydrolipoic acid is an organic compound that is the reduced form of lipoic acid. ALA, with short half-life and low bioavailability, acts as a free radical scavenger and natural cofactor of mitochondrial dehydrogenase complexes (diabetes type II down regulates the expression of lipoic acid synthase (LASY), an enzyme that is responsible for the ALA synthesis inside the mitochondria reducing glucose uptake in skeletal muscle), with potent anti-inflammatory (inhibits the inflammasome and, consequently, downregulates TNF-α, IL-6, IL-18, interferon-γ and IL-1β (probably is the cytokine responsible for cell injury in type I diabetes) levels mediated by NFκB, blocking its translocation to the nucleus and reducing proinflammatory cytokine release, to play an anti-inflammatory and immunomodulatory role; and antioxidant effed (including dihydro-lipoic aid (DHA), as its reduced form, as scavengers of the reactive oxygen species (ROS, to reduce cell damage by stimulating the production of antioxidant enzymes, such as glutathione peroxidase and superoxide dismutase 1, inside mitochondria and endoplasmic reticulum via translocation of Nfr-2, preventing the decreasing of PPAR; reduces oxidation of glutathione, vitamin C and vitamin E) and the improvement of insulin metabolic pathway by stimulating the activity of PI3K, and consequently, the phosphorylation of insulin receptor substrate-1 in adipocytes via translocation of glucose transporter proteins GLUT4 and GLUT1 via AMPK activation to plasma membrane in adipocytes (also in skeletal muscle and liver, which can mimic metformin's mechanism of action, although hepatoytes have a specific binding site for ALA. PEA (400 mg, taken with alpha lipoic acid, transpolydatin or polydatin) can improve the “sexual life of women” or “dyspareunia” associated with endometriosis.

SCFA: Acetate (2, the most produced SCFA in the gut, anaerobic fermentation of carbohydrates often entails the breakdown of monosaccharides with 5 pathways, 3 of which are found in Coprococcus), propionate (3), and butyrate (C4) are the most abundant SCFAs found in the large intestine. SCFAs serve as ligands for various G-protein coupled receptors (GPCRs, especially GPR43 or free fatty acid receptor 2 (FFAR2) and GPR41 or FFAR3; FFAR2 has a high affinity for acetate and propionate, whereas FFAR3 prefers longer fatty acid structures such as butyrate. FFAR2 and FFAR3 are not specifically expressed in intestinal epithelial cells, but ore found in tissues such as the vasculature and kidneys, as well as in various immune cells (e.g., neutrophils, monocytes, and lymphocytes); Direct activation of the vagus nerve and enteric nervous system by SCFAs may be possible, since FFAR3 is expressed in the periportal afferent neural system, as well as in the enteric neural plexus and autonomic and sensory ganglia) and hydroxycarboxylic acid receptor 2(GPR109a/HCAR2) in colonocytes. Acetate may alter (metabolites for) the neurotransmitters Glu, glutamine, GABA as well as anorexigenic neuropeptide expression in the hypothalamus. Valeric acid (a minor component of valerian flower, for which its root has sedating properties) is a minor product of the gut microbiome and can also be produced by metabolism of its esters found in food, and commercially used for flavors and perfumes, ester type lubricants, plasticizers and vinyl stabilizers Microbiota-derived butyrate may improve the integrity of the BBB by inducing higher levels of the tight junction protein occluding, and endothelial cells abundantly express proton-coupled monocarboxylate transporter (MCT-1, and to a lesser extent, MCT-4, which reaches the highest density in the distal colon) which is the likely mode of transport for SCFAs to cross the BBB. Butyrate may also induce epigenetic changes by increasing histone acetylation and histone acrotonylation in the brain. Additionally, butyrate epigenetically regulates microglial responses through the downregulation of pro-inflammatory mediators, while upregulating the expression of anti-inflammatory mediators. Propionate has been associated with reduced experimental autoimmune encephalomyelitis (EAE) and axonal damage through increased T differentiation. The combination of propionate and butyrate regulates tryptophan hydroxylase expression and modulates intracellular potassium levels in the CNS. Bacteroidetes are responsible for the production of bulk acetate and propionate (via succinate, and to a lesser extent, acrylate, and propanediol pathways, using polysaccharides, organic acids, lactate, and amino acids as substrates), whereas Firmicutes (including coprococcus) are the main butyrate producers in the human gut. Resistant starch (starch and products of starch degradation not absorbed in the small intestine), oat and wheat bran, cellulose; and pectin contribute to butyrate. Coprococcus produces all SCFAs, but C. catusis characterized by its large production of propionate. It has a unique capacity to use two separate pathways for butyrate synthesis and C. eutactus been found to be depleted in children with delayed language development and adults with Parkinson's disease, and may be used as a dysbiosis biomarker. Relative to butyrate, Faecalibacterium prausnitzii, Ruminococcaceae, and Eubacterium rectale were positioned at numbers 1, 2, and 3, respectively, whereas C. comes only at position 10 and C. catus at position 18. Microbiota-produced butyrate is oxidized to carbon dioxide by mature colonocytes for energy (ATP) generation, which is necessary for the transport of sodium (Na) enabling water absorption. Butyrate metabolism requires colonocytes to consume high levels of oxygen, creating a hypoxic epithelial surface, Disruption of this hypoxic surface has pathophysiological consequences: antibiotic treatment with streptomycin preferentially depletes the Clostridia class, thereby reducing butyrate production and increasing partial oxygen pressure, leading to diffusion of oxygen into the intestinal lumen. This creates a window of opportunity for oxygen-tolerant microbes and is associated with the uncontrolled expansion of facultative anaerobic bacteria such as Enterobacteriaceae, including pathogenic Escherichia coli or Salmonella enterica species. With the loss of butyrate-producing microbes, such as Coprococcus spp., oxygen diffuses into the lumen and supports the expansion of pathogenic oxygen-tolerant microbes. Taurocholate, a bile acid is a potent germinant for all spore formers, increasing the culturability of spores from commensal bacteria by up to 70,000-fold. Normally, acetate, propionate, and butyrate have a molar ratio of 3:1:1 accounting for >95% in human GI tract; however with depression, there are higher acetate (with diarrhea) and lower butyrate and propionate levels.

The GI microbiota secretes a wide range of metabolic products, including secondary bile acids and SCFA. Next-generation probiotics (NGPs, may also include prebiotics or its combination) may restore the immune response by modulating gut microbial changes or secreting compounds that act in the lungs. SCFA may affect human nutrition, homeostasis, and resistance and affect bacterial fitness, they may affect the host. SCFA requiring active transporters across intestinal barriers in the colon epithelium, namely monocarboxylate transporter I (MCT-1) found in colonocytes and lymphocytes, and sodium-coupled monocarboxylate transporter 1 (SMCT-1) receptors) found in colonocytes, kidneys and thyroid. The GI molar ratio of SCFA: propionate, butyrate, and acetate (also, formate) synthesis in the colon is 60:25:15, correspondingly, usually in the ionic form; however, the ratios might vary based on variables, such as nutrition and microbiota diversity, the location of fermentation, and the host genotype. Acetate is generated by many bacteria, including Akkermansia muciniphila, Lactobacillus spp., Bacteroides spp., and Bifidobacterium spp., while propionate is synthesized by Roseburia inulinivorans, Bacteroides spp., Salmonella spp. and Veillonella spp. Butyrate is produced by Roseburia, Faecalibacterium prausnitzii, Blautia, Coprococcus and Eubacterium rectale spp. Butyrate, the most studied, assists with sateity, which in turn increases leptin, a hormone that can metabolize fats, and control obesity. Those patients with CFS/ME with shorter-term disease had changes in their microbiome with regard to diversity, including a lower number of microbes known to produce butyrate; it is possible that the dysbiosis primes or sustains an aberrant immune response (autoimmunity) following disease onset. Oral microbiota including P. gingivalis, a gram-negative anaerobic bacteria that ferments a wide variety of substrates, including butyrate, can lead to periodontal disease (another study shows a disruption in the oral mucosal barrier can contribute to RA pathogenesis thereby releasing citrullinated oral bacteria (Porphyromonas sp. and Aggregatibacter sp.) into circulation, which activate inflammatory monocyte subsets that are observed in inflamed RA synovia and blood of RA patients with flares and activate anti-citrullinated protein antibodies (ACPAs) B cells, thereby promoting affinity maturation and epitope spreading to citrullinated human antigens; ciliated host cells found in the lower respiratory tract epithelium can bind more microbes on the surface; furthermore, this can be associated with 1.5-fold risk of atherosclerosis because inflammation alters HDL constituents and the concentration of LDL and HDL; RA autoimmune antigens can have homology with epitopes from proteins of the Prevotella sp. and Butyricimonas sp., found particularly in the gut microbiota, and bind at host mucosal sites, and subsequently may promote systemic autoimmunity that affects joints), halitosis and potentially reactivating latent Epstein Barr virus (EBV) and human immunodeficiency virus type 1 (HIV-1) due to butyrate's potency to suppress the activity of histone deacetylase (HDAC), as well as transmission of signals through GPCRs, particularly GPR41 (may assist with weight loss), GPR43 (expressed throughout the digestive tract, especially in immune cells), and GPR109A (most potent binding, found only in human dendritic cells (DCs), neutrophils, and macrophages in the immune system). Similarly, butyrate promoted herpes simplex virus type 1 (HSV-1) reactivation in mice. Butyrate can also assist improve sleep (tributyrin and butyrate robustly boost nonrapid eye movement sleep (NREM) in rats and mice), increases GSH, ads as a histone deacetylase inhibitor to protect chromatin to increase BDNF, to sustain and enhance long-term potentiation (LTP) inclusion, it serves an essential role in cognitive function in the hippocampus, and also inhibits pro-inflammatory cytokines (BDNF levels diminish with aging), activation of NF-κB (involved in the GPR43 interaction and IFN-β production), and has anti-caner (but sometimes pro-cancer) effects by affecting histone activation. Butyrate ameliorated aberrant behaviors, including locomotor hypoactivity, anxiety disorder, depressive behavior, impaired learning, spatial recognition memory, and effectively reduced chronic alcoholic CNS damage by suppressing microglia-mediated neuroinflammation of the GPR109A/PPAR-γ/TLR4-NF-κB signaling pathway and modulating the microbiome-gut-brain axis. A decrease in SCFA synthesis by the gut microbiota has been linked to infections with both influenza A and SARS-CoV-2 by enhancing effector T-cell activities and reducing viral levels. Secondary bile acids and butyrate are metabolites that have shown potential for the treatment of viral respiratory tract infections such as COVID-19. Butyrate reduced the levels of epithelial or mucosal ACE2 (which are less expressed in children than in adults, potentially accounting for the differential effects of Covid 19) and Tmprss2, which are proteins that allow SARS-CoV-2 to enter host cells. Butyrate increased the expression of Adam17, a metallopeptidase involved in ACE2 shedding. GPR41, and GPR43, were recently identified in the nasal mucosa, such that a topically applied nasal coating of SCFA may prevent its binding to ACE2 receptors so as to prevent or treat early infection. In addition to directly interacting with viruses and trapping viral particles, beneficial bacteria (including those that produce SCFA, such that Enterococcus faecium NCIMB 10415 could bind and separate H1N1 and H3N2 swine influenza virus) may also physically impede virus adherence to host cells by occupying viral receptors (surface glycopeptides, glycolipids, fibronectin, hexose uptake, and cell substrate adhesion, are correlated with butyrate-induced cellular flatness in marine sarcoma virus-transformed rat cells (MSV-NRK)). Butyrate reduces the risk of contracting viral infections (prevents tissue injury caused by neutrophil infiltration into the airways, which occurs when mice are infected with influenza) by inducing bone marrow-derived free fatty acid receptor 3 (FFAR3) which leads to the conversion of monocytes into alternatively activated macrophages (AAMs) instead of proinflammatory macrophages, which in turn reduces neutrophil recruitment in the lung. Latobacillus rhamnosus and Lactobacillus gasseri (produce butyrate from fiber) may prevent mice from influenza as well as RSV. Butyrate increased CD8+ T cell responsiveness to the influenza virus by enhancing their metabolism and functional activity and controls interferon (IFN) responses to viral diseases. Injecting SCFA-producing Lachnospiraceae or exogenous acetate into mice reduces respiratory syncytial virus (RSV) pulmonary viral load, prevents recruitment of inflammatory cells in the lung, and increases animal survival. High-risk Human papillomavirus (HPV) which infects keratinocytes is responsible for encoding two major oncoproteins known as E6 and E. All SCFAs alter local habitats in the gut to prevent infection, and limit Salmonella colitis in mice. the use of antibiotics to reduce the normal intestinal flora culminated in significantly lower norovirus viral titers (this virus cannot infect B cells without the presence of human blood group system antigens, HBGA-positive intestinal bacteria; and the mechanism responsible for this likely lies in the ability of the commensal microbiota to inhibit viral reproduction. Because butyrate treatment increases the survival of keratinocytes transformed with the HPV oncoprotein, commensal bacteria are likely to play a role in both the chronicity of high-risk HPV infection and the propensity for head and neck squamous cell carcinoma (SCC). Most bacteria have other non-histone proteins that protect their DNA, but new research has confirmed that histones (previously thought not to be part of bacterial composition) can be present in bacteria as well. Rotavirus (RV) infection disrupts the intestinal barrier and alters intestinal homeostasis, resulting in ER stress (ERS), via a protein kinase RNA-like ER kinase (PERK) which may induce epithelial cell death through the process of apoptosis. Butyrate reduced RV-induced apoptosis through activation of the PERK-elF2α signaling and protects human nucleus pulposus cells against oxidative stress and apoptosis caused by tert-butyl hydroperoxide and inhibited the PERK-elF2α signaling pathway in type 2 diabetic rats, which resulted in a reduction in ERS-induced islet β-cell apoptosis. Sodium butyrate also exerted its antidiarrheal effect on weaned piglets by increasing the expression of colonic tight junction protein in a GPR109α-dependent (a butyrate-specific GPCR expressed in the intestinal epithelium) manner, which prevented the piglets from developing diarrhea. Excessive levels of SCFAs in the female urogenital tract microbiota appear to increase inflammatory responses in younger women, which can inhibit neutrophil (contains GPR43) extracellular traps (NETs), possibly impacting HIV infection. Women with bacterial vaginosis have increased C16 expression on their vaginal neutrophils, and in vitro treatment of peripheral neutrophils with high amounts of butyrate caused neutrophils to show altered phenotypes (may reduce their antiviral capacity), and suppresses neutrophil death. The use butyrate or propionate supplementation and high-fiber diets in mice for autoimmune inflammatory models showed a trend to prevent the development of systemic inflammation to viral infections (as seen with stromal keratitis of the eye from herpetic lesion in mice) and other types of inflammatory responses. Chikungunya virus (CHIK) rheumatic immunopathology was shown to be altered by a high-fiber diet by downregulating the macrophage clearance-phase signature gene in mice, and facilitated tissue regeneration. CHIKV kills endothelium cells in the body, and edema is a frequent sign of CHIKV severity; butyrate inhibiting classical NF-κB pathways, and the endothelial barrier repair was suboptimal which increased the edema, and this may be context-dependent. Interferons (IFNs) are a type of cytokine released by host cells in response to viral infection and have a significant antiviral effect, and gut microbiota, depending on the virus and the environment, may stimulate or inhibit IFN signaling. The antiviral activity of acetate, produced by the microbiota, against RSV is mediated in part by increased GPR43-dependent IFN-β production. Butyrate increases the proliferation of many viruses (including hMPV, HIV, influenza A virus, and reovirus) due to its HDAC inhibitory activities. Acetate protects against RSV-induced illness by boosting IFN-1 responses and propionate prevents bronchiolitis and the establishment of asthma in adult mice. Reduced butyrate production has been attributed to deficiencies in gut microbes, such that F. prausnitzii has been associated with a range of health conditions including Crohn's disease and psoriasis, as well as a dearth of Eubacterium rectale are have been associated CFS.

Butyrate and acetate (both SCFA) regulate and maintain the healthy gut microbe population while altering the luminal environment to void pathogens and protect against diet-induced obesity. Luminal butyrate adversely affects pathogenic bacteria like Escherichia coli, Salmonella spp., and Campylobacter spp. Butyrate serves as a direct substrate to undergo β-oxidation, but also induces FGF21 in serum, liver, and adipocytes, which in turn stimulates fatty acid β-oxidation and hepatic ketone production. Orally consumed butyrate is shown to induce GLP-1 secretion, a hormone known to support the improvement of glucose tolerance and appetite and food portion control. Butyrate fed mice remain lean (despite dietary calorie load), have increased energy expenditure in the form of body heat generation, and tend to be more physically active. Butyrate is shown to reduce or inhibit the microbiome population responsible for generating propionic acid, another SCFA, which has been implicated in ASD (70% of children with autism or ASD have GI disorders and altered gene expression in the brain as a function of the resulting SCFA imbalance). Butyrate has been shown to play a role in healthy peristalsis to help normalize movement in cases of constipation or diarrhea, and improve gut lining integrity. Butyrate is a potent histone deacetylase (inhibitor of class I and class II histone deacetylases (HDACs) in rat myotubes and endothelial cells; HDAC modulation is also associated with retrieval of long-term memory to recognition including memory recall in AD, with improved protection from free radical damage associated with strained or extreme metabolic conditions and environmental toxins) inhibitor, with remodeling of chromatin towards an open and transcriptionally competent state and both molecules have been associated with improved metabolic profile and better cellular oxidative status. Butyrate induces PGC1α (which can be deficient in type II diabetic patients), which was associated with decreased oxygen consumption and fatty acid oxidation, upregulating carnitine palmitoyltransferase 1b (CPT1b), mitochondrial sirtuins (SIRT3-5), and the mitochondrial anti-oxidative genes SOD2, and catalase, which participates in cell defense, especially in cancer. In endothelial cells, butyrate suppressed gene expression and LPS-induced secretion of several pro-inflammatory cytokines GM-CSF, IFNγ, IL-1Rα, IL-2 and IL-17, and concomitantly increase in anti-inflammatory IL-4 secretion, decreased production of ROS, inhibition NF-κB with increased I-kB levels as a countermeasure for improved long-term inflammatory control, and upregulates oxidative stress resistance factors FOX03A and MT2 in mouse kidney. Butyrate has a direct effect on insulin signaling and improves L6 myotubes insulin sensitivity through an upregulation of IRS1, due to an increased histone acetylation in the proximity of the IRS1 transcriptional start site. Butyrate activates Nrf2, alleviates oxidative stress and will improve catalase activity. It is shown to stimulate neurogenesis in the ischemic brain via BDNF upregulation, and has antidepressant-like effects. Preconditioning with butyrate administration protects myocardial injury associated with ischemia by inhibiting expression of inflammatory cytokines. It protects the pulmonary artery smooth muscle cells from oxidation associated with hyperoxia and improves metabolism and muscle atrophy associated with aging. Butyrate has been shown to have a significant preventive influence on cardiovascular health, reduce serum triglycerides by as much as 50% compared to controls and lower endogenous cholesterol production. Oral sodium butyrate attenuates experimentally induced colitis, with anti-inflammatory effect and remission in Crohn's disease patients through downregulation of NF-κB and IL-1. Intralumen butyrate has been shown to directly support health of the GI lining, to exhibit trophic effects on intestinal cell proliferation and improve villi status. In addition, butyrate has been shown to be a potent promoter of intestinal immuno-regulatory T-cells, as well as a mechanism for its inhibition of cancer. Butter is one of the richest butyric acid food sources with a naturally inherent supply of 3-4% of its fat content (one tablespoon=15 ml of butter typically delivers 14 grams of fat; of which 560 mg is butyric acid).

Butyrate 150-300 mg/day (one capsule three times daily with meals, a few hours before or after taking other medications) is the most common dosage recommendation for currently available butyric acid products (found in butter 3-4%, ghee, raw milk and parmesan cheese, and properly made kombucha, or prebiotics like raw Jerusalem artichokes, raw dandelion greens, raw jicama and under-ripe bananas).

Butyrate is a small chain triglyceride (SCT, with 4 carbons) that is produced by the metabolism of fiber and resistant starch by the intestinal organisms. Butyrate regulates the intestinal mucosal immune response by regulating the functions of the intestinal mucosal immune and non-immune cells. Butyrate also influences the differentiation, recruitment and apoptosis of cells. Butyrate has been known to have many gut, neurological, and anti-inflammatory benefits but when it applies to mast cell activation syndrome, the data proves that butyrate might be one of the best mast cell stabilizing tools. Supplementation with sodium butyrate has been shown to inhibit allergen induced histamine release and shown to modulate the function of our innate immune system. Butyrate also inhibits mast cell degranulation while enhancing inflammatory mediator production. Butyrate was significantly higher among almond eaters.

Microbial production of SCFA, a prominent nutrient for the human host fulfilling various regulatory functions including maintaining intestinal homeostasis. SCFAs have epithelial growth-promoting and anti-inflammatory effects as well as a variety of other health-promoting effects. Thus, if SCFAs are reduced, inflammation and disease can take over more easily. The activation of an immune and repair response in the intestine is induced by bacterial products reaching Toll-like receptors on the inner side of the epithelial cells due to damage of the barrier function. A mucosal inflammatory response results in a reduced tight junction-mediated barrier function, correlated with water and electrolyte leakage. This condition also can be considered an attempted discharge of potential pathogens, together with a substantial number of the other intestinal inhabitants. A general immune stimulation is of advantage to the host, enabling the destruction of pathogens at an early stage. The inflammatory response is elicited by bacteria, which manage a direct contact to the mucosa, due to a destabilized mucus layer, and infiltrate the tissue. In response, an increased number of leukocytes migrate into the intestinal lumen to hinder bacterial infection.

Butyrate bidirectionally modulates the KS, which influences food intake and energy metabolism for homeostasis.

The dominate phyla of the healthy adult gut are Bacteroidetes and Firmicutes (>90%), while Actinobacteria and Proteobacteria contribute a smaller portion. Heritable and environmental factors (delivery method, age, sex, diet, use of medication and/or antibiotics, ethnicity, geographic location, socioeconomic status) account for approximately 30% of the variability in microbial diversity in the healthy human gut, leaving approximately 70% currently unexplained. A healthy microbiome can be defined by long-term diversity (ie, multiple species) and stability in the presence of dietary perturbations, medication use, pathogen colonization, or disease. Gut microbial communities break down complex polysaccharides into metabolites, vitamins, and SCFA, especially butyrate, helping promote the integrity of the epithelial barrier. If this barrier is disrupted, gut microbes, toxins, or other metabolites can escape, eliciting increased inflammatory cytokines and T-cell responses (activation, homing, tolerance) locally, systemically, and in the skin. In a case-control study of 228 participants, patients with melanoma had a different structure of microbiome, with lower abundance of multiple beneficial commensals. The gut microbiota of patients with early-stage melanoma was characterized by higher alpha diversity and a distinct microbiome structure (higher abundance of the genus Roseburia a producer of butyrate, and Roseburia was enriched in patients with early-stage compared with late-stage melanoma) compared with those with late-stage (higher relative abundance of Fusobacterium, also found in colorectal cancer patients), but it is unknown whether this is a cause or an effect (duration of exposure, magnitude of effect) to advocate whether fecal transplants or pre/probiotics to maximize responses for precision medicine strategies. When stratifying patients with melanoma taking immune checkpoint inhibitors by treatment response, many studies identify key bacterial taxa associated with successful response indicating that gut microbiome composition has a direct impact on tumor responses.

The maternal intake of sucralose was found to inhibit intestinal development, induce intestinal dysbiosis, and decrease the production of butyrate-producing bacteria and butyrate in offspring through downregulation of G-protein-coupled receptor 43 (GPR43), and to exacerbate high fat diet (HFD-induced hepatic steatosis in adulthood. For mice with induced MS, at the phylum level, there was an increase in the relative abundance of Verrucomicrobia and Proteobacteria and a reduction in Bacteroidetes was observed in animals with MS. However, at the genus level, MS increased the abundance of Akkermansia, Blautia, Corynebacterium, and Robinsoniella, while Alistipes, Barnesiella, Paraprevotella, Saccharibaderia_genera_inc_ertaesedis, and Streptococcus were reduced, with a decrease in alpha diversity. Artificial sweeteners and its dosing may affect overall appetite (caloric intake), and can have different effects in those with aging, pregnancy or lactation, metabolic or chronic disease processes.

Reduced lipopolysaccharides and increased intake of SCFAs like sodium butyrate can also regulate neuronal growth. Agathobaculum butyrici producens SR79, a bacterial organism that produces butyrate, reportedly enhances cognitive function. The daily intake of probiotics including Lactobacillus fermentum, Lactobacillus acidophilus, and Lactobacillus casei facilitate the restoration of the gut microbial balance and aid in the maintenance of intestinal homeostasis. Prebiotic supplements, such as sodium oligomannate, have demonstrated BBB penetrability, as well as the ability to bind to Δβ molecules and prevent the conversion of Δβ fibrils into plaques.

To facilitate adequate fermentation by combining both lactate-producing and butyrate-producing bacteria together in an anaerobic environment (large intestine), a significant amount of lactate will be converted to butyrate in which it is estimated to require >10\hat{ }9 cfu/g of probiotic lactic acid bacteria (LAB, homofermentive: bifidobacteria, enterococci, streptococci and Eubacterium; Lactobacilli: Lacticasebacillus paracasei, Lactiplantibacillus plantarum, Lacticasebacillus rhamnosus, and heterofermentive: Levilactobacillus brevis (Due to the low production of lactate, a larger number of heterofermentative over homofermentative LAB would be required for this stimulation); also may include Roseburia/E. rectale group; Eubacterium halli bacterium SS2/1, E. plexicaudatum and A. caccae, which are all known to be lactate-utilizing, butyrate producing; and Veillonella spp., which are known to be lactate-utilizing, but propionate-producing bacteria; also, Clostridium butyricum/tyrobutyricum L319/C. propionicum (also produces propionate) are all butyrate producing non-LAB, that utilize the lactate produced by the other microbiota) would be required to stimulate SCFA production in the large intestine.

However, butyrate, by inhibiting HDAC activity, suppressed levels of specific by type I interferon (IFN) stimulated gene (ISG) products in human and mouse cells, which may increase viral cellular infection and replication in influenza virus, reovirus, and human immunodeficiency virus (HIV), and previously retroviruses, Epstein-Barr virus (EBV), measles virus, Borna disease virus, and herpes simplex virus (HSV). In vivo, butyrate and dietary fiber were shown to be protective against the influenza virus (H1N1 influenza A) pathology in mice, despite an increase in virus titer. In contrast, butyrate and fiber were shown to be detrimental in the inflammatory disease caused by Chikungunya virus, a distinct RNA virus. Despite the critical role of type I IFNs in antiviral defense, the levels and activities of these IFNs are held in check by dozens of cellular proteins in order to limit their tissue damaging effects.

Butyrate (butyric acid) ads by controlling inflammation, supporting the integrity of the intestinal barrier, and regulating energy expenditure; some experts even claim that butyrate supplements may be used to support gut health, and possibly more effective than probiotics, such that beneficial gut bacteria digest and break down dietary fiber (fruits, vegetables, nuts including almonds; walnuts also offer cardioprotection, whole grains, and legumes), resistant starches (green bananas, potatoes, and legumes), and fermented foods (yogurt, kefir, sauerkraut, or kimchi). It can reduce sleep onset and improve sleep quality, inducing significant increases in non-rapid-eye movement sleep (NREMS) and the duration of deep sleep, improve insulin sensitivity, enhance glucose metabolism, and help regulate appetite and body weight, and contributes to the production of BDNF, which is essential for neuronal survival and growth, serves as a neurotransmitter modulator, and participates in neuronal plasticity vital for learning and memory, which improves plasticity thanks to its neuroprotective effects.

Giving butyrogenic prebiotic fibers or sodium butyrate may hold promise as a repurposed therapy to reduce THC Cannabis-related behavioral effects.

Lactobacillus genus synthesize lactic acid, which is converted to butyrate and later to acetyl-CoA, which is used in the Krebs Cyde to generate adenosine triphosphate (ATP). Notwithstanding, both processes occur mostly in the gut; not in skeletal muscle to directly delay fatigue.

Eubacterium rectale gut bacteria, with lower butyrate production, which is the primary energy source for cells that line the gut, providing up to 70% of their energy requirements, as well as acting to support for the gut immune system, and protection against diseases of the digestive tract; Butyrate, tryptophan, and other metabolites detected in the blood are important for regulating immune, metabolic, and endocrine functions) who have normalization of LGS. While species of butyrate-producing bacteria decreased, there were increased levels of nine other species in ME/CFS, including Enterocloster bolteae and Ruminococcus gnavus, which are associated with autoimmune diseases and inflammatory bowel disease, respectively.

Age-related dysbiosis of the gut microbiome as contributing to tissue-specific (such as in the liver, adipose, and intestine) as well as a global inflammatory state in the elderly; Oral dysbiosis also plays a role in mediating age-related neurodegenerative diseases, expanding the term into the “oral-gut-brain axis”; Metabolites produced by gut and oral microbes (particularly Streptococcus mutans and Porphyromonas gingivalis) have been linked to the formation of β-amyloid and its accumulation in the brain, tau protein phosphorylation, and neuroinflammation in individuals with Alzheimer's disease; SCFA, such as butyrate, are considered a primary microbial anti-inflammatory metabolite that is known to inhibit the NF-κB pathway and regulate Th17/Treg differentiation by inhibiting IL-6/signal transducer and the activator of transcription 3 (STAT3)/IL-17 pathway and promoting Foxp3. Faecalibacterium prausnitzii has been identified as a butyrate-producing bacterium with an inverse relationship with different proinflammatory markers.

A centenarians' gut ecosystem is the increase in facultative anaerobes, such as bacteria belonging to the Micrococcaceae family, the Fusobacterium, Bacillus, Staphylococcus, and Corynebacterium genera, and many members of the phylum Proteobacteria, with reduced core taxa, such as Bacteroides and Roseburia, associated with increases in Akkermansia (modulated by local levels of IFN-γ) and Bifidobacterium, which may have pro-longevity effects; Opportunistic microbes, especially the Enterobacteriaceae family, thrive in an inflamed environment and are known to increase in the elderly and are associated with a decrease in F. prausnitzii; also increased production of amino acid metabolites, such as from tryptophan (e.g., indoles that bind the aryl hydrocarbon receptor [408] and phenylalanine/tyrosine, and through the production of secondary bile acids.

Butyrate-producing bacteria, mostly from the Firmicutes phylum, ae reduced in individuals with depression, and eight studies demonstrated lower relative abundance of Coprococcus spp. in individuals suffering from depression or depression-like symptoms and in children with language development, ASD, GAD, OCD and schizophrenia.

Faecalibacterium prausnitzii (which is significantly reduced in conditions including type 2 diabetes and cardiovascular disease), can be co-isolated with Desulfovibrio piger (which has beneficial effects on Faecalibacterium prausnitzii growth and function). By combining a naturally occurring symbiosis and subsequently “training” the oxygen-sensitive bacteria in a favorable electrochemical environment, the researchers were able to isolate more oxygen-tolerant Faecalibacterium prausnitzii, increasing both bacterium biomass and the production of butyrate as a new strategy for next generation of probiotics.

Akkermansia on the other hand has been shown to use mucus as a carbon source and degrade the colonic mucus barrier, which might disrupt intestinal permeability and increase pathogen susceptibility causing inflammatory condition on the intestinal wall. In addition to the above gut microbial changes, PD patients also display reduced abundance of prominent SCFA producers. Strains of Faecalibacterium spp., Blautia spp., Prevotella spp, and Roseburia spp, that are common butyrate producers are more consistently reduced in PD patients. Butyrate has been shown to exert barrier protective, anti-inflammatory role in the GI tract and prevent dopaminergic neuron degeneration and subsequent bradykinesia in mice. Studies show that gut microbes play an important role in maintaining the epithelial barrier (including IBD, stress-related psychiatric disorders, anxiety, PTSD, depression, ASD, autoimmune disorders, and PD), particularly through butyrate production, which modulates expression of mucin-associated genes in goblet cells and regulates expression and distribution of epithelial tight junction proteins.

In subjects with features of metabolic syndrome, flavonoids present in citrus fruits, mainly hesperidin and naringin supplementation (orange and grapefruits extracts of 500 mg/day for 12 weeks) can interact with the intestinal microbiota and can thereby beneficially affect factors related to metabolic and intestinal health, resulted in increased cumulative SCFA, with increased production of butyrate, and to a lesser extent, acetate, and valerate, with reduction in fecal calprotectin level (a marker for intestinal inflammation) related to an increase in the relative abundance of the Clostridium coccoides/Eubacterium rectale duster (also, increases in Lactobacillus spp., Roseburia, Eubacterium ramulus, and Bacteroides eggerthii in other studies). SCFAs can favorably modulate energy metabolism (prevent obesity-related insulin resistance), gut health regulation, anti-inflammatory effects, and the suppression of cancer cells, and can signal to receptors involved in the control of appetite-inhibiting hormones, such as peptide YY (PYY) and glucagon-like peptide 1 (GLP1), and may thereby be able to affect food intake. In the skeletal muscle, acetate and butyrate might influence muscle fat oxidation and glucose metabolism via the activation of enzymes via AMPK and PPAR-gamma mechanisms. Butyrate plays a role in decreasing appetite and body weight by activating free fatty acid receptors (FFARs) in intestinal cells, triggering the release of GLP-1 and PYY, mitigates inflammation by enhancing the expression of interleukin10, and enhances the integrity of the intestinal barrier (also seen with valerate) while modulating the activities of intestinal microbiota and immune cells. GLP-1 has been shown to boost insulin secretion and simultaneously suppress glucagon secretion, thereby regulating blood glucose levels. Conversely, PYY decreases appetite and decelerates gastric emptying, contributing to a reduction in food intake. In addition, butyrate exhibits various beneficial effects on intestinal health.

Regular consumption of lacto-fermented vegetables may stimulate bacteria with the potential to produce SCFA including butyrate, a compound in the gut that is widely known for its positive effects on health, as well as increase production of catecholaminergic neurotransmitters.

Animals with a depleted microbiome took more cocaine and worked harder to seek drugs after abstinence, and significantly changed important neurobiological markers in reward-related brain structures. This suggests that the rewarding effects of cocaine were affected by the gut microbiome, and can be reversed with the addition of molecules produced by beneficial bacteria in the gut microbiome that are important for brain health known as the SCFA (butyrate).

In the absence of a normal microbiome, repletion of bacterially-derived (Proteobacteria) SCFA metabolites (i.e., butyrate) reversed the behavioral and transcriptional changes associated with microbiome depletion. Also, changes in gut microbiome composition drive fentanyl intake and striatal proteomic changes. Morphine tolerance (to antinocioception) is attenuated in germ-free mice and reversed by probiotics, and morphine influences the gut microbiome and its metabolomes. The gut microbiota can influence the microglia in a sex-specific manner.

Butyrate using is synthesized from dietary fiber, but may also come from protein via the lysine pathway. Beta-hydroxy-beta-methylbutyrate (HBM) is naturally produced in small amounts when your body breaks down leucine (<5%), but may also be present in avocado, grapefruit, cauliflower and catfish. HBM can slow muscle breakdown (along with strengthening exercises, which in turn can be probiotic with butyrate production), and maintain muscle mass to prevent age-related muscle loss, or sarcopenia, which affects approximately one in three adults age 50 and older and its imbalance contributes to fatigue, losses in strength and energy as well as poor mobility. Several receptors for butyrate have been identified, including GPR41, GPR43, and GPR109A. GPR41 is found in adipose tissues and immune cells. The highest expression of GPR43 has been found in immune cells, whereas GPR109A is essential for butyrate-mediated induction of IL-18 in colonic epithelium. A small portion of butyrate is transported to the liver and metabolized to produce ATP and it ae involved in lipid biosynthesis and influences glycolipid metabolism, as well as ad as a substrate for lipid biosynthesis. SCFA transport into the body occurs via 5 monocarboxylate transporter (MG) isoform s, of which MCT4/5 are more abundant in the distal colon, and MCT3 in the ileum, with an optimal pH of 5.5. Butyrate preferentially binds to GPR41 over GPR43, which has higher affinities for acetate and propionate and is found in adipose tissues, the pancreas, spleen, lymph nodes, hone marrow, and peripheral blood mononuclear cells. Butyrate directly regulates GPR41-mediated sympathetic nervous system activity to control body energy expenditure and maintain metabolic homeostasis, whereas GPR109A signaling activates the inflammasome pathway in colonic macrophages and dendritic cells, resulting in the differentiation of regulatory T cells and IL-10/8-producing T cells. Among the SCFAs, butyrate is the most potent in inhibiting HDAC enzymes, which remove acetyl groups from &N-acetyllysine on histones, allowing the histones to wrap the DNA more tightly. Many of the anticancer activities (especially colon cancer) of butyrate have been found to the mediated through HDAC inhibition, which includes inhibition of cell proliferation, induction of cell differentiation or apoptosis, and induction or repression of gene expression by downregulation of proinflammatory effectors in lamina propria macrophages as well as regulating cytokine expression in T cells. For anti-inflammatory effect, IL-10 and TGF-β are upregulated in response while activating PPAR-γ; proinflammatory cytokines IFN-γ, TNF-α, L-1β, IL4, and IL-8 are inhibited; and butyrate suppresses the NF-κB signaling pathways by rescuing the redox machinery and controlling reactive oxygen species which can protect your brain and improve its ability to adapt (plasticity) and cardiovascular disease; it also stimulates antimicrobial peptides, such as LL-37 in humans. Butyrate can modulate intestinal epithelial cell-mediated migration of neutrophils to inflammatory sites and plays a role in cell proliferation and apoptosis. Butyrate stimulates cell growth and DNA synthesis and induces growth arrest in the GI phase of the cell cyde. Butyrate has the ability to cross the blood-brain barrier, it activates the vagus nerve and hypothalamus, thus indirectly affecting host appetite and eating behavior. According to the United States Department of Agricultures (USDA), the recommended intake for dietary fiber is 25 grams per day for women and 35 grams per day for men, or about 28 grams as part mixture of soluble (butyrate-generating) and insoluble fiber sources in a 2,000-calorie daily diet. Some healthcare providers suggest avoiding butyric acid supplements if you're pregnant or breastfeeding, and it may trigger symptoms in people with bloating or a sensitive gut (food intolerance) who need lower fiber levels. A sodium butyrate supplement may help with IBS, diverticulitis and Crohn's disease, and sleep (dramatic increase in non-rapid-eye movement (NREM) sleep for four hours in mice). Exogenous butyrate may alleviate obesity related insulin resistance in bred mice through activation of adiponectin mediated pathway, stimulates the release of glucagon-like peptide-1 (GLP-1) found on intestinal cells for weight loss in those fellow fat diets. It may also stimulate peptide YY (PYY) in the intestinal wall, stimulates follicular hormone (FH) secretion from pituitary, downregulating the expression and activity of PPARγ, promoting a change from lipogenesis to lipid oxidation and can induce insulin resistance in the offspring of pregnant rats. It may control gluconeogenesis from the gut epithelium and through a gut-brain neural circuit to increase insulin sensitivity and glucose tolerance. It exerts multiple biological effects, including a glucose-dependent insulinotropic effect on pancreatic B cells, reduction in appetite, and inhibition of gastric emptying. In a rat model revealed that the HP, low carbohydrate (LC) diet induced weight loss and reduced adipose weight, and the plasma metabolites glucose, insulin, triglyceride, linoleate, palmitate, α-glycerophosphate and pyroglutamic acid and caused a significant increase in several plasma metabolites (i.e. urea, pyruvate, α-tocopherol, 2-oxoisocaproate, and (β-hydroxybutyrate with increased urinary excretion of palmitate and stearate and a reduction of pantothenate and the tricarboxylic acid (TCA or Krebs) aerobic cycle cycle (to produce nicotinamide adenine dinucleotide—NADH) intermediates citrate, 2-ketoglutarate and malate. Some bacteria (certain strains, i.e., Clostridium acetobutylicum) produce aromatic (phenylalanine, tryptophan, and tyrosine) and branched-chain (valine, leucine, and isoleucine) amino acids, which may contribute to insulin resistance and hyperglycemia; On the other hand, L-serine is the precursor to other amino acids (glycine, cysteine, tryptophan), as well as a precursor for metabolism (sphingolipids, folate, methane, sulfur, cyanoamino acid, and pyruvate), and also participates in the biosynthesis of purines and pyrimidines. It plays a fundamental role in stabilizing blood sugar concentration in the liver. Specific microbiome ecologies and amounts of proteins and enzymes can be predictive of successful weight loss diets and keeping it off, which correlated to a higher respiratory quotient (measured the ratio of inhaled oxygen to exhaled carbon dioxide, which serves as a proxy for whether carbohydrates or fats are the body's primary fuel) with a low-carb diet. SCFA, from fermentation of glycine, alanine, threonine, glutamate, lysine and aspartate can produce acetate (substrates for gluconeogenesis and lipogenesis and increases cholesterol synthesis); threonine, glutamate and lysine can produce butyrate (facilitates energy to colonocytes and regulates cell proliferation and differentiation, intestinal barrier function, as well as the transcription of numerous genes involved in mucin production and hormone secretion (i.e. PYY, GLP-1, GLP-2) that influence gut integrity and transit, appetite and glucose metabolism. inhibition of LPS-mediated inflammatory cytokine secretion by intestinal epithelial cells and other immune cells and via induction of colonic regulatory T cells partly by an epigenetic modification of the forkhead box-P3 promoter; binds GPR109a receptor expressed by intestinal macrophages and dendritic cells, thus activating production of the anti-inflammatory cytokine Il-10), and alanine and threonine can produce propionate (substrates for gluconeogenesis and lipogenesis and decreases cholesterol synthesis). SCFA are ligands for G protein-coupled receptors, namely Gpr4l (associated with reduced expression of peptide YY (PYY), a gut hormone involved in satiety and gut motility; direct administration of SCFA increases plasma PYY) and Gpr43, which are expressed in enteroendocrine L-cells of the distal small intestine and colon and regulates metabolism by inducing energy expenditure and mitochondrial function. It imparts numerous health benefits, such as—fat reduction, impeding type 2 diabetes mellitus (T2DM) progression, antihypertensive, along with anticarcinogenic effects, which can help anti-inflammatory functions by increasing the pH of the interstitial fluid, which may be indirectly evaluated by urine pH; serum tends to buffer these effects, and therefore, cannot be used as a proxy for anti-inflammatory bias on alkalinity). In gut dysbiosis, the ratio of long chain fatty acids (LCFA) which are pro-inflammatory, to SCFA is increased. Branched-chain fatty acids (BCFAs), namely isobutyrate, 2-methylbutyrate and isovalerate, are derived from branched-chain amino acids: valine, isoleucine, and leucine, and are present at much lower concentrations in the large intestine luminal content from many gram-positive bacteria; it regulates electrolyte absorption and secretion.

Low muscle strength was shown to be a significant and independent predictor of mortality risk, and a decline in muscle mass and strength is usually observed with age across different populations. Muscle protein anabolism in older adults may be negatively affected by inadequate nutritional intake or impaired response to nutrients and hormones. Lean body mass correlates with muscle mass (bone only accounts for approximately 7% of lean body mass, and its remodeling is slow lasting 4-24 months), with a lower risk of metabolic syndrome and reduced mortality risk among middle-aged women. Sarcopenia (age-related loss of muscle mass and strength) is a debilitating condition that is characterized by loss of muscle mass and strength and is associated with a range of other health outcomes including reduced physical functional performance, weakness, frailty, falls, hospitalization and death. It has been estimated that 30% of over 60s and 50% of over 80s have sarcopenia. Vital lipids, including long-chain fatty acids, were substantially depleted in the leg muscles of young genetic PCYT2-deficient mice (pail of the Kennedy pathway to synthesize two critical components of the phospholipid membrane that surrounds cells, which also had substantially thinner fibers), causing spinal problems, muscle wasting and atrophy, along with low bone density, and PCYT2 replacement muscle tissue, they found that the affected mice had significantly higher grip strength, muscle fiber size, and muscular mitochondrial function than the control group of aged mice. In both mice and people, PCYT2 declines with age, and significantly contributes to sarcopenia. The Dietary Guidelines for state that adults should aim to consume 130 g of carbohydrates per day, which should account for 45-65% of their daily caloric intake, and 46-56 grams of protein daily (based on active lifestyle). Older adults (gender differences with men require higher intake, and postmenopausal women have different protein requirements to prevent osteopenia) may require higher protein intake than that of younger populations due to age-related anabolic resistance of muscle protein synthesis. For this reason, expert consensus suggests that older adults should consume an additional 0.2-0.7 g of dietary protein per kg body weight than younger adults daily (consuming 0.8 and 1 gram of protein per pound of body weight per day to gain muscle weight; if overweight>25% body fat in men and >30% in women, then this can be reduced to around 1 gram of protein per pound of fat-free mass per day) in order to protect against muscle atrophy, and a specific protein source did not affect changes in strength outcomes. High-quality protein with essential amino acids for muscle protein synthesis and therefore maintenance of muscle mass, is important for maintenance of muscle health in older age; therefore, there is a need to understand whether replacement of animal protein with plant protein will make a significant difference in terms of muscle health outcomes.

Damaging exercise (like heavy training) can also increase protein breakdown which necessitates protein intake to drive protein synthesis higher and create a positive protein balance. There is evidence that equal amounts of protein from different sources are not met with an equal postprandial response in terms of amino acid absorption and metabolic utilization such that ingestion of animal protein may induce higher energy expenditure than plant. For example, modelling studies have found that soy protein experiences greater splanchnic extraction and nitrogen losses compared to milk protein. Proteins from animal food sources (meat, fish and dairy, the latter contain more calcium) are referred to as high-quality proteins due to the presence of all nine essential aminoacids (EAA: Phenylalanine, Valine, Threonine, Tryptophan, Methionine, teucine, Isoleucine, lysine and Histidine; BCAAs (Leucine, isoleucine and valine) at as signaling molecules that can help kick start the protein synthesis process; eating 3-5 protein feedings per day that each provide at 3-4 grams of leucine (followed by lysine, phenylalanine, tyrosine), is ideal for maximizing muscle growth, with plant proteins do not usually contain all EAA, and require significantly more consumed quantity) in high quantities as well as the greater bioavailability of these EAA. Young muscles are more sensitive to the anabolic action of EAAs compared to aging muscles with “anabolic resistance,” possibly related to declining physical activity, prolonged muscle disuse or chronic inflammation (impaired activation of mTORC1 and downstream targets implicated in translation initiation, such as ribosomal protein S6 kinase (p7056K) and eukaryotic initiation factor 4E binding protein 1(4EBP1)). A greater proportion of dietary fiber in plant protein food matrices is also expected to reduce protein digestibility (at least 10% lower at about 45-80%, vs. animal foods of about 90-95%¹). Protein quality can be summarized using the protein digestibility-corroded amino acid scare (PDCAAS, in which the validity of the reference pattern scoring based on preschool-ages (1-3 year old child) amino acid requirements with adults having slightly lower Recommended Dietary Allowances (RDAs) published in 2005, the validity of the true fecal digestibility correction and the truncation of PDCAAS values to 100% has been disputed by some experts). For acute ingestion in which 5 proteins, all with different amino acid profiles, receive identical scores of 1.0 limits its usefulness as a comparative tool: 1.00 cow's milk, whey, casein, eggs, soy protein; 0.996 mycoprotein; 0.99 potato protein concentrate; 0.95 chicken; 0.92 beef; 0.91 soy; 0.893 pea protein concentrate (isolate); 0.78 chickpeas and edamame; 0.75 black beans; 0.70-73 vegetables or legumes; 0.66 dehulled hemp seed; 0.64 fresh fruits; 0.59 cereals and derivatives; 0.597 cooked peas; 0.52 peanuts; 0.50 rice; 0.48 dried fruits; 0.525 wheat bran; 0.42 wheat; 0.25 wheat gluten. Although soy and beef protein have similar PDCAAS scores, beef has been shown to be more effective at increasing protein synthesis levels (maxed out at about 25 gm/day). Grain protein has a PDCAAS of about 0.4 to 0.5, limited by lysine. On the other hand, it contains more than enough methionine. White bean protein has a PDCAAS of 0.6 to 0.7, limited by methionine, and contains more than enough lysine. When both are eaten in roughly equal quantities in a diet, the PDCAAS of the combined constituent is 1.0, because each constituent's protein is complemented by the other. Plant and animal proteins can induce different rates of muscle protein synthesis which can affect muscle growth for the bodybuilder and muscle mass in the individual interested in longevity and quality of life. Amino acids from plant proteins are more likely to be shuttled to the liver, ultimately stimulating ureagenesis (and amino acid oxidation), with the production of urea, compared to animal proteins, which lowers the potential of plant-based proteins to increase protein synthesis, as fewer amino acids will be available to promote and contribute to protein synthesis. Current targets for carbon emissions will not be met if the current Westernized dietary pattern does not change in favor of a more plant-based diet. Plant proteins in diet may be associated with greater intolerances or allergies (gluten, celiac, casein, peanuts or nuts), whereas animal products may have dietary dairy/lactose, iodine or shellfish intolerance. and possible antibiotic/pesticide or herbicide exposure, with preservatives/processing/curing, as well as “farm” vs. “wild” caught); both sources may include inorganic or non-GMO sourcing. The proportion of animal protein and plant protein in a diverse diet does not influence the chronic response of muscle turnover, provided the total protein consumed is adequate, and usually does not affect lean body mass. Although animal protein was found to have resulted in a statistically significant gain in percent lean body mass, as well as absolute and percent lean mass among younger adults (<50 years), the clinical significance of this increase is unclear. In a retrospective study conducted among Korean adults, the average percent lean mass of individuals with no metabolic syndrome was found to be approximately 1% higher than those with the condition. Plant-based sources of protein are considered ‘complete’, especially soy, quinoa, or pistachios may have improved cardiovascular health outcomes and all-cause mortality and are environmentally sustainable. Mycelium-based mushroom protein is also high in protein, and microalgae, Spirulina and chlorella are being studied, as well as hemp (seed) derived sources (which may have a difference indigestion and gas production). A high-protein, non-animal-derived diet can the as effective as a diet that consists mainly of animal-derived protein sources, when paired with leg resistance training for 10 weeks to prevent sarcopenia. In another study, pea protein gave similar results as whey (dairy origin) protein. Plant protein intake may need to be hybridized or enriched with other nutritional compounds, such as beta-hydroxy-betamethylbutyrate (HMB in which most of the endogenous HMB is produced in the liver, considered safe but not GRAS) and vitamin D, to help maintain muscle mass among middle-aged and older adults (along with Vit K, B5 and ethanolamine, which requires gut microbiome source). A study published in 2022 found that a plant-based diet is associated with lower body weight, fat, and waist size compared to meat-eaters.

Food is nothing more than stored chemical energy, and glucose, fatty acids, and amino acids, are all on the “chopping block” for ATP energy extraction, but through different metabolic pathways. They all end up with a common 2-carbon molecule called acetyl CoA. During starvation or high-fat diets, most of the acetyl CoA molecules come from fatty acids due to the depletion of glucose. However, unlike glucose, fatty acids are not soluble and therefore cannot cross the blood-brain barrier, creating a situation that puts the energy supply to the brain under threat. Therefore, as a temporary substitute for glucose, the liver condenses two molecules of acetyl CoA to produce beta-hydroxybutyrate and acetoacetate, both soluble 4-carbon molecules called “ketone bodies.” These ketone bodies can easily cross the blood-brain barrier and serve as a temporary stopgap for the brain's preferred glucose. Ketone bodies are not used directly as fuel sources because they have to be reconverted to acetyl CoA in the cell's “powerhouse” before they are terminally burnt into carbon dioxide and water. with genetic selection, tropical dwellers can convert an 18-carbon plant mega-3 fatty acid called alpha-linolenic acid (ALA) to useful 20- and 22-carbon omega-3 fatty acids called eicosapentaenoic acid (EPA) and docosahexaenoic acid (DHA) respectively through enzymes called FADS1 and FADS2. On the other hand, Arctic dwellers, eat a high-fat and low-carbohydrate diet, and have less activity for the production of ketones and instead “increase heat in the body to stay warm in a cold climate by directing free fatty acids away from liver cells to brown fat,” due to a CPT1A gene (found in the liver and kidney has the dual responsibility for ketogenesis and regulating the import of long-chain fatty acids into mitochondria to help maintain energy) variant, from tropical dwellers. They use a unique process of fermenting animal protein to obtain calories from carbohydrates mostly in the form of glycogen, otherwise called “animal starch,” from the consumption of raw meats with increased ability to generate glucose from their high protein consumption to compensate for the lack of abundant tubers and fruits. For growing babies in Artic environments, the only sure source of glucose is in breast milk (often occurs until age 4-6), which is a disaccharide of galactose and the often-maligned glucose by low-carb proponents. The keto diet (KD) has a surprising effectiveness in weight loss and diabetes reversal, but no more so than any other fad diets or rationed junk meals.

However, in other studies a KD doubled the risk of cardiovascular events, including heart attack, stroke and blocked arteries.

Ketone bodies (KB), which are regularly produced by the liver but become more numerous when glucose, a sugar that acts as the main power source for cells, is in short supply. This can occur during exertion such as exercise, when cells are rapidly burning through fuel, or during fasting, when there is little food available to be broken down into glucose. To compensate, the liver steps up production of KB to feed the brain and other organs. KB also power immune cells, CDB+ T cell which prefer KB (b-hyroxybutyrate and acetoacetate (AcAc)) over glucose for synthesis of acetyl-CoA, to improves its function by reprogramming them to better neutralize threats and enhance immunity (and possibly cancer) through effects on histone acetylation. The low-carbohydrate high-fat diet (LCHFD), also known as the ketogenic diet (KD; 65-70% fat; 20% protein; and 5-10% carbohydrate with a preferred 50 gram maximum), has cycled in and out of popularity for decades as a therapeutic program to treat metabolic syndrome, weight mismanagement, and drug-resistant disorders as complex as epilepsy, cancer, dementia (systemic plasma ketones increase just beyond common baselines of (0.2 mM) are shown to improve brain ketone status and consequently serve neurons with an alternative energy substrate to glucose in AD 10-20 grams daily of exogenous ketone supplementation in divided doses are used with success, depression, improve cardiac health by reducing myocardial glucose uptake and increasing blood flow. It can be limited by patient non-adherence (although a calorie restriction by carbohydrate restriction is notoriously poorly tolerated unless coupled to a compensatory higher fat intake which contributes to an equicaloric outcome) due to the restrictive nature of the diet and symptoms related to energy deficit and GI adversity during the introductory and energy substrate transition phases, as well as controversial outcomes, adverse events and sustainability. Ketogenesis is an evolutionarily conserved alternative metabolic strategy to provide an energy source during starving, neonatal life of prolonged physical effort, whereas ketoacidosis with elevated ketone bodies (KB) in diabetics can be dangerous. -hydroxybutyrate (BB, or its monoester,D)-β-hydroxybutyrate, which is synthesized in the liver via the metabolism of fatty acids (e.g., butyrate), β-hydroxy β-methylbutyrate, and ketogenic amino aids through a series of reactions that metabolize these compounds into acetoacetate, which is the first ketone body that is produced in the fasting state) is a butyric acid, GRAS, and is the most abundant KB generated by the liver under starvation (not to be confused with butyrate, both SCFA, which is a beneficial metabolite by gut bacteria from ingesting fiber). R-BHB downregulates hexokinase 2 by downregulating the less needed glycolytic pathway, which causes an inhibition of insulin signaling in oxidative muscles via a 50% inhibition of insulin-mediated phosphorylation of protein kinase B. BHB has a slight pro-inflammatory effect on endothelial cells, with increased CCL2,1L-6 and IL-1 gene expression). However, there is a systemic anti-inflammatory state of the organism during caloric restriction, which has been associated to a BHB-mediated inhibition of the NLRP3 inflammasome in bone marrow derived macrophages and circulating neutrophils. BHB is produced from fatty aid oxidation-derived acetyl-coenzyme A (Ac-CoA; Oral sodium D,L-BHB at 1000 mg/kg body weight per day has been administered to children as young as two years old with cardiomyopathy and leukodystrophy from acyl-CoA dehydrogenase deficiency, responded within 1 week, and restored neurological function after two years to include walking and improved brain MRI), and transported to extrahepatic tissues for terminal oxidation. BHB, is GRAS, a water-soluble principal reversibly formed from the reduction of AcAc in the mitochondria as a signaling molecule of modulating lipolysis, oxidative stress, and neuroprotection (by increased ATP production and regulates NADP+/NADPH ratio, glutathione activity, mitochondrial permeability, glycolytic flux, apoptosis, and neuroinflammation, triggering normal synaptic neurotransmission to mitigate oxidative stress, attenuate apoptosis by supporting healthier mitochondrial activity and inhibiting apoptotic proteins, neuroinflammation, and intensify neurotrophins generation, including brain-derived neurotrophic factor (BDNF), neurotrophin-3 as well as glial cell line-derived neurotrophic factor, which associates with altered neuronal functions, especially in the senescent brain; may prevent the toxicity of neurotoxins of AD and PD). Besides, it serves as an epigenetic regulator in terms of histone methylation, acetylation, β-hydroxybutrylation to delay various age-related diseases. Endoplasmic reticulum (ER) stress is disturbed homeostasis leading to an impaired protein synthesis process such as the accumulation of unfolded and misfolded proteins, which is especially associated with the onset of metabolic disorders such as diabetes mellitus and disorderly hepaticlipid metabolism. Nonalcoholic fatty liver disease has been regarded as a novel Component of metabolic syndrome, with attenuated hepatic ketogenesis, insulin sensitivity, and abnormal fat accumulation Nutritional ketosis (NK) has been found to improve metabolic and inflammatory markers, as well as lower insulin levels and promote BHB production particularly during KD. In addition, studies support endogenous BHB administration or exogenous supplementation as effective strategies to induce a metabolic state of nutritional ketosis. It is effective for the obvious hemodynamic effects in atrophic cardiomyocytes, as well as inhibiting the loss of muscle weights, myofiber sizes and myofiber diameters in hindlimb unloaded mouse model, exerting a beneficial metabolic reprogramming effect in healthy muscle, and potentially slow muscle loss with myopathies by maintaining mitochondrial respiration and morphology within muscle tissue (with enhanced accumulation of glutamate and decreased uric acid in wasting muscles, revealing effectiveness for treating muscle atrophy). BHB reduces NLRP3 inflammasome in neutrophils and macrophages, reduced IL-1 and IL-18 in human monocytes, to inhibit inflammatory responses, and increasing BHB levels prior to ischemic/reperfusion injury results in a reduced infarct size in rodents, likely due to the signaling function of EB in addition to its role in providing energy. Sodium-glucose co-transporter-2 (SGT2) inhibitors have been shown to exert strong beneficial effects on the cardiovascular system (protective signaling is the core response, including mammalian target of rapamycin (mTOR), AMP-activated protein kinase (AMPK), sirloins, and endothelial nitric oxide synthase signaling pathway). However, it can induce cattle hepatocyte inflammatory injury and oxidative stress through the NF-κB signaling pathway, by increasing the expression of TNF-α, IL-6 and IL-1β, which can be mitigated by N-acetylcysteine (NAC, a potent antioxidant) and a 8-month ketogenic diet has been shown to have detrimental effects on skeletal muscle physiology. The best foods for boosting BHB include dairy milk (contains levels ranging from 10 to 631 μM BHB), MCT oil, coconut oil, butter, ghee and other healthy fats to increase KB, and BHB has better bioavailability if taken on an empty stomach. Butyrate in the form of sodium, calcium, or potassium butyrate (or its esters) will prompt the body to induce endogenous ketone synthesis, and serve as a ligand to stimulate receptors that the KB will also act on to contribute to the improvement of insulin and general metabolic health, anti-inflammatory and general immune system health, support neurological and cognitive health, support GI health and integrity; and serve as an energy substrate for ATP generation in parallel with the benefits that concurrent supplementation of the sister ketone body (BHB) serves as a significant synergistic farce for ketosis induction. Increased serum ketones can inhibit lipolysis, with appetite suppression and improved body mass. NK is shown in research to reduce lactate production and improve performance (time-trials) potential in some forms of activity (cycling), prevent muscle wasting (catabolism) and protect the brain and other tissues from oxidative activity. While BHB serves as an efficient agonist for the hydroxy-carboxylic acid (HCA)-2 receptor (targets for endogenous ketone and KB ligands), for example, it is not able to serve as an agonist for other HCA receptors (HCA1 is activated by lactate, and HCA3 by hydroxylated β-oxidation intermediates, especially β-hydroxy-octanoic acid). Both BA and BHB are signaling ligands for various receptors involved in neuroinflammatory control, including the HCA2 receptor. HCA receptors can be found in various tissue and cell types including adipose and macrophages, and are intimately associated with NF-κB modulation. Free fatty acid (FFAR, regulating cholecystokinin, peptide YY, and leptin, factors intimately involved in the regulation of feeding behavior and nutrient balance) and HCA receptors could very well be central targets for prevention and treatment of type 2 diabetes; obesity, and inflammation. Nrf2 is the primary transcription factor that is responsible for initiating responses to oxidative stress, which is induced by KD through mild oxidative and electrophilic stress that translocates to the nucleus and binds the antioxidant response element (ARE) to transcribe cytoprotective genes, including endogenous antioxidant peptides hemeoxygenase-1, catalase (CAT), SOD, and glutathione peroxidase (GSH/GPx) to convey neuroprotection during toxin-induced mitochondrial stress. Nrf2 induction protects cells from LPS-induced inflammatory activity and mortality and is a critical defense against elevated serum-glucose induced oxidative injury to cardiac muscle cells. The diabetic condition is associated with downregulation of Nrf2 activity via ERK, a factor speculated to be a contributor to stress-induced insulin resistance in cardiac cells, and can improve metabolic disorder and relieve renal damage. Hemoxygenase-1 expression, regulated and transcribed by Nrf2, plays an important role in the antioxidant defense mechanism alongside other common endogenous antioxidants to support recovery from injury, toxicity, and hypoxia (ischemia), particularly in neurons (stroke) and heart tissue. Overexpression of glutathione peroxidase is also associated with resistance to myocardial ischemic reperfusion injury. BHB-butyrate coupling appears to facilitate subcellular activity that counters Nrf2 downregulation associated with diabetic or other metabolic conditions that may adversely influence Nrf2 status, and may be applied to neurological disease, brain trauma, ADD, GI health and support of recovery from exercise or intensive performance training. Exogenous BHB-butyrate supplementation could be a functional strategy that induces β-oxidation and helps raise serum ketone levels indicative of ketosis (>0.2 mmol) with or without having to engage in stringent deprivation of macronutrients and consequential micronutrient limitations. Coadministration of BHB with its related butyrate molecule could appear to be an efficient way to accomplish this goal using extremely low and safe oral doses (both are designated as GRAS). As such, a carbohydrate or calorie-restrictive diet may not be necessary in order for one to benefit from therapeutic value associated with the signaling activity by this exogenous BHB-butyrate supplementation. However, a recent study found that following a ketogenic diet increases abundance of inflammatory gut species, such as E. coli. with upregulation of inflammatory cytokines such as IL-6, IFN-γ and others, and kills off butyrate producing species in the gut, such as Roseburia and Faecalibacterium. Bacillus spore-based probiotics are also shown to protect the gut from foods that are high in saturated fat, resulting in 24% reduction in serum triglycerides, and significantly lower levels of the inflammatory IL-1β. and reduced biomarkers of leaky gut. B. subtilis produces a large number of antimicrobial molecules that can lower abundance of proinflammatory species, such as E. coli and Salmonella; and B. coagulans producer of lactic acid promoting the growth of butyrate-producing species, such as Faecalibacterium prausnitizi, which can be used to off-set the gut-alkalizing effects of KD or high-fat diets. Adding serum bovine immunoglobulins (SBI) to the mix reduced levels of TNFα by 29%,-6 by 16%, and improved biomarkers of leaky gut and can provide additional benefit because it can bind to toxins in the gut and promote healing of the gut mucosal layer. BHB dramatically increased lysine β-hydroxybutyrylation (Kbhb) of histone tails, an epigenetic mark associated with fasting responses and muscle catabolic state, but does not interfere with rodent myotube formation. Myokines/hepatokines, peptides produced and released by muscle/liver; are known to mediate communication between muscle and other organs, and recent studies have also shown that these molecules may play a role in streptozotocin-induced neuronal damage. Aberrant muscle degradation in sarcopenia can be related to upregulation of muscle-specific E3 ubiquitin ligases Atrogin-1 and Murf-1 is pivotal to enhanced proteasome-dependent proteolysis with atrophy and autophagy of muscle cells. This can result from specific disease-triggered genetic programs, governed by multiple metabolic (Insulin/IGF, mTOR/Akt) and pro-/anti-inflammatory (TNF alpha/Il-6, Myostatin) factors and signaling pathways, eventually converging on the transcriptional regulators Fox0s and NF-κB. Similar to BHB, b-Hydroxy-b-methylbutyrate (HMB, a natural catabolite of leucine, a branched chain amino acid, B (AA; Exercise greatly increases energy expenditure and promotes the oxidation of BCAA by two to three-fold, aerobic=strength training>anaerobic), stimulation of muscle protein synthesis through the mTOR/S6 kinase and mTOR/4EBP1 anabolic cascades, through the growth hormone/IGF-1 axis, inhibition of proteasome-dependent proteolysis (ubiquitin proteasome and the autophagy-lysosome systems), normalization of autophagy (up-regulation of caspases, and reduce the apoptosis of myonuclei) and sarcolemmal stabilization via the HMG-CoA reductase-dependent neo-synthesis of cholesterol (decreases total cholesterol and LDL cholesterol particularly in those with hypercholesterolemia, but there may be a transient increase in cholesterol for the first 24 hr in rats, and a decrease in systolic blood pressure; it is also possible that impaired HMB synthesis and its deficiency may be expected to develop in liver disease, with cirrhosis often associated with cachexia and sarcopenia). HMB may compete with BHB of the level of the substrate, by targeting the same lysine residues with a different acyl group (hydroxymethylglutarylation) oi inhibit β0H butyryl-CoA transferase to reduce Kbhb accumulation elicited by BHB. HMB is widely used as a safe body-building ketone body (KB) supplement (also, used for dry eye, migraine, Alzheimer disease, Parkinson disease) among muscle builders (more effective than leucine supplementation) and combat sports athletes (<3 grams per day as GRAS), and considered in subjects who exercise, cachectic, prolonged corticosteroid exposure, deconditioned (may have higher density of muscle membrane cortisol receptors giving a catabolic effect) or elderly. HMB led to an increase in global histone acetylation, while preventing myotube differentiation (expression of MyoD and differentiation-specific markers MEF2 and stimulation of myogenic cell proliferation via the MAPK/ERK and PI3K/Akt pathways, unlike BHB). HMB attenuates exercise-induced muscle damage and enhances muscle hypertrophy and strength, aerobic performance, resistance to fatigue, regenerative capacity, increases mitochondrial biogenesis and fat oxidation (greater for anaerobic type II fast twitch muscles, which are more sensitive to sarcopenia due to catabolism or sepsis). In mice, it enhances calcium release from the sarcoplasmic reticulum during repetitive bouts of activity, which suggests that HMB improves excitation-construction coupling in muscle cells. It increases serum leucine levels, which is associated with decreased concentrations of alanine, glutamine, and glutamate, indicating that the conversion of leucine to alpha-ketoisocaproic acid (KIC) by BCAA aminotransferase in the muscles decreased. HMB is particularly effective in untrained individuals who are exposed to strenuous exercise and in trained individuals who are exposed to periods of high physical stress. The low effectiveness of HMB in strength-attained athletes could be due to the suppression of the proteolysis that is induced by the adaptation to training, which may blunt the effects of MB. In a study of non-exercising women who received supplements of 3 g of HMB/day for 4 weeks that there were no changes in the body composition whereas in a similar study in which the women were subjected to resistance exercise, an increase in lean tissue and a decrease in fat mass were observed. HMB may decrease cardiovascular risk factors including lowering blood pressure, binds to PPARα (almost comparable to that the prototype activator, gemfibrozil, a nuclear hormone receptor, found abundantly in the liver, helps in the reduction of triglycerides and free fatty acids via stimulation of peroxisomal β-oxidation of very-long-chain fatty acids, via interaction with the Y314 residue), increased pyramidal neurons dendritic tree in the medial prefrontal cortex in rats and improved the working and cognitive flexibility in old-age rats, increases mouse hippocampal plasticity, which is involved in spatial learning and memory via activation of cAMP response element binding protein (CREB). Moreover, oral HMB protects cognitive functions and reduces plaques in a mouse model of Alzheimer's disease (AD) via PPARα (as a non-amyloidogenic pathway via transcriptional upregulation of ADAM10, which inhibits the formation of amyloid plaques in neurons, stimulation of the TFEB-driven lysosome-autophagy pathway increases the degradation of amyloid plaques, and also increases lysosomal biogenesis and autophagy via transcriptional stimulation of TFEB), suggesting that HMB supplements may be beneficial for AD as well as other cognitive disorders. Oral administration of low-dose HMB increased the AMPA- and NMDA-mediated calcium current in hippocampal slices and enhanced memory and learning in 5× familial AD (5XFAD) mice but not XFAD mice lacking PPARα (5XFADDPPARα). HMB in a free-acid gel form resulted in more rapid and higher plasma concentrations and improved clearance of the HMB when compared with the calcium salt form; however the bioavilability favored the calcium salt in a oral formulation. A decreased glulamine level has been reported after HMB treatment, which may already be low in many patients with critical illness. HMB with black ginger (BG), but not if taken individually) combined with habitual exercise in mice improved muscle protein metabolism and energy production, with similar and possibly synergistic effects, insulting in increased mitochondrial function- and biogenesis-related genes, and reduced autophagy-related protein (Atg3, 7, 161L and Bedin1).

Exercise has been shown in both animal model and human studies to increase the relative abundance of butyrate-producing microbes and thereby increase the production of butyrate, a short chain fatty acid with systemic anti-inflammatory benefits and specific gut microbial strains modulate the expression of CBR and p opioid receptors (motility) in intestinal cells.

Exercise can bidirectionally influence the health of the microbiome, such that your microbiome can influence your exercise. Mice, specially bred for wheel-running behaviors three times as much as other mice (also had higher food consumption for their body size, higher body temperature when active, higher activity levels when housed without wheels, and altered hormone levels), developed different gut microbiomes, which was substantially reduced when given antibiotics asymptomatically (with body mass and water intake increased in both groups, but feedings were diminished in the runners), without other changes in their behaviors, or in other studies with fecal transplants from sedentary mice. Additional analysis revealed that antibiotics taken in both groups reduced motivation for physical activity in general. The researchers found release of SCFAs, especially butyrate or acetate in the gut during exercise, from the fermentation of carbohydrates in the GI tract, increases epithelial cell wall integrity, which travels through circulation and affect skeletal muscle performance, enhancing glucose uptake in skeletal muscle, as well as facilitating large neutral amino acid transporters which import essential amino acids across the BBB to where they become precursors to serotonin and catecholamines, including dopamine, which stimulates specialized autonomic nerves connecting the gut and midbrain, prompting the release in the brain of extra dopamine in the brain's reward center, creating a “runner's high.” Wheel-running behavior of these very active mice did not recover to pre-antibiotic levels even 12 days later, suggesting a sustained effect of antibiotics on altering the gut microbiome. Even at 20 days after the cessation of antibiotic treatment, 31 genera primarily belonging to the Firmicutes phylum had gone extinct, although gut microbiome diversity and community structure had recovered.

Spermidine: Spermidine (also found in hemp) is a polyamine, produced by microbiota anti-inflammatory and anti-antioxidant properties, and can also enhance respiration and metabolic function which are critical for mitigating oxidative stress, and induction of autophagy to stimulate cellular longevity. Gut microbial metabolites include SCFA, polyphenols, vitamins, tryptophan catabolites, and polyamines (including agmatine, putrescine and spermidine). In animal models, supplementation with polyamines especially spermidine, could have a beneficial effect on several age-related disorders including memory decline, neuroinflammation, and cardiovascular disease through activation of autophagy and by reduced DNA methylation levels by inducing the accumulation of dcAdoMet. Many food products included in the Mediterranean diet, which is known to be associated with increased longevity, are rich in polyamines. Increased levels of polyamines in white adipose tissue, liver or skeletal muscle could stimulates energy expenditure and confer resistance to diet-induced obesity and non-alcoholic fatty liver disease. Furthermore, the administration of either spermidine or spermine has been shown to be effective for improving glucose homeostasis and insulin sensitivity and reducing adiposity and hepatic fat accumulation in diet-induced obesity mouse models. In pancreatic islets, polyamines are mainly located in the secretory granules of the $ cells, where they have been implicated in proinsulin biosynthesis and insulin secretion, which diminishes with age. Serum putrescine levels were significantly elevated in diabetic subjects and that correlated with glycolized hemoglobin, and that serum spermine was closely associated with insulin levels. Polyamines especially spermidine can prolong life and slow down the aging process with anti-inflammatory effects on the colon, especially relevant for IBD), memory T cells and dendritic cells to sites of inflammation caused by tissue injury or infection)) in stimulated hPDLF. Spermidine, an autophagic inducer, is effective in restoring mitochondrial function, improving the elasticity of cardiomyocytes and suppressing the inflammatory response, lowers blood pressure, prevents cardiac hypertrophy and delays heart failure, as well as enhance mitophagy to prevent oxidative stress, slow aging, and extend life span. It has been suggested that dietary polyamines (particularly spermidine) might decrease the risk of CRC in postmenopausal women. In plants, spermidine can enhance hemp's polyamine content, improve resilience to dehydration with increased oxidative and photosynthetic stress, and can facilitate germination of seeds. Increased dietary spermidine intake is thought to reduce the risk of diseases like cancer (induces autophagy), metabolic disease, heart disease and neurodegeneration. Spermidine, which degrades to 4-aminobutyraldehyde and in turn is oxidized to GABA by NAD+-dependent 4-aminobutyraldehyde dehydrogenase.

Formulations: Often the carriers of other delivery systems are considered “inactive” products, but may enhance delivery, comfort and be in and of itself, efficacious.

Nasal spray formulations may be aqueous, hydroalcoholic, or nonaqueous-based solution, suspension, or emulsion systems (usually of a smaller particle size that topical sprays). Depending on the type of system, the formulation will include a range of functional excipients, including solvents and cosolvents; mucoadhesive agents; pH buffers; antioxidants; preservatives; osmolality and tonicity agents; penetration enhancers; suspending agents; and surfactants. The choice of formulation type and the excipients selected will be driven by the solubility and stability of the active drug, as well as the concentration needed to deliver an efficacious dose in a typical 100 μl spray. The nasal spray formulation may be used flexibly or alternatively as an mist/inhalant nebulizer with similar formulation properties as described above Since they are not ingested products, FDA regulation is limited for botanical products.

Oral solutions include syrups (sweetened, but do not contain suspending agents), elixirs, spirits, and tinctures, and are based on the parameters: Organoleptic (appearance, taste, flavor, smell and sweetness, in which sugar alcohols such as sorbitol can cause osmotic diarrhea, as can sugars.) Osmolality (avoid>400 Osm//kg in those with GI problems or elderly), pH (based on the product, usually will be acidic for cannabinoids, using buffering agents to help minimize common degradation), Presence of preservatives ((methyl) parabens have low aqueous solubility, with pH 4-8, and often solubilized with the aid of small amounts of a co-solvent such as alcohol or propylene glycol; sodium benzoate, best for pH<5; and potassium sorbate., best for pH<6, without requiring a co-solvent, safest in pediatrics), Dyes (optional, but often Red #3 and FD&C #40, which may also assist with preservation), Viscosity, and Suspending agent used. Common suspending agents (with variable sedimentation properties)/viscosity enhancers include sodium carboxymethylcellulose (avoid with quaternary nitrogen-containing compounds, iron salts, and metals, and may require more agitation for mixing), xanthan gum (avoid with large cationic drugs, surfactants, polymers, and preservatives), and carrageenan (avoid with pH<9 or may interact with cationic active pharmaceutical ingredients), each with unique incompatibility issues.

Topical application (usually over intact skin) consisting of any combination of a formulated spray (mist), with a carrier that facilitates even distribution as a film forming propellant, carrier, penetration enhancer, volatile solvent, water-soluble solubilizer/additive and polyoxyethylene copolymer plasticizer, to enhance skin adherence such as gellan-based fluid polymer gel, acrylate and methacrylic ester, polyhydric alcohol, Vitamin E, labrasol, and surfactants (humectants)(Pawar, Neelam, and Pawan Jalwal. “Non-Pressurized Topical Spray Pharmaceutical-Methodology of Formulation Development and Quality Control Management.” International Journal of Pharmaceutical Investigation 11, no. 3 (2021); Ter Horst, B., R. J. A. Moakes, G. Chouhan, R. L Williams, N. S. Moiemen, and L M. Grover. “A gellan-based fluid gel carrier to enhance topical spray delivery.” Ada Biomaterialia 89 (2019):166-179), salves/balms (may be almost solid, with soy, paraffin or beeswax as a carrier); paste (for oral dental use); ointment (thickest, opaque or translucent, with 80% oil as carrier); cream (thicker, opaque and viscous, more moisturizing, useful for dry skin, with 50% oil as carrier); lotion (less thick, viscous without leaving residue, useful with oily skin, with 30% or less, oil as carrier); gel (very thin, forming colloidal semisolids on standing and becoming liquid on easy agitation, for moist skin and for topical cavities such as nasal mucosa, with lipophilic cellulose, hydrophilic water or alcohol as carriers; single phase from synthetic macromolecules such as Carbomer or mucilages from natural gums such as Tragacanth; with increasing temperature they become more viscous, but they can swell with solvents, and contract with solidification related to an elastic phase) or solutions (aqueous, high viscosity, may have emulsified contents for oral rinses, external otic, which may include dilute acetic acid, found in vinegar); or infused oils (may use thin vegetable such as olive or almond as a carrier) can be sprayed on (usually as a lotion, gel or infused oil), rubbed in, or applied as patches (made from hydrocolloid or hydrogel material, such as gelatin or pectin, which are moisture absorbing, may be useful in wound care) onto the skin to act directly over the region applied, or transdermal (for systemic use, to travel through the epidermis into the bloodstream, with duration of application from several minutes to 1 day) and its effect is correlated with patch size.

Emulsions usually contain an oil phase (petrolatum or liquid petrolatum with one or more high molecular weight alcohols such as cetyl or stearyl alcohol), water phase (also contains preservatives, emulsifiers or parts of emulsifiers and humectants, as well as stabilizers, antioxidants, and pH buffers) and emulsifiers (stable, inert, free of toxic and irritant ingredients, odorless, tasteless and colorless, i.e., anionic such as sodium lauryl sulfate, triethanolamine (TEA) stearic; or cationic such as quaternary ammonium salt; or nonionic emulsifiers such as e.g., Tween, Span). Emollients/moisturizers work by forming an oily layer on the top of the skin that traps water in the skin. Lipophilic creams contain water-in-oil emulsifying agents such as lanolin, sorbitan esters, lanolin, mineral oil, petrolatum, dimethicone and monoglycerides, whereas hydrophilic creams contain oil-in-water emulsifying agents such as sodium or trolamine soaps, sulfated fatty alcohols, polysorbates, shea butter, cocoa butter and polyoxyl fatty acid and fatty alcohol esters, that may be combined together for a full effect; they may contain plant or animal derived products. Some creams consist of oil-in-water emulsions or aqueous microcrystalline dispersions of long-chain fatty acids or alcohols that are water washable and more cosmetically and aesthetically acceptable. Many products also have ingredients that soften the horny substance (keratin) that holds the top layer of skin cells together (including urea, alpha hydroxy acids such as lactic/citric/glycolic acid, and allantoin). During application, the continuous aqueous phase will evaporate, increasing the concentration of water-soluble substances in the attached layer. To prevent the deposition of drugs, and to increase absorption through the skin, added substances that are mixed with water but do not evaporate, humectants (e.g., glycerin, lecithin, and propylene glycol) are used draw water into the outer layer of skin. A better formulation is a topical that can deposit fat and other moisturizing compounds to help hydrate the skin. Formulations often combine ingredients in specific amounts according to processing instructions. To develop a stable topical formulation (emulsion), its hydrophilic-lipophilic balance (HLB) value, characteristic of the surfactant group emulsifiers, as well as pH is important. The combination of a hydrophilic and lipophilic emulsifier such as glyceryl stearate (HLB value 3.8) and PEG-100 stearate (HLB value 18.8) were found to be effective to emulsify the chosen oil phase system at a specific concentration to achieve the required HLB for the development of the stable emulsion-based system.

Lipoderm hydrogel (Hydrogenated Polyisobutene, Caprylic/Capric Triglyceride, Isodecyl Laurate, Glyceryl Linoleate, Cocos Nucifera (Coconut) Oil Ovum, Pollen Extract, Olea Europaea fruit oil, Glycine Soja oil, Persea Gratissima oil and Triticum vulgare germ oil) is a proprietary permeation-enhancing moisturizing and nourishing vehicle available in 4 options: for general permeation-enhancing topical formulations, active ingredients in salt form at high concentrations, with high molecular weight, or unstable in water (lipophilic, most useful for cannabinoids). The shelf life depends on the formulation and preservatives, but can range from 14 days if aqueous without preservatives and refrigerated to 180 days if non-aqueous, anhydrous and with preservatives.

### SUMMARY OF THE INVENTION

The present invention provides a cannabinoid composition, comprising at least 5% cannabigerol (CBG) and/or cannabigerol acid (CBGA), in a pharmaceutically effective dosage form effective for oral, transmucosal, inhalational or transdermal administration. The formulation preferably includes an absorption enhancer that increases local blood flow at the absorption interface, such as piperine, eugenol, or curcumin. Similarly, the formulation may include oleophilic diluents, surfactants, e.g., Tween-80 (polysorbate 80), etc.

In some cases, the formulation comprises a full spectrum or modified full spectrum formulation, with various terpenes, and/or bioflavonoids, etc.

Further, the formulation preferably is substantially without cannabidiol and tetrahydrocannabinol. A preferred extraction technique is solventless, so that organic solvent residue is not present.

The CBG and CBGA are preferably from Panakeia, which lacks CBGA synthase and THCA synthase activity, resulting in accumulation of high levels of CBGA. The extraction process is preferably at a low peak temperature, and otherwise selectively preserves the CBGA form, without decomposition into CBG.

The parameters of extraction through the preferred Herbolea, Essentia Scientific process, or equivalent, are specified to account for the lower boiling point of CBG (compared to CBD) as with typical CBD/CBDA extraction from Cannabis.

The full spectrum extract comprises naturally occurring cannabinoids and other non-cannabinoid components that are co-extracted with the at least one CBGA (and to a lesser extent, CBGVA) or decarboxylated derivative-type compound (CBG, and to a lesser extent CBGV). The Panakeia “full spectrum” CBG extract may be concentrated, with other additives, substitutions or deletions, or combined with other natural ingredients to enhance effect, absorption and flavor, to form a food product, nutritional supplement, or medicine. In one embodiment. The PECSA™ extract contains primarily CBGA. Some proportion may be converted to CBG. The formulation may be enriched with various components, such as other extracts from Panakeia or other botanical compounds.

An extract may contain a greater proportion of the total cannabinoid content of CBG as compared to the cannabinoid composition from which the extract was prepared primarily by purifying the plant extract further after extraction to select specifically for CBG. The therapeutic effects of PECSA™ are due to the combined ingredients as a synergistic or entourage effect, with CBGA/CBG as the primary ingredients. Although they are similar compounds, CBGA (an acid) and CBG, may have different therapeutic applications and benefits than if they were administered as an individual component.

The full spectrum oil from the Panakeia extract may be enriched by distillation. The product may be standardized to a predetermined profile, e.g., 15% CBGA, and other component standardization, by blending, and/or addition of key components.

PECSA™ (Phyto-EndoCannabinoid System Activator™), is a trademarked family of dietary supplements, which is primarily derived from Panakeia whole plant biomass, which can be extracted into an oil, powder and/or paste. It can be consumed by several routes of administration, including ingestion orally or via stomach, transmucosal (may include oropharyngeal, nasal, rectal, and possibly transurethral, vaginal or conjunctival), inhalation, transdermal, topical applications and possibly parenteral. These hemp extracts may have variable concentrations of consisting of at least 18 lasses of chemicals, at least 554 known compounds (of which, at least 80 are biologically active) that can vary, based on cultivation, harvest, extraction and storage parameters. Panakeia is a C. sativa L plant cultivar, preferably cultivated in US, and often is organically grown (if accompanied by a US Department of Agriculture-DA Organic Certification). This unique hemp plant is genetically altered (but is not considered a GMO), innovated with two specific synthase enzyme deficiencies that naturally prevents the plant metabolism of CBGA to form either CBDA (acidic form, which can be detected, along with CBD, in legal hemp flowers, up to 20%) or THCA (acidic form, which can be detected in cultivated marijuana, with THCA/THC content ranges of 25-60% in the flower, or <0.3% found in other legal hemp plants).

PECSA™ is primarily based on CBGA (primarily with CB2R binding) contained in a whole spectrum extract of a patented hemp plant, Pan2020™ (without THC or CBD), devised to incorporate a balanced CBR binding effect through exogenously taken endocannabinoids (AEA, PEA, OEA, SEA, LEA, which possess CB1R binding modulation, without known psychoactivity), along with other botanical ingredients or enhancers for improved bioavailability and synergy.

The primary “Active Ingredient” of the formulation includes cannabinoids, primarily CBGA, and its decarboxylation product, CBG, with other proprietary ingredients which can either additively and/or synergistically contribute to desired effects that may be different for each designated product. In order to “standardize” the extract (in which cannabinoid content may vary from different batches and cultivation milieu) used in formulation, the percent of total CBG/CBGA (and possibly terpene content) may be normalized from more concentrated extracts (may be derived from different batches), or diluted from the original extract oil, or with excipients.

The products may be consumed by themselves, or combined of other cannabinoids, other known nutritional supplements or foods. The products may be formulated as unit dose gel capsules, tablets (may be enteric coated), gummies, powders, tinctures, oils, emulsification, submucosal film, effervescent or suppositories, aerosolized sprays, nanoparticles, alterative inhalation products (flower, resin or vaporization cartridges), patches, transdermal creams, ointments, lotion or salves, and possibly parenteral formulations. Multidose formulations may also be provided.

A pure, or almost pure CBGA/CBG formulation may also be provided. The formulation may employ purified CBG and/or CBGA as the sole or mail cannabinoid, with additional terpenes, which can be used for vaporization/inhalation, this may be mixed with vegetable glycerin (VG), propylene glycol (PG), or medium chain triglyceride (MCT cocoanut) oil.

Other products include a combination of a CBG/CBGA-rich Cannabis extract combined with one or more additional active ingredients.

Oral absorption of CBG/CBGA and CBD/CBDA may be enhanced further with pepper (piperine, may be deodorized); CBG/CBGA and CBD/CBDA bioavailability may be further enhanced with water soluble compounds (with more rapid absorption, compared to oral tablets) using emulsification methods for fluids. This may be further improved with micro- or nanoparticle technology by approximately 30%.

Panakeia is a C. sativa L plant cultivar, originally developed in Spain in 2013, patented in the US (USPP32725) in 2020, is chemotype IV. Panakeia contains predominantly CBGA>CBG, without THC or CBD content. It has been genetically modified at chromosome 6, to have structural mutations of tetrahydrocannabinolic synthase or cannabidiolic acid synthase that effectively prevent the decarboxylation of CBGA from forming either THCA and CBDA, respectively, but still has intact cannabigerolic acid synthase to form CBG. Therefore, in the Panakeia™ strain, CBGA (or CBVGA) can ONLY convert into CBG (or CBGV, respectively), and uniquely contains CBGA (5-19%), 0.00% THC (includes no A-8, A-9, A-10, or A-0 isomers), and nearly 0.0% CBD (possible trace). GMO has become the common term consumers and popular media and is used to describe foods that have been created through genetic engineering (GE) and has been replaced by the term, “bioengineered” with FDA labeling requirements since Jan. 1, 2022. It refers to foreign DNA being introduced to a cell from an unrelated species while, in genetic modification, the plant's own genetic material is modified to produce a new characteristic that isn't possible through natural growth. Panakeia has genetic modification for its characteristics, but is not GMO, and PECSA™ products are exempt from the FDA labeling requirement “derived from bioengineering.”

With marijuana or most hemp plants, the in vivo decarboxylation of CBDA or THCA by plant enzymes results in the preferential conversion to CBD or Δ9-THC, respectively (the latter, is considered a banned, federally illegal substance by the DEA, Sch I, unless it is detected <0.3% content in legal hemp; THCA is not accounted for in the legal definition of hemp). Similarly, CBGA can also be decarboxylated irreversibly to CBG, but at much lower rates of conversion for marijuana and hemp plants. The average CBGA concentration (usually <1% overall) in CBD-dominant cultivars is 0.24%, while it is 0.61% in THC dominant cultivars, although certain legal hemp cultivars have a reported yield total CBG (mainly CBGA) of up to 20% (but with detectable CBDA, CBD, CBC, THCA and THC). However, Panakeia's unique genetics, with yields of 8-20% cannabinoids detected in the dry whole plant biomass, greatest in the flower, the major cannabinoid detected is CBGA, along with CBG, 4-20:1 (higher proportion of CBGA if the fresh dried flower is consumed without processing, or less, based storage and on the extraction method, see below), and other minor cannabinoids (2-5%). Panakeia extracts will be distinguished from other legal hemp containing CBG, by the fact that most of its “total CBG” content will be maintained in the acidic form, CBGA (which can easily be converted to CBG, if desired), which makes it a more valued product, and this distribution ratio is usually less for other hemp cultivars. The non-intoxicating CBGA found in Panakeia cannot be converted into psychoactive THCA, or even CBDA; and it is infeasible/uneconomical to accomplish this in a lab. For any hemp plant, including Panakeia, in vitro, one may convert an extraction of CBGA into CBG (which in turn, cannot be adulterated into other cannabinoids, except for its known non-psychoactive degradation/metabolic products), by heating it above 110 degrees C., with exposure to ultraviolet light or ageing. Unlike extractions from Panakeia, but can occur with other legal hemp products, THCA and CBDA and can be converted to THC (as long as Δ9-THC remains <0.3% dry weight in any of the hemp extracts, and may be converted to federally unregulated isomers or derivatives, and can also be degraded into CBN, all psychoactive) and CBD (which can, in turn, can then be converted to psychoactive federally unregulated THC derivatives, in which some of these products are sold without proper monitoring or labeling, outside of a state approved Cannabis dispensary), respectively. Proper storage (room or refrigerated temperature, low humidity and tinted jars) are required to prevent degradation of any and all of these components. Therefore, CBGA and CBG derived from Panakeia cannot be feasibly adulterated, and any of its subsequent extracts will always contain 0.00% THC and 0.0% CBD.

Cannabinoid Extraction methods: For Panakeia the “whole plant biomass” or “full spectrum” hemp constituents mainly includes minor cannabinoids from CBG(V)/CBG(V)A derivatives: CBGM, CBGAM, and probably from CBC(V)/CBC(V)A derivatives: CBL, CBTC. Panakeia or other “broad spectrum” hemp extracts are devoid of derivatives of THC(V)/THC(V)A: CBN/CBN(V)A, CBT, CBND and CBDL. Panakeia is devoid of derivatives from CBD(V)/CBD(V)A: presumably CBE, CBEA, CBM, CBF and CBO, which are usually contained other hemp products. These minor cannabinoids (associated with CBG, CBC, THC, and CBD; A and V) may be present with varying proportions in other hemp cultivars as “full spectrum” extracts. Resin is the compressed solid derived from the resinous trichomes of the plant. These compounds along with cannabinoids and monitoring of contaminants, are often identified by gas or liquid chromatography (LC/GC) with mass spectroscopy (MS) gradient times in current methodologies. Other compounds found in various parts of Cannabis plants, including Panakeia. The phyto-cannabinoids represent about 24% of the dry mass. A full spectrum extract has a ratio of extracted components approximating the whole plant. A broad spectrum extract may have selectively omitted components.

Live resin (honey in color, in which uses butane or propane solvent distillation in a lab), vs. rosin (amber colored, in which solventless heat and pressure is used to separate the resin from fresh bud, without chemicals, preserving a higher proportion of acidic precursors) are concentrates that have been extracted before the Cannabis plant has been dried or cured. Terpenes, the aromatic compounds that give Cannabis its flavor, are so volatile, they're known to dissipate even at room temperature. Working with a freshly harvested plant gives extractors the best chance of capturing robust terpenes and flavors. To preserve these fragile terpene profiles, extractors may freeze and store freshly cut Cannabis until it's ready to be extracted.

Harvested hemp is usually dried (a pre-requisite to solvent extraction) indoors commonly 15-21° C., and relative humidity (RH) is 55%-65% with low natural air flow, by using two methods: hang-drying (not uniform and may cause mold or mildew exposure in the center, takes 7-10 days with high labor costs) and tray drying (taking detached buds and leaves in thin layers, takes 3-5 days), which can preserve acidic cannabinoid and terpene content, especially at lower temperatures, unlike hot air flow. Freeze drying is more expensive, with minor effects on the volatile and bioactive compounds in plants. Other innovations include radiofrequency, ultrasonic and vacuum drying (or combinations thereof).

Ethanol, propane, butane, naphtha, and petroleum ether (temperature controlled, but combustible), or possibly water, are now less commonly used alternative solvents. All these products are often combined with olive oil (with higher lipid content) based on the fact that it extracted higher amounts of terpenes than the other solvents/methods, especially when using an extended heating time (and has smoke point temperature of about 200° C., with a boiling point of 700° C., without causing any safety issues), but the extract cannot be further concentrated thereafter by evaporation. If the ethanolic extract to remove chlorophyll, is combined with activated charcoal, this will result in a considerable reduction of cannabinoid content. Tinctures are generally made from hemp blended with carrier oil (often coconut). Extracts with vegetable oil content are not generally used for vaporization products. Naptha solvent can improve THC yield for marijuana extraction, but at the expense of reduced terpene content, and naptha (which may also potentially add impurities) and petroleum ether may be carcinogenic. For essential oils (rarely used for hemp) extraction, usually involves distillation with steam, which becomes “concrete”, which is then combined with alcohol, extracting the aromatic principles of the material, forming “absolute,” which can be useful for olfactory impact.

Most CBD oil or tinctures are extracted in US by carbon dioxide (CO₂, using supercritical-SC gas method at 31° C. and 74 isobar, sometimes with 5% ethanol added as co-solvent to SC—CO₂ to further enhance the extraction of CBD. but may not be able to remove toxins including pesticides), is preferable and considered as GRAS. Then, it is winterized (separated from unwanted plant material including chlorophyll, often encompasses the use of ethanol or butane at low temperatures) often using low temperature butane or ethanol, and decarboxylated (heated to remove the molecule's carbon tail). Molecular distillation, also known as short-path distillation, is usually the first purification step for crude oil as a condensation process followed by an evaporation of desired substances. There are three typical chromatographical technologies in the hemp industry: high-pressure liquid chromatography (HPLC, preferentially used), centrifugal partition chromatography (CPC), and flash chromatography. With HPLC, flows through a column containing packing materials are coated with specialized materials. Different eluents have different rates of adsorption and desorption on the coating materials, and thus move along the column at different rates, so different effluents can be collected separately. After purification, the oil turns into clear viscous liquid with golden color.

Each cannabinoid has its own boiling point, which allows the distiller to separate individual cannabinoids (if desired by chromatography) using vacuum pressure and heat to extract a distillate, which is the closest possible version of pure cannabinoid oil; otherwise, it may contain a full or broad (with THC virtually eliminated) spectrum of other components including trace cannabinoids and terpenes. Some lipid extractions require higher temperatures, for decarboxylation at 150 degrees C. for 10 minutes, which can also reduce some cannabinoid yields, especially the acidic forms.

Essentia Scientific's patented water-as-solvent extraction (initially the hemp biomass is dissolved in ethanol, whereas other processes dissolve it in oil (i.e., olive), then mix with alkaline pH (about 8.8, using NaOH) with agitation followed by filtration and then add acidulants to precipitate cannabinoid acids to water soluble salts to form a dry powder, without the use of heat, harsh volatile chemicals, or hydrocarbons, whereas organic solvents used for extraction, such as butane and ethanol often concentrates contaminants in the final extract). This process yields a more sustainable and safer output of 99% high purity cannabinoids with no remediation necessary, removing contaminants such as pesticides, heavy metals, microbials, and mycotoxins, which can be easily infused into any full- or broad-spectrum as a microcrystalline cannabinoid powder (including preserving acidic precursor forms of >97.5% CBGA, for a thermally stable water soluble cannabinoid inclusion complex as an isolate). CBDA is 19× more bioavailable than CBD, and may have been anticonvulsant effects. This aqueous process would tend to separate the lipophilic components of the biomass, including flavonoids and terpinoids. (essentiascientific.com) Cannabis wax is a soft, opaque concentrate that can vary in appearance, texture, and color, as determined by heat, moisture, chemical composition, and purging process. Many waxes are the result of agitating a raw extract from butane solvent into a whipped, aerated consistency. It has a high potency, fast onset, long duration of effect, with good flavor, and can be consumed in multiple formats. In the crystallization process, the purified oil is firstly dissolved in solvents (usually pentane) in a large vessel which is heated and mechanically stirred; then the temperature is gradually decreased and the agitation rate is slowed down to initiate the nucleation. The filtrated crystals are rinsed with pentane again to remove the remaining impurities, with commercial CBD isolates usually have at least 99.5% purity.

Processers can convert any hemp oil extract (including Panakeia) into water soluble droplets of fluid or powder of varying sizes, using surfactant which reduces the surface tension of a liquid in which it is dissolved (i.e., anionic lipoprotein, surfactin produced by Bacillus subtilis, and nonionic, polyethylene glycol sorbitan monooleate, also hydrophilic: sodium stearate, oleic acid/polyethoxylated sorbitan, castor oil/ethylene oxide, benzene-sulfonates and docusate), through micro-emulsions, liposomes and (micro) nanoemulsions, preferentially by using high-pressure homogenization or high-amplitude ultrasonic processing (sonication). This technique can be applied to beverage manufacturing, and pasteurization at high temperatures is avoided, so that the original cannabinoid and terpenes proportions can be maintained for the full spectrum entourage effect.

Encapsulation, emulsification and microfluidization may be used to incorporate various bioactive compounds into food and beverage systems in the industry and offer potential solutions to the development of non-psychoactive cannabinoid-containing foods. Micro-encapsulation is one possibility to improve the solubility and reduce the oxidative susceptibility of sensitive bioactive compounds, where the hydrophobic molecule is encapsulated and dispersed in aqueous phase. Sesame oil as a delivery system for CBD in medicines and revealed that adding of medium-chain triglyceride (MCi) improved the solubility of CBD and superior bioavailability. Micro-encapsulated CBD had higher CBD bioaccessibility compared to CBD-isolate without microencapsulation, and CBD bioaccessibility was improved with the incorporation into a food system made of olive oil and baby food, which might be attributed to the improved efficiency of micelle forming from hydrolyzed lipids. Nanotechnology has already been applied in drug delivery systems and can be another promising solution, in which cannabinoid-loaded lipid nanoparticles (NP) through solvent-emulsion evaporation and improved the stability of the products.

An anti-inflammatory diet or supplements, specifically omega-3 fatty acids (which are contained in hemp seed along with omega-9 fatty acids, and are found to be more stable (does not oxidize), efficiently converted, vegan, no after-taste, has less digestive difficulties, and more easily applied topically, compared to fish derived oil) can reduce menstrual cramps (dysmenorrhea), and is mediated through changes in PGE2 and PGE2a (also increased in pregnancy).

MCT oil is of low viscosity and devoid of any lipid oxidation products, and tends to be less susceptible to oxidative degradation resulting in rancidity, as well as decreased concentrations of cannabinoids and terpenes, than olive oil or hemp seed oil. However, it may be combined with small amounts of long chain fatty acids (i.e., plant derived: solid coconut oil, bees wax or caranuba) to increase its viscosity in elixirs, particularly in those with tremors using a teaspoon, during sublingual administration. Oral cannabinoids tend to naturally have a bitter, earthy taste (which may be a deterrent for children or pets), which can be improved or masked with terpenes or other combined ingredient, selectively. In most emulsified cannabinoid products, carrier oils (with a naturally nutty subtle flavor often MCT content may be further purified to 100%, used in vapes: derived from coconut-54%, or palm (kernel) oil-34%, most MCT has proportional 8-carbon chain, caprylic acid-60%, 6-carbon chain, capric acid-40%, with plant derived glycerol which is a base for all triglycerides; also without MCT: sesame, avocado, olive, grape seed oil, but can also use hemp seed oil), preservatives, and surfactants intensify the bitterness. In general, MCT have fewer calories, and are more easily absorbed, and may promote a ketogenic diet. However, diabetics and those with liver disorders should limit MCT intake due its adverse effects from hypertriglyceridemia. Another MCT, the 6-carbon chain caproic acid is seldom used due to its bitter flavor.

A strategy to improve extraction and/or bioavailability of cannabinoids and/or terpine/terpinoids (often contained together in broad spectrum extracts), as well as additive and/or synergistic nutritional or bioactive benefits is to use specific plant derived carrier oils, including unsaturated vegetable oils, MCT or glycols, the latter two are used for purer forms of cannabinoids including vapes (need to avoid larger sized oils for safe inhalation). The lipid may be one or more lipids independently selected from the group consisting of olive oil, sunflower, oil, coconut oil, sesame oil, vegetable oil, milk, butter, liposomes and hemp seed oil, black seed oil, and/or other green and/or food grade solvents such as glycerin, polyethylene glycol, ethyl acetate, d-limonene, butylene glycol, propylene glycol, ethylhexyl palmitate and/or with the addition of lecithin.

Hemp seed oil or whole seeds that are expressed or extracted from seeds contain 0% THC and trace levels of CBD, but 0% CBD if derived from Panakeia (may contain a small amount of CBGA or CBG), which can improve the lipid profile to reduce cardiovascular and cerebrovascular disease, and can help lower blood pressure reduce inflammation such as irritable bowel syndrome (IBS), rheumatoid arthritis (RA), and multiple sclerosis (MS). It may reduce symptoms of menstrual cramps and improve recovery of muscles after exercise. If taken while pregnant, it supports healthy fetal brain and eye development and may also help prevent maternal depression. If applied topically, it improves symptoms of atopic dermatitis, also known as eczema and may be useful for cradle cap, psoriasis, and acne. Panakeia (hemp) seed oil will contain 0% THC, a % CBD with trace CBGA. Hemp seed oil will enhance absorption of the Panakeia extract. It is denatured by heating above 150° C.

A summary of the potential effects of CBG/CBGA (including full spectrum extracts containing terpenes) for at least symptom control of the following conditions based on basic science (known mechanisms of action), animal, clinical studies, expert opinion and anecdotal experience (incomplete list) through diverse administration options: Neuroprotective properties are perhaps its most promising attribute: CNS disorders including neurodevelopmental disorders (often, genetic), cerebral palsy, head injury/concussion, cerebrovascular accidents, brain infections or neoplasms, multiple sclerosis, Huntington's disease and other neurodegenerative conditions, for cognitive enhancing effects and ameliorating movement disorders; Reduction of intraocular pressure (glaucoma); Antimicrobial and antibiotic: antibacterial effect (methicillin-resistant Staphylococcus aureus-MRSA), anti-viral (i.e., Covid or prion), anti-parasitic or anti-fungal; Inhibits growth of many cancer cells including colon and breast with apoptosis; Inflammations of the digestive system (Crohn's disease, ulcerative colitis, or other infectious/inflammatory bowel diseases), anti-spasmotic, motility disorders, functional bowel disorders (i.e., irritable bowel syndrome), dysbiosis, oral hygiene and gastro protectant effects; Anti-inflammatory for autoimmune arthritis or osteoarthritis (degenerative) arthrosis, or soft tissue inflammations; Analgesic (nociception-somatic and visceral, neuropathic-central and peripheral, and nocioplastic, particularly if chronic or mixed); Inhibits the uptake of GABA (anxiety, muscle tension), panic disorders, PTSD, mood, chronic fatigue, sleep disorders and spasticity; 5HT1A effects for psychosis/thought disorders and depression; Mitigates adverse effects of THC, including reduction of psychoactivity and substance use disorders (addiction) including tobacco (nicotine); Stimulates appetite (cachexia), especially in conjunction with chemotherapy and eating disorders; Stimulates bone formation and healing for fractures and metabolic bones diseases including osteopenia/osteoporosis; Anticonvulsant effects (i.e., Dravet syndrome, febrile seizures, epilepsy); Overactive bladder with urinary dysfunction; Reproductive medicine (libido enhancement for both sexes) and hormonal balance; Gynecologic disorders (endometriosis, menstrual dysfunction and vaginosis); Antioxidant for cardiovascular (including blood pressure and ischemia), respiratory, renal, hepatic, metabolic (sugar and fat metabolism) protection as well as energy or temperature regulation; Wound healing or skin disorders, skin rejuvenation, antipruritic, prevention of sun (UV) exposure; and Optimizing nutrition (in conjunction with other hemp-derived contents) and for detoxifying or cleansing regimens. There are fewer potential drug-drug interactions with a Panakeia extract to be concerned with for these considerations. In a survey of 127 patients using “CBG”-predominant (>50%) products, the most common conditions described were anxiety (51.2%), chronic pain (40.9%), depression (33.1%), and insomnia/disturbed sleep (30.7%). Mild side effects and possible withdrawal symptoms (listed below under “informed consent-warnings”) were also described for this population.

Cannabinoid products combined with other known supplements or drugs conveying allied benefits and/or improved bioavailability, that the effects would be at least additive (or more likely synergistic). If integration of several component products is considered, it is predicted that for optimal efficacy, each individual ingredient's dosing requirement would be minimized, such that there would be less anticipated adverse events. The combination of products devised can be customized based on the intended goals (also taking into account gender, age, socioeconomic factors, or medical conditions) for overall longevity and wellness, or for more specific symptom(s) control to a targeted consumer. CBGA/CBG can potentially mitigate the adverse effects of marijuana (THC), amongst other beneficial attributes, as can occur with CBD/CBDA.

The “portion size” designated for CBG or CBGA and/or CBG is 5 mg, similar to literature pertaining to THC content. However, subject to various risks, a higher dose may be provided. Further, the dosage may be repeated periodically, e.g., every 4 h, 6 h. 8 h, 12 h, 24 h, etc. A 10% concentration of liquid oil extract will contain 1000 mg of CBG/CBGA in 10 ml (200 portions) with <±20% variance, and a batch with lower content may be normalized or standardized with concentrated CBGA/CBG. A rapidly dissolving sublingual dosage form, similar to a breath strip or nitroglycerine tablet, may be employed, especially with an absorption enhancer, which can act by increasing local circulation and/or increasing permeability.

The formulation may be used in veterinary applications, generally modifying the human formulation according to best practices and known pharmacology. These products cannot be used in animal feed. Dogs metabolize cannabinoids differently than humans. CBD has the potential for liver toxicity in animals. Industrial hemp derived nutritionals (without cannabinoids) have also been used in livestock feeds. Dose adjustments need to be made based on the species and animal size. CBD may have a tolerance effect, and have a biphasic action: there is an initial dose dependent response until increasing the dose beyond the level of maximal effect will, however, lead to decreased clinical efficacy as well as increased negative side effects. Since cats cannot metabolize many terpenes, full spectrum products containing them may pose a high risk for toxicity. Cannabis (THC) intoxication in pets most commonly affects dogs (96%) and is uncommon in cats (3%); therefore, safety and storage is important, similar to precautions with children.

In animal studies, the peak brain concentrations were shortest for the varin acidic derivatives, CBGVA and CBDVA (15 minutes), followed by varin derivatives, CBGV, CBDV, THCV (30 minutes), followed by CBGA (45 minutes) and longest with CBG (2 hours).

The acid form of the cannabinoid, i.e., CBGA, CBGVA, CBDA, CBDVA, THCA, THCVA, etc., can be preserved and stabilized by forming a pharmaceutically acceptable ester or amide, such as the adduct or methanol, ethanol, glycerol, glycine, other amino acids, methylamine, ethylamine, etc. For example, Anderson, Lyndsey L., et al. “Olivetolic acid, a cannabinoid precursor in Cannabis sativa, but not CBGA methyl ester exhibits a modest anticonvulsant effect in a mouse model of Dravet syndrome.” Journal of Cannabis research 4.1 (2022):1-9 reports tests of the CBGA methyl ester.

The tinier and more dispersed the cannabinoid molecules become, the more bioavailable they are, with up to 35% sublingually absorbed CBD. Bioavailability can be enhanced with self-emulsifying drug delivery systems (SEDDS). These involve mixtures of oils, surfactants, and solvents that produce nano or micro sized droplets when they come into contact with an aqueous solution such as in the gut demonstrated greater bioavailability (about 31-34% higher compared to a reference oromucosal spray), solubility, and faster time to peak plasma concentrations in humans, with high inter-individual variations. Some of the proprietary innovations allow more consistent standardizations and are based on colloidal suspension of nanoparticles, biodegradable nano-biospheres for time-released absorption, or using tetramethylpyrazine (TMP), a plant-derived compound from the Ligusticum species, acting synergistically to change the physiochemical properties of cannabinoids. Some of these products may add alcohol for dissolution, or incorporate other vitamins, nutritional supplements or other botanicals for synergistic effects, with deodorizing options to improve flavor for oral ingestion. Co-crystals can be “fine-tuned” using various inert or pharmacologically active co-formers, which may provide a more predictable pharmacokinetic profile and subsequently reduce side effects associated with high intra- and inter-personal variability.

“TurboCBD” claims to result in increased circulating CBD levels compared to control CBD, and contains American ginseng, Ginkgo biloba, and organic hemp oil, produced using DehydraTECH™ delivery technology. Preveceutical's “Sol-Gel” is exploring an intranasal CBD formulation to increase bioavailability. A CBD gel, “Zygel” is being promoted for transdermal application in phase 2 trials. Botanix pharmaceuticals are exploring a number of gel formulations for transdermal application in indications such as acne, psoriasis and dermatitis. Kalytera has developed an L-valine-ester derivative of CBD for topical delivery, which is in pre-clinical stages, as well as a bi-sulphate derivative of CBD for oral delivery which claims to be water soluble, a bi-phosphate CBD derivative aimed for intra-tracheal delivery via a novel aerosolized formulation, and an intravenous (IV) formulation. GW Pharmaceuticals list an IV formulation in phase 1 trial for neonatal hypoxic-ischemic encephalopathy (NHIE). A sublingual formulation by Diverse Biotech Inc., and an oral liquid by Emerald Health Pharmaceuticals containing a pure synthetic CBD are being studied. Complexation of CBD with cyclodextrins (CD) has also been investigated as a potential method to increase the water solubility and subsequently improve the bioavailability of sublingually delivered CBD, which has been comparable to CBD delivered in an ethanol solution sublingually. Two formulations of CBD and CDs are currently in development by Medexus pharmaceuticals and Vireo health LLC. These companies propose complexes of CBD and CDs will increase the aqueous solubility and subsequently improve bioavailability. A nanoemulsion preparation of CBD (CBD-NE) consisted of vitamin E acetate, ethanol, Tween-20, and distilled water, to improve the poor solubility and absorption of CBD compared in an olive oil solution. These technologies to improve CBD bioavailability can be applied to CBG products. The bioavailability may be enhanced with product emulsification, co-crystals, solid salts using maleic acid or nanoparticles.

Aquaphor (Petrolatum (41%), with Mineral Oil, Ceresin, Lanolin Alcohol, Panthenol, Glycerin, Bisabolol) is an ointment, that is also similar properties to Vaseline (100% petroleum jelly), with pH of 6.8, and ads as a moisturizer and skin protectant, and may be combined with cannabinoids, e.g., CBG, CBGA, full spectrum oil, for topical use, often combined with other skin care botanical ingredients.

The bioavailability of CBD can be increased by using supplemental terpenes (Limonene, Alpha-pinene, Menthol, Myrcene and Beta-caryophyllene). However, when consumed with some supplemental herbs (Chamomile flowers; Spicy peppers-capsaicin; turmeric and black pepper-piperine), the compound's bioavailability increases even more. Today, nanotechnology is used to create new types of CBD products, which are essentially emulsified. The process of emulsification breaks down CBD molecules into small ones. In short, the process micronizes them. It is carried out using an oil or water-based product. The tinier and more dispersed the CBD molecules become, the more bioavailable CBD is, with up to 35% sublingually absorbed.

Co-administration of dietary lipids or pharmaceutical lipid excipients has the potential to substantially increase the bioavailability to orally administered Cannabis and Cannabis-based medicines by 2.5-β-fold. Sesame oil, which is mostly composed of long-chain triglycerides (LCT) has been used. Glycocholate has also been used. The carrier oil may include Panakeia or any hemp seed derived oil.

Cannabinoid cartridges and vape pens or atomizers are becoming increasingly popular, using potent Cannabis concentrates instead of e-liquid. The term “atomizer” may also be a broad reference to three types that contains the heating e-coils powered by battery, and wick or element that generates a controlled temperature, low enough to avoid burning to prevent a cocktail of toxic residue from combustion (each with unique vaporization temperature between 157 and 220 degrees Celsius, ideally 210-235 degrees Celsius for most products, although terpenes vaporize as low as 83 degrees Celsius), and the lower the power, the more specific the combustion parameters (over 230 degrees Celsius) with a discrete vapor output (too much power can have the vapor taste burnt). Drip tip atomizers are the original atomizer, in which the Cannabis oil is manually dripped onto the heating element one drop at a time, without cartridges. Cartomizers are the cheapest, disposable and most common heating element. Clearomizers are refillable and hold the most oil in their clear tanks (to visually determine its capacity).

The boiling points (which may be comparable to vaporization temperatures in degrees Celsius; may vary by source) are: CBGA 180, CBG 105, CBDA 125, CBD 170, CBCA 120, CBC 220, THCA 120, Δ9-THC 157, Δ8-THC 175, CBN 185, CBGVA 550, CBGV 230, CBDVA 180, CBDV 165, THCVA/THCV 220 (varins tend to have higher points), terpenes 106-265, most near 160. Thus, a lower atomizer temperature may be more ideal for CBG inhalation, but higher for CBGA or its varins; and boiling points tend to be lower for all acid precursors, except CBGA.

Nebulizing is using powered aerosolized liquid, usually aqueous based, often coupled sodium electrolytes to help with the misting process, adding small amounts of ethanol to dilute the oil and emulsifiers like sodium lauryl sulfate making it hydrophilic or other (micro)nanoencapsulation methods (caution using conventional hydrophobic oils with nebulizers due to its irritating effects on the lungs) producing drops, one to five microns in size, by bubbling air or oxygen (often compressed, “jet”) through it, that can be inhaled. It is the only pulmonary delivery method that doesn't require heating or combustion of the Cannabis product and keeps the entire contents intact with a high absorption and rapid acting. And unlike edibles, dosage can be controlled, too, which is especially beneficial for those using Cannabis medically. The devices can be more expensive, as well as the emulsified concentrated product, but appears to be a safer and reliable option for delivery, but they are less portable and noisy; however, they may be more publicly allowed.

A steam mist, ultrasonic (using vibrations of the piezoelectric element for vaporization) or mesh technology (less wastage) nebulizer containing hydrophilic cannabinoids (may have larger particles) could also provide some absorption, with inconsistent dosing, and inefficient due to wastage, The most common type of inhaler is a metered-dose inhaler (MDI), delivering short bursts of medicine quickly and easily when a user inhales deeply, via a portable, handheld device that is manually operated. Dry powder inhalers (also called DPIs) use a powdered form of product that users inhale directly, without the assistance of a spray. Oromucosal spray technology is also available in a branded cannabinoid medication; this can be applied intranasal devices, with a greater proportion submucosal absorption, although some particles (preferably emulsified) may be inhaled. Absorption varies depending on the conditions of the patient's internal nasal membrane and some enzymes present in nasal tissues may be deactivated by CBD, compromising our ability to metabolize other active ingredients. It may be used for sinusitis symptoms.

It is predicted that CBG, advantageously, will have less interactive effects on the drug/food products as cited above, because its cytochrome P450 liver metabolism is less prominent than CBD (but it may still carry the FDA approved “grapefruit warning”), but more than THC; similarly, these precautions may also apply to CBC. However, it is believed that these potential interactions may be minimized by CBG/CBGA's other known multiple mechanisms of action of that promotes homeostasis, which can counterbalance these effects. For example, in a study of rats who were given chemotherapy, it was shown that CBG increased appetite (without antiemetic effects), which attenuated the anticipated weight loss; however, in humans, the CBG PPAR binding may counteract this effect by increasing metabolism, and therefore, may have an overall net weight loss result.

CBG may be consumed in the form of candy, cookies, breakfast cereal, chocolate, gummies, caramels, chewing gum, peanut brittle, vape cartridges (carts), dabs, shatter, distillate, tinctures (used advantageously in submucosal or topical absorption, usually incorporating some alcohol, but may use vinegar), and infused beverages.

A conservative recommended starting protocol daily dose of CBD is 20 to 30 mg (in naïve, or approximately 0.5 mg CBD per kg of body weight per day or 30 mg of CBD for a 60-kg person, but 10 mg/d in extremely sensitive or risky individuals). Daily doses can be used in any way: either as a single dose in the evening or the dose being evenly distributed throughout the day (morning, mid-day and evening). If he/she does not experience the desired effect, titrating up the dose by 10-30 mg/d, may be tried every 10 days. Experts do not recommend taking daily doses of 70 mg CBD or more. CBG is more potent than CBD, based on expert opinion of therapeutic dosing ranges. In contrast, a medical Cannabis specialist, Dr. Russo recommends using 10-20 mg of CBG per dose, several times per day, for those with anxiety.

Incidental dosing of individuals who consume Cannabis products, such as CBD oil at 300 mg per day, include 12 mg/day (recently FDA has considered cannabinoid products in 5 mg increments, which would represent 2.5 units) of CBG. Alternatively, there are published contents of full spectrum CBD oil which can contain CBG at 4%. If oral intake is 200 mg/day of this product, it would include 8 mg/d of CBG (1.8 units). Some patients may consume high dose full spectrum CBD oil at 2500 mg/d (CBG=100 mg/d or 20 units) without adverse events. There have been clinical and animal studies conducted at higher CBG dose ranges, in which side effects were minimal and no toxicity was observed. It has been extrapolated that to meet the “reasonably be expected to be safe under the conditions of use recommended or suggested in the labeling” standard, and expert opinion/clinical experience, for most adults is <50 milligrams/day of CBG and/or CBGA (=10 “portions”), preferably in divided doses (twice or thrice daily) as an initial recommended dosing limit.

Proportions of CBD (isolated or full spectrum) may be added into the Panakeia extract for additional benefit and/or synergy as another variation. CBG-infused products such as Cannabis gummies, liquids (oil, tinctures, concentrate, capsules, topical solutions such as salves, lip balms, lotions), foods (edibles such as baked goods, coffee, chocolates, gums, and candies), beverages (emulsified in water, carbonated fluids, naturally caffeinated tea or coffee, energy and nutritional drinks), beauty (cosmetics and toothpaste) and skincare products (sunscreen, wound and anti-aging), and dog treats may be provided.

The “portion size” designated for CBG and/or CBGA is 5 milligrams (mg), similar to literature published by the US Gov't pertaining to THC content. They are metabolized in the liver (Phase I and II), and excreted primarily in the urine (with some active metabolites). Because most cannabinoids are lipophilic, they have along half-life (1-2 days, and up to 5 days for high chronic dosing), and detection can remain in adipose tissue or hair for several weeks. Ingestion of cannabinoids with lipids or high calorie meals improves bioavailability. CBGA, through our unique full spectrum extraction process is orally absorbed significantly better than CBG. Orally ingested CBG/CBGA will deliver long-lasting but relatively mild effects, whereas if inhaled, there are much stronger effects that persist for a significantly shorter duration. CBG/CBGA, as well as CBD and its acidic precursor, CBDA, bioavailability may be further enhanced with pepper (piperine), or water-soluble compounds (with more rapid absorption, compared to oral tablets) using emulsification methods for fluids. This can be further improved with nanoparticle technology.

Cordyceps sinensis extract, combined with Panakeia extract, mutually enhances the beneficial effects as an antioxidant, which may be clinically applicable in circulatory and respiratory disturbances.

The Product may be modified with additional additions that can enhance the beneficial effect including Echinacea, PEA, Melaleuca cajuputi Oil, resveratrol or liverwort; cannabidiol (CBD) which has additional endocannabinoid binding, etc.

Ethanol, Dimethyl sulfoxide (DMSO), dimethyl isosorbide, isopropyl myristate and propylene glycol as well as nanoparticles (incorporating oleic acid and eucalyptol) can be used as chemical penetration enhancers, whereas sunscreen formulations (incorporating octylmethoxycinnamate (OMC) and butylmethoxydibenzoylmethane (BMBM)) may require an opposite approach. DMSO is a most often in used as a gel or cream form (often with concentrations of about 25%), and is FDA approved for interstitial cystitis as a bladder installation, and also has been taken orally. DMSO is easily absorbed to treat wounds, burns, and other injuries, and facilitates anything on the skin to be absorbed so that it has been used as a vehicle for administering topical products. DMSO (45.5% w/w) along with ethanol, purified water, propylene glycol, and hydroxypropyl cellulose has been used in a prescription topical anti-inflammatory diclofenac solution. Another topical didofenac gel (OTC) contains carbomer homopolymer Type C, cocoyl caprylocaprate, fragrance, isopropyl alcohol, mineral oil, polyoxyl 20 cetostearyl ether, propylene glycol, purified water, and strong ammonia solution.

Herbolea™ Extraction method: Although all Panakeia extracts can be obtained from standard extraction methods, a preferred extraction method employs the process disclosed in U.S. Pat. No. 10,973,864, an environmentally friendly solvent-less method. The Bio-Herbolea™ process does not require drying (starting moisture content should be above 30%, and this process requires a critical amount of fluid content for its success). The Cannabis flower is first milled and comminuted involving a micronization step of the plant material occurs is to reduce particle sizes and increase the surface of material reading in the following step. All parts of the plant, such as stems (with high lignin content) and buds are usually trimmed, and it can be discarded, recycled, or used for organic fertilizer. Ideally, a well harvested sativa L will have more abundant flowers, with less hemp seeds, and an average plant weight is 450 gm (wet) or 295 gm (dry). Milling can be performed on wet or dried material. Distilled water is added (if the plant biomass is too dry), and carrier oil (often olive or sunflower, which are inert to the enzymatic process) are added to the plant material to form a homogeneous mixture or slurry. It is then combined with enzymes (often comprising 3% of plant biomass consisting primarily of cellulase, or hemicellulose, but also beta-glucanase, pectinase, beta-mannanase, alpha-amylase and protease, adjusted at pH 5.6 with monohydrate citric acid, and temperature controlled at 50-55° C. agitated through stirring or other agitation methods for at least 30 min) to dissolve the plant material including lignin and chlorophyll, with dissolution of the lipid-based extraction and importantly, providing additional stabilization of phyto-cannabinoids and terpenes/terpenoids. The pH of this crude (raw) extract can be adjusted for optimal enzymatic activity (i.e., pH=4.5), with temperature is set in the range of 30-55° C. This process is facilitated before or after adding enzymes by steam and/or energy waves (ultrasound/sonication or microwaves), which can maintain the original cannabinoid ratios of the whole plant, favoring CBGA content, through a highly efficient process, resulting in a greater proportion of terpenes (about 2.5× that of dry flowers, and may be designated as high-terpene full-spectrum extract-HTFSE; The terpene fraction, acts as a naturally occurring solvent, and can referred as “terp sauce” or “the terpene fraction,” refers to a runny, terpene-rich concentrate. As cannabinoids and terpenes separate from one another, extractors are left with solid cannabinoid compounds and a watery mixture of the aromatic terpenes. Sometimes sauce products are labeled with the acronym HTFSE, short for high-terpene full-spectrum extract). This process results in a high extraction yield (at least 70%, or more preferably at least 90%). The mixture obtained is then separated via density separation (i.e., centrifugation usually 2300 rotations per minute-rpm for 30 seconds) or pressing (French press) in which the lightest phase contains non-polar compounds (cannabinoids 25%, terpenes 0.1%) solubilized in oil, followed by a higher density aqueous phase containing water soluble compounds (including carbohydrates), and then these fractions are additionally separated using an ultra-filtration technique and waxes free (which is 10-15× more efficient than alternative extractions). In case of lipid extract obtained from Cannabis, the extract can be optionally heated at higher temperatures to decarboxylate acid form cannabinoids (mainly CBGA, which is actually more desirable by most manufacturers) to the desired extent, forming CBG. The discarded “sludge” contains many other beneficial hemp products, except cannabinoids or terpenoids, and may be utilized for its nutritional values, including protein, as hemp derived “cake.” This byproduct may also be considered GRAS, and may be additionally extracted to yield 25% cannabinoid-free hemp oil.

Herbolea™ has also developed a solvent-less extraction technology, Hydrocan, (WO2021037343AI). The oil extract (of lightest density containing cannabinoids and terpenoids) is further distilled with specific temperature (maximum evaporation temperatures are between 120° C. to 260° C.) and vacuum specifications (minimum pressures of 0.001-0.04 millibar-mbar, usually 0.023 mbar), and separating from said vacuum distillation, forming a very highly concentrated cannabinoids solid (maintained in acidic form) known as Distallac (without terpenes or other phytochemicals), which can be in cake form. This distillate is then mixed and pH balanced with an aqueous solution and filtered, in which a decolorized cannabinoids product (almost lax the concentration of the full spectrum lipid extract) are selectively extracted with >90% efficiency, known as Distillac. It can be further filtered (using polishing and charcoal) to reduce pathogens (bacteria, algae and fungi) as well as produce pure crystallized (<80% efficiency) CBGA. Hemp extractions may also contain other phyto-cannabinoids, but THCA, THCVA, THC, THCV, CBDA, CBDVA, CBD, CBDV will not be present, if derived from the Panakeia plant. CBGA, in powder form, predictably has a longer shelf life than decarboxylated CBG, if stored at room temperature or below, no light exposure and vacuum packed. It can then be converted to CBG and combined with other phytochemicals for the full spectrum Panakeia extract or PECSA™ based products based on subsequent distillations, which may also affect terpene content. This concentrated cake powder can be used for oral, spray or topical use. However, if MCT containing oil (i.e., coconut) was used instead of other oils to first dissolve the hemp in the Herbolea's initial extraction, then this cake can be safely dissolved for vapes, or it can be further extracted for form an isolate, powdered or crystalline form, resembling the appearance of quartz.

Extraction procedures can be used to isolate or substantially purify cannabigerols substantially free of other cannabinoids and other non-cannabinoid components such as terpenes.

Vegetable oils containing oleic acid (a monounsaturated omega-9 fatty acid) are preferably added to the crude extract for lipophilic dissolution. The commonly used oils with oleic content: 55-80% of olive oil (extra virgin), 70% avocado, 15-20% of grape seed oil and sea buckthorn oil. Newer extractions can concentrate oleic acid: sunflower or safflower (need a cultivated oil producing variety>70% content); canola (60%, previously known as rapeseed), algal (90%); soybean, palm, corn and hempseed oils contain about 10-40% oleic acid, which can improve fatty metabolism, with cardiovascular, metabolic, cognitive and mental health benefits. An even more concentrated Hydrocan™ granular paste (patent pending contains no oil) which is solventless, water-based, environmentally friendly, rapid concentration technology to selectively purify all cannabinoids while simultaneously remediating any psychoactive cannabinoids (not present in Panakeia) below quantification levels. The product can be used directly in vaporized formulations, or converted to crystalline forms as an almost pure isolate, predominantly CBGA (can be easily converted to CBG) which may be used for pharmaceutical research purposes. Other technologies have been developed which increase efficiency include microwave, high pressure homogenization, high voltage electrical discharges, pulsed electronic fields or ultrasound.

Formulations: The technology may be used to co-extract other botanical additives (hops, mint, Echinacea, etc.) which allows for unique formulations which provide full spectrum biologic agents, in natural proportions (with possible modifications by selective addition of components, and selective removal of undesired components), as well as the capacity to process other botanicals as stand-alone products.

In order to standardize the full spectrum CBG/CBGA product, which has natural variability due to the species variation, growing conditions, harvesting, post-harvest conditions and drying, etc., a target mixture may be defined, and additional botanicals added to the Cannabis, or the complementary botanical extracts added to the extracted product. In either case, an assay is performed of the Panakeia material, to determine its CBG/CBGA potency, terpene profile, and other target components. A database is then accessed, using linear programming, to cost efficiently find available botanical materials that, when extracted, will complement the Panakeia extract to achieve the target profile. In some cases, purified terpenes and terpenoids may be added to the extract, though preferably the product represents a raw extract from combined botanicals or combined extracts of raw botanicals. Greenhouse cultivation may be used as an effective means of maintaining standardization in some of the patent application claims.

An oil, concentrate, or extract is a product derived from Cannabis flower (or other botanicals) that is processed into a concentrated form, but each type of Cannabis oil is unique. Cannabis oils are efficient, with less product required to achieve the desired effect. Extracts are refined. Essential oils and cannabinoids are separated from plant material to create a smooth Panakeia oil (full spectrum CBG/CBGA, which includes other cannabinoids terpenes and flavonoids), which are products that are formulated as a tincture (sublingual), capsule form (oral), topical or vaporizer (inhaled). A tincture is a liquid concentrate procured through alcohol extraction, which pulls out many of the plant's beneficial cannabinoids.

Live resin and other products labeled “live” (like live rosin) are concentrates that have been extracted before the Cannabis plant has been dried or cured. Terpenes, the aromatic compounds that give Cannabis its flavor, are so volatile, they're known to dissipate even at room temperature. Working with a freshly harvested plant gives extractors the best chance of capturing robust terpenes and flavors. To preserve these fragile terpene profiles, extractors may freeze and store freshly cut Cannabis until it's ready to be extracted.

A Cannabis concentrate can either be full spectrum, containing a vast array of complex combinations of cannabinoids and terpenes in the natural ratio, or an isolate, which is a precise formulation of a single ingredient in crystalline or powdered form. The terpene fraction, acts as a naturally occurring solvent. Over time, the solid cannabinoid molecules separate from the liquid terpenes and leave behind rigid cannabinoid structures that look similar to quartz. Sauce, sometimes called “terp sauce” or “the terpene fraction,” refers to a runny, terpene-rich concentrate. As cannabinoids and terpenes separate from one another, extractors are left with solid cannabinoid compounds and a watery mixture of the aromatic terpenes. Sometimes sauce products are labeled with the acronym HTFSE, short for high-terpene full-spectrum extract. That means it's a terpene-rich concentrate that still maintains a well-rounded cannabinoid profile. While hemp oil is federally legal (<0.3% THC), and is widely advertised online, and hemp Panakeia oil contains full spectrum CBG (but not THC or CBD), whereas Cannabis (indica)-derived CBD is only available at a state dispensary and is federally illegal. The terpene content and specific molecules may be adjusted to provide a fragrance and flavor that is appealing, and not overwhelming to the consumer.

A raw or crude extract may still contain many terpenes, fats, and lipids. However, it can be further refined by distillation. Good, dean distillate usually tests up to 90% or higher in total cannabinoids. Pure distillate is virtually flavorless and is popularly used as a base ingredient for other Cannabis products like edibles and topical applications.

The CBG oil or extract (including flowable powder) may be administered with an absorption enhancer, such as black pepper (Piper Nigrum) containing (E)-β-caryophyllene [(E)-BCP] and piperine, and/or white pepper.

The shelf life of cannabinoid products can vary based on the formulation (quality, all ingredients including terpenes, extraction process, stabilization methods (powder or emulsifications will tend to last longer than liquid) and storage (best if cool or refrigerated except vapes, dark and dry) and packaging (sealed, vacuum packed, presence of preservatives or moisture absorbers) parameters. Acidic precursors will tend to oxidize more over time, and cannabinoids may lose their potency, but would not be toxic. Clues that a product has expired and should be discarded include lack of effect, skunky or rancid taste, odor or flavor (most of the limitation in shelf life is due to carrier oil, which can last 1-2 years, artificial color or flavors last longer), and appearance (colloidal murky with increased viscosity and/or yellow/brown discoloration of oil products). Some essential oils will covey a longer shelf-life due to their antimicrobial effects. In general, the “best by” or expiration date will be 1 year from manufacture for oils, vapes or commercialized, mass produced sealed products including vacuum sealed beverages (may need to be agitated prior to using). If it is combined with preserved foods, the shelf-life would be 3 months, or preservative-free edibles about 1 week, with refrigeration. Topical formulations may last 1-2 years. Cannabis flower (dried) can last 3-6 months with proper handling. The best ways to store edibles (gummies, hard candy or chocolate formulations tend to last 3-6 months) is in the form of cannabutter (may be emulsified with soy lecithin or sunflower oil) or oil, but freezing is an option for all products except oil formulations.

This product can be used as an additive to other food and beverages that enhance the nutritional value include inhalant (vaporized or aerosolized), alcoholic or nonalcoholic beverages, snacks, tinctures, topical applications, chewables, chocolates, coffee, toppings and spices, sweeteners, cakes, popcorn and desserts. The vaporization product may be optimized for the resin oil, burn temperature, rise time and quantity taken, taking into account therapeutic dosing and its intervals of administration and safety of the carrier products into the alveoli.

Suppositories release drugs in the body either by melting at body temperature (favors systemic effects), or by disolving in aqueous body fluids (favors local effects) with higher absorption related to submucosal environment, and dissolution, bypassing enterohepatic metabolism. The melting type of bases are fatty substances and include PCCA MBK™ (Fatty Acid using hydrogenated vegetable oil and PEG-8-distearate as a surfactant or may add Mucolox 10% for mucoadhesion) and Cocoa Butter NF (better if gently and controlled warming and product must be able to partition out of the base for absorption to occur using surfactant). For this reason, water-soluble, ionized forms of drugs are usually preferred in fatty bases for systemic use or fatty bases may be used for a local effect due to rapid melting upon body temperature. The dissolving type of suppository contains polyethylene glycol (PEG) and includes PCCA Base A (Polyethylene Glycol 1450 MW, NF, with a more rigid structure and useful for urethral application) or bases that are combinations of Polyethylene Glycol NF are useful for both hydrophilic or lipophilic products inserted rectally or vaginally. For fatty, melting bases, such as Cocoa Butter NF, the drug must be able to partitioned out of the base for absorption to occur. For this reason, water-soluble, ionized forms of drugs are usually preferred in fatty bases. The rectal rocket (a proprietary mold to treat both internal and external regions) suppository mold may be lubricated with Mineral Oil, Light NF; Medium Chain Triglycerides NF; or Polysorbate 80 NF (PCCA #30-1075) 2%/Glycerin LISP (Natural)(PCCA #30-2865) (dissolve polysorbate 80 2% in glycerin) to help facilitate removal of suppository from mold. These delivery systems are not considered dietary supplements by definition, and the FDA may consider botanicals or even GRAS products administered externally as a drug delivery system.

Terpenes may be administered through a vaporizer; including linalool and pinene, also synergistically myrcene and beta-caryophyllene, but also limonene, and eugenol (<5% dilution), with added MCT derived from either coconut or palm kernel oil along with water soluble propylene glycol or vegetable glycerin.

This Product can be modified with deletions of certain terpenes, for example, Guaiol (pro-anxiety and stimulant), phytoestrogens (apigenin) or other Cannabis plant containing compounds that can potentially result in adverse events, side effects and intolerance. This can improve the odor, taste or flavor of the patented product. Specific terpenes may be removed by affinity binding to bound to specific receptors, antibodies, affibodies, adnectins, affimers, affitins, anticalins, atrimers, fynomers, armadillo repeat protein molecules, Kunitz domain inhibitor molecules, knottins, designed ankyrin repeat proteins, etc. Non-specific or less specific affinity separation may be employed, both to remove specific terpenes or families of terpenes (i.e., β-myrcene), or to concentrate the desired terpenes, such as resin bears, polysaccharide and modified polysaccharide gels (e.g., Sepharose, alginates), and other known separation techniques. For example, apigenin may be selectively reduced by affinity separation techniques, based on its binding to estrogen receptors, or other specific binding biomolecules such as antigen-binding fragments (Fab) or other known separation techniques.

This product can be further modified with deletions of Chlorophyll. Chlorophyll is a pro-oxidant oil soluble green pigment for photosynthesis, which can interfere with oxidative stability, rancidity and flavor) as well as reducing metal and phospholipid content, which also can affect crude oil quality. However, the extracted chlorophyll can be converted to chlorophyllin, the water-soluble form, used as a supplement, is better absorbed, may be beneficial to prevent intestinal epithelial damage and infiltration of inflammatory cells and reduces autolysosomal flux, a process that uses lysosome to degrade and remove toxic molecules and organelles to modulate autophagy in inflammatory bowel diseases or infectious colitis. Pheophorbide A, is a nontoxic degradation product, and a derivative of pheophytin from chlorophyll (including hemp) catabolism by chlorophyllase, when both the central magnesium has been removed and the phytol tail has been hydrolyzed. It is used as a photosensitizer and can prevent viral (positive-strand RNA viruses such as West Nile, HCV, SARS-CoV-2 and other coronaviruses) entry into cells by modulating the virus' lipid membrane, with a small synergistic effect with remdesivir (used to treat Covid).

Panakeia Based Products: Panakeia extract may be used as a food preservative, alone or in conjunction with other components. CBG may be combined with various compounds, such as SCT (butyrate), pectin, probiotics, MCT (coconut or palm oil), caryophyllene, DMSO, alginate, aloe, mango, melatonin, sunscreens, toothpaste, AEA, PEA, SEA, OEA, eugenol, piperine, turmeric, minerals (Mg, Mn, Zn, Se), vitamin D, B complex, folate, vitamin C (ascorbic acid), EDTA (calcium), cinnamon, quercetin, resveratrol, salvia, catechin (EGCG), cordyceps, liverwort, St. John's wort, echinacea, lions mane, black seed (sesame, hemp, olive) oil, lactobacillus (which can in turn, increase SCFA especially butyrate), chitosan, spermidine, and/or omega-3 oil (flaxseed).

Objects: It is therefore na object to provide a pharmaceutical formulation comprising: at least one cannabinoid selected from the group consisting of CBG, CBGA, CBGV, and CBGVA, or pharmaceutically salt, ester or amide thereof, e.g., in an amount of 1, 2, 2.5, 3, 4, 5, 6, 7, 8, 9, 10, 15, 20, 25, 30 or more mg, or at least 3 μmol, substantially without CBDA synthase products and TCHA synthase products of the at least one cannabinoid; and at least one additional active composition selected from the group consisting of an endocannabinoid (e.g., 5, 10, 15, 20, 25, 30, 40, 50 mg or more 2-AG, AEA, PEA, OEA, SEA), dextromethorphan (e.g., 1, 3, 5, 7.5, 10, 15, 20, 25, 30, 40, 50 or more mg), melatonin (0.5, 0.1, 0.25, 0.5, 1, 1.5, 2, 2.5, 3, 3.5, 4, 4.5, 5 or more mg), 5-hydroxy tryptophan (0.5, 1, 1.5, 2, 2.5, 3, 3.5, 4, 4.5, 5, 7.5, 10, 12.5, 15, 20, 25 or more mg), and serotonin (0.5, 1, 1.5, 2, 2.5, 3, 3.5, 4, 4.5, 5, 7.5, 10, 12.5, 15, 20, 25 or more mg). The pharmaceutical formulation may be provided in a unit dose form or metered dose (e.g., acceptable mist, vapor, or inhalable formulation).

The pharmaceutical formulation may further comprise at least 0.5 μMoles of at least one of butyrate, methyl butyrate, beta hydroxy butyrate, beta hydroxy methylbutyrate, and salts, esters, and amides thereof. The pharmaceutical formulation may comprise at least 1 mg of the at least one cannabinoid, and the at least one additional active composition selected from the group consisting of at least 25 mg of the endocannabinoid, at least 5 mg of the dextromethorphan, and at least 50 μg of the melatonin, 5-hydroxy tryptophan, or serotonin, per unit dose or metered dose. The pharmaceutical formulation may further comprise at least one of piperine, curcumin, capsaicin, resveratrol, quercetin, Echinacea, and caffeine. The at least one cannabinoid may comprise a full spectrum or broad spectrum extract from a botanical source.

It is also an object to provide a cannabinoid composition, comprising: at least one cannabinoid selected from the group consisting of CBG, CBGA, CBGV, and CBGVA, or pharmaceutically salt, ester or amide thereof, in combination with terpenes comprising limonene, linalool, pinene, humulene, β-caryophylline, bisabolene, terpinolene, myrcene, the cannabinoid composition being substantially without cannabidiol and tetrahydrocannabinol; and at least one additional component selected from the group consisting of an endocannabinoid, dextromethorphan, melatonin, 5-hydroxy tryptophan, and serotonin. At least one enhancer may be provided selected from the group consisting of curcumin, resveratrol, quercitin, piperine, and Cordyceps sinensis.

The at least one additional component may comprise an N-alkylamide endocannabinoid. The at least one cannabinoid may comprise an extract of Cannabis plant comprising at least 5, 6%, 7%, 8%, 9% or 10% by weight cannabigerol and cannibigerolic acid, with natural Cannabis terpenes and flavonoids comprising components having a boiling point of less than 125 C, and lacking detectable cannabidiol and tetrahydrocannabinol, and substantially without extraction solvent residual.

It is a further object to provide a method of treating a human, comprising administering to a human a pharmaceutically acceptable formulation in oral, inhalant, enteral or transdermal form, comprising: at least 4 mg of at least one cannabinoid selected from the group consisting of CBG, CBGA, CBGV, and CBGVA, or a pharmaceutically acceptable salt, ester or amide thereof, substantially without CBDA synthase products and TCHA synthase products of the at least one cannabinoid; and at least one additional active composition selected from the group consisting of an endocannabinoid, dextromethorphan, melatonin, 5-hydroxy tryptophan, and serotonin. The method may be effective to at least one of: alleviate a symptom of a coronavirus infection; down regulate at least one of Transmembrane Serine Protease 2 (TMPRSS2) expression and angiotensin-converting enzyme 2 (ACE2) expression; and reduce levels of at least one of interleukin (IL)-6′, interleukin (IL)-8, interleukin (IL1)-1, TNF-α, IFN-γ, PPARγ, and pro-inflammatory cytokines. The at least one cannabinoid may be provided in a full spectrum or broad spectrum botanical extract comprising terpenes.

It is therefore an object to provide a pharmaceutically acceptable formulation in unit dose form comprising: at least 1 mg of at least one cannabinoid selected from the group consisting of CBG, CBGA, CBGV, and CBGVA, or a pharmaceutically acceptable salt or ester thereof, substantially without CBDA synthase products and TCHA synthase products of the at least one cannabinoid; and at least one additional active composition selected from the group consisting of: at least 25 mg of an endocannabinoid; at least 5 mg dextromethorphan; and at least 50 μg melatonin, 5-hydroxy tryptophan, or serotonin.

It is a further object to provide a method of treating a human, comprising administering a pharmaceutically acceptable formulation comprising at least one cannabinoid selected from the group consisting of CBG, CBGA, CBGV, and CBGVA, or a pharmaceutically acceptable salt or ester thereof, substantially without CBDA synthase products and TCHA synthase products of the at least one cannabinoid, Cannabis terpenes, and Cannabis flavonoids together comprising at least 5% by weight of the at least one cannabinoid, to the person, for at least one of: treatment of a coronavirus infection or an upper respiratory tract viral infection; down regulation of at least one of Transmembrane Serine Protease 2 (TMPRSS2) expression and angiotensin-converting enzyme 2 (ACE2) expression; and reduction of levels of at least one of interleukin (IL)-6′, interleukin (IL)-8, interleukin (IL)-1β, TNF-α, IFN-γ, PPARγ, and pro-inflammatory cytokines. The formulation may further comprise an endocannabinoid, dextromethorphan, melatonin, 5-hydroxy tryptophan, or serotonin for example.

The cannabinoid may comprise at least 1, 2, 3, 4, 5, 6, 7, 8, 9, or 10 mg of CBGA or a pharmaceutically acceptable salt or ester thereof. The cannabinoid may comprise at least 1, 2, 3, 4, 5, 6, 7, 8, 9, or 10 mg of at least one cannabinoid selected from the group consisting of CBG, CBGA, CBGV, and CBGVA or a pharmaceutically acceptable salt or ester thereof. The cannabinoid may comprise at least 1, 2, 3, 4, 5, 6, 7, 8, 9, or 10 mg of CBGA and/or CBGVA or a pharmaceutically acceptable salt or ester thereof.

The pharmaceutically acceptable formulation may comprise at least 25 mg of an endocannabinoid and/or at least 5 mg dextromethorphan and/or at least 50 μg melatonin, 5-hydroxy tryptophan, or serotonin. For example, it may contain 100 mg, 200 mg, 300 mg, 400 mg, 500 mg, 600 mg, 700 mg, 800 mg, 900 mg, 1000 mg, 1100 mg, 1200 mg, 1500 mg PEA, and/or at least 200 mg of an acyl ethanolamide endocannabinoid (PEA, OEA, LEA or SEA in combination or alone), and/or 10 mg dextromethorphan and/or 1 mg melatonin, 5-hydroxy tryptophan, or serotonin (5-hydroxytrypamine).

The pharmaceutically acceptable formulation may include at least 50 mg, or 100 mg, 150 mg, or 200 mg, or 250 mg, or 300 mg, or 400 mg, or 500 mg SCFA, preferably, butyrate, but also acetate, valerate, propionate). Alternative exogenous butyrate forms include its derivatives that can be found endogenously in humans: HMB (not GRAS), or BHB (GRAS).

The pharmaceutically acceptable formulation may further comprise at least 500 mg of pectin or a pectin derivative, such as modified citrus pectin, up to a dose of 5000 mg per day.

The pharmaceutically acceptable formulation may further comprise at least 1 mg of at least one of piperine, curcumin, capsaicin, resveratrol, quercetin, anandamide, Echinacea, and caffeine.

The at least one cannabinoid may be a full spectrum or broad spectrum extract from a botanical source. It may be extracted from plant biomass in a process substantially without use of organic extraction solvent and without exceeding 150° C. It may be a full spectrum extract or broad spectrum extract processed with affinity chromatography to selectively reduce at least one non-cannabinoid receptor binding component, e.g., processed to selectively deplete phytoestrogens.

The unit dose form may comprise at least 5%, 7.5%, 10%, 12.5%, 15%, 18%. 20%, 22.5%, 25%, 27%, 28.5%, or 30% by weight of CBG, CBGA, CBGV, and CBGVA, and may be principally in the acid/salt, ester or amide form.

It is also an object to provide a pharmaceutically acceptable formulation comprising: at least one cannabinoid selected from the group consisting of CBG, CBGA, CBGV, and CBGVA, or a pharmaceutically acceptable salt or ester thereof, substantially without CBDA synthase products and TCHA synthase products of the at least one cannabinoid; and at least one additional active composition selected from the group consisting of an endocannabinoid, dextromethorphan, melatonin, 5-hydroxy tryptophan, and serotonin. The pharmaceutically acceptable formulation may be in a unit dose or metered dose form comprising, per unit dose or metered dose: at least 1 mg of the at least one cannabinoid, and the at least one additional active composition selected from the group consisting of at least 25 mg of the endocannabinoid, at least 5 mg of the dextromethorphan, and at least 50 μg of the melatonin, 5-hydroxy tryptophan, or serotonin. The pharmaceutically acceptable formulation may be in a pharmaceutically acceptable vapor or inhalable formulation. The at least one cannabinoid may be a full spectrum or broad spectrum extract from a botanical source. The formulation may further comprise at least one of piperine, curcumin, capsaicin, resveratrol, quercetin, anandamide, and caffeine.

It is a further object to provide a pharmaceutically acceptable formulation comprising at least one cannabinoid selected from the group consisting of CBG, CBGA, CBGV, and CBGVA, or salt, ester or amide thereof, substantially without CBDA synthase products and TCHA synthase products of the at least one cannabinoid; and at least one of: (a) an acyl ethanolamide, having a ratio of the acyl ethanolamide to the at least one cannabinoid of at least 25:1; (b) an opioid kappa receptor agonist, having a ratio of the opioid kappa receptor agonist (including menthol) to the at least one cannabinoid of at least 1:20; and/or (c) a 5-HT1 receptor agonist, having a ratio of the 5-HT1 receptor agonist to the at least one cannabinoid of at least 1:100.

A further object provides a pharmaceutically acceptable formulation comprising a whole (broad spectrum; intrinsically it does not contain any THC) Cannabis extract comprising at least one cannabinoid selected from the group consisting of CBG, CBGA, CBGV, and CBGVA, or salt, ester or amide thereof, substantially without CBDA synthase products and TCHA synthase products of the at least one cannabinoid, which is selectively depleted of at least one component by affinity chromatography.

Another object provides a pharmaceutically acceptable formulation comprising an esterified extract of Cannabis, comprising CBGA acyl ester, substantially without CBDA synthase products and TCHA synthase products CBGA.

A further object provides a pharmaceutically acceptable formulation in unit dosage form, comprising CBGA acyl ester in an amount of at least 2.5 mg per unit dose, optionally with at least 200 mg PEA or another endocannabinoid; at least 5 mg dextromethorphan or another non-addictive or low-addictive opiate, e.g., kappa receptor binding ligand; at least 500 μg melatonin, 5-HT, 5-HTN, tryptophan, tyrosine, or L-DOPA; or at least 50 mg of endocannabinoids including anandamide, 2AG, OEA, SEA, PEA).

A still further object provides a pharmaceutically acceptable formulation comprising at least one cannabinoid selected from the group consisting of CBG, CBGA, CBGV, and CBGVA, or salt, ester or amide thereof, and at least one further component selected from the group consisting of piperine, curcumin, capsaicin, black seed, sesame seed, hemp seed or olive oil, SCFA (or SCT including butyrate), Echinacea (alkylamides or coneflowers), cinnamon, ginger, ascorbic acid, MCT coconut oil, MCT palm oil, vitamin D, vitamin E, vitamin K, vitamin A, vitamin B1 (thiamin), vitamin B2 (riboflavin), vitamin B3 (niacin), vitamin B5 (pantothenic acid), vitamin B6 (pyridoxine), vitamin B7 (biotin), vitamin B9 (folic acid), vitamin B12 (cobalamin), lithium, zinc, magnesium, manganese, copper, selenium, chromium, lithium, vanadium, iodine, apple cider vinegar, eugenol, caryophyllene, PEA, EDTA, DMSO, melatonin, GABA, glutamate, theanine, arginine, taurine, proline, leucine, valine, ornithine, quercetin, coenzyme 10, dextromethorphan (or Nuedexta, a proprietary combination product combined with quinidine sulfate to delay its metabolism, FDA approved to treat pseudobulbar affect), quinine, glutathione, N-acetyl cysteine, resveratrol, licorice, geranium, mango, ginger, dove, nutmeg, cumin, saffron, menthol, lavender, hibiscus, lipoic acid, probiotics, cordyceps mushroom fungi, corydalis, fermentation products, citrus pectin, pectin derivative, green tea, ashwagandha, Salvia, ginseng, gingko, kava kava, St John's wort, flaxseed oil, sunflower oil, grape seed oil, grapefruit oil, banana, lime, lemon, orange, oatmeal, rose hips, primrose, peppermint, wintergreen, witchhazel, feverfew, oregano, cilantro, cherry extract, blueberry extract, cranberry, bilberry, huckleberry, lecithin, carnitine, lutein, PABA, inositol, ubiquinol, creatine, bacopa, DHEA, ribose, stevia (Truvia®), erythritol, enlulose, allulose, brazzein, monellin, algae, kelp alginate, phosphatidal choline, phosphatidyl serine, tryptophan, 5-HTN, 5-HT, valerian, tyrosine, phenylalanine, DOPA, methyleneblue, red yeast extract, milk thistle, fisetin, friedlin, avocado extract, fulvic acid, aloe vera, dark chocolate, cocoa, pineapple enzyme, papaya extract, fermented papaya extract, cats claw, lion mushroom mane, linolenic acid, olenic acid, cetoleic acid, genistein, Huperzine A, passionflower extract, S-adenosyl methionine (SAM), Boswellia, Hops extract, lycopene, saw palmetto, hoodia, docosahexaenoic acid (DHA), eicosapentaenoic acid (EPA), diindolylmethane, caffeine, coffee, guarana,

Another object provides a method of treating or preventing COVID-19 or a respiratory virus infection, comprising administering a pharmaceutically acceptable formulation comprising at least one cannabinoid selected from the group consisting of CBG, CBGA, CBGV, and CBGVA, or a salt, ester or amide thereof, substantially without CBDA synthase products and TCHA synthase products of the at least one cannabinoid, in an amount of at least 5 mg.

It is a further object to provide a method of treating a human severe acute respiratory coronavirus infection, comprising administering a pharmaceutically acceptable full spectrum extract of a plant of genus Cannabis comprising at least 5% by weight cannabigerol, Cannabis terpenes, and Cannabis flavonoids, to a person having the human severe acute respiratory coronavirus infection.

It is also an object to provide a method of treating a human severe acute respiratory coronavirus infection, comprising administering a pharmaceutically acceptable cannabinoid formulation, comprising cannabigerol, in combination with limonene, linalool, pinene, humulene, β-caryophylline, bisabolene, terpinolene, myrcene, and substantially without cannabidiol and tetrahydrocannabinol, to a person having the human severe acute respiratory coronavirus infection.

It is a further object to provide a full spectrum extract of a plant of genus Cannabis comprising at least 5% by weight cannabigerol, Cannabis terpenes, and Cannabis flavonoids, substantially without cannabidiol, tetrahydrocannabinol, and extraction solvent residue.

The cannabinoid composition may be provided in a pharmaceutically acceptable inhalable formulation for efficacious prophylaxis or treatment of respiratory coronavirus infection in humans.

It is another object to provide a cannabinoid composition, comprising: cannabigerol, in combination with limonene, linalool, pinene, humulene, β-caryophylline, bisabolene, terpinolene, myrcene, and substantially without cannabidiol and tetrahydrocannabinol; at least one enhancer selected from the group consisting of curcumin, resveratrol, quercitin, and piperine; and at least one N-alkylamide.

It is also an object to provide a full spectrum cannabinoid formulation, comprising: an extract of Cannabis plant comprising at least 5%, 6%, 7%, 8%, 9%, or 109% by weight cannabigerol and cannibigerolic acid, with natural Cannabis terpenes and flavonoids comprising components having a boiling point of less than 125 C, and lacking detectable cannabidiol and tetrahydrocannabinol, and substantially without extraction solvent residual.

The composition may further comprise at least one absorption enhancer selected from the group consisting of curcumin, resveratrol, quercitin, and piperine. The composition may further comprise at least one N-alkylamide.

The cannabinoid composition may be provided in a pharmaceutically acceptable formulation for efficacious down regulation of Transmembrane Serine Protease 2 (MPRSS2) and/or angiotensin-converting enzyme 2 (ACE2) expression in lung cells and elsewhere in body.

The cannabinoid composition may be provided in a pharmaceutically acceptable formulation for efficacious reduction of interleukin (IL)-6, interleukin (IL)-8, interleukin (IL1)-3, TNF-α, IFN-γ, and/or PPARγ.

The cannabinoid composition may be provided in a pharmaceutically acceptable formulation for efficacious reduction of pro-inflammatory cytokines in lung tissue in response to SARS-Cov2 or other respiratory virus infection.

The cannabinoid composition may be provided in a pharmaceutically acceptable inhalant formulation.

The cannabinoid composition or formulation may further comprise Cordyceps sinensis extract.

It is also an object to provide a method of processing an extract of a plant of genus Cannabis comprising contacting the extract with a separate phase having phytoestrogen binding molecules, to thereby sequester the phytoestrogens in the separate phase and form a phytoestrogen-depleted extract. The extract may be a full spectrum extract, and the phytoestrogen-depleted extract is substantially a full spectrum extract depleted of phytoestrogens. The extract may comprise at least 5% by weight cannabigerol. The extract may be a full spectrum extract, and the phytoestrogen-depleted extract is substantially a full spectrum extract depleted of phytoestrogens.

It is a further object to provide a method of processing an extract of a plant of genus Cannabis comprising contacting the extract with a separate phase having a biomolecular receptor or high-affinity binding peptide for a cannabinoid, terpene or flavonoid component, to thereby sequester the cannabinoid, terpene or flavonoid component bound to the biomolecular receptor or high-affinity binding peptide in the separate phase and form a depleted extract. The depleted extract may be substantially without cannabidiol, tetrahydrocannabinol, and extraction solvent residue. It is another object to provide a Cannabis extract, having affinity depletion of phytoestrogen components or other receptor or high affinity binding peptide specific components.

### DETAILED DESCRIPTION OF THE PREFERRED EMBODIMENTS

Example 1: The full spectrum extract from the Panakeia plant with any additional modifications, including adding or deleting natural ingredients, changing concentrations has been designated as Bazelet Health Systems, Inc. proprietary Phyto-EndoCannabinoid System Activating compound (PECSA™) which is comprises all of the naturally occurring cannabinoids and other non-cannabinoid components that are co-extracted with the at least one CBGA (and to a lesser extent, CBGVA) or decarboxylated derivative-type compound (CBG, and to a lesser extent CBGV). In other PECSA™ iterations, the Panakeia “full spectrum” CBG/CBGA extract is concentrated, with other additive, substitutions or deletions, or combined with other natural ingredients to enhance effect, absorption and flavor, to form the novel proprietary food product. In one embodiment. The PECSA™ extract will contain primarily CBGA, or converted in some proportion to CBG, or be enriched from products from the Panakeia plant including higher concentrations of CBG. In other words, the extract contains a greater proportion of the total cannabinoid content as CBG as compared to the cannabinoid composition from which the extract was prepared primarily by purifying the plant extract further after extraction to select specifically for CBG (through patented methods for the exact extraction parameters for optimization of product yield). The therapeutic effects of PECSA™ are due to the combined ingredients as a synergistic or entourage effect, with CBGA/CBG as the primary ingredients. Although they are similar compounds, CBGA (an acid) and CBG, may have different therapeutic applications and benefits than if they were administered as an individual component.

Conditions for which Panakeia full spectrum extract and/or PECSA™ may improve the management of serious diseases or conditions associated with morbidity that has substantial impact on day-to-day functioning: pain (somatic-musculoskeletal, visceral, neuropathic and nociceptive), mood, anxiety, PTSD and sleep disorders, neurodegenerative disease (including dementias, Huntington and Parkinson disease), ischemic disease, brain injury (including acquired) or damage, age related inflammatory or autoimmune disease, cachexia, nausea and vomiting, glaucoma, movement disorders/spasticity, rheumatoid (or other autoimmune) arthritis, bone disease and osteopenia/osteoporosis, asthma, allergy, psoriasis, Inflammatory bowel disease (Crohn's disease), systemic lupus erythematosus, hypertension, diabetes, neurogenic bladder dysfunction, cancer, nephritis and renal ischemia, pelvic pain (including endometriosis), periodontal disease and gingivitis, skin conditions (including acne and eczema) as well as respiratory illness (i.e., Covid-19).

The PECSA™ full spectrum extract (with additional modification, including additives or deletions) may be provided in dosage forms, e.g., oral unit dosage forms, providing a CBG/CBGA content up to 1200 mg/day, and may be used for the management of many serious conditions or diseases as discussed above.

Example 2: Panakeia Extraction (Herbolea Process, or equivalent). Herbolea Biotech SRI also has an enzyme-assisted lipid-based extraction technology, see U.S. Pat. No. 10,973,864. The method for preparing a cannabinoid concentrate comprises of the following steps: providing a lipid extract containing cannabinoid acids of at least 20% by weight percent on total cannabinoids weight.

In an exemplary process, to extract the phytocannabinoids or terpenes containing plant material such as hemp or Cannabis can be fresh (preferred) or dried. The Cannabis flower is first milled and comminuted involving a micronization step of the plant material occurs is to reduce particle sizes and increase the surface of material reacting in the following step. All parts of the plant, such as stems (with high lignin content) and buds are usually trimmed, and it can be discarded recycled for organic fertilizer. Ideally, a well harvested sativa L will have more abundant flowers, with less hemp seeds, and an average plant weight is 450 gm (wet) or 295 gm (dry). Milling can be performed on wet or dried material.

Distilled water is added (if the plant biomass is too dry), along with enzymes (usually cellulose) and carrier oil (often sunflower) are added to the plant material to form a homogeneous mixture or slurry; temperature (usually <55 C) and pH (usually 4.5). It is then mixed with hydrolyzing enzymes (5-8%, based on the plant appearance, with lower amount if the plant has robust flowers), with higher concentrations required to dissolve the plant matrix, using cellulitic enzymes (primarily cellulase, hemicellulase, etc.) are added to the plant material to form an aqueous slurry. Conditions might vary according to the specific enzyme or enzymatic cocktail used to dissolve the plant material including lignin and chlorophyll. The mixture may be agitated through stirring or other agitation methods for at least 30 min to let the enzymes degrade the plant material. Ultrasound/sonication or microwaves or steam explosion may be used before or after adding enzymes to the mixture to reduce the time necessary to achieve plant material dissolution and high cannabinoids lipid-extraction yield. Water to plant ratio is critical to achieve plant material degradation through enzymatic activity; newly harvested plant material can also be used directly, avoiding pre-drying step during which degradation and/or losses of phytocannabinoids and terpenes, especially monoterpenes, can occur; in such case, little to no water can be used. Lipids can be added to the mixture any time without significantly modifying enzymatic activity; a suitable lipids-to-plant material ratio to obtain high phyto-cannabinoid content and high extraction yield (at least 70%, or more preferably at least 90%). The mixture obtained is then separated via density separation (i.e., centrifugation usually 2300 rotations per minute-rpm for 30 seconds) or pressing (French press) and/or filtration to recover a lipid fraction highly enriched with cannabinoids and waxes free. In case of lipid extract obtained from Cannabis, the extract can be optionally heated at higher temperatures to decarboxylate acid form cannabinoids (mainly CBGA) to the desired extent.

The use of enzymes drastically enhances the lipid-based extraction of phytocannabinoids and terpenes/terpenoids, including volatile monoterpenes, allowing for a significant reduction of the lipid solvent-to-plant material ratio (i.e., 10-15 times compared to traditional Romano-Hazekamp method), while still achieving a high cannabinoids extraction yield (i.e., 90%), hence the possibility to safely and directly obtain a waxes-free lipid extract, having a phytocannabinoid and terpene content appropriate for and compatible with therapeutic applications dosage, where the terpene fingerprint of the plant material is faithfully reproduced (the proportion of cannabinoids, terpenes and other phytochemicals is preserved, so CBGA remain the predominant contents). Furthermore, it has also been found that the use of enzymes dramatically increases the stability of phytocannabinoids and terpenes/terpenoids in the extract, allowing to achieve a shelf-life appropriate for and compatible with pharmaceutical applications with no addition of preservatives. In this step cannabinoids and terpenes are released. The pH of the mixture can be adjusted for optimal enzymatic activity (i.e., pH=4.5). Temperature is set in the range of 30-55° C.

In addition to that, the solid fraction generated by the process shows a phytocannabinoids content significantly reduced. In the case of hemp seeds (if using Panakeia, low concentrations of CBGA, and if using standard hemp plants, low concentrations of CBGA, CBD or CBDA, but also contain a protein rich “cake”), the cannabinoids content was greatly reduced compared to mechanical expeller, therefore making the protein-rich solid fraction compliant with safety guidelines for feed and food product applications. Panakeia hemp seed, if utilized for the carrier oil may be derived with specific plants harvested for the seed yield and have a different terpene distribution. The extraction process may include the entire Panakeia plant biomass; or the Panakeia seeds separated, and the seed oil extracted with an oil yield of about 25%.

A nonpolar solvent (usually water) is then added to facilitate the extraction and direct infusion, and the carrier oil helps solubilize the active ingredients (cannabinoids and terpenes), which yields a full spectrum extract, (if scaled for 100 kg oil which is >90% efficient), known as bioherbolysis. This allows higher efficiency without concentrating at first step, giving subsequent flexibility to further concentrate at higher levels of CBGA spectrum. The mixture is then placed in a centrifuge in which the slurry is centrifuged at high speeds (>4000 rpm, usually 4500 rpm). The lightest phase contains non-polar compounds (cannabinoids 25%, terpenes 0.1%) solubilized in oil, followed by a higher density aqueous phase containing water soluble compounds (including carbohydrates), and then these fractions are separated using an ultra-filtration technique.

Example 3 Hydrocan Process. Herbolea also developed a solvent-less extraction technology. See, WO2021037343Δ1. The oil extract (of lightest density) is then distilled with specific temperature (maximum evaporation temperatures are between 120° C. to 260° C.) and vacuum specifications (minimum pressures of 0.001-0.04 millibar-mbar, usually 0.023 mbar), and separating from said vacuum distillation a distillate containing the cannabinoid concentrate to form a cannabinoid acid solid distillate known as Hydrocan (without terpenes or other phytochemicals), which can be in cake form. This distillate is then mixed and pH balanced with an aqueous solution and filtered, in which a decolorized cannabinoids product (almost 1 Ox the concentration of the full spectrum lipid extract) are selectively extracted with >90% efficiency, known as Distillac (if scaled for 10.1 Kg Concentrate of CBGA). It can be further filtered (using polishing and charcoal) to reduce pathogens (bacteria, algae and fungi) as well as produce pure crystallized (<80% efficiency) CBGA.

This also contains other cannabinoids, but not THCA, THCVA, THC, THCV, CBDA, CBDVA, CBD, CBDV, CBGA, in powder form, predictably has a longer shelf life than decarboxylated CBG, if stored at room temperature or below, no light exposure and vacuum packed. It can then be converted to CBG and combined with other phytochemicals for the full spectrum Panakeia extract or PECSA™ based products based on subsequent distillations to incorporate or eliminate any terpenes, which can enhance the taste, odor or flavor.

A preferred protocol for all of the extraction of the full spectrum CBGA/CBG product are based on patented extraction from Herbolea, though modified for the present application to Panakeia.

The lipid extract containing cannabinoids may be obtained by putting in contact with a biological material containing cannabinoids with liquid paraffin, which can selectively extract cannabinoids in their acid forms more efficiently than neutral forms. Therefore, if liquid paraffin is utilized to obtain a lipid extract, it is possible to obtain a distillate, having a higher purity, even if the cannabinoids in the starting biological material have gone through partial decarboxylation. Where decarboxylation is not a primary concern, the paraffin is not necessary.

The method may also be described by obtaining the lipid extract containing cannabinoids from a plant material containing cannabinoids by means of the steps of: a. comminuting a biological material containing cannabinoids; b. mixing the comminuted plant material with enzymes to form a mixture to which water and lipids or solvents are optionally added; c. agitating the mixture at a temperature range of 1 to 80° C.; and d. separating the mixture into a lipid phase, an aqueous phase, and a solid phase; wherein the lipid phase comprises the lipid extract.

Enzymes may also be used to process the plant material, including one or more enzymes independently selected from the group consisting of Oxidoreductases, Transferases, Hydrolases, Lyases, Isomerases, and Ligases, cellulase, hemicellulase, xylanase, glucanase, β-glucanase, pectinase, glucuronyltransferase, lipase, amylase, alpha-amylase, beta-amylase, phospholipase, arabanase, galacto-mannanase, beta-mannanase, protease and phytase. In an embodiment, said enzyme is cellulase. In another embodiment, said enzyme is beta-glucosidase. In another embodiment, said enzyme is hemicellulase. In another embodiment, said enzyme is xylanase. In yet another embodiment, said enzyme is glucanase. In yet another embodiment, said enzyme is pectinase. In still another embodiment, said enzyme is amylase. In yet another embodiment, said enzyme is lipase or phospholipase. In said another embodiment, said enzyme is glucuronosyltransferase or alcohol dehydrogenase. In yet another embodiment, said enzyme is arabinanase. In still another embodiment, said enzyme is phytase. In a further embodiment, said enzyme is protease. Preferably, said enzyme is a mix or a cocktail of cellulase, β-glucanase, pectinase, β-mannanase, alpha-amylase and protease; wherein the amount of enzyme is 3% of the weight of plant material; and the pH of the mixture is adjusted to pH 5.6 with monohydrate citric acid.

Preferably, the cannabinoid concentrate comprises less than 1 ppm of organic solvent selected from a group consisting of Acetone, Benzene, Butane, Chloroform, Cydohexane, Dichloromethane, Ethanol, EthylAcetate, Ethylbenzene, Heptane, Hexane, Isobutane, Isopropanol, Methanol, Pentane, Propane, Toluene, m-Xylene, o-Xylene, p-Xyleneheptane or a mixture thereof.

Example 4 Cannabis Plant Harvesting. The “Panakeia” Cannabis sativa L. plant is cultivated from a seed, and the seedling is grown in a climate-controlled environment with temperature ranges from 45-100° F., and humidity that ranges from 20-100%, with sunlight or simulated sunlight for >18 hours per day, and with fertilized soil that is pesticide and herbicide free. Cannabis sativa L. rapidly sequesters carbon and bolsters soil systems. It is estimated that an average Panakeia plant weighs 450 gm (wet), with a required growing surface area of 2 ft for indoor growing environments and outdoor growing environments of 2,000-3,500 plants per acre are estimated.

Current studies have shown the mineral accumulating capabilities of Cannabis sativa L. as a “cover crop” and its potential as a promising method of bioremediation. Cannabis sativa L. taproot and root-ball, aids in the structuring of soils and retention of water which prevents desertification, while bolstering soil systems and aiding in the global effort to reduce carbon emissions and creating habitat for the evolution and expansion of complex soil food webs. This makes Panakeia Cannabis Sativa L. safe for use in water conservation, harvesting and improving soil structure. A “hemp seed cake” has been shown to be optimal for livestock feed, increasing overall health, quality of meat and reducing methane off gassing by 10%. Oil seeds contain 25-35% lipids with unique and perfectly balanced fatty acid profiles, characterized by an over 80% amount of polyunsaturated fatty acids, with the essential fatty acids of ratio (omega 3/omega 6 as 1:4), as suggested for optimal human nutrition.

The biomass produced in these areas will be the future industrial feedstock of the globe. High in alpha cellulose, hemi cellulose and lignin, micronized hemp herd can be utilized for its multifaceted attributes and applications including fabrication of unique graphene-like nanomaterial, or as a building material, “hempcrete”, consisting of two major compounds (Hemp shiv, and a Lime-based binder for good thermal and insulation properties.

Typically, the Cannabis plants are traditionally hung upside down to dry from a “clothesline” while blocking most of the light with a sensor monitored climate controlled: 70° F.±10, humidity: 50%±5 and good air flow, using a fan (although this may lower humidity, but one needs to avoid over-drying). There may be optimization for altitude. For smaller components, an herb dryer has been used to set any loose buds or smaller branches on. As the Cannabis dries, the CBGA found in the kiefs and buds may spontaneously convert to CBG. The buds that are dried too quickly will experience a more significant decomposition, with less concentration of cannabinoids than those that are allowed to dry more slowly.

Curing is a continuation of the drying process, but in a slower, controlled environment, such as in sealed mason jars, and occurs for up to two months. Curing is to be avoided if the CBGA is desired to be retained. Once the Cannabis is dry, it may still need time for CBGA to CBG spontaneous conversion. Proper curing stops the degradation process before volatile compounds like terpenes and cannabinoids evaporate or transform into less favorable compounds. Additionally, cannabinoid synthesis (the process of creating those valuable chemicals) continues to take place even after harvest. During the curing process, bacteria work to break down the chlorophyll in the plant material. Chlorophyll is what makes the plants nice and green in color, but also contributes to a harsh smoking experience. The containers are stored in a dark, temperate place, with occasional burping to allow oxygen into the jar and release moisture or other off-gassing substances.

An oil, concentrate, or extract is any product derived from Cannabis flower that is processed into a concentrated form, but each type of Cannabis oil is unique. Cannabis oils are efficient, with less product required to achieve the desired experience. Extracts are refined. Essential oils and cannabinoids are separated from plant material to create a smooth Cannabis full spectrum oil, which are products that are sold as a tincture (sublingual), capsule form (oral), topical or vaporizer (inhaled). A tincture is a liquid concentrate procured through alcohol extraction, which pulls out many of the plant's beneficial cannabinoids. A topical dosage form may comprise a cream, foam, gel, lotion, or ointment base in which a cannabinoid (and other components) is dissolved, suspended, or otherwise provided. See en.wikipedia.org/wiki/Topical_medication.

For example, 36.7 lbs of the Panakeia CBG hemp strain was extracted using cryo ethanol extraction (−80° C.). The resulting emulsion was filtered through a 20 μm filter, and fed into the rotovap to remove the ethanol and decarboxylate the CBGA. The initial temperature was set to 101° C., and the pressure was 0.06 MPa. When most of the ethanol was extracted, the temperature was increased to 120° C. to ensure full decarboxylation with the pressure remaining the same. The resulting oil had more of a citrus, floral scent than typical CBD oil. The consistency in the rotovap also appeared to have a lower viscosity that what is typical of CBD. Once the material was decanted from the rotovap into jars, it became apparent that there were two distinct layers indicating that there were two oils present with different densities. The top layer was thick, black and sticky to the touch much like CBD. The bottom layer was thinner, golden in color and had a slick (like olive oil) feel.

Other cannabinoid extraction processes can be applied to Panakeia. Various processes to extract phytocannabinoids and/or terpenes/terpenoids have been developed. The following major extraction processes are known:

1. Cold pressing for producing hemp seed oil. Hemp seed oil is rich in nutrients and is a good addition to any diet, but only contains small amounts of cannabinoids (<2%, in the case of industrial hemp), as it is made from just the seeds of the plant. Hemp seed oil can certainly be added to CBD supplements as a base for these products. However, cold pressing is not useful to produce an oil high in cannabinoids, as cannabinoids are mostly contained in the stalks and buds that cannot be directly processed by a normal press or expeller.

2. The Rick Simpson Method for Cannabis Oil is a popular extraction method for extracting CBD oil, which uses petroleum or naphtha as solvents. This method, although efficient in extracting the active compounds from the Cannabis plant (mostly done with plants high in THC), usually leads to products that have a lower concentration of terpenoids and other cannabinoids such as CBD, while effectively yielding higher concentrations of THC. The main drawback of such method is that residuals from the solvents may remain and potentially interfere with one's immune function as described by Romano and Hazekamp (“Cannabis Oil: chemical evaluation of an upcoming Cannabis-based medicine”, 2013).

3. Extraction with ethanol can be used for extracting the full range of cannabinoids from the Cannabis plant, and it is safer than the Rick Simpson method. On the other hand, ethanol has a low selectivity, and it extracts undesired chlorophyll and waxes, so the final product has an unpleasant taste. Chlorophyll can be removed by filtering the extract, but this additional step also removes a significant proportion of the cannabinoids, therefore leading to less potent extract. Furthermore, stability of cannabinoids as well as N-alkylamides in ethanol extracts is low (Citti et al., 2015 and Spelman, 2009).

4. Extraction with Sonication/ultrasonic waves: C. Da Porto, (Ultrasound-assisted extraction of volatile compounds from industrial Cannabis sativa L. inflorescences, 2014) describes procedures for extracting THC and terpenes from hemp by using ultrasonic waves. The use of ultrasonic increased the extraction of THC, but after 15 min of treatment the overall efficiency of extraction was still not satisfactory.

5. Super Critical CO₂ extraction (U.S. Pat. No. 9,186,386 B2, U.S. Pat. No. 6,403,126 B1) can bean efficient method to obtain a highly enriched cannabinoids oil (>60%). At such level of concentration, the product is not directly consumed but it is diluted with vegetable oils such as olive oil to reach β-5%. The method uses safe solvents, but it requires complex equipment and expertise, is energy demanding, and the product obtained is very expensive. Additionally, it requires the initial Cannabis material to be dried, adding a step that is time consuming and has negative effects on important compounds such as volatile monoterpenes. Furthermore, the process itself is subject to significant losses in terms of monoterpenes extraction yield, hindering the entourage effect of the extracts. Additionally, it has high selectivity for toxic components which might be present in pesticides, therefore a risk associated to their presence in concentrated form in the final product might be present. Moreover, the product of SC—CO₂ extraction may have a significantly different chemotypic fingerprint from that of Cannabis flower (Sexton, 2017). Finally, the stability of cannabinoids extracted with CO₂ diluted in olive oil is inferior to that obtained with their direct extraction in olive oil as described by Cannazza (“Medicinal Cannabis: Principal cannabinoids concentration and their stability evaluated by a high-performance liquid chromatography coupled with diode array and quadrupole time of flight mass spectrometry method”, 2016).

6. Winterization may be performed after supercritical fluid extraction and encompasses the use of ethanol or butane at low temperatures (U.S. Pat. No. 9,186,386 B2, U.S. Pat. No. 6,403,126 B1). Such process presents several drawbacks such as the high investment required, the need for highly skilled technicians to utilize complex equipment, the use of flammable and harmful organic solvents to winterize the crude extract, the high energy consumption. It is very challenging to completely remove organic solvents used in combination with CO₂ during the extraction step or to remove chlorophyll in the winterization step. The technical challenge to overcome has led policymakers to set content limits for organic solvents, some of which are known cancerogenic compounds, as high as 5.000 ppm (source Health Canada). Additionally, supercritical C02 has high selectivity for toxic components which might be present in pesticides, therefore a risk associated to their presence in concentrated form in the final product might be present. Furthermore, as heat is required to dry the biomass and remove the solvents as well as it is generated through the C02 extraction step, it is very difficult to well-preserve heat-sensitive acidic forms that can decarboxylate. The cannabinoids content achieved with such process is not sufficiently high to go directly into a crystallization step. An intermediate distillation step is often required. Finally, supercritical CO₂ cannot extract with the same efficiency acidic forms of cannabinoids due to higher molecular weight compared to the neutral forms. All these aspects make the whole process not an ideal option to extract and concentrate acidic forms of cannabinoids. In the vaping sector, for instance, the possibility to utilize concentrates having a high content of CBDA instead of CBD is helpful to avoid the formation of crystals in the vaping cartridges.

A more recent alternative technique is represented by cryogenic-ethanol, a process in which a biomass that has been previously dried is extracted at very low temperatures (−40° C.) to avoid extraction of chlorophyll and waxes into the solvent. The cannabinoids-enriched ethanol solution is then evaporated to recover the solvent. Such activity is energy intensive, and it can be very time consuming, considering the large volumes of solvents to be evaporated (up to 20 times biomass weight). Furthermore, the use of organic solvents inherently results in safety, health and environmental issues.

As to the cannabinoid isolates, today CBFD crystals are obtained from concentrates generated with one of the techniques earlier described by means of purification steps, such as distillation followed by chromatography, and then a crystallization step by means of heptane or hexane (GB 2393182, WO2016153347A1). Chromatography is required to eliminate impurities before entering the crystallization step, especially if the starting biomass contain low level of cannabinoids such as hemp. Chromatography can be a very time consuming and costly process and presents some limitations in scaling up. Furthermore, chromatographic purification methods such as flash chromatography can have a high environmental impact since they typically involve large quantities of harmful or toxic solvents run at high flow rates.

7. Extraction with microwaves. Koturevic et al. (A rapid method for the extraction of cannabinoids from Cannabis sativa using microwave heating technique, 2014) described the possibility to use microwaves to assist the extraction of cannabinoids by organic solvents. Few organizations such as New Brunswick Innovation Research Chair in Medical Technologies (NBIRC), Radient Technologies and Scientus Pharma announced partnerships with Cannabis producers to develop microwaves-assisted cannabinoids extraction methods. Technical data are still limited, nevertheless technical limitations might derive from the step of separation of solvent from plant material, the recovery of solvent that remains adsorbed in the vegetable matrix, the ratio solvent to plant material and, finally, the possibility to reach high concentration in extracts in case non-volatile solvents are used (i.e., vegetable oils).

8. Romano-Hazekamp method is based on the extraction of cannabinoids from pre-heated, dried Cannabis inflorescences using vegetable oils (i.e., olive oil) as solvents. The method can be used for extracting the full range of cannabinoids from the Cannabis plant and it has the advantage of being very safe for consumption. Furthermore, it is considered the most sustainable process from an environmental point of view. (Cannabis Oil: chemical evaluation of an upcoming Cannabis-based medicine, Luigi L Romano, Arno Hazekamp, 2013). The drawbacks of this simple and increasingly popular method are that in order to achieve a satisfactory cannabinoids extraction yield, the extraction with vegetable oils has to take place at 98° C. for a prolonged time (1-2 h) and the quantity of oil to be added as solvent to the plant material is from 4 to 10 times the quantity of plant material, accordingly the level of cannabinoids content in the oil achievable is less than 1%, and more than 50% of volatile mono-terpenes is lost due to prolonged high temperature treatment. Finally, the stability of cannabinoids in the vegetable oil is very low, with a degradation in just two weeks of over 15% and over 20% for storage at 4° C. and ambient temperature respectively, as described by Pacifici.

WO 2018/130682 relates to an enzyme-assisted lipid-based extraction method for obtaining a lipid-soluble extract containing phytocannabinoids and/or terpenoids and/or terpenes. WO2015/070167 describes a method to purify cannabinoids by (i) contacting plant matter containing cannabinoids with a vegetable oil, (ii) heat the obtained lipid extract to fully decarboxylate the cannabinoids, (iii) distillate the decarboxylated cannabinoids.

9. Steam distilling and hydro-distillation are traditional methods for monoterpenes extraction. Steam distilling involves suspending a basket of herb above a vessel of boiling water. The steam passes through the perforated basket and penetrates the plant material. Only volatile compounds such as monoterpenes are soluble in the steam. Hydro distillation is similar to steam distilling except that the herb is placed directly in the boiling water. The methods are not suitable for non-volatile substances such as cannabinoids or heavier terpene compounds.

Another “standard” CBD extraction process, which can also be used for Panakeia, but would likely convert CBGA into CBG during the process. However, if alternative hemp plants are processed with CBDA/CBD content including small amounts of TCHA/THC (<0.3%) were used, this could convert to CBD and THC, respectively, but this process cannot insure that at some point in the extraction/purification/concentration process that THC content will remain <0.3% in all steps, per Federal requirements: step 1 comprises heating chopped Cannabis (2-3 mm) at 100-150° C. for sufficient time to allow decarboxylation. step 2 comprises CO₂ extraction using: a) a coarse powder (the particles are passed through a 3 mm mesh); b) a packing density of 0.3; and c) super-critical conditions of 600 bar at 35° C. for 4 hours, although other combinations of temp and pressure ranging from 10-35° C. and 60-600 bar (both super critical and sub critical conditions) could, it is acknowledged, be used; and step 3 comprises conducting an ethanolic precipitation at −20° C. for 24 hours and removing the waxy material by filtration.
1 Biomass goes into knife crusher with milling (0.2 mm) of product.
2 Placed in Malaxer with T=55° C., pH=5, with distilled water, citric acid, enzymes and carrier oil to form slurry.
3 Placed in Decanter at T=40-55 C for separation, lipid cake extracted.
4 Placed in Clarifier Raw lipid extracts at T=40-55 C to form full spectrum oil.

An alternate process to increase hemp-CBD or Panakeia concentration is: In mixing reactor, Full spectrum oil is mixed with NaOH, water. The mixture is placed in a Centrifugal separator (6 cubic meters per hour), 60 meter head, to separate out alkaline water, discarding exhausted oil. Alkaline water is placed in static mixer for recovery using HCl, for acid solution for flocculation. Acidic water further diluted with distilled neutral water, and filtrated with vibrating screen. Maximum degradation of CBD occurred when samples were stored at 37° C. for 30 days with average values up to 20%. The effect of light was lower, but still significant with averages values up to 15% degradation after 30 days.

For hemp harvest, with fresh dry biomass, ideally curing at 15-20 degrees C. at 60% humidity, its CoA is usually assayed 1-2 weeks after harvest, although it may be tested as long as 12 weeks after harvest, with moisture content of 10-12%, which diminishes over time due to evaporation; higher moisture may result in mold contamination.

In normal Cannabis, CBG content may increase with ageing or heat, with correspondingly less CBGA content, and therefore the “total CBG” content can “creep up” by <12% in the biomass over time because of a greater proportion of CBG. (i.e., a yield of 10 gram (gm) “total CBG” per 100 gm hemp biomass with 95% CBGA content, would be 8.83 gm, or 8.8%, whereas if this CBGA was converted entirely to CBG over time, or with heating, the biomass would be 10 gm, or 10%). Over time, cannabinoid concentration may also trend higher due to greater effects of drying with increased concentration (with a lower biomass). The same principles hold true for calculations of total CBD or THC content based on CBDA/CBD and THCA/THC, respectively.

More commonly the Panakeia plant yield is approx. 6-8% total CBG/CBGA which has no detectable THC or insignificant, but may be detectable, CBD by LC/MS certified lab. Based on this analysis, there was no detectable other cannabinoids. Terpene analysis by Molecular Science Corp: trans-Caryophyllene 0.14 mg/g; Caryophyllene Oxide 0.03 mg/g; alpha-Humulene 0.03 mg/g; Eucalyptol 0.01 mg/g; cis-Nerolidol 0.01 mg/g; Limonene 0.01 mg/g; α-Pinene 0.01 mg/g; Borneol 0.01 mg/g.

The cannabinoid certificate of analysis (CoA) may be obtained based on the entire plant biomass (including leaves, with lower total cannabinoid concentrations) vs. the flower bud content. The “total CBG” content of CoA is calculated as % CBG+(% CBGA×0.877), based on the difference of the molecular weight of the acidic form.

A sample analysis performed by Crest Lab on recently harvested Panakeia reveals: water content 11.7%, with CBGA 6.8%, CBG 0.1%, without detectable THCA, THC, THCV, CBDA, CBD, CBDV, CBC, CBN, heavy metals, pesticides, mycotoxins, below threshold aerobic bacteria, fungi and yeast, bile tolerant gram negative, E. coli, salmonella.

A sample analysis from Americanna Laboratories using a “dry” Panakeia 81 g flower reveals 5.4% moisture, with 6.52% total of available “total” CBG (based on detected CBGA=7.22% which converting by a factor of 0.877 to CBG, in addition to pure CBG=0.192%).

Example 5: A unit dose caplet is formulated with Panekeia Hydrolac 10% (10 mg total cannabinoid), piperine 10 mg, cinnamon 25 mg, PEA 200-600 mg in olive or black seed oil for total of 500-1000 mg.

Example 6: A unit dose gummy is formulated with Panekeia Hydrolac 10% (10 mg), piperine 5%, cinnamon 5%, PEA 200 mg lecithin, HM pectin, citric acid, and sugar, for a total of 1000 mg.

Example 7: A beverage (240 ml) is formulated with Panekeia Hydrolac 10% (30 mg), piperine 15 mg, cinnamon 15 mg, turmeric 20 mg, Vit D 1001 U, butyrate 50 mg, resveratrol 250 mg, quercetin 500 mg, PEA 200 mg, melatonin 2 mg, EDTA 5 mg.

Example 8: Beverage (240 ml) Panekeia Hydrolac 10% (30 mg), piperine 15 mg, cinnamon 15 mg, turmeric 20 mg, Vit D 1001 U, butyrate 150 mg, resveratrol 250 mg, quercetin 500 mg, PEA 400 mg, melatonin 2 mg, Lactobacillus 2 billion colony forming units, using a standard units or portions of flowable powder packet or measured liquid to a functional beverage.

Example 9: A topical ointment: Panekeia 10%, piperine 5%, capsaicin 0.1%, DMSO 5%, Aloe 5%, mango extract 5%, Vit E 2%, alginate 2%, in an ointment base, e.g., www.medisca.com/Pages/ProductDetails.aspx?StockCode=0937&C=B&C2=109

Example 10: Topical ointment: Panekeia 10%, piperine 5-7%, capsaicin 0.1%, Aloe 5%, mango extract 5%, Vit E 2%, alginate linked β-D-mannuronate (M) sodium hydrogel 1% or DMSO 20%.

Example 11: PECSA 80% CBG (no CBGA) full spectrum vape, 2% Beta-Caryophyllene or 2% Limonene from the herbolysis/hydrocan process (for beverages, OTC, and commercialized food products and topical/cosmetic creams which requires quantitative labeling of ingredients).

Example 12: PECSA 30% (˜50/50 CBG/CBGA) full spectrum distillate with pepper, cinnamon (may have varying proportions based on ingestion route and which product it is combined with), with sesame seed oil base from the herbolysis/hydrocan process (for ingestible tablets/capsules, SL spray, gummies, and non-commercialized topicals which requires semiquantitative labeling).

Example 13: PECSA 20% (approx 25% CBG/75% CBGA full spectrum extract oil (may optionally be decolorized) with pepper, cinnamon, with sesame seed oil base (may have varying proportions based on ingestion route and which product it is combined with) from the herbolysis process (only tinctures, non-commercialized food additives (i.e., brownies, gelatin), toothpaste, gum which requires semiquantitative labeling).

Example 14: PECSA with 10-20 mg CBG plus citicoline 300.

Example 15: PECSA with 10-20 mg CBG plus Apoaequorin 10 mg.

Example 16: PECSA with 10-20 mg CBG with Ibuprofen 200 mg.

Example 17: PECSA with 10-20 mg CBG with Naproxen Sodium 220 mg.

Example 18: PECSA containing 5-10 mg CBG, plus acetaminophen 500 mg.

Example 19: PECSA containing 10 mg CBG plus St. John's wort 900 mg (for depression).

Example 20: PECSA containing 10 mg CBG plus 10 mg delta 8 THC and 10 mg CBD.

Example 21: Skin Cream containing PECSA with 100-500 mg CBG (5-10%) plus lipophilic cream.

Example 22: Skin Cream containing PECSA with 100-500 mg CBG (5-10%) in lipophilic base with 2.5% Retinol and Hyaluronic Acid.

Example 23: PECSA containing 5-25 mg CBG plus Cetirizine 5 mg for allergies.

Example 24: PECSA containing 2.5-5 mg CBG plus low dose fluticasone propionate 50 mcg in a nasal spray for daily use in each nostril for Allergic rhinitis.

Example 25: CBG5 mg plus Acetaminophen 650 mg, Dextromethorphan HBr 20 mg, Doxylamine Succinate 12.5 mg, Phenylephrine HCl 10 mg, per 15 ml for cough/cold.

Example 26: CBG5 mg plus Acetaminophen 325 mg, Dextromethorphan 15 mg, Doxylamine Succinate 6.25 mg, in caplets.

Example 27: CBG5 mg plus Acetaminophen 325 mg, dextromethorphan 10 mg, phenylephrine 5 mg; per 15 mL.

Example 28: CBG5 mg plus Acetaminophen 325 mg, dextromethorphan 10 mg, phenylephrine 5 mg, guaifenesin 200 mg; per 15 mL.

Example 29: CBG5 mg, plus Acetaminophen 325 mg, Dextromethorphan HBr 10 mg, Phenylephrine HCl 5 mg.

Example 30: CBG5 mg plus Diphenhydramine HCl 50 mg (in each 30 ml dose cup or 2 tablespoons).

Example 31: PECSA containing 10 mg CBG plus melatonin 3 mg dissolved in tincture of 8-10% alcohol for sleep.

Example 32: CBG5-30 mg plus psyllium fiber 10 g.

Example 33: PECSA containing 5-10 mg CBG, plus alginic acid, sodium bicarbonate, aluminum hydroxide, magnesium carbonate and optionally bismuth subsalicylate.

Example 34: PECSA containing 5-10 mg CBG plus famotidine 10 mg.

Example 35: PECSA containing 5-10 mg CBG plus calcium carbonate 500 mg.

Example 36: PECSA containing 5-10 mg CBG plus Calcium Citrate 650 mg, Vitamin D3 25 mcg (1000 II).

Example 37: PECSA containing 5-10 mg CBG plus Melatonin 5 mg in a fast dissolve tablet.

Example 38: PECSA containing 5-10 mg CBG plus caffeine 330 mg.

Example 39: Energy drink, 355 ml, containing CBG 5-10 mg, caffeine 120 mg, B vitamin mixture, taurine, guarana, ginseng, glucuronolactone, green tea leaf extract, sugar.

Example 40: Iced Tea, CBG5-10 mg, Water, High Fructose Corn Syrup, Citric Acid, Black Tea Powder, Phosphoric Acid, Sodium Benzoate (Preserves Freshness), Potassium Sorbate (Preserves Freshness), calcium disodium EDTA.

Example 41: Chocolate bars 1.5 oz each, with 7.5 mg CBG (5 mg CBG/30 gm).

Example 42: India Pale Ale beer, treated with hops plus Panakeia to stop fermentation, 5-8% ethanol.

Example 43: capsule: CBG 5-10 mg, PEA 400 mg, β-caryophyllene 10 mg, cinnamon 25 mg, pepper (piperine) 10 mg.

Example 44: teabag: Panakeia 500 mg, echinacea 1000 mg.

Example 45: Vaporizer base PECSA full spectrum containing CBG5 mg per dose with propylene glycol and vegetable glycerin.

Example 46: Avape formulation: Panekeia Distillac 95% (10 mg), caryophylline 5 mg, myrcene 5 mg, Eugenol 1 mg, MCT coconut 5 ml.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

EXAMPLE: Orally bioavailable formulations. US 20200397744 (Rehman et al.), provides a composition for oral administration comprising a cannabinoid in combination with a stilbenoid or derivative thereof and a solubility enhancing agent. The process comprises mixing and/or bonding a cannabinoid oil with the stilbenoid or the derivative thereof and generating cocrystals of the cannabinoid and the stilbenoid; lyophilizing the cocrystals to form a powder; adding a solubility enhancing agent to the powder; and formulating the powder into the oral dosage form, e.g., by pressing into tablets. The powder may be generated from the co-crystals of the cannabinoid with pterostilbene or the powder may be formed by subsequently mixing the lyophilized cannabinoid with the stilbenoid before forming the tablet. The tablets provide high bioavailability of the cannabinoid. The stilbenoid or derivative thereof is selected from the group consisting of: resveratrol, piceatannolin, pinosylvin, astringin, piceid, oxyresveratrol, amelopsin A, amelopsin B, vitisin A, combretastatin, combretastatin B-1, isonotholaenic acid, combretastatin A-1, combretastatin A-4, gnetudeistol E, pinostilbene, pterostilbene, isoharpontigenin, gnetudeistol D, 4-methoxyresveratrol, rhaponticin, and rhapontigenin, cavicularin, i-hydroxyphenanthrene and juncusol. The solubility enhancing agent may be a carbohydrate, e.g., mannitol. The use of pterostilbene to ameliorate oxidative stress and improve working memory and compositions containing pterostilbene are described in US 20090069444. Pterostilbene formulations may using cyclodextrins, e.g., U.S. Pat. No. 7,592,328.U.S. Pat. No. 9,474,725, describes compositions infused with lipophilic active agents to provide enhanced bioavailability.

CBG is extracted, and optionally purified in the form of an oil. A reactor vessel is charged with solid pterostilbene and the CBG oil in a suitable crystallization solvent. The solution is heated and then cooled to promote formation of cocrystals. The cocrystals are then collected, washed and dried under vacuum. A solubility-enhancing agent, which may be a carbohydrate such as mannitol for example, is then added to the cocrystals. The cocrystals are then lyophilized to produce a powder. The powder may then be pressed into tablets. The tablets may be provided for oral or sublingual administration.

Alternatively, a base material containing mannitol, silicone dioxide, sorbitol, crospovidone, microcrystalline cellulose, copovidone, sucralose, and flavoring may be mixed with pterostilbene for five minutes at low speed. A heated CBG oil is added to the base material and pterostilbene mixture and mixed at low speed for 5 minutes. The resulting liquid mixture is then freeze-dried. The dried mixture was fed into a granulator to generate a granulated mixture, and then, e.g., tableted into tablets, each containing 2.5, 4, 5, 7.5, or 10 mg of cannabinoid (CBG), 15-50 mg of pterostilbene or other stilbene derivative, 300-800 mg of mannitol, 5-10 mg of sorbitol, 1-2 mg of crospovidone, 1-3 mg of microcrystalline cellulose, 1-2 mg of sucralose, and 50-100 mg of a flavoring agent.

### REFERENCES

Each reference cited herein, including any supplemental information, is expressly incorporated herein by reference in its entirety:
Alam, Safaet, Md Sarker, Moklesur Rahman, Sadia Afrin, Fahmida Tasnim Richi, Chao Zhao, Jin-Rong Zhou, and Isa Naina Mohamed. “Traditional Herbal Medicines, Bioactive Metabolites, and Plant Products Against COVID-19: Update on Clinical Trials and Mechanism of Actions.”Frontiers in Pharmacology 12 (2021):1248.
Alexander S P H, Mathie A, Peters J A. Guide to Receptors and Channels (GRAC), 4th edition. Br J Pharmacol. 2009; 58 (Suppl 1):S1-S254.
Alexander, DeVries, Kigerl, Dahlman, Popovich; Stress exacerbates neuropathic pain via glucocorticoid and NMDA receptor activation; Brain, Behavior, and Immunity, 23 (2009), pp. 851-860
Alexander, Jessica K., A. Courtney DeVries, Kristina A. Kigerl, Jason M. Dahlman, and Phillip G. Popovich. “Stress exacerbates neuropathic pain via glucocorticoid and NMDA receptor activation.”Brain, behavior, and immunity 23, no. 6 (2009): 851-860.
Al-Nimer, Marwan S M, Talar A. Merza Mohammad, and Riyadh A. Alsakeni. “Serum levels of serotonin as a biomarker of newly diagnosed fibromyalgia in women: Its relation to the platelet indices.”Journal of Research in Medical Sciences: The Off. J. of Isfahan University of Medical Sciences 23 (2018).
Al-Nimer, Mohammad, Alsakeni; Serum levels of serotonin as a biomarker of newly diagnosed fibromyalgia in women: Its relation to the platelet indices; Journal of Research in Medical Sciences: The Official Journal of Isfahan University of Medical Sciences, 23 (2018), p. 71
Aminilari, Mahmood, Natasha Kithulegoda, Patricia Strachan, James MacKillop, Li Wang, Sushmitha Pallapothu, Samuel Neumark et al. “Benefits and Concerns regarding Use of Cannabis for Therapeutic Purposes Among People Living with Chronic Pain: A Qualitative Research Study.”Pain Medicine 23, no. 11(2022):1828-1836.
Anand P, Thomas S G, Kunnumakkara A B, Sundaram C, Harikumar K B, Sung B, et al. Biological activities of curcumin and its analogues (Congeners) made by man and Mother Nature. Biochem Pharmacol. 2008; 76:S1590-S1611.
Anand, Jones, Korchev, Bloom, Pacchetti, Anand, Sodergren; CBD effects on TRPV1 signaling pathways in cultured DRG neurons; Journal of Pain Research, 13 (2020), pp. 2269-2278
Anderson, Lyndsey L., Michael Udoh, Declan Everett-Morgan, Marika Heblinski, lain S. McGregor, Samuel D. Banister, and Jonathon C. Arnold. “Olivetolic acid, a cannabinoid precursor in Cannabis sativa, but not CBGA methyl ester exhibits a modest anticonvulsant effect in a mouse model of Dravet syndrome.” Journal of Cannabis research 4, no. 1 (2022): 1-9.
Appiah-Kusi, E., N. Petros, R. Wilson, M. Colizzi, M. G. Bossong, L. Valmaggia, V. Mondelli, P. McGuire, and S. Bhattacharyya. “Effects of short-term cannabidiol treatment on response to social stress in subjects at clinical high risk of developing psychosis.”Psychopharmacology 237(2020): 1121-1130.
Arnold, Hudson, Keck, Auchenbach, Javaras, Hess; Comorbidity of fibromyalgia and psychiatric disorders; J. Clin. Psychiatry, 67(2006), pp. 1219-1225 Arnold, William R., Austin T. Weigle, and Aditi Das. “Cross-talk of cannabinoid and endocannabinoid metabolism is mediated via human cardiac CYP2J2.”Journal of inorganic biochemistry 184(2018):88-99.
Audu, B. S., P. C. Ofojekwu, A. Ujah, and M. N. O. Ajima. “Phytochemical, proximate composition, amino acid profile and characterization of Marijuana (Cannabis sativa L.)”The Journal of Phytopharmacology 3, no. 1(2014):35-43.
Banni S, Di Marzo V. Effect of dietary fat on endocannabinoids and related mediators: Consequences on energy homeostasis, inflammation and mood. Mol Nutr Food Res. 2009; 54:582-S92.
Baraldi, Pier Giovanni, Giulia Saponaro, Allan R. Moorman, Romeo Romagnoli, Delia Preti, Stefania Baraldi, Emanuela Ruggiero et al. “7-Oxo-[1, 4]oxazino [2, 3, 4-ij] quinoline-6-carboxamides as selective CB2 cannabinoid receptor ligands: structural investigations around a novel class of full agonists.” Journal of Medicinal Chemistry 55, no. 14(2012): 6608-6623.
Bayrak, Muharrem. “Metabolic syndrome, depression, and fibromyalgia syndrome prevalence in patients with irritable bowel syndrome: a case-control study.” Medicine 99, no. 23(2020).
Benzinou, Chivre, Ward, Lecoeur, Dina, Lobbens, Durand, Delplanque, Horber, Heude, Balkau, Borch-Johnsen, Jorgensen, Hansen, Pedersen, Meyre, Froguel; Endocannabinoid receptor 1 gene variations increase risk for obesity and modulate body mass index in European populations; Human Molecular Genetics, 17(2008), pp. 1916-1921
Bergamaschi, Queiroz, Chagas, de Oliveira, De Martinis, Kapczinski, Quevedo, Roesler, Schröder, Nardi, Martín-Santos, Hallak, Zuardi, Crippa; Cannabidiol reduces the anxiety induced by simulated public speaking in treatment-naïve social phobia patients; Neuropsychopharmacology, 36 (2011), pp. 1219-1226
Bisht K, Wagner K H, Bulmer A C. Curcumin, resveratrol and flavonoids as anti-inflammatory, cyto- and DNA-protective dietary compounds. Toxicology. 2009 (doi:10.1016/j.tox.2009.11.008).
Black, Stockings, Campbell, Tran, Zagic, Hall, . . . , Degenhardt; Cannabinoids for the treatment of mental disorders and symptoms of mental disorders: a systematic review and meta-analysis; Lancet Psychiatry (2019)
Blake, Wan, Malek, DeAngelis, Diaz, Lao, Chow, O'Hearn; A selective review of medical Cannabis in cancer pain management; Annals of Palliative Medicine, 6 (2017), pp. S215-s222
Blankman, Simon, Cravatt; A comprehensive profile of brain enzymes that hydrolyze the endocannabinoid 2-arachidonoylglycerol; Chemistry & Biology, 14(2007), pp. 1347-1356
Blanton, Barnes, McHann, Bilbrey, Wilkerson, Guindon; Sex differences and the endocannabinoid system in pain; Pharmacology Biochemistry and Behavior, 202 (2021), Article 173107
Boehnke, Gagnier, Matallana, Williams; Substituting Cannabidiol for opioids and pain medications among individuals with fibromyalgia: A large online survey; The Journal of Pain, 22 (2021), pp. 1418-1428
Bolognini D, Costa B, Maione S, Comelli F, Marini P, Di Marzo V, et al. The plant cannabinoid Δ9-tetrahydrocannabivarin can decrease signs of inflammation and inflammatory pain in mice. Br J Pharmacol. 2010; 160:677-687.
Boocock D J, Patel K R, Faust G E, Normolle D P, Marczylo T H, Crowell J A, et al. Quantitation of trans-resveratrol and detection of its metabolites in human plasma and urine by high performance liquid chromatography. J Chromatogr B Analyt Technol Biomed Life Sci.2007; 848:S182-5187.
Borrelli F, Fasolino I, Romano B, et al. Beneficial effect of the non-psychotropic plant cannabinoid cannabigerol on experimental inflammatory bowel disease. Biochemical Pharmacology. 2013; 85(9):1306-1316.
Bruce, Foster, Shattell; Perceived efficacy of medical Cannabis in the treatment of co-occurring health-related quality of life symptoms; Behavioral Medicine, 47(2021), pp. 170-174
Capasso R, Borrelli F, Cascio M G, Aviello G, Huben K, Zjawiony J K, et al. Inhibitory effect of salvinorin A, from Salvia divinorum, on ileitis-induced hypermotility: cross-talk between kappa-opioid and cannabinoid CB(1) receptors. Br J Pharmacol. 2008; 155:5681-5689.
Carvalho, Renata K., Monica L. Andersen, and Renata Mazaro-Costa. “The effects of cannabidiol on male reproductive system: A literature review.”Journal of Applied Toxicology 40, no. 1 (2020):132-150.
Chaves, Bittencourt, Pelegrini; Ingestion of a THC-Rich Cannabis Oil in People with Fibromyalgia: A Randomized, Double-Blind, Placebo-Controlled Clinical Trial; Pain Medicine, 21(2020), pp. 2212-2218
Chen, N.; Zhou, M.; Dong, X.; Qu, J.; Gong, F.; Han, Y. et al. Epidemiological and clinical characteristics of 99 cases of 2019 novel coronavirus pneumonia in Wuhan, China: a descriptive study. Lancet 2020, 395(10223), 507-513.
Chicca A, Raduner S, Pellati F, Strompen T, Altmann K H, Schoop R, et al. Synergistic immunomo pharmacological effects of N-alkylamides in Echinacea purpurea herbal extracts. Int Immunopharmacol. 2009; 9:5850-S858.
Colasanti B K. A comparison of the ocular and central effects of tetrahydrocannabinol and cannabigerol. Journal of Ocular Pharmacology and Therapeutics. 1990; 6(4):259-269.
Console-Bram, Brailoiu, Brailoiu, Sharir, Abood; Activation of GPR18 by cannabinoid compounds: A tale of biased agonism; British Journal of Pharmacology, 171 (2014), pp. 3908-3917
Coronaviridae Study Group of the International Committee on Taxonomy of V The species severe acute respiratory syndrome-related coronavirus: classifying 2019-nCoV and naming it SARS-CoV-2. Nat Microbiol 2020, 5(4), 536-544.
Cravatt, Giang, Mayfield, Boger, Lerner, Gilula; Molecular characterization of an enzyme that degrades neuromodulatory fatty-acid amides; Nature, 384(6604)(1996), pp. 83-87, 10.1038/384083a0
Cremon, Stanghellini, Barbaro, Cogliandro, Bellacosa, Santos, . . . , Barbara; Randomised clinical trial: The analgesic properties of dietary supplementation with palmitoylethanolamide and polydatin in irritable bowel syndrome; Alimentary Pharmacology & Therapeutics, 45 (2017), pp. 909-922
Crippa, Derenusson, Ferrari, Wichert-Ana, Duran, Martin-Santos, . . . , Hallak; Neural basis of anxiolytic effects of cannabidiol (CBD) in generalized social anxiety disorder: A preliminary report; Journal of Psychopharmacology, 25 (2011), pp. 121-130
Crocq; History of Cannabis and the endocannabinoid system; Dialogues in Clinical Neuroscience, 22 (2020), pp. 223-228
Dalle, Carbonare, Del, Giudice, Stecca, Colavito, . . . , Leon; A saturated N-Acylethanolamine other than N-Palmitoyl ethanolamine with anti-inflammatory properties: A neglected story . . . ; Journal of Neuroendocrinology, 20(2008), pp. 26-34
De Petrocellis L, Di Marzo V. Non-CB(1), Non-CB(2) receptors for endocannabinoids, plant cannabinoids, and synthetic cannabimimetics: focus on G-protein-coupled receptors and transient receptor potential channels. J Neuroimmune Pharmacol. 2010; 5:103-121.
De, Almeida, Devi; Diversity of molecular targets and signaling pathways for CBD; Pharmacology Research & Perspectives, 8 (2020), Article e00682
Deb, Subrata, and Scott Arrighi. “Potential effects of COVID-19 on cytochrome P450-mediated drug metabolism and disposition in infected patients.”European Journal of Drug Metabolism and Pharmacokinetics (2021):1-19.
Devane, Dysarz, Johnson, Melvin, Howlett; Determination and characterization of a cannabinoid receptor in rat brain; Mol. Pharm., 34 (1988), pp. 605-613
Di Marzo V, Bisogno T, Petrocellis L. Endocannabinoids and related compounds: walking back and forth between plant natural products and animal physiology. Chem Biol. 2007; 14:5741-5756.
Di Marzo V, Sepe N, De Petrocellis L, Berger A, Crozier G, Fride E, et al. Trick or treat from food endocannabinoids. Nature. 1998; 396:5636-5637.
Di Marzo V. Targeting the endocannabinoid system: to enhance or reduce? Nat Rev Drug Discov. 2008; 7:5438-5455.
DiTomaso E, Beltramo M, Piomelli D. Brain cannabinoids in chocolate. Nature. 1996; 382:5677-S678.
Dina, Levine, Green; Enhanced cytokine-induced mechanical hyperalgesia in skeletal muscle produced by a novel mechanism in rats exposed to unpredictable sound stress; European Journal of Pain, 15(2011), pp. 796-800
Dinh, Carpenter, Leslie, Freund, Katona, Sensi, Kathuria, Piomelli; Brain monoglyceride lipase participating in endocannabinoid inactivation; Proceedings of the National Academy of Sciences of the United States of America, 99 (2002), pp. 10819-10824
Doohan, Peter T., Lachlan D. Oldfield, Jonathon C. Arnold, and Lyndsey L. Anderson. “Cannabinoid interactions with cytochrome P450 drug metabolism: a full-spectrum characterization.”The AAPS Journal 23, no. 4(2021):91.
Drosten, C., et al., Identification of a novel coronavirus in patients with severe acute respiratory syndrome. N. Engl. J. Med 2003, 348, 1967-1976.
DuPont M S, Day A J, Bennett R N, Mellon F A, Kroon P A. Absorption of kaempferol from endive, a source of kaempferol-β-glucuronide, in humans. Eur J Clin Nutr. 2004; 58:5947-5954.
Dvora Izgelov, Abraham J Domb, Amnon Hoffman, The effect of piperine on oral absorption of cannabidiol following acute vs. chronic administration, doi:10.1016/j.ejps. 2020.105313. Epub 2020 Mar. 19, 2020 May 30; 148:105313. PMID: 32198013DOI:10.1016/j.ejps.2020.105313
El-Ghiaty, Mahmoud A., Sherif M. Shoieb, and Ayman O S El-Kadi. “Cytochrome P450-mediated drug interactions in COVID-19 patients: Current findings and possible mechanisms.”Medical hypotheses 144(2020):110033.
Elmes, Kaczocha, Berger, Leung, Ralph, Wang, . . . , Deutsch; Fatty acid-binding proteins (FABPs) are intracellular carriers for Δ9-tetrahydrocannabinol (THC) and cannabidiol (CBD); The Journal of Biological Chemistry, 290 (2015), pp. 8711-8721
Ethan B Russo, Carrie Cuttler, Ziva D Cooper, Amanda Stueber, Venetia L Whiteley, Michelle Sexton, Survey of Patients Employing Cannabigerol-Predominant Cannabis Preparations: Perceived Medical Effects, Adverse Events, and Withdrawal Symptoms, Cannabis Cannabinoid Res, 2021 Sep. 27. doi: 10.1089/can.2021.0058. PMID:34569849 D01:10.1089/can.2021.0058
Faouzi, Malika, Clay Wakano, Mahealani K. Monteilh-Zoller, Ram P. Neupane, John G. Starkus, Jayanti Bhandari Neupane, Aaron J. Cullen, Brandon E.
Johnson, Andrea Fleig, and Reinhold Penner. “Acidic cannabinoids suppress proinflammatory cytokine release by blocking store-operated calcium entry.” Function 3, no. 4(2022):zgac033.
Farha M A, El-Halfawy O M, Gale R T, et al. Uncovering the hidden antibiotic potential of Cannabis. ACS Infect Dis. 2020; 6(3):338-346.
Fichna J, Schicho R, Andrews C N, Bashashati M, Klompus M, McKay D M, et al. Salvinorin A inhibits colonic transit and neurogenic ion transport in mice by activating kappa-opioid and cannabinoid receptors. Neurogastroenterol Motil. 2009; 21:S1326-Sel28.
Fitzcharles, Baerwald, Ablin, Häuser; Efficacy, tolerability and safety of cannabinoids in chronic pain associated with rheumatic diseases (fibromyalgia syndrome, back pain, osteoarthritis, rheumatoid arthritis); Der Schmerz, 30 (2016), pp. 47-61
Fitzcharles, Rampakakis, Sampalis, Shir, Cohen, Starr, Häuser; Use of medical Cannabis by patients with fibromyalgia in Canada after Cannabis legalisation: A cross-sectional study; Clinical and Experimental Rheumatology, 39 (130)(2021), pp. 115-119
Fiz, Durán, Capella, Carbonellà, Farré; Cannabis Use in Patients with Fibromyalgia: Effect on Symptoms Relief and Health-Related Quality of Life; PLOS One, 6(4)(2011), p.e18440, 10.1371/journal.pone.0018440
Freeman, Craft, Wilson, Stylianou, ElSohly, Di Forti, Lynskey; Changes in delta-9-tetrahydrocannabinol (THC) and cannabidiol (CBD) concentrations in Cannabis over time: Systematic review and meta-analysis; Addiction, 116 (2021), pp. 1000-1010
Gaisey, & Narouze. (2021). Dronabinol (Marinol®) S. N. Narouze Cannabinoids and pain (105-107). Cham: Springer International Publishing.
Garcea G, Jones D J, Singh R, Dennison A R, Farmer P B, Sharma R A, et al. Detection of curcumin and its metabolites in hepatic tissue and portal blood of patients following oral administration. Br J Cancer.2004; 90:S1011-S1015.
Gertsch J, Leonti M, Raduner S, Racz I, Chen J Z, Xie X Q, et al. Beta-caryophyllene is a dietary cannabinoid. Proc Natl Acad Sci USA. 2008; 105:59099-59104.
Gertsch J, Raduner S, Altmann K H. New natural noncannabinoid ligands for cannabinoid type-2 (CB2) receptors. J Recept Signal Transduct Res. 2006; 26:5709-5730.
Gertsch J. Anti-inflammatory cannabinoids in diet: towards a better understanding of CB(2) receptor action? Commun Integr Biol. 2008; 1:S26-S28.
Gertsch J. How scientific is the science in ethnopharmacology: historical perspectives and epistemiological problems. J Ethnopharmacol. 2009; 122:5177-S183.
Gertsch, Jürg, Roger G. Pertwee, and Vincenzo Di Marzo. “Phytocannabinoids beyond the Cannabis plant-do they exist?.” British journal of pharmacology 160, no. 3 (2010):523-529.
Giorgi, Bongiovanni, Atzeni, Marotto, Salaffi, Sarzi-Puttini; Adding medical Cannabis to standard analgesic treatment for fibromyalgia: a prospective observational study; Clinical Experimental Rheumatology, 38 (123)(2020), pp. 53-59
Gonsiorek, Lunn, Fan, Narula, Lundell, Hipkin; Endocannabinoid 2-arachidonyl glycerol is a full agonist through human type 2 cannabinoid receptor: Antagonism by anandamide; Molecular Pharmacology, 57(2000), pp. 1045-1050
Granja, Aitor G., Francisco Carrillo-Salinas, Alberto Pagani, María Gómez-Cañas, Roberto Negri, Carmen Navarrete, Miriam Mecha et al. “A cannabigerol quinone alleviates neuroinflammation in a chronic model of multiple sclerosis.”Journal of Neuroimmune Pharmacology 7 (2012):1002-1016.
Guerrero-Alba, Barragán-Iglesias, González-Hernández, Valdez-Moráles, Granados-Soto, Condés-Lara, . . . , Marichal-Cancino; Some prospective alternatives for treating pain: The endocannabinoid system and its putative receptors GPR18 and GPR55; Frontiers in Pharmacology (2019), p. 9
Habib, Artul; Medical Cannabis for the treatment of fibromyalgia; Journal of Clinical Rheumatology, 24 (2018), pp. 255-258
Hanus̆, Meyer, Muñoz, Taglialatela-Scafati, Appendino; Phytocannabinoids: A unified critical inventory; Natural Product Reports, Vol. 33, The Royal Society of Chemistry (2016), pp. 1357-1392
Harrison, Simpson; Nabilone (Cesamet); S. N. Narouze (Ed.), Cannabinoids and pain, Springer International Publishing, Cham (2021), pp. 109-112
Hausman-Kedem, Menascu, Kramer; Efficacy of CBD-enriched medical Cannabis for treatment of refractory epilepsy in children and adolescents— An observational, longitudinal study; Brain and Development, 40 (2018), pp. 544-551
Hill, Shirley Y., Robert Schwin, Barbara Powell, and Donald W. Goodwin. “State-dependent effects of marihuana on human memory.” Nature 243, no. 5404(1973):241-242.
Hong, Fan, Kemmerer, Evans, Li, Wiley; Reciprocal changes in vanilloid (TRPV) and endocannabinoid (CB1) receptors contribute to visceral hyper algesia in the water avoidance stressed rat; Gut, 58(2009), pp. 202-210, 2008/10/20
Hong, Zheng, Wu, Snider, Owyang, Wiley; Corticosterone mediates reciprocal changes in CB 1 and TRPV1 receptors in primary sensory neurons in the chronically stressed rat. In; Gastroenterology, 140(2011), pp. 627-637. e624, 2010/11/09
Huang, C.; Wang, Y.; Li, X.; Ren, L.; Zhao, J.; Hu, Y.; et al. Clinical features of patients infected with 2019 novel coronavirus in Wuhan, China. Lancet 2020, 395(10223), 497-506.
Huff, Hannah C., Archit Vasan, Pritam Roy, Aayush Kaul, Emad Tajkhorshid, and Aditi Das. “Differential Interactions of Selected Phytocannabinoids with Human CYP2D6 Polymorphisms.” Biochemistry 60, no. 37(2021):2749-2760.
Hui, D. S.; I Azhar, E.; Madani, T. A.; Ntoumi, F.; Kock, R.; Dar, O.; Ippolito, G.; Mchugh, T. D.; Memish, Z. A.; Drosten, C.; Zumla, A.; Petersen, E. The continuing 2019-nCoV epidemic threat of novel coronaviruses to global health The latest 2019 novel coronavirus outbreak in Wuhan, China. Int J of Infec Dis 2020, 91, 264-266.
Huynh Thi Phuong Loan, Nguyen Thi Thanh Hai, Dr. Le Trung Hieu, Prof. Tran Thai Hoa, Dr. Bui Thi Phuong Thuy, Prof. Duong Tuan Quang, Dr. Nguyen Thanh Triet, Evaluation of the Inhibitory Activities of COVID-19 of Melaleuca cajuputi Oil Using Docking Simulation, Chemistry Select 2020, 5, 6312-6320, chemistry-europe.onlinelibrary.wiley.com/doi/10.1002/slct.202000822.
Ibeas, Bih, Chen, Nunn, Bazelot, Dallas, Whalley; Molecular targets of Cannabidiol in neurological disorders; Neurotherapeutics, 12 (2015), pp. 699-730
Ibrahim, Porreca, Lai, Albrecht, Rice, Khodorova, Davar, Makriyannis, Vanderah, Mata, Malan; CB2 cannabinoid receptor activation produces antinociception by stimulating peripheral release of endogenous opioids; Proceedings of the National Academy of Sciences of the United States of America, 102 (2005), pp. 3093-3098
Izgelov, Dvora, Abraham J. Domb, and Amnon Hoffman. “The effect of piperine on oral absorption of cannabidiol following acute vs. chronic administration.” European Journal of Pharmaceutical sciences 148(2020):105313.
Jennings, Okine, Olango, Roche, Finn; Repeated forced swim stress differentially affects formalin-evoked nociceptive behaviour and the endocannabinoid system in stress normo-responsive and stress hyper-responsive rat strains; Progress in Neuro-Psychopharmacology and Biological Psychiatry, 64(2016), pp. 181-189
Jesus, Redivo, Gasparin, Sotomaior, de Carvalho, Genaro, . . . , da Cunha; Cannabidiol attenuates mechanical allodynia in streptozotocin-induced diabetic rats via serotonergic system activation through 5-HT1A receptors; Brain Research, 1715 (2019), pp. 156-164
Ji, Nackley, Huh, Terrando, Maixner; Neuroinflammation and central sensitization in chronic and widespread pain; Anesthesiology, 129 (2018), pp. 343-366
Jiang, Nie, Li, Zhang; Association of cannabinoid type 1 receptor and fatty acid amide hydrolase genetic polymorphisms in Chinese patients with irritable bowel syndrome; Journal of Gastroenterology and Hepatology, 29 (2014), pp. 1186-1191
Juhasz, Lazary, Chase, Pegg, Downey, Toth, Stones, Platt, Mekli, Payton, Anderson, Deakin, Bagdy; Variations in the cannabinoid receptor 1 gene predispose to migraine; Neuroscience Letters, 461(2009), pp. 116-120
Kandemir, Kevser, Merve Tomas, David Julian McClements, and Esra Capanoglu. “Recent advances on the improvement of quercetin bioavailability.” Trends in Food Science & Technology 119 (2022):192-200.
Karl, Tim, David Cheng, Brett Garner, and Jonathon C. Arnold. “The therapeutic potential of the endocannabinoid system for Alzheimer's disease.” Expert opinion on therapeutic targets 16, no. 4(2012): 407-420.
Karsak, Meliha, Martine Cohen-Solal, Jan Freudenberg, Agnes Ostertag, Caroline Morieux, Uwe Kornak, Julia Essig et al. “Cannabinoid receptor type 2 gene is associated with human osteoporosis.” Human molecular genetics 14, no. 22 (2005): 3389-3396.
Karschner, Darwin, McMahon, Liu, Wright, Goodwin, Huestis; Subjective and physiological effects after controlled Sativex and oral THC administration; Clinical Pharmacology and Therapeutics, 89 (2011), pp. 400-407
Kaufmann, Schelling, Eisner, Richter, Krauseneck, Vogeser, Hauer, Campolongo, Chouker, Beyer, Thiel; Anandamide and neutrophil function in patients with fibromyalgia; Psychoneuroendocrinology, 33 (2008), pp. 676-685
Kaur, Singh, Singh, Bhatti, Bhatti; Ameliorative effect of imperatorin in chemically induced fibromyalgia: Role of NMDA/NFkB mediated downstream signaling; Biochemical Pharmacology, 166(2019), pp. 56-69
King A R, Dotsey E Y, Lodola A, Jung K M, Ghomian A, Qiu Y, et al. Discovery of potent and reversible monoacylglycerollipase inhibitors. Chem Biol. 2009; 16:1045-1052.
Kiralan, M., V. GUI, and S. Metin Kara. “Fatty acid composition of hempseed oils from different locations in Turkey.” Spanish Journal of Agricultural Research 8, no. 2 (2010): 385-390.
Kiso, Watabiki, Sekizawa; ASP8477, a fatty acid amide hydrolase inhibitor, exerts analgesic effects in rat models of neuropathic and dysfunctional pain. In; European Journal of Pharmacology, 881(2020), p. 173194
Klooker, Leliefeld, Van Den Wijngaard, Boeckxstaens; The cannabinoid receptor agonist delta-9-tetrahydrocannabinol does not affect visceral sensitivity to rectal distension in healthy volunteers and IBS patients; Neurogastroenterology and Motility, 23 (2011), pp. 30-e32
Kogan, Natalya M., Yarden Lavi, Louise M. Topping, Richard Williams, Fiona E. McCann, Zhanna Yekhtin, Marc Feldmann, Ruth Gallily, and Raphael Mechoulam. “Novel CBG Derivatives Can Reduce Inflammation, Pain and Obesity.” Molecules 26, no. 18 (2021):5601.
Korem, Duek, Xu, Harpaz-Rotem, Pietrzak; Cannabinoid receptor 1 rs1049353 variant, childhood abuse, and the heterogeneity of PTSD symptoms: Results from the National Health and resilience in veterans study; Chronic Stress, 5 (2021), 24705470211011075
Korte G, Dreiseitel A, Schreier P, Oehme A, Locher A, Goeran H, et al. An examination of anthocyanins' and anthocyanidins' affinity for cannabinoid receptors. J Med Food. 2009; 12:S1407-S1410.
Korte G, Dreiseitel A, Schreier P, Oehme A, Locher S, Geiger S, et al. Tea catechins' affinity for human cannabinoid receptors. Phytomedicine. 2010; 17:S19-S22.
Krcevski-Skvarc, Wells, Häuser; Availability and approval of Cannabis-based medicines for chronic pain management and palliative/supportive care in Europe: A survey of the status in the chapters of the European pain federation; European Journal of Pain, 22 (2018), pp. 440-454
Ksiazek, T. G. et al.; A novel coronavirus associated with severe acute respiratory syndrome. N. Engl. J. Med 2003, 348, 1953-1966.
Kwilasz, Abdullah, Poklis, Lichtman, Negus; Effects of the fatty acid amide hydrolase inhibitor URB597 on pain-stimulated and pain-depressed behavior in rats; Behavioural Pharmacology, 25(2014), pp. 119-129
Landis-Piwowar K R, Dou Q P. Polyphenols: biological activities, molecular targets, and the effect of methylation. Curr Mol Pharmacol. 2008; 1:5233-5243.
Laprairie, Bagher, Kelly, Denovan-Wright; Cannabidiol is a negative allosteric modulator of the cannabinoid CB receptor; British Journal of Pharmacology, 172 (2015), pp. 4790-4805
Lauckner, Jensen, Chen, Lu, Hille, Mackie; GPR55 is a cannabinoid receptor that increases intracellular calcium and inhibits M current; Proceedings of the National Academy of Sciences of the United States of America, 105(2008), pp. 2699-2704
Lenoir, Camille, Jean Terrier, Yvonne Gloor, Fransois Curtin, Victoria Rollason, Jules Alexandre Desmeules, Youssef Daali, Jean-Luc Reny, and Caroline
Flora Samer. “Impact of SARS-CoV-2 Infection (COVID-19) on Cytochromes P450 Activity Assessed by the Geneva Cocktail.” Cliical Pharmacology& Therapeutics 110, no. 5(2021):1358-1367.
Leonti M, Casu L, Raduner S, Cottiglia F, Floris C, Altmann K-H, et al. Falcarinol is a covalent cannabinoid CB1 receptor antagonist and induces pro-allergic effects in skin. Biochem Pharmacol. 2010 doi:10.1016/j.bcp.2010.02.015 accepted.
Lin, Ko, Chow, Hsiao, Liu, Wang, Chen; Salivary cortisol is associated with cognitive changes in patients with fibromyalgia; Sci. Reports, 11 (2021), p. 1311
Lomazzo, Bindila, Remmers, Lerner, Schwitter, Hoheisel, Lutz; Therapeutic potential of inhibitors of endocannabinoid degradation for the treatment of stress-related hyperalgesia in an animal model of chronic pain; Neuropsychopharmacology, 40 (2015), pp. 488-501
Lu, Ogdie, Jürvelin, Moilanen, Loo, McCracken, McGough, Yang, Peltonen, Nelson, Cantor, Smalley; Association of the cannabinoid receptor gene (CNR1) with ADHD and post-traumatic stress disorder. American journal of medical genetics; Part B, Neuropsychiatric Genetics: the official publication of the International Society of Psychiatric Genetics, 147B (2008), pp. 1488-1494
Magalhães, Clarissa B., Douglas R. Riva, Leonardo J. DePaula, Aline Brando-Lima, Vera Lúcia G. Koatz, José Henrique Leal-Cardoso, Walter A. Zin, and Débora S. Faffe. “In vivo anti-inflammatory action of eugenol onlipopolysaccharide-inducedlung injury.” J. of Applied Physiology 108, no. 4 (2010): 845-851.
Magalhães, Isadora de Fátima Braga, Kátia da Silva Calabrese, Ana Letícia Marinho Figueiriêdo, Ana Lucia Abreu-Silva, and Fernando Almeida-Souza. “The Use of Plants' Natural Products in Breast Cancer: Have We Already Found the New Anticancer Drug?.” In Breast Cancer-Evolving Challenges and Next Frontiers. IntechOpen, 2021.
Malabadi, Ravindra B., Neelambika T. Meti, and Raju K. Chalannavar. “Role of herbal medicine for controlling coronavirus (SARS-CoV-2) disease (COVID-19).” Internal Journal of Research and Scientific Innovations 8, no. 2 (2021): 135-165.
Malabadi, Ravindra B., P. Kolkar, T. Meti, and K. Chalannavar. “Traditional Herbal Folk Medicine Used For Controlling Corona Virus (SARS-COV-2) DISEASE (COVID-19).” Internal Journal of Innovation Scientific Research and Review. 2021i3, no. 7:1507-1517.
Maor Y, Morowitz M, Gallily R, Mechoulam R (2005) Cannabigerol-dimethyl heptyl (CBG-DMH), a synthetic cannabinoid with hypotensive and vasorelaxant properties. Presented at the symposium of the International Cannabinoid Research Society, Clearwater, FL, June 2005
Marcu, Jahan. “The legalization of cannabinoid products and standardizing Cannabis-drug development in the United States: a brief report.” Dialogues in Clinical Neuroscience (2020).
Markuu, P. Sleep research in 2020: COVID-19-related sleep disorders. The Lancet Neurology 2021, 20(1), 15-17.
Masataka; Anxiolytic effects of repeated Cannabidiol treatment in teenagers with social anxiety disorders; Frontiers in Psychology, 10 (2019), p. 2466
Maurelli S, Bisogno T, De Petrocellis L, Di Luccia A, Marino G, Di Marzo V. Two novel classes of neuroactive fatty acid amides are substrates for mouse neuroblastoma 'anandamide amidohydrolase. FEBS Lett. 1995; 377:582-S86.
Mazza; Medical Cannabis for the treatment of fibromyalgia syndrome: A retrospective, open-label case series; J. Cannabis Research, 3 (2021), p. 4
McGilveray; Pharmacokinetics of cannabinoids; Pain Research & Management, 10 (2005), Article 242516
Mechoulam R. Interview with Prof. Raphael Mechoulam, codiscoverer of THC. Interview by Stanley Einstein. Int J Addict. 1986; 21:S579-S587.
Millar, Sophie Anne, Ryan Francis Maguire, Andrew Stephen Yates, and Saoirse Elizabeth O'Sullivan. “Towards better delivery of cannabidiol (CBD).” Pharmaceuticals 13, no. 9(2020):219.
Millar, Stone, Yates, O'Sullivan; A systematic review on the pharmacokinetics of Cannabidiol in humans; Frontiers in Pharmacology, 9 (2018), p. 1365
Mishra, Raghav Kumar, and Shio Kumar Singh. “Reproductive effects of lipid soluble components of Syzygium aromaticum flower bud in male mice.” Journal of Ayurveda and integrative medicine 4, no. 2 (2013):94.
Morales, Hurst, Reggio; Molecular targets of the phytocannabinoids: a complex picture; Prog. Chem. of Organic Natural Prod., 103 (2017), pp. 103-131
Mota, Sumner, Lowe, Neumeister, Uddin, Aiello, . . . , Pietrzak; The rsl049353 polymorphism in the CNR1 gene interacts with childhood abuse to predict posttraumatic threat symptoms; The Journal of Clinical Psychiatry, 76 (2015), pp. e1622-el623
Muller, Morales, Reggio; Cannabinoid ligands targeting TRP channels; Frontiers in Molecular Neuroscience, 11(2019), p. 487
Munro, Thomas, Abu-Shaar; Molecular characterization of a peripheral receptor for cannabinoids; Nature, 365 (1993), pp. 61-65
Nachnani, Rahul, Wesley M. Raup-Konsavage, and Kent E. Vrana. “The Pharmacological Case for Cannabigerol.” Journal of Pharmacology and Experimental Therapeutics 376, no. 2(2021):204-212.
Nadolska, Krystyna, and Roman Goś. “Possibilities of applying cannabinoids' in the treatment of glaucoma.” Klinika oczna T10, no. 7-9 (2008): 314-317.
Nakano, Yukako, Masataka Tajima, Erika Sugiyama, Vilasinee Hirunpanich Sato, and Hitoshi Sato. “Development of a novel nanoemulsion formulation to improve intestinal absorption of cannabidiol.” Medical Cannabis and Cannabinoids 2, no. 1(2019): 35-42.
Nelson, Kathryn M., Jonathan Bisson, Gurpreet Singh, James G. Graham, Shao-Nong Chen, J. Brent Friesen, Jayme L. Dahlin, Matthias Niemitz, Michael A. Walters, and Guido F. Pauli. “The essential medicinal chemistry of cannabidiol (CBD).”Journal of medicinal chemistry 63, no. 21(2020): 12137-12155.
Nguyen, Long Chi, Dongbo Yang, Vlad Nicolaescu, Thomas Best, Shaonong Chen, J. Brent Friesen, Nir Drayman et al. “Cannabidiol inhibits SARS-CoV-2 replication and promotes the host innate immune response.” bioRxiv(2021).
O'Sullivan; Cannabinoids go nuclear: Evidence for activation of peroxisome proliferator-activated receptors; British Journal of Pharmacology, 152 (2007), pp. 576-582
Oboh, Ganiyu, Ifeoluwa A. Akinbola, Ayokunle O. Ademosun, David M. Sanni, Oluwatoyin V. Odubanjo, Tosin A. Olasehinde, and Sunday I. Oyeleye. “Essential oil from clove bud (Eugenia aromatica Kuntze) inhibit key enzymes relevant to the management of type-2 diabetes and some pro-oxidant induced lipid peroxidation in rats pancreas in vitro.” Journal of oleo science (2015): essl4274.
Oesch S, Gertsch J. Cannabis receptor ligands as potential anticancer agents—high hopes for new therapies? J Pharm Pharmacol. 2009; 61:5839-5853.
Oh, Parikh, Khurana, Cognata Smith, Vetticaden; Effect of food on the pharmacokinetics of dronabinol oral solution versus dronabinol capsules in healthy volunteers; Clinical Pharmacology: Advances and Applications, 9 (2017), pp. 9-17
Ohno-Shosaku, Maejima, Kano; Endogenous cannabinoids mediate retrograde signals from depolarized postsynaptic neurons to presynaptic terminals; Neuron, 29 (2001), pp. 729-738
Orrego-González E, Londono-Tobón L, Ardila-González J, Polania-Tovar D, Valencia-Cárdenas A, Velez-Van Meerbeke A. Cannabinoid effects on experimental colorectal cancer models reduce aberrant crypt foci (Acd) and tumor volume: a systematic review. Evidence-Based Complementary and Alternative Medicine. 2020; 2020:1-13.
O'Sullivan S E. Cannabinoids go nuclear: evidence for activation of peroxisome proliferator-activated receptors. Br J Pharmacol. 2007; 152:576-582.
Pagano E, Montanaro V, Di Girolamo A, Pistone A, Altieri V, Zjawiony J K, Izzo A A, Capasso R. Effect of Non-psychotropic Plant-derived Cannabinoids on Bladder Contractility: Focus on Cannabigerol. Nat Prod Commun. 2015 June; 10(6):1009-12. PMID:26197538.
Paland, Nicole, Antonina Pechkovsky, Miran Aswad, Haya Hamza, Tania Popov, Eduardo Shahar, and Iga1 Louria-Hayon. “The Immunopathology of COVID-19 and the Cannabis Paradigm.” Frontiers in Immunology 12 (2021):327.
Pan, Banks, Fasold, Bluth, Kastin; Transport of brain-derived neurotrophic factor across the blood-brain barrier; Neuropharmacology, 37(1998), pp. 1553-1561
Park, Choi, Cho, Lee, Kim, Choi, Chung; Cannabinoid receptor 1 gene polymorphism and irritable bowel syndrome in the Korean population: A hypothesis-generating study; Journal of Clinical Gastroenterology, 45 (2011), pp. 45-49
Patel, Hillard; Pharmacological evaluation of cannabinoid receptor ligands in a mouse model of anxiety: Further evidence for an anxiolytic role for endogenous cannabinoid signaling; The Journal of Pharmacology and Experimental Therapeutics, 318 (2006), pp. 304-311
Patsenker, Eleonora, and Felix Stickel. “Cannabinoids in liver diseases.” Clinical Liver Disease 7, no. 2 (2016):21.
Peiris, J. S.; Lai, S. T.; Poon, L. L.; Guan, Y.; Yam, L. Y.; Lim, W.; Nicholls, J.; Yee, W. K.; Yan, W. W.; Cheung, M. T.; Cheng, V. C.; Chan, K. H.; Tsang, D. N.; Yung, R. W.; Ng, T. K.; Yuen. K. Y. Coronavirus as a possible cause of severe acute respiratory syndrome. Lancet 2003, 361, 1319-1325.
Pertwee R G. Cannabinoid pharmacology: the first 66 years. Br J Pharmacol. 2006; 147:5163-S171.
Pertwee R G. Emerging strategies for exploiting cannabinoid receptor agonists as medicines. Br J Pharmacol. 2009; 156:5397-5411.
Pertwee R G. Receptors targeted by synthetic cannabinoid receptor agonists and antagonists. Curr Med Chem. 2010; 17:1360-1381.
Pertwee R G. The therapeutic potential of drugs that target cannabinoid receptors or modulate the tissue levels or actions of endocannabinoids. AAPS J. 2005; 7:E625-E654.
Perucca, Bialer; Critical aspects affecting Cannabidiol Oral bioavailability and metabolic elimination, and related clinical implications; CNS Drugs, 34 (2020), pp. 795-800
Pieracci, Ylenia, Roberta Ascrizzi, Valentina Terreni, Luisa Pistelli, Guido Flamini, Laura Bassolino, Flavia Fulvio, Massimo Montanari, and Roberta Paris. “Essential Oil of Cannabis sativa L: Comparison of Yield and Chemical Composition of 11 Hemp Genotypes.” Molecules 26, no. 13 (2021): 4080;
Placzek, Kate, “The Distinction Between DHEA and DHEA-S & Why Both Are Important For a Healthy Brain” (2017), www.zrtlab.com/blog/archive/difference-dhea-dheas-brain-health/
Poyatos, L., Pérez-Acevedo, A. P., Papaseit, E., Pérez-Mañá, C., Martin, S., Hladun, O., Siles, A., Torrens, M., Busardo, F. P. and Farré, M., 2020. Oral administration of Cannabis and Δ-9-tetrahydrocannabinol (THC) preparations: a systematic review. Medicina, 56(6), p. 309.
Prather P L, Seely K A, Levi M S. Notice of retraction. J Pharmacol Exp Ther. 2009; 331:1147.
Pryce, Gareth, Zubair Ahmed, Deborah J R Hankey, Samuel J. Jackson, J. Ludovic Croxford, Jennifer M. Pocock, Catherine Ledent et al. “Cannabinoids inhibit neurodegeneration in models of multiple sclerosis.” Brain 126, no. 10 (2003): 2191-2202.
Raduner S, Majewska A, Chen J Z, Xie X Q, Hamon J, Faller B, et al. Alkylamides from Echinacea are a new class of cannabinomimetics. Cannabinoid type-2 receptor-dependent and independent immunomodulatory effects. J Biol Chem. 2006; 281:S14192-SiS206.
Radwan, Mohamed M., Suman Chandra, Shahbaz Gul, and Mahmoud A. ElSohly. “Cannabinoids, Phenolics, Terpenes and Alkaloids of Cannabis.” Molecules 26, no. 9 (2021):2774.
Rahimi, R., Ghiasi, S., Azimi, H., Fakhari, S., & Abdollahi, M. (2010). A review of the herbal phosphodiesterase inhibitors; Future perspective of new drugs. Cytokine, 49(2), 123-129.doi:10.1016/j.cyto.2009.11.005
Ramirez, Cristina Lujan, Maria Alejandra Fanovich, and Maria Sandra Churio. “Cannabinoids: extraction methods, analysis, and physicochemical characterization.” In Studies in natural products chemistry, vol. 61, pp. 143-173. Elsevier, 2019.
Rey, Purrio, Viveros, Lutz; Biphasic effects of cannabinoids in anxiety responses: CB and GABA(B) receptors in the balance of GABAergic and glutamatergic neurotransmission; Neuropsychopharmacology: official publication of the Am. Coll. Neuropsychopharmacology, 37(2012), pp. 2624-2634
Rios J L. Effects of triterpenes on the immune system. J Ethnopharmacol. 2010; 128:1-14.
Rock, E. M., Goodwin, J. M., Limebeer, C. L. et al Interaction between non-psychotropic cannabinoids in marihuana: effect of cannabigerol (CBG) on the anti-nausea or anti-emetic effects of cannabidiol (CBD) in rats and shrews. Psychopharmacology 215, 505-512(2011).doi.org/10.1007/s00213-010-2157-4
Rollinger J M, Schuster D, Danzl B, Schwaiger S, Markt P, Schmidtke M, et al. In silico fishing for rationalized ligand discovery exemplified on constituents of Rutagraveolens. Planta Med. 2009; 75:S195-S204.
Romero-Sandoval, E. Alfonso, Jack E. Fincham, Ashley L. Kolano, Brandi N. Sharpe, and P. Abigail Alvarado-Vázquez. “Cannabis for chronic pain: challenges and considerations.” Pharmacotherapy: The Journal of Human Pharmacology and Drug Therapy 38, no. 6 (2018): 651-662.
Ross R A. The enigmatic pharmacology of GPR55. Trends Pharmacol Sci. 2009; 30:S156-S163.
Rubin, Lemberger, Warrick, Crabtree, Sullivan, Rowe, Obermeyer; Physiologic disposition of nabilone, a cannabinol derivative, in man; Clinical Pharmacology and Therapeutics, 22 (1977), pp. 85-91
Russo; Clinical endocannabinoid deficiency (CECD): Can this concept explain therapeutic benefits of Cannabis in migraine, fibromyalgia, irritable bowel syndrome and other treatment-resistant conditions?; Neuro Endocrinology Letters, 25 (2004), pp. 31-39
Ryberg, Larsson, Sjögren, Hjorth, Hermansson, Leonova, . . . , Greasley; The orphan receptor GPR55 is a novel cannabinoid receptor; British Journal of Pharmacology, 152(2007), pp. 1092-1101
Sagar, Gaw, Okine, Woodhams, Wong, Kendall, Chapman; Dynamic regulation of the endocannabinoid system: Implications for analgesia; Molecular Pain, 5(2009), p. 59
Sagy, Bar-Lev Schleider, Abu-Shakra, Novack; Safety and efficacy of medical Cannabis in fibromyalgia; Journal of Clinical Medicine, 8 (2019), p. 807
Salvinorin A, a diterpene in Salvia divinorum, produces CB1-mediated effects in the gastrointestinal tract of rodents. Salvinorin A primarily acts as a kappa-opioid receptor agonist and is inactive as a ligand for CB1 and CB2 (Capasso et al., 2008); it may interact with a putative CB1-kappa-opioid receptor heterodimer (Fichna et al., 2012).
Sarchielli, Mancini, Floridi, Coppola, Rossi, Nardi, . . . , Calabresi; Increased levels of Neurotrophins are not specific for chronic migraine: Evidence from primary fibromyalgia syndrome; The Journal of Pain, 8 (2007), pp. 737-745
Savinainen, Jurvinen, Lame, Laitinen; Despite substantial degradation, 2-arachidonoylglycerol is a potent full efficacy agonist mediating CB(1) receptor-dependent G-protein activation in rat cerebellar membranes; British Journal of Pharmacology, 134 (2001), pp. 664-672
Schley, Legler, Skopp, Schmelz, Konrad, Rukwied; Delta-9-THC based monotherapy in fibromyalgia patients on experimentally induced pain, axon reflex flare, and pain relief; Current Medical Research and Opinion, 22 (2006), pp. 1269-1276
Seegehalli M. Anil, Nurit Shalev, Ajjampura C. Vinayaka, Stalin Nadarajan, Dvora Namdar, Eduard Belausov, Irit Shoval, Karthik Ananth Mani, Guy Mechrez & Hinanit Koltai Cannabis compounds exhibit anti-inflammatory activity in vitro in COVID-19-related inflammation in lung epithelial cells and pro-inflammatory activity in macrophages, Nature Research, (2021) 11:1462 doi.org/10.1038/s41598-021-81049-2
Seely K A, Levi M S, Prather P L. The dietary polyphenols trans-resveratrol and curcumin selectively bind human CB1 cannabinoid receptors with nanomolar affinities and function as antagonists/inverse agonists. J Pharmacol Exp Ther. 2009; 330:531-539.
Seth Crawford (2021), “Triploid Seed R&D at Oregon CBD”, www.youtube.com/watch?v=tpehlYztWzs
Sharifian-Dorche, M.; Huot, P.; Osherov, M. et al. Neurological complications of coronavirus infection; a comparative review and lessons learned during the COVID-19 pandemic. J Neurol Sci2020, 417, 117085.
Shen, Yang, Qian, Hou; The role of peripheral cannabinoid receptors type 1 in rats with visceral hypersensitivity induced by chronic restraint stress; Journal of Neurogastroenterology and Motility, 16 (2010), pp. 281-290; 2010/07/27. Korean Society of Neurogastroenterology and Motility
Shrestha R, Kim S C, Dyer J M, Dixon R A, Chapman K D. Plant fatty acid (ethanol) amide hydrolases. Biochim Biophys Acta. 2006; 1761:5324-5334.
Singh, Mrinalini, Rajkumar Tulsawani, Praveen Koganti, Amitabh Chauhan, Manimaran Manickam, and Kshipra Misra. “Cordyceps sinensis increases hypoxia tolerance by inducing heme oxygenase-1 and metallothionein via Nrf2 activation in humanlung epithelial cells.” BioMed Res. Int. 2013 (2013).
Skrabek, Galimova, Ethans, Perry; Nabilone for the treatment of pain in fibromyalgia. In; The Journal of Pain, 9 (2008), pp. 164-173
Slomski A. Trials Test Mushrooms and Herbs as Anti-COVID-19 Agents. JAM Published online Nov. 3, 2021. doi:10.1001/jama.2021.19388
Smith, Stanley, Foss, Boles, McKernan; Rare genetic variants in the endocannabinoid system genes CNR and DAGLA are associated with neurological phenotypes in humans; PLoS One, 12 (2017), Article e0187926
Soni, Tej Prakash, Anil Kumar Gupta, Lalit Mohan Sharma, Harish Singhal, Shantanu Sharma, and Ravindra Singh Gothwal. “A Randomized, Placebo-Controlled Study to Evaluate the Effect of Bio-Enhanced Turmeric Formulation on Radiation-Induced Oral Mucositis.” Orl 84, no. 2 (2022):103-113.
Spelman K, Iiams-Hauser K, Cech N B, Taylor E W, Smirnoff N, Wenner C A. Role for PPARgamma in IL-2 inhibition in T cells by Echinacea-derived undeca-2E-ene-8,10-diynoic acid isobutylamide. Int Immunopharmacol. 2009; 9:S1260-S1264.
Starowicz, Finn; Cannabinoids and pain: Sites and mechanisms of action; Advances in Pharmacology, 80 (2017), pp. 437-475
Steffens, Zentner, Honegger, Feuerstein; Binding affinity and agonist activity of putative endogenous cannabinoids at the human neocortical CB1 receptor; Biochemical Pharmacology, 69(2005), pp. 169-178
Stella, Schweitzer, Piomelli; A second endogenous cannabinoid that modulates long-term potentiation; Nature, 388 (1997), pp. 773-778
Stensson, Gerdle, Ernberg, Mannerkorpi, Kosek, Ghafouri; Increased anandamide and decreased pain and depression after exercise in fibromyalgia; Medicine and Science in Sports and Exercise, 52 (2020), pp. 1617-1628
Stensson, Ghafouri, Ernberg, Mannerkorpi, Kosek, Gerdle, Ghafouri; The relationship of Endocannabinoidome lipid mediators with pain and psychological stress in women with fibromyalgia: A case-control study; The Journal of Pain, 19 (2018), pp. 1318-1328
Suardfaz, Estivill-Torrs, Goicoechea, Bilbao, Rodrfguez, de Fonseca; Analgesic properties of oleoylethanolamide (OEA) in visceral and inflammatory pain; PAIN, 133(2007), pp. 99-110
Syed, Bui, Beavers, Farb, Ficorilli, Chesterfield, Kuo, Bokvist, Barrett, Efanov; Regulation of GPR119 receptor activity with endocannabinoid-like lipids; American Journal of Physiology. Endocrinology and Metabolism, 303 (2012), pp. E1469-E1478
Tagne, Alex Mabou, Yannick Fotio, Lin Lin, Erica Squire, Faizy Ahmed, Tarif Ibne Rashid, Elnaz Karimian Azari, and Daniele Piomelli. “Palmitoylethanolamide and hemp oil extract exert synergistic anti-nociceptive effects in mouse models of acute and chronic pain.” Pharmacological research 167(2021):105545.
Tham, Yilmaz, Alaverdashvili, Kelly, Denovan-Wright, Laprairie; Allosteric and orthosteric pharmacology of cannabidiol and cannabidiol-dimethylheptyl at the type 1 and type 2 cannabinoid receptors; British Journal of Pharmacology, 176(2019), pp. 1455-1469
Thors L, Alajakku K, Fowler C J. The ‘specific’ tyrosine kinase inhibitor genistein inhibits the enzymic hydrolysis of anandamide: implications for anandamide uptake. Br J Pharmacol. 2007; 150:5951-5960.
Thors L, Belghiti M, Fowler C J. Inhibition of fatty acid amide hydrolase by kaempferol and related naturally occurring flavonoids. Br J Pharmacol. 2008; 155:5244-5252.
Tiwari, Anshuly, Kakasaheb R. Mahadik, and Satish Y. Gabhe. “Piperine: A comprehensive review of methods of isolation, purification, and biological properties.” Medicine in Drug Discovery 7(2020):100027.
Tramèr, Carroll, Campbell, Reynolds, Moore, McQuay; Cannabinoids for control of chemotherapy induced nausea and vomiting: Quantitative systematic review; Bmj, 323(2001), pp. 16-21
Trametes versicolor(mushroom, often has algae symbiosis), Fomitopsis officinalis (mushroom), modified Qing Fei Pai Du Tang (herbal) are currently undergoing clinical trials in US for prevention, as adjunctive treatment/symptoms control, or to facilitate recovery of Covid-19.
Valdeolivas S, Navarrete C, Cantarero I, Bellido M L, Mufoz E, Sagredo O. Neuroprotective properties of cannabigerol in huntington's disease: studies in r6/2 mice and β-nitropropionate-lesioned mice. Neurotherapeutics. 2015; 12(1):185-199.
van de Donk, Niesters, Kowal, Olofsen, Dahan, Van Velzen; An experimental randomized study on the analgesic effects of pharmaceutical-grade Cannabis in chronic pain patients with fibromyalgia; PAIN, 160 (2019), pp. 860-869
Varatharaj, A. et al.; Coro Nerve Study Group. Neurological and neuropsychiatric complications of COVID-19 in 153 patients: a UK-wide surveillance study. Lancet Psychiatry 2020, 7(10), 875-882.
Wall, Sadler, Brine, Taylor, Perez-Reyes; Metabolism, disposition, and kinetics of delta-9-tetrahydrocannabinol in men and women; Clinical Pharmacology and Therapeutics, 34(1983), pp. 352-363
Wang, Bo, Anna Kovalchuk, Dongping Li, Rocio Rodriguez-Juarez, Yaroslav Ilnytskyy, Igor Kovalchuk and Olga Kovalchuk, “In search of preventive strategies: novel high-CBD Cannabis sativa extracts modulate ACE2 expression in COVID-19 gateway tissues” 22 Nov. 2020, Aging-US. DOI: 10.18632/aging. 202225
Wang, Q., Wei, H., Deng, C., Xie, C., Huang, M. and Zheng, F., 2020. Improving stability and accessibility of quercetin in olive oil-in-soy protein isolate/pectin stabilized O/W emulsion. Foods, 9(2), p. 123.
Ware, Fitzcharles, Joseph, Shir; The effects of nabilone on sleep in fibromyalgia: Results of a randomized controlled trial; Anesthesia and Analgesia, 110(2)(2010), pp. 604-610
Watabiki, Tsuji, Kiso, Ozawa, Narazaki, Kakimoto; In vitro and in vivo pharmacological characterization of ASP8477: A novel highly selective fatty acid amide hydrolase inhibitor; European Journal of Pharmacology, 815(2017), pp. 42-48
Weber, Schley, Casutt, Gerber, Schueopfer, Rukwied, Konrad; Tetrahydrocannabinol (Delta 9-THC) treatment in chronic central neuropathic pain and fibromyalgia patients: Results of a multicenter survey; Anesthesiology Research and Practice (2009)
Webster, R. G. Wet markets—a continuing source of severe acute respiratory syndrome and influenza? Lancet 2004, 363, 234-236.
Wilsey, Marcotte, Deutsch, Gouaux, Sakai, Donaghe; Low-dose vaporized Cannabis significantly improves neuropathic pain; The Journal of Pain, 14 (2013), pp. 136-148
Woelkart K, Dittrich P, Beubler E, Pinl F, Schoop R, Suter A, et al. Pharmacokinetics of the main alkamides after administration of three different Echinacea purpurea preparations in humans. Planta Med. 2008; 74:5651-5656.
Wong, Camilleri, Busciglio, Carlson, Szarka, Burton, Zinsmeister; Pharmacogenetictrial of a cannabinoid agonist shows reduced fasting colonic motility in patients with nonconstipated irritable bowel syndrome; Gastroenterology, 141 (2011), pp. 1638-1647. e1637
Wong, Camilleri, Eckert, Carlson, Ryks, Burton, Zinsmeister; Randomized pharmacodynamic and pharmacogenetic trial of dronabinol effects on colon transit in irritable bowel syndrome-diarrhea; Neurogastroenterology and Motility: the Official Journal of the European Gastrointestinal Motility Society, 24 (2012), pp. 358-e169
Woo, P. C.; Lau, S. K.; Yuen, K. Infectious diseases emerging from Chinese wet-markets: zoonotic origins of severe respiratory viral infections. Curr. Opin. Infect. Dis 2006, 19, 401-407.

Xu, G. R., Zhang, C., Yang, H X., Sun, J. H., Zhang, Y., Yao, T. T., Li, Y., Ruan, L., An, R. and Li, A. Y., 2020. Modified citrus pectin ameliorates myocardial fibrosis and inflammation via suppressing galectin-3 and TLR4/MyD88/NF-κB signaling pathway. Biomedicine & Pharmacotherapy, 126, p. 110071.

Yassin, Oron, Robinson; Effect of adding medical Cannabis to analgesic treatment in patients with low back pain related to fibromyalgia: an observational cross-over single centre study; Clinical Experimental Rheumatology, 37(116)(2019), pp. 13-20
Yilmaz-Oral, D., Onder, A., Gur, S., Carbonell-Barrachina, Á. A., Kaya-Sezginer, E., Oztekin, C. V., & Zor, M. (2020). The beneficial effect of dove essential oil and its major component, eugenol, on erectile function in diabetic rats. Andrologia. doi:10.1111/and. 13606
Yin H, Chu A, Li W, Wang B, Shelton F, Otero F, et al. Lipid G protein-coupled receptor ligand identification using beta-arrestin Path Hunter assay. J Biol Chem. 2009; 284:S12328-S12338.
Yorgancioglu, Ali et al., “Potential Applications Of Fibre-Type Industrial Hemp Residues For Leather Industry”, V International Leather Engineering Congress Innovative Aspects for Leather Industry (2019, Turkey)
Zendulka, Ondrej, Gabriela Dovrtelová, Kristýna Nosková, Miroslav Turjap, Alexandra Sulcová, Lumir Hanus, and Jan Jurica. “Cannabinoids and cytochrome P450 interacions.” Current drug metabolism 17, no. 3 (2016):206-226
Zhang, Lihua, Xin Zheng, Xueke Bai, Qing Wang, Bowang Chen, Haibo Wang, Jiapeng Lu et al. “Association between Use of Qingfei Paidu Tang and Mortality in Hospitalized Patients with COVID-19: A national retrospective registry study.” Phytomedicine 85 (2021):153531.
Zhao, Kaidi, and Jagdish Singh. “Mechanisms of percutaneous absorption of tamoxifen by terpenes: eugenol, D-limonene and menthone.” Journal of controlled release 55, no. 2-3(1998):253-260.
Zhou, F.; Yu, T.; Du, R.; Fan, G.; Liu, Y.; Liu, Z. et al. Clinical course and risk factors for mortality of adult inpatients with COVID-19 in Wuhan, China: a retrospective cohort study.
Zimmer A, Racz I, Klauke A L, Markert A, Gertsch J. Beta-caryophyllene, a phytocannabinoid acting on CB2 receptors. 2009. IACM5th Conference on cannabinoids in medicine, 2-3. October, Cologne, Germany.
Zou, Kumar; Cannabinoid receptors and the endocannabinoid system: Signaling and function in the central nervous system; International Journal of Molecular Sciences, 19 (2018), p. 833
Zuo, Kranzler, Luo, Covault, Gelernter; CNR1 variation modulates risk for drug and alcohol dependence; Biological Psychiatry, 62 (2007), pp. 616-626.
academic.oup.com/advances/article/9/1/21/4849000; article-abstract/13/2/530/6382126; rheumatology/article/38/8/779/2898713
acslabcannabis.com/blog/extraction/methods-of-cbd-extraction-storage-and-tincture-blending/
aip.scitation.org/doi/pdf/10.1063/5.0096877
ajp.psychiatryonline.org/doi/10.1176/appi.ajp.2021.20081202
alert.psychnews.org/2019/07/nabaximols-nasal-spray-may-reduce.html
alsnewstoday.com/news/probiotic-prevents-neurodegeneration-als-worm-models/
alsoorganics.com/blogs/cbd-stories/cbd-and-pesticides.
alz-journals.onlinelibrary.wiley.com/doi/epdf/10.1002/alz.12789; 10.1002/trc2.12348; 10.1002/alz.13078
alzres.biomedcentral.com/track/pdf/10.1186/s13195-021-00920-6.pdf
app.sermo.com/post/950101
are.uconn.edu/wp-content/uploads/sites/2327/2020/02/ZwickOR66_Feb25.pdf
area52.com/what-is-cbgv/
asm.org/Artides/2022/March/Microbiology-of-Endurance-The-Microbiome-s-Role-in
asthmaandallergies.org/food-allergies/adverse-reactions-to-food-additives/
alashiomed.com/blog/potato-resistant-starch/
bazelethealth.com/
biomedican.com/which-is-better-chd-or-chg/
bioresources.cnr.ncsu.edu/resources/antibacterial-properties-of-hemp-and-other-natural-fibre-plants-α-review/
blog.scrcivf.com/chd-and-fertility
blog.sonomechanics.com/blog/the-role-of-carrier-oils-in-water-soluble-chd-and-thc-formulations; water-soluble-Cannabis-oil-microemulsion-liposomes-or-nanoemulsion
bloommedicinals.com/Cannabis-education/Cannabis-bioavailability-whats-the-most-effective-method/
bmjopen.bmi.com/content/12/12/e060753
bpspubs.onlinelibrary.wiley.com/doi/10.1111/bph.15661; 10.1111/bph.12445
business.dutchie.com/post/the-activists-guide-to-Cannabis-organizations-α-comprehensive-list
can-acn.org/docs/CAN2022-Abstracts-Final.pdf
cannabishealthnews.co.uk/2022/07/21/new-study-highlights-dangers-of-vaping-cannabinoid-acetates/
cannabislifenetwork.com/chd-is-everywhere/; chg-is-safe-gets-green-light-for-dinical-trials-and-therapy; do-cannabinoids-chg-or-chd-affect-thc-tolerance
cannabismd.com/basics/chd/why-people-are-using-nebulizers-for-chd-oil/
cannabisnow.com/cultivar/
cannabusiness.law/thc-analogs-α-family-divided/
cannatechtoday.com/chd-oil-food-preservative/
cannigma.com/history/what-does-religion-say-about-Cannabis/; physiology/keto-diet-and-Cannabis/
cbdgenesis.com/blogs/news/hest-thcv-strains/; collections/thcv/
chdorade.com/news/hemp-delta-9-thc-products-lah-study-consumer-safety-and-legality/; most-influential-Cannabis-organizations/
cbdscanner.co.uk/chd-and-your-sperm-count/
cbgcode.com/weight-loss-with-chg/
cdn.intechopen.com/pdfs/37170/InTech-Good_manufacturing_practices_gmp_for_medicinal_products.pdf
cdn.sanity.io/files/Ovv8moc6/psychtimes/3339ed149a63fc98f69e9a01976379b4c77e9d70.pdf/PSY0821_ezine.pdf; 46d02565c709e221d39a42b79c472a767c9783fd.pdf/PSY0522_Ezine.pdf
cellandhioscience.biomedcentral.com/articles/10.1186/s13578-022-00826-2 cfah.org/chd-drug-interactions/
chemrxiv.org/engage/chemrxiv/article-details/62c5fd7el4201f3bbe287333 cleanleaf.com/the-stages-of-Cannabis-growth.php
www.dinicaladvisor.com/home/topics/rheumatology-information-center/aspirin-use-and-bone-health-unpacking-the-controversy/
clinicalhypertension.biomedcentral.com/articles/10.1186/s40885-020-00147-x
coek.info/pdf-chapter-2-alkaloids-in-Cannabis-sativa-.html
commons.emich.edu/cgi/viewcontent.cgi?article=1326&context=honors
content.govdelivery.com/accounts/MEDACF/bulletins/2af5867
corporate.dukehealth.org/news/stopping-herbal-products-abruptly-may-cause-withdrawal-symptoms
crohnsandcolitis.ca/About-Crohn-s-Colitis/IBD-Journey/Treatment-and-Medications/JAK-inhibitors; 2022/08/23/consuming-green-vegetables-and-certain-supplements-suppresses-inflammatory-bowel-disease/
cropwatch.unl.edu/2019/hemp-production-fiber-or-grain
dailycbd.com/en/cannabinoids/metabolism/; cbdv; does-cbd-oil-expire; how-to-make-delta-8-thc;/
dailymed.nlm.nih.gov/dailymed/druglnfo.dm
dhss.delaware.gov/dph/hsp/medmarconditions.html
d-nb.info/l176433377/34
doh.dc.gov/sites/default/files/dc/sites/doh/publication/attachments/Medical Cannabis Adverse Effects and Drug Interactions_0.pdf
doi.org/10.1016/j.jneuroim.2010.12.005; 10.1016/j.psychres.2022.l14573; 10.1186/s13643-022-01951-2; 10.3390/plantsl1101357; 10.2147/CEG.S85574
draxe.com/nutrition/butyric-acid/; erythritol
dreliaz.org/breaking-through-biofilms-the-hidden-barriers-to-optimal-health/
eightysixbrand.com/comparing-the-newcomers-thco-vs-hhc/
elevate-holistics.com/medical-condition/medical-marijuana-for-gout/
elevate-holistics.com/what-is-cbg/
elifesciences.org/articles/73246
en.wikipedia.org/wiki/Cannabidiol; Cannabigerovarin; Cannabinoid; Cannabis_drug testing; Caryophyllene; Convention_on_Psychotropic_Substances; Cordycepin; Entourage_effect; Hops_and_cannabinoids; Legal_history_of_Cannabis_in_the_United_States; List_of_anti-Cannabis_organizations; Pheophorbide; TRPV1; Oleamide; Protein_Digestibility_Corrected_Amino_Acid_Score
extension.usu.edu/crops/research/ten-frequently-asked-questions-about-growing-Cannabis
eybna.com/terpene/cannabichromenic-acid/; cannabigerolic-acid
eyewiki.aao.org/Cannabinoids_for_Glaucoma
farmerandchemist.com/blogs/news/chda-and-chga
faseb.onlinelibrary.wiley.com/doi/epdf/10.1096/fi.201903093R
frontiersmag.wustl.edu/2021/07/17/endocannabinoids-or-endorphins-the-truth-behind-the-runners-high/
fusioncbdproducts.com/blogs/news/difference-between-full-spectrum-and-broad-spectrum-cbd-it-s-you
future4200.com/t/increase-the-viscosity-of-mct-oil/177089/4
futurism.com/neoscope/superpowered-thc-substance
fve.org/cms/wp-content/uploads/001_CBD_Cannabis_adopted.pdf
genomemedicine.biomedcentral.com/articles/10.1186/s13073-022-01148-1
ggs-greenhouse.com/blog/advantages-of-growing-hemp-in-α-greenhouse; marijuana/blog/outdoor-vs-greenhouse-hemp-production-which-is-best
globalcannabiscompliance.bakermckenzie.com/2020/07/31/what-you-need-to-know-before-importing-exporting-legal-Cannabis-products/
greenfaithministry.com/
greenliving.lovetoknow.com/dangers-food-additives-preservatives
health.develanddinic.org/benefits-of-black-pepper/; butyrate-benefits; pregnancy-does-acetaminophen-heighten-risks-for-autism-adhd
healthnewshub.org/can-the-keto-diet-affect-your-heart-health/
hempaccopackaging.com/wp-content/uploads/2020/08/CBG-Hemptown-White-Paper.pdf
hempfoundation.net/proven-anti-badterial-properties-of-hemp-driving-demand-in-increasingly-health-conscious-world/
hempindustrydaily.com/2018-farm-bill-report/; cbd-manufacturers-seeking-market-edge-tout-absence-of-common-weedkiller; european-commission-adds-cbg-as-legal-ingredient-for-cosmetics-skin-care
hemptoday.net/cbd-products-flagged/
higheryieldsconsulting.com/22-ways-hemp-is-used-for-commercial-and-industrial-products/
highgradehempseed.com/blog/what-are-feminized-seeds
howard-autism.org/wp-content/uploads/2020/05/Medical-Cannabis-in-Children.pdf
ibcnj.com/medical-Cannabis-mast-cell-activation-syndrome/
ilesol.com/cannabigerol-cbg-mother-of-cannabinoids/
irp-cdn.multiscreensite.com/9b253dc9/files/uploaded/CBD %200il %20and %20 (in)Fertility.pdf
jamanetwork.com/journals/jama/fullarticle/1758733; 2661569; 2786023; 2786653; 2796948; 2811556; jamadermatology/fullarticle/2808971; 2808975; jama-health-forum/fullarticle/2795319; jamainternalmedicine/fullarticle/649500; jamanetworkopen/fullarticle/2792539; 2794440; 2794987; 2797718; 2800693; jamaneurology/fullarticle/2797021; 2795863; 2797247; jamapsychiatry/fullarticle/2801422
jcannabisresearch.biomedcentral.com/articles/10.1186/s42238-019-0011-; 10.1186/s42238-020-00030-4; 10.1186/s42238-021-00093-x; 10.1186/s42238-022-00152-x; 10.1186/s42238-020-00034-0.pdf; 10.1186/s42238-022-00140-1.pdf; 10.1186/s42238-020-00027-z; 10.1186/s42238-022-00152-x
jlb.onlinelibrary.wiley.com/doi/10.1189/jlb.3RU0115-021R
joe.bioscientifica.com/view/journals/joe/248/2/JOE-20-0444.xml
journals.physiology.org/doi/full/10.1152/ajpgi.90374.2008
journals.plos.org/plosone/article?id=10.1371/journal.pone.0246990; 10.1371/journal.pone.0272492; 10.1371/journal.pone.0274698
journals.sagepub.com/doi/pdf/10.1177/1557988319892464
jpet.aspetjournals.org/content/319/1/150; 376/2/204
khn.org/news/article/colorado-psychedelics-mushrooms-ballot-initiative/; epilepsy-unregulated-cbd-seizures
klingecorp.com/blog/the-importance-of-temperature-control-in-hemp-processing/
labblog.uofmhealth.org/body-work/more-than-half-of-people-using-Cannabis-for-pain-experience-multiple-withdrawal-symptoms; rounds/vaping-marijuana-associated-more-symptoms-of-lung-damage-than-vaping-or-smoking-nicotine
leafwell.com/blog/drying-and-curing-Cannabis/; flavonoid-cannflavins; minor-cannabinoids; what-are-the-benefits-side-effects-of-medical-Cannabis-for-children; what-is-cannabichromene-cbc-cannabinoid
legionathletics.com/animal-protein-vs-plant-protein/
libguides.law.uga.edu/c.php?g=522835&p=3575350
link.springer.com/article/10.1007/BF02245145; 10.1208/s12248-021-00616-7; 10.1007/s11357-022-00559-7.pdf; 10.1007/s42452-019-0759-7.pdf
longevity.technology/lifestyle/spermidine-the-supplement-that-benefits-health-and-lifespan/
mediabros.store/blogs/news/how-Cannabis-wax-is-made
medicalxpress.com/news/2021-12-epileptic-seizure-frequency-fell-kids.html; 2022-09-non-opioid-compounds-squelch-pain-sedation.html; 2023-07-fermented-vegetables-positively-impact-gut.html; 2023-07-ketone-bodies-alternative-cellular-fuels.html #google_vignette
medicine.wustl.edu/news/uncovering-genetic-roots-of-marijuana-use-disorder/
medlineplus.gov/druginfo/natural/1126.htmll
merryjane.com/culture/what-is-cbm-and-what-does-this-uncommon-cannabinoid
microbiomejournal.biomedcentral.com/articles/10.i186/s40168-017-0254-x
mjbizdaily.com/states-struggle-to-set-rules-for-hemp-intoxicants-such-as-delta-8-thc/; wholesale-marijuana-hemp-oil-prices-fall-nationwide
molecularautism.biomedcentral.com/track/pdf/10.i186/s13229-020-0313-1.pdf www.sciencedirect.com/science/article/pii/S0165178115003042
molecularneurodegeneration.biomedcentral.com/articles/10.1186/s13024-022-00515-1
multiplesdlerosisnewstoday.com/news-posts/2023/02/08/berberine-potential-natural-ms-treatment-mouse-model/
mvets.law.gmu.edu/2019/05/13/marijuana-discharges-α-level-playing-field/
my.clevelandclinic.org/health/articles/22618-oxytocin
nap.nationalacademies.org/download/10490
nativenewsonline.net/advertise/branded-voices/can-cbd-help-with-dementia
naturallivingfamily.com/cbd-Cannabis-oil-uses/
neurogan.com/blogs/news/cbd-tolerance
neurosciencenews.com/addiction-reward-system-21493/; astrocytes-cognitive-processing-21674; genetics-stress-ptsd-21687; als-probiotics-22116/; gut-bacteria-diabetes-22173/
news.berkeley.edu/2019/02/27/yeast-produce-low-cost-high-quality-cannabinoids/
news.harvard.edu/gazette/story/2021/04/thc-and-chd-content-on-labels-of-medicinal-products-may-be-inaccurate/
news.osu.edu/a-pain-reliever-that-alters-perceptions-of-risk/
nida.nih.gov/news-events/news-releases/2022/08/marijuana-and-hallucinogen-use-among-young-adults-reached-all-time-high-in-2021
nohoartsdistrict.com/how-to-find-Cannabis-seeds-with-high-thc/
norml.org/marijuana/fact-sheets/normls-guide-to-delta-8-and-other-novel-cannabinoids/
observer.com/2020/12/whats-cbg-legal-Cannabis-benefits-uses/
onestop.md.gov/tags/6021cfcOf8fe400104854c76
onlinelibrary.wiley.com/doi/10.1002/jmv.24292; 10.1111i/jpn.13616; 10.1002/lio2.479; 10.1111/jcpt.12679; 10.1111i/joim.13574; 10.1002/acn3.51078
oregoncbdseeds.com/blog/?post=triploid-seed-rd-at-Oregon-cbd
originalfarm.com/what-is-cannabichromene-cbc/
origins.osu.edu/article/illegalization-marijuana-brief-history?language_content_entity=en
oxytocin-alzheimers-cognition-21710; sleep-immune-inflammation-21466
parkinsonsnewstoday.com/news/damage-gut-microbiome-leads-parkinsons-symptoms-study/; inflammatory-gut-bacteria-genes-may-be-parkinsons-biomarkers/
pdf.sciencedirectassets.com/282671/1-s2.0-S2212877823X00088/1-s2.0-S2212877823000893/main.pdf
pharmahemp.com/wp-content/uploads/2022/05/Cannabis_and_cbd_under_the_scope_ENG.pdf; pharma-hemp.com/cbd-oral-care-blog/
programs.ifas.ufl.edu/media/programsifasufledu/hemp/files/journal-artid es/Devel opment-of-Cannabinoids-in-Flowers-of-Industrial-Hemp.pdf
psycnet.apa.org/record/1992-19188-001
pubmed.ncbi.nlm.nih.gov/10729327; 11331425; 11779138; 11897159; 12435806; 15670582; 15670582; 16083701; 16400887; 16988594; 17484726; 17621270; 17828286; 18622408; 19838548; 20410309; 20614687; 20614687; 21266173; 21324315; 21716094; 21928146; 22103420; 2224182; 22462722; 23601425; 24051945; 26176651; 26987641; 2824983; 28349316; 2849049; 29112398; 29876791; 29986533; 30150937; 30258398; 30691004; 30993303; 31313338; 31396087; 31481004; 31494273; 31529613; 31803950; 31881191; 32036242; 32198013; 32357565; 32485547; 32758877; 32858172; 3297423; 33919780; 33924979; 33998890; 34035994; 34263407; 34569849; 34624678; 34830256; 35007072; 35221321; 35246858; 35326140; 35566134; 35609718; 35617670; 35683028; 35921510; 35987236; 36009504; 36012124; 36080415; 3620017; 402665; 6090909; 6865895; 8055263; 9217128; 27117004; 28954820; 32390638; 32877215; 34302829; 34553667; 35228494; 36709599; PMC7386961; PMC10386153
pubs.acs.org/doi/10.1021/acs.jnatprod.1c00513; 10.1021/cn500206a; 10.1021/ja907062v; abs/0.1021/acs.jnatprod.9b00876; pdf/10.1021/acs.jafc.1c01487; pdf/10.1021/acsinfecdis.9b00419; pdf/10.1021/cn500206a
purecraftcbd.com/blogs/news/cbd-and-fertility-for-men-and-women; the-benefits-of-cbd-oil-for-the-keto-diet
reader.elsevier.com/reader/sd/pii/S0920121120305763; 51319016421001183; 51360138520301874; S2095311920634852; S2772566922000052
recovery.org/phenibut/legal-status/
releases/archives/2021/9/federal-study-finds-Cannabis-beneficial-
religion.fandom.com/wiki/Religious_and_spiritual_use_of_Cannabis
revistagastrocol.com/index.php/rcg/article/view/407
rupress.org/jgp/article/150/8/1081/43759/Structure-function-and-allosteric-modulation-of
sativaisticated.com/wp-content/uploads/2020/07/Influences-of-Phytocannabinoids-on-Drug-Drug-Interactions-and-Their-Clinical-Implications-Medical-Cannabis-Medical-Marijuana-Research-Study-for-Pharmacokinetics.pdf
satechdaily.com/7-natural-nootropics-for-better-brain-performance-which-is-best/; what-happens-if-your-body-dock-is-out-of-whack-disrupted-circadian-rhythms-linked-to-cancer/
search.bvsalud.org/global-literature-on-novel-coronavirus-2019-ncov/resource/en/covidwho-1059599
secretnaturecbd.com/blogs/cbd/cbga-cannabigerolic-acid-guide
slate.com/technology/2022/09/delta-8-thc-cbd-cannabinoids-legal-farm-bill.html
slumbercbn.com/is-cbn-legal-for-sale-in-the-us/
smw.ch/article/doi/smw.2020.20340
sniflmd.com/pdf/Dietary_Supplement-%20Nasal_Measures_vl.pdf
sorsetech.com/why-do-most-Cannabis-products-taste-so-bad/
systematicreviewsjournal.biomedcentral.com/articles/10.1186/s13643-022-01951-2
tanasi.com/blog/cbg-vs-cbga-definitions-benefits-and-products/
terpenesandtesting.com/category/science/cultivar-v-chemovar-debating-the-debasement-of-Cannabis-vernacular/
theeverygirl.com/butyrate-for-gut-health/
thehill.com/changing-America/well-being/prevention-cures/3622526-study-links-common-asthma-medication-to-changes-in-brain/
thejournalofheadacheandpain.biomedcentral.com/track/pdf/10.1186/s10194-022-01399-8.pdf
thelodgecannabis.com/blog/live-resin-vs-live-rosin/
themusclephd.com/plant-vs-animal-protein/
translationalneurodegeneration.biomedcentral.com/articles/10.1186/s40035-020-00221-2
vaping360.com/best-cbd-hemp-flower-bud/cbg-flower/; learn/cbd-oil-prices/
vapingdaily.com/what-is-vaping/does-vape-juice-expire/
vapingfacts.health.nz/the-facts-of-vaping/side-effects-of-vaping.html
voi.id/en/tulisan-seri/3022/marijuana-and-religion
watermark.silverchair.com/ns-2020-0080c.pdf
wayofleaf.com/Cannabis/strains/dougs-varin-strain
wires.onlinelibrary.wiley.com/doi/epdf/10.1002/wfs2.1450
ww2.uthscsa.edu/artt/AddictionJC/2021-10-12-Tanda.pdf
wwmt.com/amp/news/local/michigan-state-police-stopping-thc-blood-tests-due-to-possible-discrepancies
www.aaaai.org/tools-for-the-public/conditions-library/allergies/marijuana-Cannabis-allergy
www.academia.edu/79025796/Role_of_PUFA_the_precursors_of_endocannabinoids_in_human_obesity_and_type_2_diabetes
www.accessdata.fda.gov/drugsatfda_docs/label/2018/2103651bl.pdf
www.additudemag.com/Cannabis-use-disorder-marijuana-adhd/
www.aging-us.com/article/202225/text
www.ahajournals.org/doi/10.1161/JAHA.122.026484; 10.1161/STROKEAHA.l17.019664
www.ahvma.org/wp-content/uploads/Cannabis-in-Veterinary-Medicine-A-Critical-Review-Vol-61__LR-3.pdf
www.ajconline.org/action/showPdf?pii=S0002-9149%2822%2900541-0
www.alchimiaweb.com/blogen/cannabigerol-cbg/; increase-sugars-Cannabis/
www.allbud.com/marijuana-strains/sativa-dominant-hybrid/k1
www.ama-assn.org/system/files/α22-call-to-order.pdf
www.americanbar.org/groups/business_law/publications/blt/2020/02/Cannabis-banking/
www.archives-pmr.org/article/S0003-9993(18) 30154-0/fulltext
www.arnoldporter.com/en/perspectives/blogs/enforcement-edge/2022/02/dni-issues-darifying-guidance
www.avma.org/sites/default/files/2021-03/APH-CannabisResources-Report-20201207.pdf
www.azolifesciences.com/article/Does-Cannahis-Interact-with-the-Gut-Microbiome.aspx
www.bbc.co.uk/bbcthree/article/8b1389df-9153-477d-al8d-56a50178163e
www.henzinga.com/markets/Cannabis/22/07/28124558/legal-Cannabis-plastic-waste-and-sustainability-issues-to-take-into-account-to-take-care-of-our-
www.betterhealth.vic.gov.au/health/conditionsandtreatments/food-additives
www.biologicalpsychiatryjournal.com/action/showPdf
www.biorxiv.org/content/10.1101/2020.02.04.934919vl.full
www.bloomberg.com/press-releases/2021-04-16/bazelet-oglesby-enters-the-georgia-Cannabis-market-with-its-patented-th-free-panakeia-plants
www.blueskyhearing.com/tinnitus-articles/the-link-between-cannabinoids-and-tinnitus/
www.bmj.com/company/newsroom/recent-Cannabis-use-Iinked-to-extremes-of-nightly-sleep-duration/
www.boston.com/news/local-news/2022/10/06/osha-trulieve-worker-asthma-death-Cannabis/
www.britannica.com/plant/hemp
www.buzzfeednews.com/article/katiecamero/Cannabis-industry-worker-health-risks
www. (ambridge.org/core/journals/gut-microbiome/article/butyrateproducing-and-sporeforming-badterial-genus-coprotoccus-as-α-potential-biomarker-for-neurological-disorders/1182937AD49AC8894Δ025324EC0C5013
www. (annabisdinicians.org/2020/09/08/a-molecular-basis-for-the-anti-inflammatory-and-anti-fibrosis-properties-of-cannabidiol/
www.cannabisequipmentnews.com/news/news/22236091/hemp-oil-triggers-supplement-warning
www.cannahislifenetwork.com/what-is-α-tolerance-hreak/
www.cannaconnection.com/blog/19130-how-to-store-edibles-and-how-long-do-they-stay-potent
www.chdwhitelabelexpo.com/news/blog.asp?blog_id=23943
www.chii-chd.com/blog/chd-bioavailability-explained
www.cdc.gov/marijuana/data-statistics.htm
www.cell.com/cell-reports/pdfExtended/S2211-1247(23) 00728-3; immunity/fulltext/S1074-7613(23) 00314-X; iscience/pdf/S2589-0042(20) 30488-0.pdf; 50896-6273(22) 00910-2;/action/showPdf?pii=S1360-1385%2820%2930187-4; trends/plant-science/fulltext/S1360-1385(20) 30187-4
www.chiasisters.co.nz/pages/hemp-protein-prebiotics-and-gut-health
www.childrenscolorado.org/health-professionals/professional-resources/charting-pediatri(s-podcast/medical-marijuana-in-children/
www.chpa.org/news/2021/09/future-dietary-supplements-discussed-chpas-rsq
www.christianity.com/wiki/christian-life/what-is-chd-oil-is-it-okay-for-christians-to-use.html
www.cnn.com/2020/05/28/world/tel-arad-shrine-israel-Cannabis-study-scn/index.html
www.cnn.com/2022/10/08/politics/biden-marijuana-possession-pardons/index.html
www.coindesk.com/layer2/sinweek/2022/08/31/can-crypto-save-the-Cannabis-industry/
www.conflabs.com/why-0-877/
www.consumerreports.org/chd/is-it-safe-to-vape-chd/
www.contemporarypediatrics.com/view/examining-cannabidiol-use-children
www.cpn.or.kr/journal/view.html?doi=10.9758/cpn.2017.15.4.301
www.curednutrition.com/blog/hemp-hearts-gut-health/
www.customsmobile.com/rulings/docview?doc_id=NY %20N308348&highlight=3824.99.28
www.dailydot.com/irl/legal-psychedelics-drugs/
www.dea.gov/drug-information/drug-scheduling; sites/default/files/2020-06/Kratom-2020_0.pdf
www.degruyter.com/document/doi/10.1515/cclm-2021-0658/html; 10.1515/jbcpp-2015-0080/html
www.delta8us.com/thcv
www.dlapipercultivate.com/2022/05/federal-agencies-airrent-positions-on-hemp-derived-chd-products/
www.doximity.com/articles/11b5c779-a89c-4729-9393-90dd013c4d4f
www.drpamelali.ca/cannabidiol-impact-dental-health/
www.efaidnbmnnnibpcajpcgldefindmkaj/
www.epa.gov/pesticide-registration/pesticide-products-registered-use-hemp
www.eurekaselect.com/article/29453
www.evopure.co.uk/chd-bioavailability/
www.extractlabs.com/chd-guides/what-is-cbc/
www.fda.gov/consumers/consumer-updates/fda-101-dietary-supplements; drugs/pharmaceutical-quality-resources/facts-about-current-good-manufacturing-practices-cgmps; files/food/published/Guidance-for-Industry-Frequently- Asked-Questions-About-Medical-Foods-Second-Edition-%28PDF %29.pdf; food/cfsan-constituent-updates/fda-responds-three-gras-notices-hemp-seed-derived-ingredients-use-human-food; food/dietary-supplements/new-dietary-ingredients-ndi-notification-process; food/food- ingredients-packaging/generally-recognized-safe-gras; food/food-ingredients-packaging/overview-food-ingredients-additives-colors; food/information-consumers-using-dietary-supplements/questions-and-answers-dietary-supplements; media/119426/download; media/119427/download; media/119428/download; media/131878/download; news-events/congressional-testimony/hemp-production-and-2018-farm-bill-07252019; news-events/press-announcements/fda-issues-warning-letters-companies-illegally-selling-cd-and-delta-8-thc-products; news-events/public-health-focus/fda-regulation-Cannabis-and-Cannabis-derived-produas-including-cannabidiol-cd; regulatory-information/search-fda-guidance-documents/considering-whether-fda-regulated- product-involves-application-nanotechnology; regulatory-information/search-fda-guidance-documents/complementary-and-alternative-medicine-products-and-their-regulation-food-and-drug-administration; regulatory-information/search-fda-guidance-documents/guidance-industry-questions-and-answers-regarding-adverse-event-repodting-and-recordkeeping-dietary; safety/medical-product-safety-information/medwatch-forms-fda-safety- reporting
www.fiercehealthcare.com/practices/marijuana-legalization-leaves-dodtors-wondering-what-do-we-tell-our-patients; providers/study-doximity-survey-found-physicians-overwhelmed-amount-reading-needed-stay-date
www.fightaging.org/archives/2019/11/modulating-gut-microbe-populations-to-generate-more-butyrate-thus-raising-bdnf-levels-and-improving-cognitive-function/
www.flexport.com/data/hs-code/530210-true-hemp-raw-or-retted
www.forbes.com/health/body/best-cbd-oil/; health/body/hemp-seed-oil-vs-cbd-oil/; health/body/how-long-does-cbd-stay-in-your-system/; health/body/what-are-flavanol-3s/; sites/amandasiebert/2020/09/24/Cannabis-for-christians-gods-greenery-combines-hemp-chd-with-biblical-ingredients/; sites/dariosabaghi/2021/12/14/european-union-increases-th-level-for-industrial-hemp-why-does-it-matter; sites/dariosabaghi/2022/01/18/what-is-the-o-acetate-and-why-is-it-getting-attention/
www.foriawellness.com/products/intimacy-lubricant
www.foxbusiness.com/markets/god-is-getting-into-the-cbd-industry
www.frontiersin.org/articles/10.3389/fchem.2021.694905; fddsv.2022.898035; fendo.2012.00121; fimmu.2020.00413; fncel.2016.00294; fneur.2020.00377; fneur.2020.543275; fnins.2015.00225; fnut.2021.664197; fphar.2018.00420; fphar.2019.00821; fphar.2020.00628; fphar.2020.618184; fphar.2021.626010; fphar.2021.777804; fphar.2021.793750; fphys.2018.01699; fpls.2019.01265; fpsyg.2021.687106; fpsyt.2021.597725; fpsyt.2021.597725; fpsyt.2021.620073; 10.3389/fcimb.2022.857035; l0.3389/fmicb.2022.926710; 10.3389/fnmol.2023.1086285; l0.3389/fnut.2019.00024; 10.3389/fphar.2021.627557; 10.3389/fphar.2022.831052; 10.3389/fphar.2023.1l45666; 10.3389/fvets.2018.00311
www.frontiersin.org/research-topics/27135/aminobiogenic-potential-of-fermented-food-microbiota
www.ftc.gov/system/files/ftc_gov/pdf/2022-04-04%20DAP %20Extract %20Labs.pdf
www.fundation-Canna.es/en/cannabinoid-produdtion-recombinant-organisms-future-reality-or-science-fiction
www.gale.com/open-access/marijuana
www.genengnews.com/topics/omics/microbiome/gut-bacteria-influences-immune-cell-behavior/
www.globenewswire.com/en/news-release/2022/04/06/2417574/0/en/Bazelet-Health-announces-PECSA-its-new-federally-legal-non-GMO-plant-based-Cannabis-ingredient-for-US-and-export-markets.html
www.globenewswire.com/en/news-release/2022/04/28/2431068/0/en/Bazelet-Health-Announces-a-U-S-Scientific-Achievement-and-Scientific-Proof-of-Cannabis-America-its-O-THC-Tri-Purpose-Cannabis-Plant-for-Global-Markets.html; 2022/06/07/2457453/0/en/Bazelet-to-Supply-Its-Federally-Legal-Cannabis-Genetics-to-DEA-Approved-Research-Entities-for-Rigorous-Scientific-Research-on-the-Clinical-Effects-of-Cannabigerol-CBG.html
www.gotquestions.org/CBD-oil.html
www.govexec.com/defense/2022/01/declassified-report-detail s-afghan-air-force-reliance-contractors/360881/
www.grandviewresearch.com/industry-analysis/cannabidiol-cbd-market
www.greenmatters.com/p/hemp-fabric-advantages-disadvantages
www.gvbbiopharma.com/cbga-guide/
www.health.harvard.edu/blog/cbd-and-other-medications-proceed-with-caution-202101121743
www.health.state.mn.us/people/Cannabis/practitioners/docs/dosingreport.pdf
www.healthcanal.com/best-cbd-oil/vape-oil
www.healthline.com/health/aquaphor-vs-vaseline; health/cbd-for-allergies; health/cbg-oil; health/full-spectrum-vs-broad-spectrum-cbd; health/how-long-do-edibles-take-to-kick-in; health/is-weed-α-depressant; health/marijuana-withdrawal; health/what-is-dmso; nutrition/6-health-benefits-of-hemp-seeds; nutrition/calcium-disodium-edta; nutrition/echinacea; nutrition/grapefruit-and-medications
www.healthyagingpoll.org/reports-more/report/arthritis-and-joint-pain
www.hempbassadors.ca/en/paper/
www.hemptonnect.co.nz/blog-2/2020/4/hemp-seed-oil-versus-fish-oil
www.herbolea.com/distillac; hydrocan; technology; news/herbolea-inks-deal-with-leading-hemp-cultivator-in-new-york-state
www.hh.um.es/Abstrats/Vol_35/35_8/35_8_789.htm
www.highsnobiety.com/p/marijuana-health-henefits-eating/
www.hopkinsmedicine.org/health/wellness-and-prevention/what-does-vaping-do-to-your-lungs
www.ingentaconnect.com/content/ben/cdm/2016/00000017/00000003/a00003
www.insideprecisionmedicine.com/topics/precision-medicine-topic/gut-badteria-implicated-in-weight-gain/
www.insider.com/guides/health/terpenes
www.intechopen.com/chapters/78043
www.ipsf.org/article/compounding-corner-april-2019-compounding-creams-and-gels
www.irochelating.com/news/foods-that-contain-edta.html
www.isdi.org/wp-content/uploads/2020/04/ISDI-Foods-For-Special-Medical-Purposes-brochure.pdf
www.jhsph.edu/research/centers-and-institutes/center-of-excellence-in-regulatory-science-and-innovation/training/Iris %20Case %202021%20CERSI %20Writing %20Competition.pdf
www.jillcarnahan.com/2023/02/08/leaky-gut-syndrome-could-α-complex-protein-named-zonul in-be-to-blame/
www.jimmunol.org/content/186/5/3188
www.karger.com/Article/FullText/515042; 517120; 521370; 489287; 522472
www.kevinmd.com/2023/07/the-ketogenic-diet-α-historical-perspective-and-survival-consequences.html
www.keygrowsolutions.com/green-dragon-sugar-signaling-in-Cannabis-production
www.law.cornell.edu/uscode/text/21/813
www.leafly.com/learn/growing; news/lifestyle/is-Cannabis-against-your-religion; news/scence-tech/peak-thc-chd-levels-for-Cannabis-strains; news/science-tech/predicting-Cannabis-strain-effects-from-thc-cbd-levels; news/science-tech/thcp-dhdp-study-reveals-identificaton-two-new-cannabinoids; strains/k2
www.liebertpub.com/doi/10.1089/can.2016.0009; 10.1089/can.2017.0028
www.lifespan.io/news/vital-muscle-enzyme-dedines-with-aging/
www.listerineprofessional.com/studentsandfaculty/studentresources/not-all-rinses-are-the-same
www.livescience.com/35219-11-effects-of-oxytocin.html
www.lorannoils.com/preserve-it-mold-inhibitor-1-oz-6070-0500
www.lostoakfarms.com/store/p/forbidden-v-cbdv-hemp-flower
www.marijuanamoment.net/dumping-chd-oil-on-strawberries-extends-their-shelf-life-and-stops-mold-growth-new-study-finds/; federal-aviation-administration-darifies-marijuana-and-cbd-policy-for-pilots; feds-expose-religious-discrimination-against-marijuana-consumers-in-other-countries-while-ignoring-u-s-policy; house-panel-dires-military-to-review-marijuana-penalties-for-soldiers-and-study-medical-Cannabis-as-opioid-alternative; marijuana-consumers-who-caught-covid-had-better-outcomes-and-mortality-than-nonusers-study-finds/
www.marketwatch.com/story/Cannabis-stocks-cool-off-after-previous-days-rally-as-optimism-around-rescheduling-fades-11665158767? mod=mw_quote_news
www.masscann.org/education-village/peace-and-love-the-spirituality-of-Cannabis/religious-use-of-Cannabis/
www.maximumyield.com/fermented-cannabinoids-a-breakthrough-for-mother-nature/2/18368 www.maximumyield.com/cannabinoid-biosynthesis-growing-cannabinoids-without-the-plant/2/18145
www.mayodinic.org/healthy-lifestyle/consumer-health/expert-answers/is-chd-safe-and-effective/faq-20446700
www.mdlinx.com/article/does-late-night-eating-affect-longeviy-research-sheds-light/33qWTPfGsv7J6YResdK0w; journal-summary/role-of-melatonin-in-the-management-of-sleep-and-circadian-disorders/; building-a-better-painkiller; by-oxidizing-thc-to-create-an-electric-airrent-α-teams-fuel-cell-sensor-can-reveal-the-presence-of; Cannabis-regulations-inadequate-given-rising-health-risks-of-high-potency-products; college-student-fear-of-missing-out-fomo-associated-with-illicit-behavior; could-greater-prescription-of-medical-Cannabis-be-a-solution-to-the-opioid-crisis; discovery-explains-cancer-chemotherapy-resistance-offers-solution; discovery-of-endocannabinoid-gene-mutation-leads-to-identification-of- new-rare-pediatric; how-a-brain-area-implicated-in-alzheimers-may-be-vulnerable-to-degeneration; legalized-Cannabis-linked-to-fewer-synthetic-cannabinoid-poisonings; microbiome-disruption-of-gut-microbial-balance-is-associated-with-increased-mortality-after-kidney; study-examines-current-state-regulations-public-health-risks-of-growing-medicinal-and-recreational; study-finds-damage-in-the-lungs-of-chronic-e-cigarette-users; study-shows-inexpensive-readily-available-chemical-may-limit- impact-of-covid-19; news/an-important-step-toward-next-generation-probiotics/iRJLEJLdrnQnxNwkQsZfO; news/a-novel-powerful-tool-to-unveil-the-communication-between-gut-microbes-and-the-brain/50avDZEuKEPrPDQ9WWIAtW; news/biomarkers-predict-weight-loss-suggest-personalized-diets/6LcjwvuzdLCGJtGzjXofli; news/gut-bugs-control-the-body-and-8217-s-thermostat/5W5uwV2eEhCGwBi9gNvCsd; www.mdlinx.com/news/how-gut-bacteria-evade-the-immune-system/4rxKyjYw4As9uJtyToxa20; news/researchers-discover-exploiting-microbiome-bacteria-in-patients-with-lung-infections-improves-low/6eUoyAWR80rQ82VhNoDXut; news/salmonella-exposure-α-risk-for-colon-cancer-finds-study/7iEt5 g6w901j3MxCoSP4DC; news/using-prebiotics-to-help-treat-parkinson-s-disease/6ZaaBZhfQFayurPUnjX68; changes-in-gut-microbiome-alters-rewarding-effects-of-cocaine-and-cravings/5PctrUgPFVp2LyGwGYDids
www.mdpi.com/1420-3049/25/7/1567; 1420-3049/26/9/2774/pdf; 1424-8247/15/3/359; 2076-3921/10/9/1417; 2077-0383/9/6/1925; 2079-9284/7/2/43/pdf; 2223-7747/1l/10/1357; 2227-9059/9/9/1270; 2227-9717/9/7/1180; 2072-6643/13/10/3420; www.mdpi.com/2072-6643/15/14/3265
www.medicalnewstoday.com/articles/265922; 308044; atrial-fibrillation-Cannabis-and-other-drugs-linked-to-increased-afib-risk; endocannabinoid; foods-like-meat-sugar-and-coffee-may-worsen-menstrual-cramps-research-shows; hemp-protein; marijuana-and-glaucoma; best-vegan-protein-powder; best-vegan-protein-powder; me-ds-how-the-gut-microbiome-may-impact-the-immune-response; type-2-diabetes-gut-bateria-linked-to-insulin-sensitivity
www.medpagetoday.com/gastroenterology/inflammatoryboweldisease/102556; meetingcoverage/asa/101365; neurology/alzheimersdisease/101062
www.medscape.com/viewarticle/982156; 987701
www.mountsinai.org/health-library/herb/echinacea
www.napra.ca/sites/default/files/2022-07/NAPRA_Compounding_Competencies Jul2022_EN_Final.pdf
www.nass.usda.gov/Newsroom/2022/02-17-2022.php
www.nationalacademies.org/ocga/testimonies/116-session-/the-health-effects-of-Cannabis-and-cannabinoids
www.naturalproductsinsider.com/claims/medical-foods-between-rock-and-hard-place; regulatory/key-lawmaker-o-sponsors-cbd-supplements-bill
www.nature.com/articles/25267; 525S10a; d41586-022-03368-2; npp2017126; npp20l7130; s41380-022-01741-1; s41380-022-01830-1; s41386-018-0076-y; s41398-022-02112-8; s41467-022-32594-5.pdf; s41467-022-33395-6; s41467-022-33843-3; s41575-022-00698-4; s41598-019-56785-1; s41598-021-02770-6; s41598-021-81049-2; s41598-021-87740-8.pdf; s41598-022-15134-5; s43856-022-00202-8; s41368-022-00212-1; s41386-023-01661-w; s41392-022-00918-y; s41522-022-00360-6; s41586-022-05620-1; s41591-023-02227-5; s41598-022-25303-1; s41598-022-26847-y; s41598-023-27784-0; s42003-022-04380-y; s42255-022-00716-4; s44161-022-00204-8; s41420-022-00876-y
www.ncbi.nlm.nih.gov/books/NBK423845/; NBK425740; NBK425742; NBK507184; NBK5225; NBK546328; NBK92752/
www.ncbi.nlm.nih.gov/pmc/artidles/PMC10529306; PMC1136308/; PMC1572356; PMC1573233; PMC160903; PMC2219540; PMC2291677; PMC2503660; PMC2659017; PMC2719736; PMC2779044; PMC2815025; PMC2931553; PMC2944423; PMC3034734; PMC3037578; PMC3165946; PMC3165946; PMC3165957; PMC3207709; PMC3223967; PMC3249407; PMC3372428; PMC3382450; PMC3388539; PMC3423237; PMC3481530; PMC3481530; PMC3492530; PMC3525862; PMC3525862; PMC3533417; PMC3549323; PMC3566541; PMC3579248; PMC3601579; PMC3698539; PMC3705914; PMC7230191; PMC8573844; PMC3820295; PMC3820295; PMC3954479; PMC4128345; PMC4139007; PMC4276990; PMC4318341; PMC4322067; PMC4363290; PMC4375466; PMC4389556; PMC4604192; PMC4669115; PMC4737462; PMC4789136; PMC4805070; PMC4813193; PMC4882496; PMC4918871; PMC4940133; PMC4940815; PMC5009397; PMC5094513; PMC5132083; PMC5209363; PMC5314139; PMC5314139; PMC5342457; PMC53598; PMC5429331; PMC5473390; PMC5544121; PMC5576596; PMC5576600; PMC5628559; PMC5665515; PMC5672138; PMC5771053; PMC5792153; PMC5812006; PMC5859479; PMC5870061; PMC5877694; PMC5908414; PMC5964033; PMC5968020; PMC6021502; PMC6027320; PMC6073490; PMC6150799; PMC6156479; PMC6196295; PMC6214864; PMC6222489; PMC6277878; PMC6288804; PMC6361048; PMC6397040; PMC6438767; PMC6455058; PMC6513729; PMC6745571; PMC6790068; PMC6831725; PMC6866665; PMC6893937; PMC6963522; PMC7000163; PMC7023045; PMC7036282; PMC7052834; PMC7148385; PMC7150944; PMC7181184; PMC7204604; PMC7246936; PMC7279038; PMC7300966; PMC7321064; PMC7324885; PMC7345649; PMC7388834; PMC7409346; PMC7442575; PMC7464709; PMC7468657; PMC7480712; PMC7498943; PMC7522354; PMC7527511; PMC7558665; PMC7605020; PMC7605027; PMC7687025; PMC7693730; PMC7701971; PMC7719758; PMC7723146; PMC7754931; PMC7759141; PMC7763054; PMC7767453; PMC7835327; PMC7863932; PMC7874763; PMC7931196; PMC7949482; PMC8037087; PMC8123278; PMC8151290; PMC8157570; PMC8180479; PMC8196152; PMC8363108; PMC8417625; PMC8467477; PMC8525668; PMC8573844; PMC8778347; PMC8861494; PMC9322760; PMC9406052; PMC9406356; PMC2964429; PMC3731629; PMC5094513; PMC5409757; PMC5524138; PMC5531397; PMC5566641; PMC5902005; PMC6121872; PMC6346118; PMC6346118; PMC6358922; PMC6363527; PMC6866665; PMC7058978; PMC7400376; PMC7445536; PMC7926405; PMC8399199; PMC8404091; PMC8517709; PMC9029443; PMC9725099; PMC9816051; PMC6991146; PMC4916793
www.nccih.nih.gov/health/Cannabis-marijuana-and-cannabinoids-what-you-need-to-know
www.ncsl.org/bookstore/state-legislatures-magazine/marijuana-deep-dive.aspx
www.neurogan.com/blog/cbg-dosage/
www.newscientist.com/artidle/2358189-chronic-fatigue-syndrome-linked-to-lower-levels-of-some-gut-bacteria/
www.news-medical.net/news/20210805/Could-α-plant-metabolite-in-liverwors-be-α-potent-antiviral-against-SARS-CoV-2-and-other-RNA-viruses.aspx
www.news-medical.net/news/20221017/Increased-TMPRSS2-expression-in-tongue-tissue-of-females-and-alcohol-drinkers-identified-as-α-potential-risk-factor-for-SARS-CoV-2-infection.aspx
www.news-medical.net/news/20221026/Dangers-of-recreationalmedical-Cannabis-use.aspx; 20221109/Melatonin-could-be-α-potential-therapy-for-long-COVID-symptoms.aspx; 20230104/The-metabolic-links-between-diet-induced-changes-in-the-gut-and-NASH.aspx; 20230105/Current-research-on-gut-microbial-modulation-with-culinary-herbs-and-spices.aspx; 20230112/Reshaping-the-gut-microbiome-could-be-α-possible-way-to-prevent-or-treat-neurodegeneration.aspx; 20230206/What-do-gut-microbiota-have-to-do-with-Alzheimers-and-dementia.aspx; 20230410/Antioxidant-rich-phenolic-compounds-A-key-to-disease-prevention-gut-health-and-brain-homeostasis.aspx
www.nih.gov/news-events/news-releases/probiotic-markedly-reduces-s-aureus-colonization-phase-2-trial; studies-find-microbiome-changes-may-be-signature-meds; news-events/nih-research-matters/erythritol- cardiovascular-events; news-events/nih-research-matters/gut-microbes-may-affect-motivation-exercise.
www.npr.org/2018/07/27/632261920/d-c-has-had-more-than-300-suspected-k2-overdoses-in-2-weeks; 2022/01/05/1070212871/usda-bioengineered-food-label-gmo; 2022/10/10/1127708285/marijuana-pardon-biden-black-people-war-on-drugs-harm
www.nutraingredients.com/News/Promotional-Features/The-Role-of-ECS-in-Mood-Sleep-and-Stress-Rel ated-Health-Concerns
www.nutraingredients-asia.com/Artide/2020/05/12/Boosting-bioavailability-PEA-absorption-up-increases-by-1.75-times-using-novel-technology
www.nutraingredients-usa.com/Artide/2016/04/28/ProbioTech-wants-probiotic-suppository-to-be-counted-as-supplement; News/Promotional-Features/Levagen-R-PEA-Is-the-New-Solution-for-Athlete-and-Ageing-Musdes; Product- innovations/BioPerine-Increases-Ingredient-Bioavailability
www.nutritionnews.abbott/healthy-living/aging-well/why-hmb-is-important-for-aging-muscles/
www.nwsci.com/customer/docs/SKUDocs/RMR/Technical %20Data_Extractions_03.28.18.pdf
www.nytimes.com/2005/04/17/us/how-one-pill-escaped-the-list-of-controlled-steroids.html
www.nytimes.com/2022/09/06/well/mind/melatonin-dependency.html
www.opss.org/article/hemp-and-dod-policy.
www.oxygenconcentratorsupplies.com/articles/what-is-the-difference-between-α-nebulizer-and-an-inhaler/
www.painmedicinenews.com/Online-First/Artide/10-22/Pain-Signaling-May-Also-Protect-Intestinal-Lining/68461
www.painnewsnetwork.org/stories/2019/2/27/study-thc-more-effective-than-cbd-in-treating-pain; 2021/10/29/more-americans-using-Cannabis-to-manage-chronic-pain-than-opioids; 2022/10/21/lab-mice-agree-delta-8-just-like-taking-delta-9; 2022/10/31/smoking-marijuana-more-effective-than-cbd-extracts-in-treating-back-pain; 2022/10/6/biden-pardons-thousands-convicted-of-marijuana-possession; 2022/7/7/nearly-half-of-cbd-oils-mislabeled 2022/9/2/heavy-metals-and-plastic-residue-found-in-many-cbd-products; 2022/9/23/hemp-derived-delta-8-raises-health-concerns; 2022/9/28/medical-Cannabis-helps-chronic-pain-patients-stop-or-reduce-use-of-rx-opioids-4uhdN
www.pama.org/the-amt-lawyer/amts-and-cbds/
www.pccarx.com/Blog/choosing-the-right-suppository-base; products/PCCALIPODERM % C2% AE/30-3338/PROPRIETARYBASES
www.pnas.org/doi/pdf/10.1073/pnas.0402935101; 10.1073/pnas.1800779115; 10.1073/pnas.98.3.793; 10.1073/pnas.1509795112
www.practicalpainmanagement.com/resources/diagnostic-tests/false-positive-screen-marijuana
www.prnewswire.com/news-releases/leading-global-medical-Cannabis-company-khiron-publishes-dinical-study-on-the-effectiveness-of-its-products-at-controlling-chronic-pain-301517698.html
www.projectcbd.org/environment/wasted-plastic-packaging-Cannabis-biomass-throw-away-vapes; medicine/do-Cannabis-suppositories-work; sites/projectcbd/files/downloads/cannabinoid-boiling-points-thc-cbd.pdf
www.psychiatrictimes.com/view/synthetic-cannabinoids-cathinones; the-impact-of-Cannabis-use-on-medic
www.psychologytoday.com/us/blog/the-athletes-way/202102/runner-s-high-depends-endocannabinoids-not-endorphins
www.publish.csiro.au/ch/pdf/CH21006
www.quantamagazine.org/mobile-genes-from-the-mother-shape-the-babys-microbiome-20230117/
www.quora.com/Is-it-possible-to-have-withdrawals-from-CBD-oil
www.raps.org/news-and-articles/news-articles/2022/8/foods-for-special-medical-purposesmedical-foods-a
www.realsimple.com/how-to-improve-gut-health-naturally-6833619
www.royalqueenseeds.com/blog-α-brief-history-of-Cannabis-use-in-world-religions-n624; blog-bioavailability-the-story-of-how-Cannabis-enters-the-body-n943; blog-Cannabis-nasal-sprays-do-they-really-work-nl204; blog-growing-Cannabis-plants-seeds-versus-clones-n27
www.rxlist.com/liverwort/supplements.htm; pennsaid-drug.htm
www.salk.edu/news-release/active-ingredient-in-Cannabis-protects-aging-brain-cel|s/
www.samhsa.gov/marijuana
www.sartorius.com/en/applications/applied-industries/Cannabis-testing/moisture-analysis
www.scielo.org.co/scielo.php?script=sci_artext&pid=S0120-99572020000100104&lng=e&nrm=iso&tlng=en
www.science.org/content/article/gut-microbe-linked-depression-large-health-study
www.science.org/doi/10.1126/science.abn7065; 10.1126/stke.3362006twl69; 10.1126/scitranslmed.abg8476; 10.1126/sciadv.add5163
www.sciencealert.com/babies-may-not-be-born-with-α-microbiome-after-all-scientists-say
www.sciencedirect.com/science/article/abs/pii/S0899900723001090; S0924224416303612 0024320576903362; 0031942277801027; S000293431930453X; S0006295218303046; S0014299916303661; S0092867422004652; S0361923022000041; S0925521420309881; S1525505021007757; S002239562100546X; S0023643816302857; S162013418300047; S0273230020300738; S0753332218375668; S0753332221010635; S0924224422001984; S1936523318305990; S2665906921000040; S2772566922000015; S2772566922000015; S2772566922000039; 50753332223002020; S2772753X22000120
www.sciencedirect.com/topics/neuroscience/beta-adrenergic-receptor; topics/neuroscience/endocannabinoids; topics/neuroscience/gpr55; topics/nursing-and-health-professions/nasal-spray; topics/pharmacology-toxicology-and-pharmaceutical-science/panaxynol; topics/pharmacology-toxicology-and-pharmaceutical-science/cannabichromene; topics/pharmacology-toxicology- and-pharmaceutical-science/friedelin; topics/pharmacology-toxicology-and-pharmaceutical-science/acceptable-daily-intake; biochemistry-genetics-and-molecular-biology/symbiosis; chemistry/endocannabinoid; article/marijuana-skunk-smell-Cannabis-pot-plant-chemistry
www.scientificamerican.com/article/covid-can-cause-strange-eye-and-ear-symptoms/; some-of-the-parts-is-marijuana-rsquo-s-Idquo-entourage-effect-rdquo-scientifically-valid
www.sdlabs.com/leaky-gut-syndrome-cannabinoids-and-the-endocannabinoid-system-ecs-as-α-therapeutic-target/
www.scotsman.com/health/is-cbd-halal-or-haram-α-guide-for-2022-3565363
www.sefaria.org/sheets/12977?lang=bi
www.silverfernbrand.com/blogs/news/ways-the-keto-diet-may-be-decimating-the-microbiome.
www.spandidos-publications.com/10.3892/ijmm.2016.2779; 10.3892/or.2017.6042
www.spglobal.com/marketintelligence/en/news-insights/latest-news-headlines/chd-companies-look-overseas-to-source-hemp-as-us-canada-growing-rules-relax-55507665
www.statnews.com/2021/02/03/jazz-pharma-to-acquire-gw-pharma-for-7-2-billion-adding-cbd-based-treatment-for-severe-epilepsy/
www.statnews.com/2022/09/20/cbd-food-and-drink-regulation/
www.sydney.edu.au/news-opinion/news/2021/10/05/mother-of-all-cannabinoids-anti-seizure-compounds-Cannabis.html
www.tandfonline.com/doi/abs/10.1080/01904169709365252; 10.1080/10408444.2020.1762539; 10.1300/J175v02n03_02; 10.1080/10826084.2022.2107673; 10.1080/15563650.2022.2099887; 10.1080/17476348.2020.1787836; 10.1080/19490976.2021.1997559; 10.2147/JEP.S162059
www.thegrowthop.com/Cannabis-health/what-the-heck-is-nebul izing-and-is-it-the-next-big-thing-for-consuming-Cannabis
www.theguardian.com/lifeandstyle/2023/jan/17/meditation-could-have-positive-impact-on-gut-overall-health-microbiome
www.thepermanentejournal.org/doi/epdf/10.7812/TPP/19.200
www.the-scientist.com/critic-at-large/body-heal-thyself-34917
www.the-scientist.com/news-opinion/gut-microbiomes-offer-insights-into-chronic-fatigue-syndrome-70968
www.thevirginoliveoiler.com/blogs/julies-blog/mct-oil-vs-evoo-blog-29
www.thorne.com/take-5-daily/article/plants-as-medicine-are-cannabinoids-the-next-breakthrough-in-plant-medicine
www.transformationstreatment.center/resources/drug-overdose/how-many-people-have-died-from-marijuana/
www.unodc.org/documents/scientific/Synthetic_Cannabinoids.pdf
www.usatoday.com/story/news/health/2022/08/31/marijuana-use-outpacing-cigarette-use-us/7947821001/; 2022/10/29/hemp-delta-9-thc-regulation-explained/10595313002/
www.uspharmacist.com/article/Cannabis-edibles-and-pediatric-toxicity-risk?wc_mid=6220:706453&wc_rid=6220:642757; pros-and-cons-of-pharmacy-compounding
www.veganfirst.com/article/the-benefits-of-eating-hemp-hearts-daily-can-surprise-you
www.veriheal.com/blog/the-bioavailability-of-Cannabis-through-various-delivery-methods/; conditions/chronic-itch-pruritus/
www.verywellhealth.com/does-weed-help-with-adhd-benefits-vs-risks-5210315
www.vfw.org/media-and-events/latest-for-ptsd-treatment
www.walshmedical media.com/open-access/endocannabinoid-stimulated-release-of-nitric-oxide-and-its-mitochondrial-influence-triggering-vascular-pathology-2153-2435-1000378.pdf
www.washingtonpost.com/politics/2022/10/24/marijuana-same-sex-marriage-legalization/; 2022/03/31/christian-Cannabis-craig-gross;
   wellness/2023/01/10/meal-timing-big-meals; wellness/2023/03/08/exercise-microbiome-motivation
www.webmd.com/connect-to-care/addiction-treatment-recovery/facts-about-marijuana-withdrawal; diet/what-to-know-about-sodium-benzoate; heart-disease/resveratrol-supplements; pain-management/news/20210429/cbd-how-much-pain-relief-is-real-how-much-is-placebo; vitamins/ai/ingredientmono-1439/cannabidiol-cbd; vitamins/ai/ingredientmono-1602/cannabigerol-cbg; vitamins/ai/ingredientmono-294/quercetin; vitamins-and-supplements/dmso-uses-and-risks; vitamins/ai/ingredientmono-37/liverwort;
www.wellandgood.com/best-cbd-lube/
www.wikihow.com/Identify-Female-and-Male-Marijuana-Plants
www.wikileaf.com/thestash/atomizer-vaping/
www.wsj.com/artiles/can-intermittent-fasting-help-combat-youth-obesity-11673962097? mod=wsjhp_columnists_pos3 AU20l6210070; AU2019211188; AU2021203749; AU638795; AU643757; BRl12017015536; CA2035711; CA2036307C; CA2977421; CA3089490; CNi10878010; EP0444451; EP0445781; EP0445781B1; EP3247371; EP3556376; EP3746419; ES2801005T3; F195369B; F195369C; GB2393182; HU208132; HU211967; IHU215125; 1E72953; 1E910492; IE910700; IL110447; IL97313; IL97426; JP3157179; KR0183396; KR100191258; PT96871; RU2052457; RU2073670; WO2020249184; WO2016116628; WO2016118540; WO2016153347; WO20180130682; WO2019145552; WO2020171713; WO2020252396; WO2021037343; WO2021047491; WO2021141955; WO2021037343; WO2016059411; US 20030021752; 20040049059; 20040106625; 20040127501; 20040147767; 20040186111; 20050042172; 20050061314; 20050165088; 20050266108; 20060167283; 20060199824; 20070032544; 20070077660; 20080017191; 20080031977; 20080103193; 20080153835; 20090035396; 20090068143; 20090318526; 20100004254; 20100008985; 20100029739; 20100119606; 20100168448; 20100204312; 20100210686; 20100210860; 20100222437; 20100239693; 20100249223; 20100286098; 20100292345; 20110021617; 20110038958; 20110046081; 20110092583; 20110098348; 20110206816; 20110281885; 20120004217; 20120004251; 20120015954; 20120043242; 20120046351; 20120046352; 20120059062; 20120095088; 20120107300; 20120263804; 20130011484; 20130059018; 20130209483; 20130251824; 20130280343; 20130334045; 20130337477; 20140039043; 20140065243; 20140107192; 20140142112; 20040143126; 20140221469; 20140228438; 20140243405; 20140248379; 20140271940; 20140287068; 20140298511; 20140302121; 20140314757; 20140343136; 20140357708; 20140377382; 20140378539; 20150038567; 20150057341; 20150057342; 20150086653; 20150105455; 20150126754; 20150132400; 20150136158; 20150150844; 20150152018; 20150181924; 20150181925; 20150190442; 20150203434; 20150231108; 20150245991; 20150265636; 20150265637; 20150265720; 20150297653; 20150297654; 20150313868; 20150320698; 20150320720; 20150324534; 20150324942; 20150342922; 20150359188; 20150361469; 20150366154; 20150374770; 20160000843; 20160010126; 20160015683; 20160018424; 20160038437; 20160039591; 20160044934; 20160053220; 20160074451; 20160081927; 20160081975; 20160081976; 20160088809; 20160088870; 20160106705; 20160136128; 20160151328; 20160157524; 20160161459; 20160166498; 20160166786; 20160171164; 20160184237; 20160184258; 20160184259; 20160206610; 20160211693; 20160214920; 20160220593; 20160228385; 20160228787; 20160235661; 20160243460; 20160250270; 20160256410; 20160256411; 20160256435; 20160264917; 20160279183; 20160298151; 20160300289; 20160309774; 20160310443; 20160324091; 20160324776; 20160326130; 20160331720; 20160331913; 20160335543; 20160338974; 20160340629; 20160345477; 20160346339; 20160355853; 20160355854; 20160360721; 20160367479; 20160367522; 20160370337; 20160374958; 20160376211; 20170000744; 20170020941; 20170020942; 20170020943; 20170020944; 20170020945; 20170021025; 20170021026; 20170021029; 20170027978; 20170042791; 20170045450; 20170049830; 20170056368; 20170059480; 20170060907; 20170079933; 20170095518; 20170112161; 20170112801; 20170112855; 20170119728; 20170127727; 20170135984; 20170136196; 20170143642; 20170143664; 20170152530; 20170157343; 20170159080; 20170172201; 20170172977; 20170189373; 20170202170; 20170209409; 20170212049; 20170224841; 20170224842; 20170232210; 20170233778; 20170234897; 20170247650; 20170252300; 20170265494; 20170266127; 20170266128; 20170266153; 20170266397; 20170273937; 20170274030; 20170283837; 20170291120; 20170298399; 20170304217; 20170312261; 20170312474; 20170319607; 20170333503; 20170333505; 20170336341; 20170339907; 20170340562; 20170348276; 20170348277; 20170360745; 20170360861; 20170361525; 20170362195; 20170367386; 20170368020; 20170368021; 20170368022; 20180000727; 20180000857; 20180000879; 20180016203; 20180021247; 20180021438; 20180035689; 20180036278; 20180039741; 20180042845; 20180042975; 20180043114; 20180043115; 20180064055; 20180064645; 20180064772; 20180069078; 20180071210; 20180071246; 20180071654; 20180073043; 20180074045; 20180077967; 20180078504; 20180078523; 20180078593; 20180092392; 20180093054; 20180098552; 20180098962; 20180099236; 20180110730; 20180110753; 20180116240; 20180117161; 20180125980; 20180126003; 20180127327; 20180128843; 20180133272; 20180137125; 20180140787; 20180141277; 20180143212; 20180147141; 20180147247; 20180155748; 20180161285; 20180162828; 20180169035; 20180170846; 20180179564; 20180184705; 20180193304; 20180193393; 20180193399; 20180193403; 20180199531; 20180214790; 20180221304; 20180221333; 20180221396; 20180222879; 20180224411; 20180228788; 20180228854; 20180230499; 20180233558; 20180233559; 20180236017; 20180237368; 20180243259; 20180258439; 20180263952; 20180264059; 20180264121; 20180264122; 20180271827; 20180271924; 20180280459; 20180280473; 20180280637; 20180282250; 20180284145; 20180284402; 20180289665; 20180295804; 20180296493; 20180296498; 20180297726; 20180300457; 20180311180; 20180311181; 20180311184; 20180311205; 20180318237; 20180318361; 20180318529; 20180320209; 20180325861; 20180333446; 20180334692; 20180338930; 20180339973; 20180343812; 20180344661; 20180344662; 20180344663; 20180344676; 20180344684; 20180344686; 20180344786; 20180344790; 20180344860; 20180344954; 20180346866; 20180352848; 20180353461; 20180353558; 20180360739; 20180360757; 20180360770; 20180360894; 20180360896; 20180362429; 20180369191; 20180369192; 20180371507; 20180376644; 20190000795; 20190008823; 20190008845; 20190010106; 20190010107; 20190010110; 20190015346; 20190015382; 20190022158; 20190022229; 20190029993; 20190030044; 20190030062; 20190030101; 20190030103; 20190030165; 20190030170; 20190033210; 20190035890; 20190035891; 20190037909; 20190038663; 20190038853; 20190038995; 20190046440; 20190046499; 20190046598; 20190054176; 20190060220; 20190060225; 20190060250; 20190060251; 20190060252; 20190060300; 20190060381; 20190062144; 20190070124; 20190070396; 20190076349; 20190076355; 20190077782; 20190078168; 20190083387; 20190083622; 20190083902; 20190085279; 20190085347; 20190090402; 20190090438; 20190090527; 20190091144; 20190091198; 20190100731; 20190105298; 20190105619; 20190110981; 20190111093; 20190117617; 20190117778; 20190119694; 20190124864; 20190125660; 20190125779; 20190133966; 20190133992; 20190134121; 20190134123; 20190134125; 20190134532; 20190142788; 20190142851; 20190142888; 20190151771; 20190153460; 20190153461; 20190160393; 20190161763; 20190167583; 20190167740; 20190167749; 20190175523; 20190177294; 20190177674; 20190183003; 20190183004; 20190183005; 20190183006; 20190183007; 20190183008; 20190183009; 20190183010; 20190183848; 20190183850; 20190183853; 20190183914; 20190185946; 20190192422; 20190192429; 20190192691; 20190192794; 20190192810; 20190192993; 20190194585; 20190201463; 20190201809; 20190209487; 20190209633; 20190214145; 20190216869; 20190216870; 20190221298; 20190224118; 20190224141; 20190224142; 20190224327; 20190225975; 20190226912; 20190230882; 20190231711; 20190231737; 20190231833; 20190232194; 20190240191; 20190241536; 20190241537; 20190241633; 20190246591; 20190247299; 20190247325; 20190247358; 20190254213; 20190254326; 20190254969; 20190255014; 20190256486; 20190257746; 20190261657; 20190262304; 20190269865; 20190270691; 20190275095; 20190275268; 20190275270; 20190276420; 20190281871; 20190282502; 20190282643; 20190298683; 20190300888; 20190305084; 20190307695; 20190307719; 20190307720; 20190307825; 20190307826; 20190314296; 20190314325; 20190314326; 20190314327; 20190314739; 20190316144; 20190321306; 20190321330; 20190321355; 20190321425; 20190321426; 20190328699; 20190328807; 20190328884; 20190331656; 20190336472; 20190336521; 20190338301; 20190343760; 20190350949; 20190352662; 20190352679; 20190355472; 20190358169; 20190358195; 20190358197; 20190364957; 20190364968; 20190364969; 20190365664; 20190365667; 20190365702; 20190366230; 20190366231; 20190367953; 20190369127; 20190373679; 20190374501; 20190374502; 20190374552; 20190376929; 20190380388; 20190382325; 20190382326; 20190382708; 20190382807; 20190382814; 20190388384; 20190390408; 20200000111; 20200000765; 20200000766; 20200001200; 20200002306; 20200008428; 20200008442; 20200009107; 20200009109; 20200010786; 20200016092; 20200016095; 20200017889; 20200017900; 20200020449; 20200022386; 20200022925; 20200022945; 20200022946; 20200022948; 20200025728; 20200030250; 20200030281; 20200030282; 20200035118; 20200035119; 20200035120; 20200035121; 20200037638; 20200038305; 20200038366; 20200038367; 20200038421; 20200039908; 20200046007; 20200046639; 20200046675; 20200046722; 20200046836; 20200047082; 20200048214; 20200048215; 20200048664; 20200051455; 20200054245; 20200054701; 20200054702; 20200060305; 20200061004; 20200061021; 20200061023; 20200061136; 20200061138; 20200061195; 20200063170; 20200063171; 20200063202; 20200068899; 20200068919; 20200069567; 20200069581; 20200069604; 20200069605; 20200069606; 20200069618; 20200069638; 20200069639; 20200069776; 20200069896; 20200071285; 20200071732; 20200078297; 20200078316; 20200078332; 20200078333; 20200078427; 20200079715; 20200079751; 20200084989; 20200085739; 20200085740; 20200085959; 20200086228; 20200093075; 20200093755; 20200093876; 20200094003; 20200100433; 20200101034; 20200101041; 20200107510; 20200108018; 20200108019; 20200108027; 20200108044; 20200108045; 20200108046; 20200108082; 20200108101; 20200115306; 20200120987; 20200121606; 20200121616; 20200121637; 20200122052; 20200123511; 20200128864; 20200131146; 20200138072; 20200138730; 20200138736; 20200138737; 20200138772; 20200138773; 20200138774; 20200138885; 20200146361; 20200146998; 20200146999; 20200147034; 20200147035; 20200155469; 20200155629; 20200155635; 20200157017; 20200163900; 20200163931; 20200163932; 20200164012; 20200165219; 20200165544; 20200165641; 20200170272; 20200170283; 20200170944; 20200170950; 20200170962; 20200170994; 20200171083; 20200172459; 20200172503; 20200172841; 20200172917; 20200178572; 20200179269; 20200179342; 20200179426; 20200181050; 20200181631; 20200188298; 20200188301; 20200188323; 20200188325; 20200188347; 20200188349; 20200188461; 20200188812; 20200190002; 20200190428; 20200191480; 20200193861; 20200193862; 20200196504; 20200196656; 20200197329; 20200197359; 20200197462; 20200197464; 20200197466; 20200197521; 20200199055; 20200206126; 20200206151; 20200206185; 20200206415; 20200206430; 20200211688; 20200214996; 20200215022; 20200215024; 20200215026; 20200215047; 20200215136; 20200215137; 20200219167; 20200222359; 20200222361; 20200222543; 20200222828; 20200222829; 20200224231; 20200229411; 20200229510; 20200230185; 20200230267; 20200237622; 20200237658; 20200237659; 20200237660; 20200237661; 20200237662; 20200237666; 20200237679; 20200237714; 20200237840; 20200239428; 20200239916; 20200245666; 20200246404; 20200246405; 20200246406; 20200246722; 20200253919; 20200253921; 20200253922; 20200253923; 20200253924; 20200253947; 20200253948; 20200254038; 20200254039; 20200254040; 20200254041; 20200254104; 20200254271; 20200254407; 20200255389; 20200261387; 20200261404; 20200261405; 20200261406; 20200261407; 20200261824; 20200268646; 20200268681; 20200268708; 20200268817; 20200269156; 20200270623; 20200273578; 20200276115; 20200276116; 20200276117; 20200276118; 20200276119; 20200276155; 20200276324; 20200281278; 20200281889; 20200281890; 20200282062; 20200283283; 20200283807; 20200286597; 20200288659; 20200289458; 20200289459; 20200289654; 20200289768; 20200290791; 20200290988; 20200291434; 20200292564; 20200297025; 20200297690; 20200297948; 20200299216; 20200305265; 20200306463; 20200306665; 20200315253; 20200315256; 20200315957; 20200315988; 20200316015; 20200316016; 20200316151; 20200323162; 20200323163; 20200323164; 20200323773; 20200323791; 20200323936; 20200324063; 20200325091; 20200329601; 20200329658; 20200329775; 20200330339; 20200330378; 20200330379; 20200330382; 20200330423; 20200330424; 20200330425; 20200330537; 20200334737; 20200338150; 20200338151; 20200339528; 20200340026; 20200340145; 20200345585; 20200345657; 20200345684; 20200345686; 20200345816; 20200352172; 20200352191; 20200352199; 20200352216; 20200352249; 20200352901; 20200360239; 20200360292; 20200360337; 20200361841; 20200367548; 20200368639; 20200370073; 20200372993; 20200375882; 20200375886; 20200375907; 20200375918; 20200375938; 20200375940; 20200376156; 20200376241; 20200383331; 20200383371; 20200383935; 20200390267; 20200390697; 20200390711; 20200390721; 20200390739; 20200390838; 20200396961; 20200397743; 20200397842; 20200398180; 20200398182; 20200398183; 20200398184; 20200399194; 20200402662; 20200405657; 20200405685; 20200405686; 20200405980; 20200406205; 20200406206; 20200408740; 20210000166; 20210000742; 20210000759; 20210000789; 20210000791; 20210000899; 20210003488; 20210007407; 20210008004; 20210008025; 20210008026; 20210008027; 20210008138; 20210008139; 20210009548; 20210015740; 20210015746; 20210015759; 20210015786; 20210015836; 20210016020; 20210016202; 20210017145; 20210022305; 20210023005; 20210023022; 20210023044; 20210023045; 20210023053; 20210023156; 20210023316; 20210030697; 20210030712; 20210030768; 20210030777; 20210030779; 20210030824; 20210030979; 20210032650; 20210038493; 20210038513; 20210038558; 20210038559; 20210038560; 20210038666; 20210038805; 20210039013; 20210040492; 20210040512; 20210045311; 20210045434; 20210045452; 20210045457; 20210045933; 20210045943; 20210045998; 20210046017; 20210046041; 20210046111; 20210046261; 20210047583; 20210051995; 20210052545; 20210052753; 20210053902; 20210055050; 20210057073; 20210059307; 20210059935; 20210059949; 20210059960; 20210059975; 20210059976; 20210059978; 20210065860; 20210068444; 20210068450; 20210069103; 20210069170; 20210069198; 20210069271; 20210069611; 20210069675; 20210071186; 20210071209; 20210077389; 20210077394; 20210077411; 20210077422; 20210078967; 20210079314; 20210082307; 20210084971; 20210085596; 20210085638; 20210085666; 20210085671; 20210085887; 20210085888; 20210089946; 20210092972; 20210093529; 20210093539; 20210093552; 20210093559; 20210093561; 20210093652; 20210093690; 20210093724; 20210100741; 20210100746; 20210100755; 20210106637; 20210106929; 20210107884; 20210112743; 20210113489; 20210113490; 20210115513; 20210121403; 20210121416; 20210121435; 20210121794; 20210128450; 20210128518; 20210128519; 20210128521; 20210128522; 20210128534; 20210128658; 20210130250; 20210130755; 20210131953; 20210136973; 20210136974; 20210137137; 20210137833; 20210137877; 20210142876; 20210144947; 20210145036; 20210145043; 20210145077; 20210145764; 20210145841; 20210145851; 20210145910; 20210146274; 20210147770; 20210153562; 20210153565; 20210153566; 20210153568; 20210153569; 20210154139; 20210154169; 20210154252; 20210154420; 20210154596; 20210161201; 20210161833; 20210162143; 20210162318; 20210163387; 20210163438; 20210169125; 20210169759; 20210169790; 20210169791; 20210169792; 20210169795; 20210169806; 20210169816; 20210169820; 20210169823; 20210170301; 20210170302; 20210172091; 420210177013; 20210177036; 20210177037; 20210177038; 20210177041; 20210177042; 20210177043; 20210177044; 20210177738; 20210177739; 820210177740; 820210177748; 20210177754; 20210177800; 20210177858; 20210177914; 20210172; 7901; 864978; 820210186081; 20210186418; 20210186860; 20210186870; 20210186893; 20210188798; 20210189287; 20210189402; 20210189444; 20210193280; 920210193281; 20210199382; 9359625318; 20210195908; 20210196629; 20210196637; 20210196644; 20210196669; 20210196670; 20210196774; 20210196775; 20210197156; 20210204503; 20210204585; 20210204590; 20210205191; 20210205224; 20210205235; 20210205236; 20210205428; 20210206554; 20210207154; 20210211867; 20210212381; 20210212929; 20210212946; 20210212950; 20210212961; 20210212981; 20210212983; 20210212984; 20210213083; 20210213091; 20210213373; 20210219511; 20210219512; 20210219573; 20210219589; 20210219597; 20210219608; 20210219622; 20210220272; 20210220282; 20210220292; 20210220307; 20210220323; 20210220324; 20210228497; 20210228502; 20210228513; 20210228534; 20210228536; 20210230645; 20210235716; 20210235728; 20210235752; 20210235758; 10202; 102688; 6510207198; 1027199; 0210236458; 20210236459; 20210236573; 20210236575; 20210236687; 20210236954; 20210236955; 20210238117; 20210238561; 20210241918; 20210244078; 20210244095; 20210244653; 20210244684; 20210244686; 20210244703; 20210244924; 20210245073; 20210251157; 20210251276; 20210251917; 20210251918; 20210251946; 20210251947; 20210251948; 20210251949; 20210251950; 20210251976; 20210252087; 2021012; 10254030; 20210254083; 4973587; 5013837; 5068234; 5607933; 5817651; 86403126; 6949582; 7279493; 7572812; 7582635; 7622140; 7700368; 7799786; 7807711; 8008300; 8034843; 8324408; 8343553; 8349842; 8410064; 8481085; 8536177; 8604037; 8673368; 8758826; 8790719; 8846409; 8895078; 8980941; 9017737; 9034395; 9035130; 9044390; 9084771; 9095554; 9125859; 9186386; 9205063; 9254272; 9259449; 9265724; 9308175; 9326967; 9333229; 935825959625; 9370164; 9376367; 9394510; 9421187; 9434721; 9474725; 9512391; 9526715; 9526792; 9532593; 9555019; 9565865; 9585867; 9587212; 9597279; 9642317; 9649349; 9655936; 9655910; 9675654; 9675656; 9730911; 9732009; 9744200; 9765000; 9802880; 9809521; 9814695; 9815810; 69822384; 9827281; 9827282; 9827322; 9833408; 9833461; 9839241; 9839612; 9844518; 9844530; 9849108; 9854828; 9861609; 9861611; 9867859; 9879292; 9888703; 9895342; 9907823; 9918961; 9937146; 9937147; 9937218; 9937219; 9950976; 9955716; 9956173; 9956174; 9956498; 9962341; 9972680; 9974739; 9980996; 9981203; 10016363; 10028919; 10028987; 10046018; 10052339; 10059684; 10064905; 10064950; 10080736; 10080851; 10089818; 10084044; 10092538; 10092611; 10092852; 10093949; 10098867; 10103225; 10117883; 10117891; 10118006; 10143706; 10155018; 10155176; 10155708; 10172786; 10172809; 10172897; 10188628; 10189762; 10195159; 10202322; 10206888; 10207198; 10207199; 10214753; 10220063; 10226433; 10231948; 10238705; 10238745; 10239808; 10246431; 10258580; 10258601; 10265292; 10265295; 10272051; 10279000; 10285971; 10296714; 10301242; 10307392; 10308626; 10309894; 10319475; 10328111; 10328216; 10336978; 10350165; 10368502; 10369178; 10374036; 10378020; 10381440; 10383816; 10383892; 10384985; 10392635; 10405560; 10406186; 10408786; 10413521; 10413523; 10420809; 10435727; 10441552; 10441617; 10451480; 10456357; 10458908; 10471223; 10472652; 10486083; 10499584; 10502750; 10507404; 10512282; 10512614; 10512615; 10512629; 10512856; 10517848; 10517911; 10525093; 10526306; 10537592; 10538790; 10542770; 10543176; 10543190; 10548840; 10548931; 10555906; 10555927; 10555928; 10561634; 10561694; 10563211; 10568863; 10568864; 10568865; 10568920; 10569189; 10576157; 10582676; 10583160; 10588974; 10588979; 10595555; 10596124; 10596147; 10596159; 1101371; 1017689; 10597348 10604464; 10610512; 10617722; 10617834; 10617974; 10624872; 10624940; 10625175; 10632064; 10632432; 10633681; 10634689; 10639339; 10653787; 10654823; 10668044; 10668130; 10669248; 10675252; 10675264; 11034639; 1035010; 10688190; 10688191; 10695301; 10695316; 10702495; 10702565; 10704066; 10709088; 10709670; 10709747; 10709748; 10716766; 10716819; 10722490; 10724048; 10729665; 1108211; 1084770; 10737198; 10738268; 10743568; 10745644; 10751299; 10751300; 10751380; 10756180; 10757944; 10757945; 10765658; 10765713; 10765965; 10773184; 10777091; 10780075; 10780442; 10787674; 10787675; 10792318; 10792584; 10793498; 10795976; 10799450; 10799467; 10799546; 10799812; 10806707; 10807931; 10813889; 10813963; 10820624; 10821084; 10821147; 10822320; 10830780; 10835501; 10835839; 10837031; 10842772; 10842773; 10842786; 10843991; 10849852; 10849876; 10851076; 10851077; 10857107; 10857482; 10864172; 10864189; 10864239; 10864458; 10865157; 10870632; 10871467; 10878717; 10881637; 10888665; 10888666; 10894224; 10894780; 10894952; 10897915; 10897925; 10898463; 10905730; 10907163; 10912807; 10918686; 10925853; 10927477; 10933013; 10933016; 10933017; 10933073; 10933082; 10934554; 10940173; 10945953; 10946054; 10946306; 10954209; 10954534; 10959978; 10960322; 10961174; 10966924; 10966946; 10966953; 10973255; 10973864; 10974165; 10975379; 10975395; 10976293; 10980743; 10980772; 10980773; 10981850; 10981856; 10982243; 10987321; 10988785; 10991463; 10993977; 11000486; 11000818; 11007170; 11007238; 11013685; 11013715; 11017689; 11020355; 11026881; 11026915; 11026985; 11027218; 11028345; 11028417; 11034639; 11035010; 11040017; 11040295; 11040932; 11046664; 11046978; 11052055; 11058646; 11071761; 11072568; 11076539; 11077086; 11077089; 11077150; 11078145; 11078247; 11078502; 11083211; 11084770; 11085047; 11091455; PP27475P2; PP30434; PP30668; PP31535; PP31707; PP31724; PP31752; PP31874; PP31917; PP31918; PP32473; PP32725P2; PP33000; PP33162; PP33182; PP33210; PP33211; PP33212; and PP33332.

What is claimed is:

1. A pharmaceutical formulation comprising:
at least one cannabinoid selected from the group consisting of CBG, CBGA, CBGV, and CBGVA, or pharmaceutically salt, ester or amide thereof, substantially without CBDA synthase products and TCHA synthase products of the at least one cannabinoid; and
at least one additional active composition selected from the group consisting of an endocannabinoid, gamma amino butyric acid, and dehydroepiandrosterone.

2. The pharmaceutical formulation according to claim 1, provided in a unit dose or metered dose form.

3. The pharmaceutical formulation according to claim 2, wherein the at least one cannabinoid comprises at least 2.5 mg of the at least one cannabinoid selected from the group consisting of CBG, CBGA, CBGV, and CBGVA or a salt, ester or amide thereof.

4. The pharmaceutical formulation according to claim 2, wherein the at least one additional active composition comprises at least 25 mg of an endocannabinoid selected from the group consisting of LEA, PEA, SEA, and OEA.

5. The pharmaceutical formulation according to claim 2, wherein the at least one additional active composition comprises at least 10 mg of anandamide.

6. The pharmaceutical formulation according to claim 2, wherein the at least one additional active composition comprises at least 10 mg of dehydroepiandrosterone.

7. The pharmaceutical formulation according to claim 2, further comprising at least 0.5 μMoles of at least one of butyrate, methyl butyrate, beta hydroxy butyrate, beta hydroxy methylbutyrate, and salts, esters, and amides thereof.

8. The pharmaceutical formulation according to claim 2, comprising at least 1 mg of the at least one cannabinoid, and the at least 10 mg of the one additional active composition per unit dose or metered dose.

9. The pharmaceutical formulation according to claim 1, further comprising at least one of piperine, curcumin, capsaicin, resveratrol, and echinacea.

10. The pharmaceutical formulation according to claim 1, wherein the at least one cannabinoid comprises a full spectrum extract from a botanical source.

11. The pharmaceutical formulation according to claim 1, in a dental hygiene formulation and having antimicrobial effects after administration to oral mucosa.

12. The pharmaceutical formulation according to claim 1, in a pharmaceutically acceptable mist, vapor, or inhalable formulation.

13. A cannabinoid composition, comprising:
at least one cannabinoid selected from the group consisting of CBG, CBGA, CBGV, and CBGVA, or pharmaceutically salt, ester or amide thereof, in combination with at least three terpenes selected from the group consisting of limonene, linalool, pinene, humulene, β-caryophylline, bisabolene, terpinolene, and myrcene, the cannabinoid composition being substantially without cannabidiol and tetrahydrocannabinol; and
at least one additional component selected from the group consisting of an endocannabinoid, gamma amino butyric acid, and dehydroepiandrosterone.

14. The cannabinoid composition according to claim 13, further comprising at least one of the group consisting of curcumin, resveratrol, and piperine.

15. The cannabinoid composition according to claim 13, wherein the at least one additional component comprises an N-alkylamide endocannabinoid.

16. The cannabinoid formulation according to claim 13, further comprising at least one of butyrate, methyl butyrate, beta hydroxy butyrate, beta hydroxy methylbutyrate, and salts, esters, and amides thereof.

17. The cannabinoid composition according to claim 13, wherein the at least one cannabinoid comprises an extract of Cannabis plant comprising at least 5% by weight CBG and CBGA, with natural Cannabis terpenes and flavonoids comprising components having a boiling point of less than 125 C.

18. The cannabinoid composition according to claim 13, further comprising a spray, patch, cream, foam, gel, lotion, emollient, or ointment base in a topical dosage form.

19. A method of treating a human, comprising administering to a human a pharmaceutically acceptable formulation in oral, inhalant, enteral or transdermal form, comprising:
at least 4 mg of at least one cannabinoid selected from the group consisting of CBG, CBGA, CBGV, and CBGVA, or a pharmaceutically acceptable salt, ester or amide thereof, substantially without CBDA synthase products and TCHA synthase products of the at least one cannabinoid; and
at least one additional active composition in an amount of at least 1 mg, selected from the group consisting of an endocannabinoid, dextromethorphan, melatonin, 5-hydroxy tryptophan, serotonin, gamma amino butyric acid, dehydroepiandrosterone, and lipoic acid.

20. The method according to claim 19, wherein the wherein the pharmaceutically acceptable formulation is effective to at least one of:
alleviate a symptom of a coronavirus infection;
down regulate at least one of Transmembrane Serine Protease 2 (MPRSS2) expression and angiotensin-converting enzyme 2 (ACE2) expression; and
reduce levels of at least one of interleukin (IL)-6′, interleukin (IL)-8, interleukin (IL)-1β, TNF-α, IFN-γ, PPARγ, and pro-inflammatory cytokines.

21. The method according to claim 19, wherein the at least one cannabinoid is provided in a full spectrum or broad spectrum botanical extract comprising terpenes.
